(19) United States
(12) Patent Application Publication
(10) Pub. No.: US 2021/0395763 A1
(43) Pub. Date: Dec. 23, 2021

(54) IMPROVED PRODUCTION OF TERPENOIDS USING ENZYMES ANCHORED TO LIPID DROPLET SURFACE PROTEINS

(71) Applicant: Board of Trustees of Michigan State University, East Lansing, MI (US)

(72) Inventors: Björn Hamberger, Okemos, MI (US); Radin Sadre, Lansing, MI (US); Christoph Benning, East Lansing, MI (US); Jacob David Bibik, Lansing, MI (US)

(21) Appl. No.: 17/266,133

(22) PCT Filed: Aug. 8, 2019
(86) PCT No.: PCT/US2019/045730

§ 371 Date: Feb. 5, 2021

Related U.S. Application Data
(60) Provisional application No. 62/716,076, filed on Aug. 8, 2018.

(51) Int. Cl. C12N 15/82; C07K 14/415

(52) U.S. Cl. CPC C12N 15/8243; C07K 14/415; C12N 15/8217

(57) ABSTRACT

Methods and expression systems are described herein that are useful for production of terpenes and terpenoids.

This application claims benefit of priority to the filing date of U.S. Provisional Application Ser. No. 62/716,076, filed Aug. 8, 2018, the contents of which are specifically incorporated herein by reference in their entity.

### GOVERNMENT FUNDING

This invention was made with government support under DE-FC02-07ER64494 and under DE-SC0018409 awarded by the U.S. Department of Energy. The government has certain rights in the invention.

### BACKGROUND

Plant-derived terpenoids have a wide range of commercial and industrial uses. Examples of uses for terpenoids include specialty fuels, agrochemicals, fragrances, nutraceuticals and pharmaceuticals. However, currently available methods for petrochemical synthesis, extraction, and purification of terpenoids from the native plant sources have limited economic sustainability. For example, terpenoid biotechnology in photosynthetic tissues has remained challenging at least in part because any engineered pathways must compete for precursors with highly networked native pathways and their associated regulatory mechanisms.

### SUMMARY

Described herein are methods and expression systems that provide high yields of terpenoids and related compounds in cells having terpene synthases and other enzymes anchored to cellular lipid droplets. The methods enhance precursor flux through targeting of enzymes that can synthesize terpene precursors to native and non-native compartments to provide for increased terpenoid production. By producing lipophilic products (e.g., terpenoids) at the surface or within the lipid droplet, the anchored terpenoid biosynthetic enzymes facilitate sequestration of terpenoid products within the lipid droplets. The methods can efficiently produce industrially relevant terpenoids in photosynthetic tissues. For example, in some experiments yields of terpenoids of more than 300 micrograms terpenoids per gram fresh weight (0.03% fresh weight) can be obtained.

Fusion proteins are described herein including those that have a lipid droplet surface protein linked in-frame to one or more of the following fusion partners: a monoterpene synthase, diterpene synthase, sesquiterpene synthase, sesterterpene synthase, triterpene synthase, tetraterpene synthase, polyterpene synthase, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), or patchoulol synthase.

Expression systems are also described herein that include at least one expression vector having a first nucleic acid segment encoding a lipid droplet surface protein and at least one second nucleic acid segment encoding one or more of the following proteins: monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), or patchoulol synthase, wherein the first nucleic segment, the at least one second nucleic acid segment, or a combination thereof are operably linked to a heterologous promoter.

Methods are also described herein. For example, such a method can include: (a) incubating or cultivating one or more host cells, host tissues, host seeds, or host plants, each comprising expression system comprising at least one expression vector comprising a a first nucleic acid segment encoding a lipid droplet surface protein and at least one second nucleic acid segment encoding one or more of the following proteins: monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-Co A reductase (HMGR), rnevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), or patchoulol synthase, wherein the first nucleic segment, the at least one second nucleic acid segment, or a combination thereof are operably linked to a heterologous promoter; and (b) isolating lipids from the host cell, host tissue, host seed, or host plant.

For example, one of the methods described herein involves (a) incubating a population of host cells comprising an expression system that includes at least one expression cassette having a heterologous promoter operably linked to a nucleic acid segment encoding a fusion protein that includes lipid droplet surface protein (LDSP) linked in-frame to a monoterpene synthase, diterpene synthase, sesquiterpene synthase, sesterterpene synthase, triterpene synthase, tetraterpene synthase, or a polyterpene synthase; and (b) isolating lipids from the population of host cells. The method expression system can also include an expression cassette comprising a promoter operably linked to a nucleic acid encoding a WRI1 transcription factor. In addition, the expression system can include expression cassettes that can express geranylgeranyl diphosphate synthase (GGDPS) enzymes, 1-deoxy-D-xylulose 5-phosphate synthase (DXS), 3-hydroxy-3-methylglutaryl-CoA reductase (HMGR), farnesyl diphosphate synthase (FDPS), cytochromes P450, cytochrome P450 reductase, other terpenoid synthesizing enzymes, and combinations thereof.

In some cases, methods of producing terpenes and/or terpenoids can include, for example, (a) incubating a population of host cells comprising an expression system that includes: (i) an expression cassette (or expression vector) having a heterologous promoter operably linked to a nucleic acid segment encoding a geranylgeranyl diphosphate synthase (GGDPS) enzyme, (ii) an expression cassette (or expression vector) having a heterologous promoter that is active in plant plastids operably linked to a nucleic acid segment encoding a 1-deoxy-D-xylulose 5-phosphate synthase (DXS) enzyme, (iii) an expression cassette (or expression vector) having a heterologous promoter operably linked to a nucleic acid segment encoding an abietadiene synthase (ABS) enzyme, or (iv) a combination thereof; and (b) isolating lipids from the population of host cells. In addition, the expression system can include expression cassettes that can express 3-hydroxy-3-methylglutaryl-CoA reductase (HMGR), farnesyl diphosphate synthase (FDPS), cytochromes P450, cytochrome P450 reductase, other terpenoid synthesizing enzymes, and combinations thereof.

In some cases, methods of producing terpenes and/or terpenoids can include, for example, (a) incubating a population of host cells comprising an expression system that includes: (i) at least one expression cassette (or expression vector) having a heterologous promoter that operably linked to a nucleic acid segment encoding a 3-hydroxy-3-methylglutaryl-CoA reductase (HMGR) enzyme; (ii) at least one expression cassette (or expression vector) having a heterologous promoter that operably linked to a nucleic acid segment encoding a geranylgeranyl diphosphate synthase (GGDPS) enzyme; (iii) at least one expression cassette (or expression vector) having a heterologous promoter that operably linked to a nucleic acid segment encoding an abietadiene synthase (ABS) enzyme; or (iv) a combination thereof; and (b) isolating lipids from the population of host cells. In addition, the expression system can include expression cassettes that can express 1-deoxy-D-xylulose 5-phosphate synthase (DXS), 3 farnesyl diphosphate synthase (FDPS), cytochrome P450, cytochrome P450 reductase, other terpenoid synthesizing enzymes, and combinations thereof.

### DESCRIPTION OF THE FIGURES

FIG. 1A-1C illustrates engineered lipid droplet triacylglycerol (TAG) and patchoulol production in N. benthamiana leaves. FIG. 1A illustrates that triacylglycerol accumulation is increased through expression of Arabidopsis thaliana WRINKLED1 (producing AtWRI1(1-397) protein, which has a deletion of the C-terminal region) and enhanced through co-expression of a Nannochloropsis oceanica lipid droplet surface protein (NoLDSP). FIG. 1B illustrates patchoulol production that was engineered to occur in the cytosol in the absence and presence of AtWRI1(1-397) and NoLDSP. FIG. 1C illustrates patchoulol production that was engineered in the plastid in the absence and presence of AtWRI1(1-397) and NoLDSP. To enhance farnesyl diphosphate (FDP) availability for patchoulol production, a cytosolic, de-regulated 3-hydroxy-3-methylglutaryl-CoA reductase (HMGR) from Euphorbia lathyris (ElHMGR159-582, missing residues 1-158), a plastid-localized Plectranthus barbatus (Coleus forskohlii) 1-deoxy-D-xylulose 5-phosphate synthase (PbDXS, CfDXS, plastid), and an Arabidopsis thaliana farnesyl diphosphate synthase (AtFDPS) (localized in the cytosol or plastid) were expressed in transient assays. The different construct combinations are indicated below each bar (●, was included; −, was not included) and in the schematic diagram next to each graph. Average levels with standard deviation (SD) (n=6) and SD (n=8) for TAG and patchoulol, respectively, are shown. Statistically significant differences are indicated in the bars identified by the letters a-e (P<0.05). MEV pathway, mevalonic acid pathway; MEP pathway (2-C-methyl-D-erythritol 4-phosphate pathway), methylerythritol 4-phosphate pathway; LD, lipid droplet.

FIG. 2A-2F illustrate engineered diterpenoid production in Nicotiana benthamiana leaves. FIG. 2A illustrates production of diterpenoids (abietadiene and its isomers) in the plastids of N. benthamiana leaves, where Abies grandis abietadiene synthase (AgABS) was expressed with a variety of different enzymes. FIG. 2B illustrates production of diterpenoids (abietadiene and its isomers) in the plastids of N. benthamiana leaves when Abies grandis abietadiene synthase (AgABS) was expressed with a variety of different enzymes and/or a truncated WRINKLED (WRI1) and/or a Nannochloropsis oceanica lipid droplet surface protein (NoLDSP). N FIG. 2C illustrates production of diterpenoids (abietadiene and its isomers) in the cytosol of N. benthamiana leaves when cytosolic Abies grandis abietadiene synthase (AgABS) is expressed with a variety of enzymes and/or truncated WRINKLED (WRI1) and/or a Nannochloropsis oceanica lipid droplet surface protein (NoLDSP). To enhance GGDP availability for diterpenoid production in FIGS. 2A-2C, truncated 3-hydroxy-3-methylglutaryl-CoA reductase (HMGR) from Euphorbia lathyris (ElHMGR¹⁵⁹⁻⁵⁸², expressed in the cytosol), 1-deoxy-D-xylulose 5-phosphate synthase from Plectranthus barbatus (also called Coleus forskohlii) (PbDXS; expressed in plastids), and distinct geranylgeranyl diphosphate synthases (GGDPSs) (cytosol or plastid) were included in transient assays. The protein combinations are indicated below each bar (black circle, was included; minus, was not included) and in the scheme next to each graph. The production of diterpenoids was engineered in the plastid (FIG. 2A-2B) and in the cytosol (FIG. 2C) in the absence and presence of AtWRI1¹⁻³⁹⁷ and NoLDSP. Average diterpenoid levels with SD (n=4), SD (n=8) and SD (n=6) are shown in FIGS. 2A, 2B, and 2C, respectively. Statistically significant differences are indicated by letters a-f (P<0.05). MEV pathway, mevalonic acid pathway; MEP pathway, methylerythritol 4-phosphate pathway; LD, lipid droplet. FIG. 2D-2E illustrate that diterpenoids were sequestered in isolated lipid droplet fractions. FIG. 2D shows floating lipid droplet layers after gradient centrifugation of isolated lipid droplet fractions from N. benthamiana leaves expressing either plastid:AgABS alone or in combination with AtWRI1(1-397) and NoLDSP (without and without YFP-tag). FIG. 2E graphically illustrates diterpenoid content in the isolated lipid droplet fractions with the bars representing average values and SD for three biological replicates (n=3). Statistically significant differences are indicated by the letters a-c (P<0.05). FIG. 2F illustrates that expression of (YFP)-tagged Nannochloropsis oceanica lipid droplet surface protein (LDSP), LDSP-fused ABS⁸⁵⁻⁸⁶⁸ protein, LDSP-fused CYP720B4³⁰⁻⁴⁸³ protein, and LDSP-fused CaCPR⁷⁰⁻⁷⁰⁸ protein promotes clustering of small lipid droplets in N. benthamiana leaves engineered for triacylglycerol accumulation. In the LDSP-fused ABS⁸⁵⁻⁸⁶⁸ protein (LD:AgABS⁸⁵⁻⁸⁶⁸), the LDSP replaces the transit peptide (residues 1-84) of the ABS enzyme to provide a cytosolic version of the ABS enzyme. The LDSP-fused CYP720B4³⁰⁻⁴⁸³ protein (LD:PsCYP720B4³⁰⁻⁴⁸³) is the cytochrome P450 (CYP720B4) from Picea sitchensis without residues 1-29. The CaCPR⁷⁰⁻⁷⁰⁸ is cytochrome P450 reductase (CaCPR) from Camptotheca acuminata without residues 1-69. Confocal laser scanning microscopy merged images are shown for N. benthamiana leaves (yellow, YFP signal; red, chlorophyll fluorescence; scale bar 2 μm).

FIG. 3A-3B illustrate triacylglycerol (TAG) yield in N. benthamiana leaves engineered for the co-production of terpenoids and lipid droplets. FIG. 3A illustrates the impact of engineering patchoulol production on the amounts of lipids (TAG) in N. benthamiana leaves that express a P. cablin patchoulol synthase in the cytosol or plastids (plastid:PcPAS) in addition to other enzymes. FIG. 3B illustrates the impact of engineering diterpenoid production in either plastids or in the cytosol on the amounts of lipids (TAG) produced in N. benthamiana leaves that express a variety of enzymes in addition to Abies grandis abietadiene synthase (AgABS), which can synthesize diterpenes. TAG accumulation was initiated through ectopic expression of WRINKLED1 (AtWRI1¹⁻³⁹⁷) and further enhanced through co-expression of NoLDSP. The different construct combinations are indicated below each bar (●, was included; −, was not included). Average TAG levels with SD (n=6) are shown. Statistically significant differences are indicated by a-d (P<0.05).

FIG. 4 illustrates localization of heterologously-expressed yellow fluorescent protein (YFP)-tagged fusion proteins including YFP-tagged Nannochloropsis oceanica lipid droplet surface protein (LDSP), YFP-tagged LDSP-fused AgABS⁸⁵⁻⁸⁶⁸ (LD:AgABS⁸⁵⁻⁸⁵⁸, missing residues 1-84), YFP-tagged LDSP-fused CYP720B4 protein (LD:PsCYP720B4(30-483) missing residues 1-29), and YFP-tagged LDSP-fused CPR protein (LD:CaCPR(70-708), missing residues 1-69)). The AgABS(85-868) protein was truncated to remove the plastid targeting sequence while the PsCYP720B4(30-483) and CaCPR(70-708) proteins were truncated to remove the membrane anchoring domain. Note that AtWRI1(1-397) was co-produced and leaf samples were stained with Nile red to visualize neutral lipids in lipid droplets. This experiment was replicated twice. Confocal laser scanning microscopy images are shown (the lighter signal is yellow produced by YFP fluorescence; the darker signal is red produced by chlorophyll fluorescence; scale bar 10 μm). The expressed YFP-proteins are indicated in each line. LD, lipid droplet. Channels: YFP yellow fluorescent protein (scale bar 20 μm). NR Nile red (scale bar 20 μm), YFP NR, enlarged merge YFP and NR (scale bar 5 μm).

FIG. 5A-5D illustrate lipid droplets are useful engineering platforms for the production of functionalized diterpenoids. FIG. 5A graphically illustrates diterpenoid and diterpenoid acid production when the following terpenoid biosynthesis enzymes were targeted to lipid droplets as fusion proteins with Nannochloropsis oceanic lipid droplet surface protein (LD): LD:PsCYP720B44(30-483) and LD:CaCPR(70-708), and different combinations with other enzymes were also expressed as indicated below each bar (black circle, was included; minus, was not included). FIG. 5B graphically illustrates diterpenoid and diterpenoid acid production when the following terpenoid biosynthesis enzymes were targeted to lipid droplets as fusion proteins with Nannochloropsis oceanica lipid droplet surface protein (LD): LD:PsCYP720B44(30-483) and LD:CaCPR(70-708), and different combinations with other enzymes were also expressed as indicated below each bar (black circle, was included; minus, was not included). FIG. 5C graphically illustrates diterpenoid and diterpenoid acid production when the following terpenoid biosynthesis enzymes were targeted to lipid droplets as fusion proteins with Nannochloropsis oceanica lipid droplet surface protein (LD): LD:AgABS(85-868), LaPsCYP720B44(30-483), and LD:CaCPR(70-708), and different combinations with other enzymes were also expressed as indicated below each bar (black circle, was included; minus, was not included). As shown, production of native or modified AgABS led to accumulation of diterpenoids, and when native or modified PsCYP720B4 was co-produced, conversion of diterpenoids to diterpenoid acids was also observed. For FIGS. 5A-5C, data were analyzed by Shapiro-Wilk, Brown-Forsythe ANOVA (diterpenoids P<0.0184, P<0.0001, P<0.0001; diterpenoid acids P<0.0001, P<0.0001, P<0.0001) and Welch ANOVA (diterpenoids P<0.0509, P 0.0002, P<0.0001; diterpenoid acids P<0.0001, P<0.0001, P 0.0002) followed by t-tests (unpaired, two-tailed, Welch correction). Results are presented as individual biological replicates and bars representing average levels with SD (N indicated below each bar). Statistically significant differences are indicated by a-d based on t-tests (P<0.05). The experiments relating to FIGS. 5A-5C were replicated twice. FIG. 5D schematically illustrates the conversion of abietadiene to abietic acid when LD:AgABS(85-868) (NoLDSP-AgABS), LD:PsCYP720B44(30-483) (NoLDSP-PsCYP) and LD:CaCPR(70-708) (NoLDSP-CaCPR) were produced. LD, lipid droplet; e−, electron from NADPH.

FIG. 6 illustrates LC/MS analysis of extracts from N. benthamiana leaves producing AtWRI1(1-397) with NoLDSP, ElHMGR(159-582), cytosol:MtGGDPS, LD:AgABS(85-868), and ER:PsCYP720B4. Extracted ion chromatograms m/z 301.217 are shown in acquisition function 1 (0 V) and function 2 (20-80 V). Compounds 1-4 were subjected to MS/MS analysis. The elution order and MS/MS data were consistent with compound 1-3 and compound 4 being formate adducts of tetrahexosyl diterpenoid acid isomers and trihexosyl diterpenoid acid, respectively (see FIGS. 7-8).

FIG. 7 illustrates LC/MS/MS analysis of tetrahexosyl diterpenoid acid isomers in N. benthamiana leaf extracts where the leaves transiently expressed AtWRI1¹⁻³⁹⁷ with NoLDSP, ElHMGR(159-582), cytosol:MtGGDPS, LD:AgABS(85-868), and ER:PcCYP720B4. Accurate masses and MS/MS spectra of compounds 1-3 are consistent with formate adducts of tetrahexosyl diterpenoid acid isomers [M+formate]⁻ m/z 995.4 (fragments: [M−formate]⁻ m/z 949.4, [M−formate-partial loss of dihexosyl]⁻ m/z 667.3 and [M−formate-tetrahexosyl]⁻ m/z 301.2).

FIG. 8 illustrates LC/MS/MS analysis of a trihexosyl diterpenoid acid (compound 4) in N. benthamiana leaf extracts where the leaves transiently expressed AtWRI1¹⁻³⁹⁷ with NoLDSP, ElHMGR(159-582), cytosol:MtGGDPS, LD:AgABS(85-868), and ER:PcCYP720B4. Elemental composition and MS/MS spectrum of compound 4 are consistent with a formate adduct of trihexosyl diterpenoid acid [M+formate]⁻ m/z 833.3 (fragments: [M−formate]⁻ m/z 787.4, [M−formate-dihexosyl]⁻ m/z 463.3 and [M−formate-trihexosyl]⁻ m/z 301.2).

FIG. 9 is a schematic diagram illustrating lipid droplet scaffolding of squalene biosynthesis enzymes farnesyl diphosphate synthase (FPPS) and squalene synthase (SQS), the final two steps of squalene biosynthesis. Lipid droplet formation is induced by expression of AtWRI1(1-397) and by expression of variations of NoLDSP alone or as LDSP-fusions with either FPPS or SQS.

FIG. 10 graphically illustrates casbene levels generated during a screen of 1-deoxy-D-xylulose 5-phosphate synthase (DXS) and DXS alternatives that were co-expressed with Coleus forskohlii GGPPS (CfGGPPS) and a casbene synthase (CasS). Vertical bars represent upper and lower value limits. The interquantile range between the first and third quantile represented by the box. Middle horizontal bar represents the median value and red cross represents the average value.

FIG. 11 graphically illustrates results of screening squalene synthases for optimal activity. The graph shows squalene yields as determined by GC-FID for various squalene synthases, where the relative yields are reported as the ratio of squalene to the internal standard, n-hexacosane. As illustrated, a Mortierella alpina squalene synthase with 17 amino acids truncated from the C-terminus had the highest squalene synthase activity.

FIG. 12 graphically illustrates results of screening of farnesyl diphosphate synthase (FPPS) candidates to optimize squalene synthesis. The graph shows squalene yields as determined by GC-FID for various farnesyl diphosphate synthases, where the relative yields are reported as the ratio of squalene to an internal standard.

FIG. 13A-13B graphically illustrates that linkage to lipid droplet surface protein to enzymes involved in squalene biosynthesis can improve squalene accumulation. FIG. 13A shows that expression of squalene synthase fused to lipid droplet surface protein can improve squalene synthesis compared to when squalene synthase is in soluble (non-fused form. FIG. 13B shows that fusion of squalene synthase or FPPS can improve squalene accumulation.

FIG. 14 illustrates improved capacity of the lipid droplet scaffolding platform by providing contributions from the MEP pathway and the plastidial squalene biosynthesis pathway.

FIG. 15 illustrates that fusions of lipid droplet surface protein Agrobacterium-mediated transient expression performed on leaves of poplar NM6 to expand LD scaffolding to new species. Top row: images of wild type, not infiltrated poplar leaves. Middle row: images of leaf transiently expressing eYFP-NoLDSP fusion gene from pEAQ vector. Bottom row: images of leaf transiently expressing AtWRI1¹⁻³⁹⁷ linked to eYFP-NoLDSP by the “self-cleaving” LP4/2A hybrid linker, which is cleaved during translation to form the two separate protein products. Punctae shown in bottom row images indicate formation of lipid droplets in leaves of poplar NM6.

### DETAILED DESCRIPTION

Described herein are methods for high-yield synthesis of lipid compounds, including terpenes, terpenoids, steroids and biofuels (oils) in engineered lipid droplet-accumulating plant cells. For example, the systems and methods described herein can facilitate production of products such as terpenoids, carotenoids, withanolides, ubiquinones, dolichols, sterols, and biofuels. To do this, one or more of the enzymes that synthesize such products can be fused to a lipid droplet surface protein (LDSP), or a portion thereof. Such a LDSP-synthetic enzyme fusion protein is anchored on lipid droplet organelles within host cells. As the anchored synthetic enzymes make their hydrophobic, and sometimes volatile, products, these products accumulate in the lipid droplets. Hence, hydrophobic and volatile products are sequestered in a hydrophobic environment where they do not injure the cell. Instead, the hydrophobic and volatile products remain solubilized within the lipid droplets (rather than being lost by vaporization). In addition, the concentration of hydrophobic and volatile products within the lipid droplets facilitates their separation and purification away from other cellular materials. For example, lipids useful as biofuels (e.g. squalene and related compounds) can be made in commercially relevant plant species where the lipids are concentrated within lipid droplets that can readily be isolated from plant materials.

To optimize such production, the availability of precursors for such terpenoid products can also be enhanced by engineering the cells to also express de-regulated, robust enzymes from the mevalonic acid (MEV) pathway or the methylerythritol 4-phosphate pathway (MEP). The enzymes can be expressed or transported into the same intracellular compartments or into intracellular compartments that optimize terpenoid synthesis.

### Lipid Droplet Surface Protein (LDSP)

As illustrated herein, fusion of synthetic enzymes with lipid droplet surface protein (LDSP), or a portion thereof, can increase manufacture of various terpenoid products. Hence, the LDSP or a portion thereof can be linked in frame with a fusion partner such as a terpene synthase. The LDSP can localize and stabilize fusion partner enzymes within or at the surface of lipid droplets. The lipid droplets can absorb and concentrate/sequester lipophilic products such as terpenoids.

Cytosolic lipid droplets are dynamic organelles typically found in seeds as reservoirs for physiological energy and carbon in form of triacylglycerol (oil) to fuel germination. They are derived from the endoplasmic reticulum (ER) where newly synthesized triacylglycerol accumulates in lens-like structures between the leaflets of the membrane bilayer. After growing in size, the lipid droplets can bud off from the outer membrane of the endoplasmic reticulum.

A mature lipid droplet is typically composed of a hydrophobic core of triacylglycerol surrounded by a phospholipid monolayer and coated with lipid droplet associated proteins such as oleosins involved in the biogenesis and function of the organelle. These oleosins contain surface-oriented amphipathic N- and C-termini essential to efficiently emulsify lipids and a conserved hydrophobic central domain anchoring the oleosins onto the surface of lipid droplets. One type of lipid droplet associated protein is a lipid droplet surface protein.

An amino acid sequence for the full-length Nannochloropsis oceanica lipid droplet surface protein (NoLDSP, JQ268559.1) sequence is shown below as SEQ ID NO:1.

| | |
|---|---|
| 1 | MAGPIMTSAP SATTPTGKTM PFKQPFKTVA TLSAKTGNIT |
| 41 | KPIDPAISKT IDFVYNGYST VKTKVDKAPK VNPYLLIAGG |
| 81 | LVLSCIISMC LLVPAVIFFP VTIFLGVATS FALIALAPVA |
| 121 | FVFGWILISS APIQDKVVVP ALDKVLANKK VAKFLLKE |

Such an LDSP polypeptide can be fused to enzymes such as those involved in the synthesis of terpenes and terpenoids. When a LDSP polypeptide is fused to another protein or enzyme, (LD) or LD is used with the protein or enzyme name.

A nucleic acid sequence for the full-length N. oceanica lipid droplet surface protein (NoLDSP, JQ268559.1) sequence is shown below as SEQ ID NO:2.

| | |
|---|---|
| 1 | TTTAAAGGAA AAACAACAGA CCACCACCAA TCTCAGCCCG |
| 41 | CATCAACAAT GGCCGGCCCC ATCATGACCT CTGCGCCCTC |
| 81 | CGCGACCACG CCCACGGGCA AGACAATGCC GTTCAAGCAG |
| 121 | CCTTTCAAGA CTGTGGCCAC GCTGTCCGCC AAGACTGGCA |
| 161 | ACATTACCAA GCCCATCGAC CCTGCCATCT CCAAGACCAT |
| 201 | TGACTTCGTC TACAATGGTT ACTCGACGGT CAAGACCAAG |
| 241 | GTTGACAAGG CCCCTAAGGT AAACCCCTAC CTGCTCATTG |
| 281 | CCGGCGGCCT CGTCCTCTCG TGCATCATCT CCATGTGCCT |
| 321 | GCTCGTCCCG GCCGTGATCT TCTTCCCCGT CACCATCTTC |
| 361 | CTGGGTGTCG CTACGTCGTT TGCGCTCATT GCATTGGCCC |
| 401 | CCGTGGCTTT TGTGTTCGGG TGGATCCTGA TCTCCTCTGC |
| 441 | TCCGATCCAG GATAAGGTGG TGGTGCCCGC CTTGGACAAG |
| 481 | GTGCTGGCCA ATAAGAAGGT GGCGAAGTTC CTCCTCAAGG |
| 521 | AGTAAGAAAG ATCCAAGAGA GACGAGTAGA GATTTTTTTT |
| 561 | T |

Expression cassettes and expression vectors can have a nucleic acid segment that includes a segment with SEQ ID NO:2 and/or a segment encoding an LDSP protein with SEQ ID NO:1.

The LDSP can have one or more deletions, insertions, replacements, or substitutions without loss of LDSP activities. Such LDSP activities include localizing and stabilizing enzymes within or at the surface of lipid droplets. The LDSP can have, for example, at least 60%, or at least 70%, or at least 80%, or at least 90%, or at least 95%, or at least 97%, or at least 98%, or at least 99% sequence identity to a sequence described herein.

The systems and methods described herein are useful for synthesizing terpenes, terpenoids, and compounds made from terpenes and terpenoids. A variety of enzymes useful for making such compounds can be used in native or modified forms and are described hereinbelow. Many of the enzymes are part of the mevalonate pathway or the mevalonic acid pathway

### Mevalonate (MEV) Pathway

The mevalonate pathway, also known as the isoprenoid pathway or HMG-CoA reductase pathway, is an essential metabolic pathway present in eukaryotes, archaea, and some bacteria. The pathway produces the two five-carbon building blocks for terpenes (isoprenoids): isopentenyl pyrophosphate (IPP) and dimethylallyl pyrophosphate (DMAPP).

Isoprenoids are a diverse class of over 30,000 biomolecules such as cholesterol, heme, vitamin K, coenzyme Q10, steroid hormones and molecules used in processes as diverse as protein prenylation, cell membrane maintenance, the synthesis of hormones, protein anchoring and N-glycosylation.

The mevalonate pathway is shown below, beginning with acetyl-CoA and ending with the production of IPP and DMAPP.

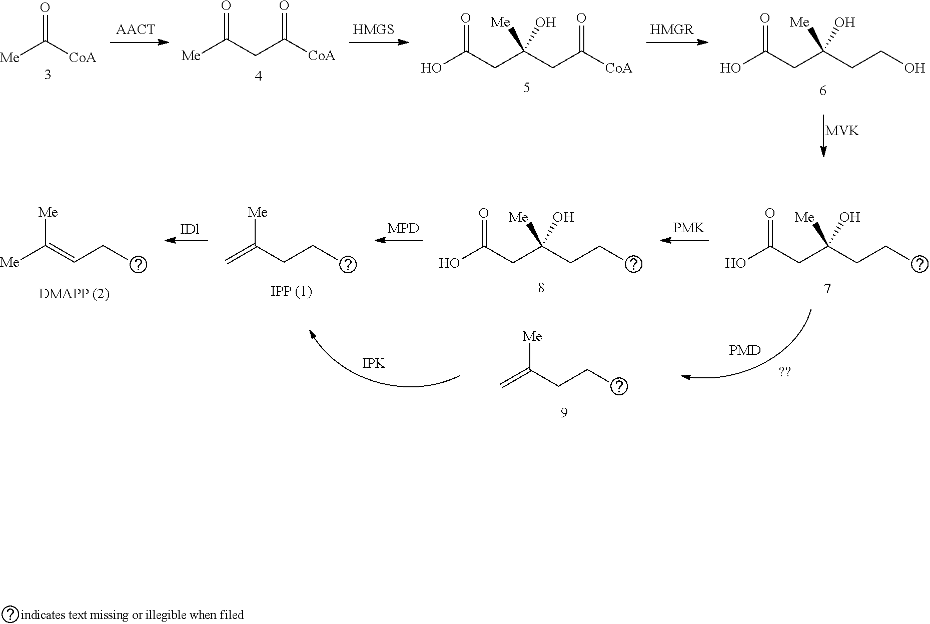

MEV pathway starts with the condensation of two molecules of acetyl-CoA (3) by acetyl-coenzyme A acetyltransferase to form acetoacetyl-CoA (4). Further condensation with a third molecule of acetyl-CoA by HMG-CoA synthase produces 3-hydroxy-3-methyl-glutaryl-CoA (HMG-CoA, 5), which is then reduced by HMG-CoA reductase (HMGR) to give mevalonic acid (6). Following two consecutive phosphorylation steps catalyzed by mevalonic acid kinase (MVK) and phosphomevalonate kinase (PMK), the resulting mevalonate-5-diphosphate (8) is converted to isopentenyl pyrophosphate (1) in an ATP-coupled decarboxylation reaction catalyzed by mevalonate-5-diphosphate decarboxylase (MPD). While the plastidic MEP pathway (described below) results in the synthesis of both IPP and DMAPP, the cytosol-localized mevalonate pathway produces only IPP. IPP can be isomerized to DMAPP by isopentenyl diphosphate isomerase (or IPP:DMAPP) isomerase (IDI).

Grochowski et al. (J. Bacteriol. 188:3192-3198 (2006)) identified an enzyme from Methanocaldococcus jannaschii capable of phosphorylating isopentenyl phosphate (9) to isopentenyl pyrophosphate (1). A modified MEV pathway was thus proposed in which mevalonate-5-phosphate (7) is decarboxylated to 9 and then phosphorylated by isopentenyl phosphate kinase (IPK) to form isopentenyl pyrophosphate (1). However, the proposed phosphomevalonate decarboxylase (PMD, 7→9 conversion) has yet to be identified.

While the plastidic MEP pathway (described below) results in the synthesis of both IPP and DMAPP, the cytosol-localized mevalonate pathway produces only IPP. IPP can be isomerized to DMAPP by isopentenyl diphosphate isomerase (IDI), a divalent metal ion-requiring enzyme found in all living organisms.

### Methylerythritol Phosphate (MEP) Pathway

For decades, the mevalonic acid pathway was thought to be the only IPP and DMAPP biosynthetic pathway. However, the incompatibility of many isotopic labeling results relating to the MEV pathway had been puzzling. Efforts to resolve such discrepancies eventually led to the discovery of the 2-C-methyl-D-erythritol 4-phosphate (MEP) pathway, also known as the 1-deoxy-D-xylulose 5-phosphate (DXP), or non-mevalonate pathway.

In plants, the MEP pathway is active in plastids. Reactions proceeding by the MEP pathway are shown below.

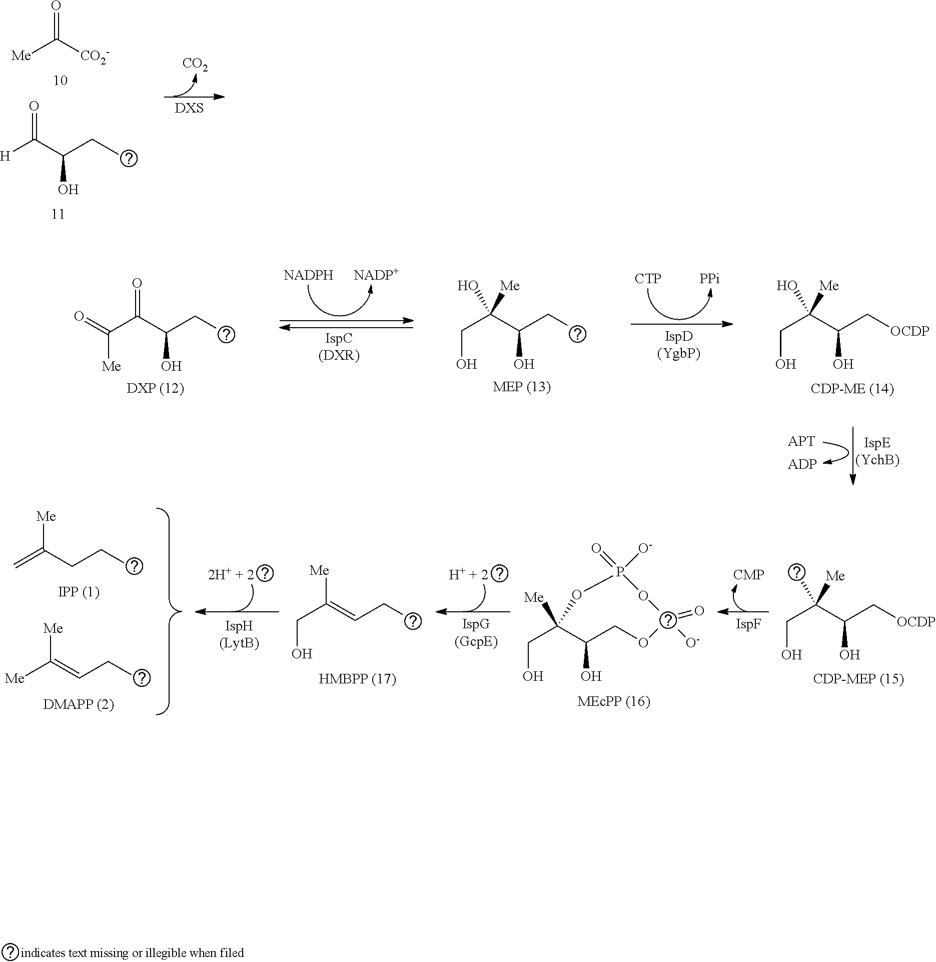

The MEP pathway is initiated with a thiamin diphosphate-dependent condensation between D-glyceraldehyde, 3-phosphate (11) and pyruvate (10) by 1-deoxy-D-xylulose 5-phosphate synthase (DXS) to produce 1-deoxy-D-xylulose 5-phosphate (DXP, 12), which is then reductively isomerized to methylerythritol phosphate (13) by DXP reducto-isomerase (DXR/IspC). Subsequent coupling between methylerythritol phosphate (13) and cytidine 5′-triphosphate (CTP) is catalyzed by CDP-ME synthetase (IspD) and produces methylerythritol cytidyl diphosphate (CDP-ME, 14). An ATP-dependent enzyme (IspE) phosphorylates the C2 hydroxyl group of 14, and the resulting 4-diphosphocytidyl-2-C-methyl-D-erythritol-2-phosphate (CDP-MEP, 15) is cyclized by 2-C-methyl-d-erythritol 2,4-cyclodiphosphate synthase (IspF) to 2-C-methyl-D-erythritol-2,4-cyclodiphosphate (MEcPP, 16), 1-Hydroxy-2-methyl-2-(E)-butenyl 4-diphosphate synthase (IspG) catalyzes the ring-opening of the cyclic pyrophosphate and the C₃-reductive dehydration of MEcPP (16) to form 4-hydroxy-3-methyl-butenyl 1-diphosphate (HMBPP, 17). The final step of the MEP pathway is catalyzed by 4-hydroxy-3-methylbut-2-enyl diphosphate reductase (IspH) and converts HMBPP (17) to both IPP (1) and DMAPP (2). Thus, unlike the MEV pathway, IPP:DMAPP isomerase (IDI) is not essential in many MEP pathway utilizing organisms. Any of the enzymes of the MEV and MEP pathways can be employed in the systems and methods described herein.

### Enzymes

A variety of enzymes can be used to make terpenoids. In some cases, fusion of those enzymes to lipid droplet surface proteins can increase lipid and terpenoid production with host cells and host plants. For example, sequestration of a desired product in lipid droplets can increase production of a product and facilitate isolation of that product. Such sequestration of a product be optimized by fusing or linking enzymes in the final steps of synthesizing the product to a lipid droplet surface protein. Enzymes that provide precursors for the final product may not, in some cases, need to be fused or linked to a lipid droplet surface protein. For example, if the desired product is patchoulol or squalene, fusion of patchoulol synthase or squalene synthase, respectively, to a lipid droplet surface protein can help sequester the patchoulol or squalene within lipid droplets. Use of lipid droplets to collect desirable products can also prevent modification of the products into undesired side products, because the lipid droplets can shield the products from modification by other cellular enzymes.

As described above, in plants the C5-building blocks for terpenoids, dimethylallyl diphosphate (DMADP) and isopentenyl diphosphate (IDP), are synthesized by two compartmentalized pathways. The mevalonic acid pathway converts acetyl-CoA by enzyme activities located in the cytosol, endoplasmic reticulum and peroxisomes, providing precursors for a wide range of terpenoids with diverse functions such as in growth and development, defense and protein prenylation. The enzyme 3-hydroxy-3-methylglutaryl-CoA reductase (HMGR) catalyzes the rate-limiting step in the mevalonic acid pathway. As illustrated herein, truncation of the catalytic domain of HMGR by N-terminal truncation can improve the flux of precursors into terpenoid biosynthesis.

In the plastid, the 2-C-methyl-D-erythritol 4-phosphate (MEP) pathway uses pyruvate and D-glyceraldehyde 3-phosphate to provide precursors for the biosynthesis of terpenoids related to development, photosynthesis and defense against biotic and abiotic stresses. The enzyme 1-deoxy-D-xylulose 5-phosphate synthase (DXS) is rate-limiting in the MEP pathway. Constitutive overproduction of DXS can enhance terpenoid production in some plant species tested. For example, when DXS is expressed in plastids, DXS overexpression can improve production of sesquiterpenes via a sesquiterpene-synthesizing enzyme, especially when farnesyl diphosphate synthase (FDPS) is also produced in plastids, for to provide farnesyl pyrophosphate building blocks.

Head-to-tail condensation of DMADP and IDP affords linear isoprenyl diphosphates, such as farnesyl diphosphate (FDP, C15) or geranylgeranyl diphosphate (GGDP, C20) catalyzed by farnesyl diphosphate synthase (FDPS) and geranylgeranyl diphosphate synthase (GGDPS), respectively. In Nicotiana benthamiana, both DXS and GGDPS were required to enhance terpenoid synthesis. Cytosolic sesquiterpene synthases and plastidial diterpene synthases convert FDPS and GGDPS, respectively, into typically cyclic terpenoid scaffolds, contributing to the enormous structural diversity among terpenoids in the plant kingdom. Such terpenoid scaffolds often undergo further stereo- and regio-selective functionalization catalyzed by ER membrane-bound monooxygenases, such as cytochromes P450 (CYPs), which utilize electrons provided by co-localized NADPH-dependent cytochrome P450 reductases (CPRs).

Terpenoid biotechnology in photosynthetic tissues has remained challenging because the engineered pathways must compete for precursors with highly networked native pathways (and their associated regulatory mechanisms).

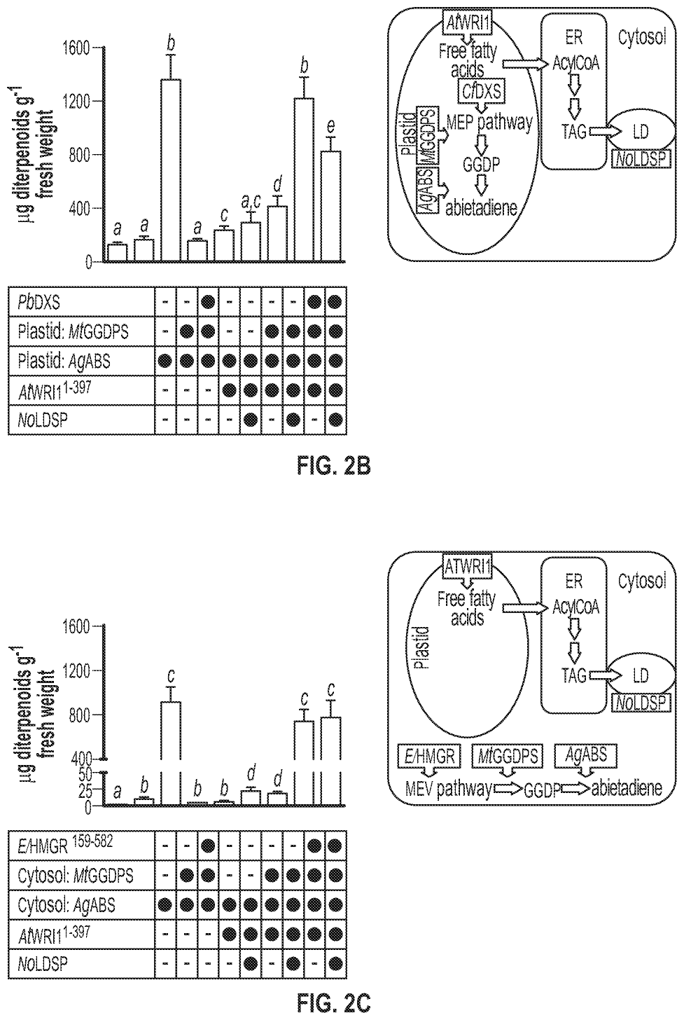

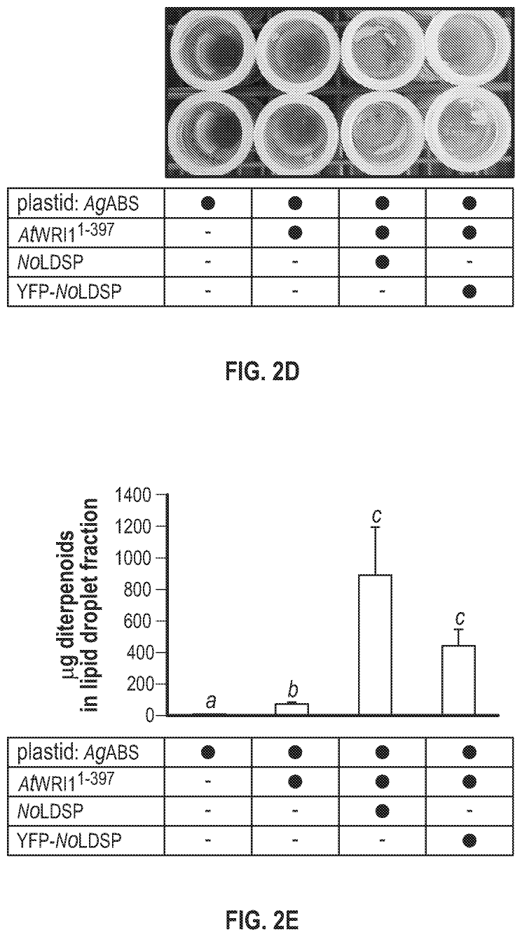

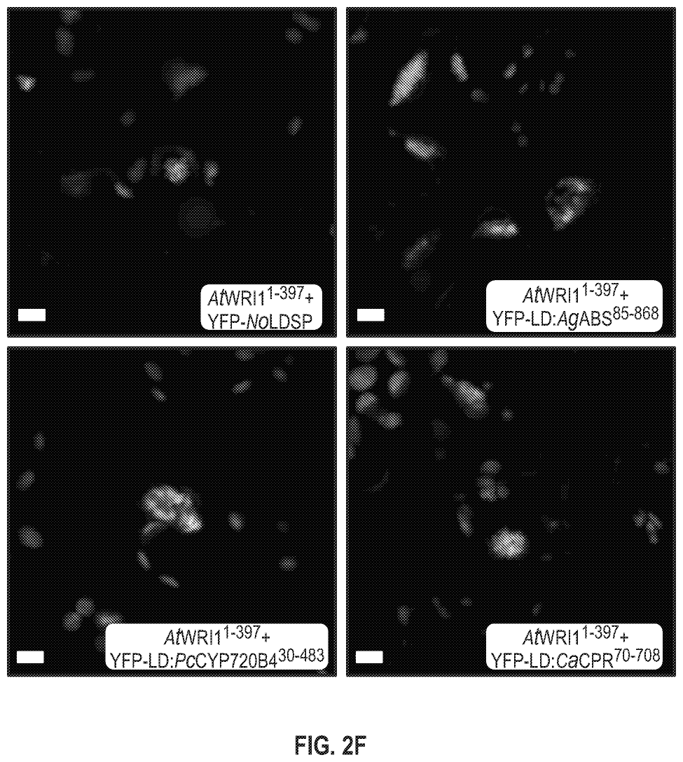

Examples of enzymes that can produce useful precursors and/or facilitate terpene synthesis include Plectranthus barbatus (Coleus forskohlii) 1-deoxy-D-xylulose 5-phosphate synthase (PbDXS), 3-hydroxy-3-methylglutaryl-CoA reductase (HMGR) from Euphorbia lathyris (ElHMGR or a truncated ElHMGR159-582), geranylgeranyl diphosphate synthase (GGDPS), farnesyl diphosphate synthase (FDPS), or combinations thereof. As illustrated herein a type I enzyme such as Methanothermobacter thermautotrophicus (MtGGDPS, type I) can be a robust alternative to type II GGDPS enzymes that can increase precursor availability for diterpenoid synthesis and circumvent potential negative feedbacks observed as illustrated herein (see, FIGS. 2A-2B). The methods and expression systems described herein are useful for manufacture of terpenes, diterpenes, sesquiterpenes, triterpenoids, and combinations thereof. For examples, the methods and expression systems described herein are also useful for manufacture of FDPS-dependent sesquiterpenoids, triterpenoid or combinations thereof.

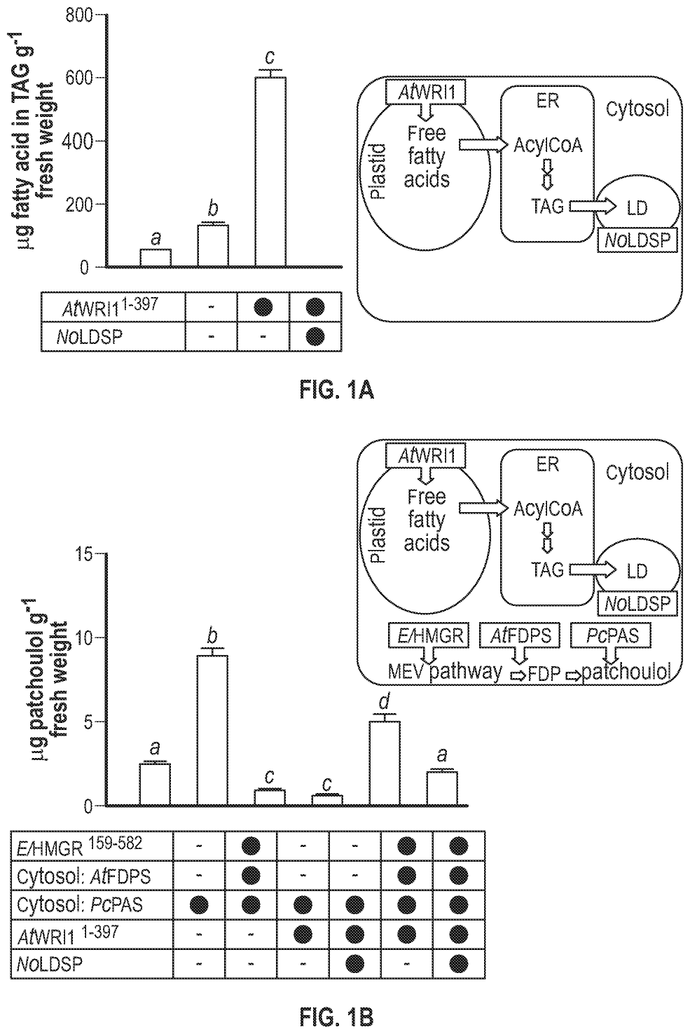

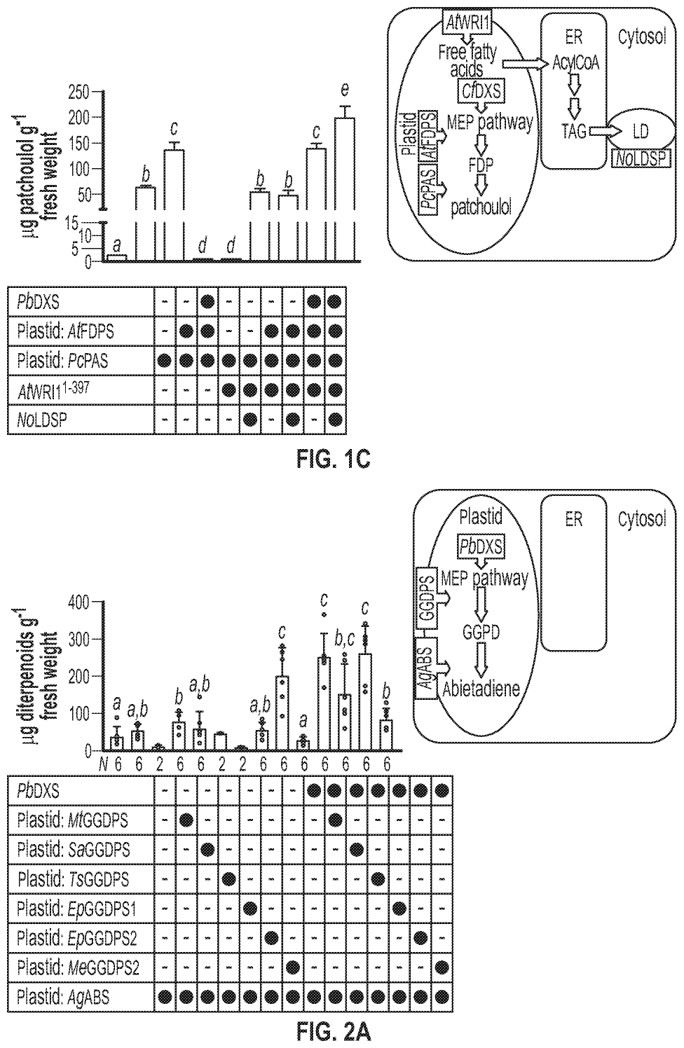

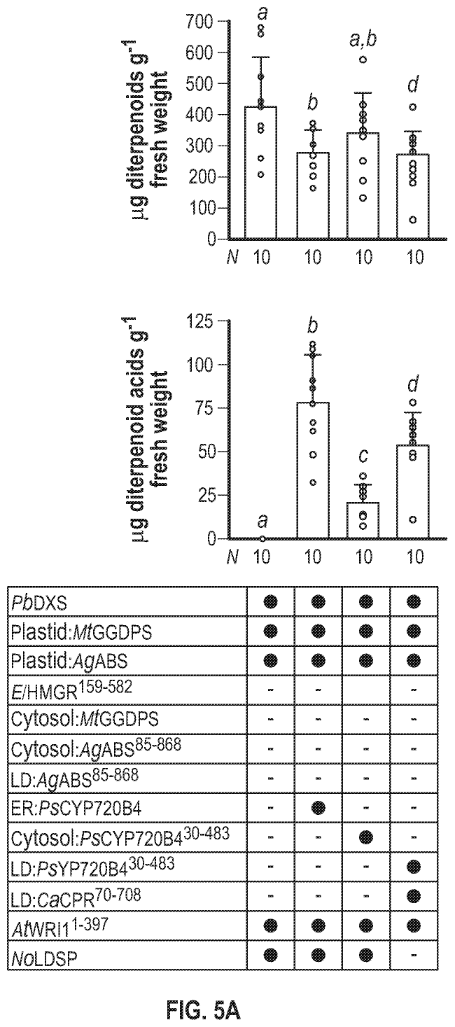

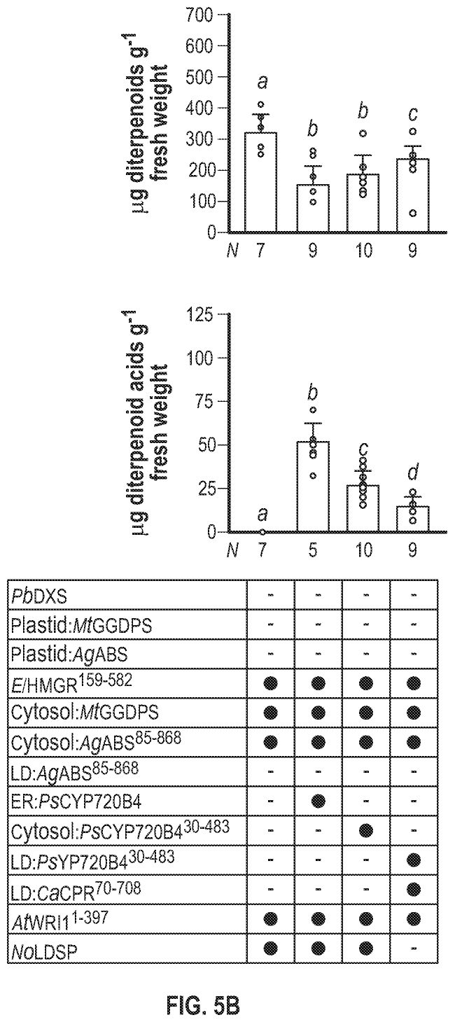

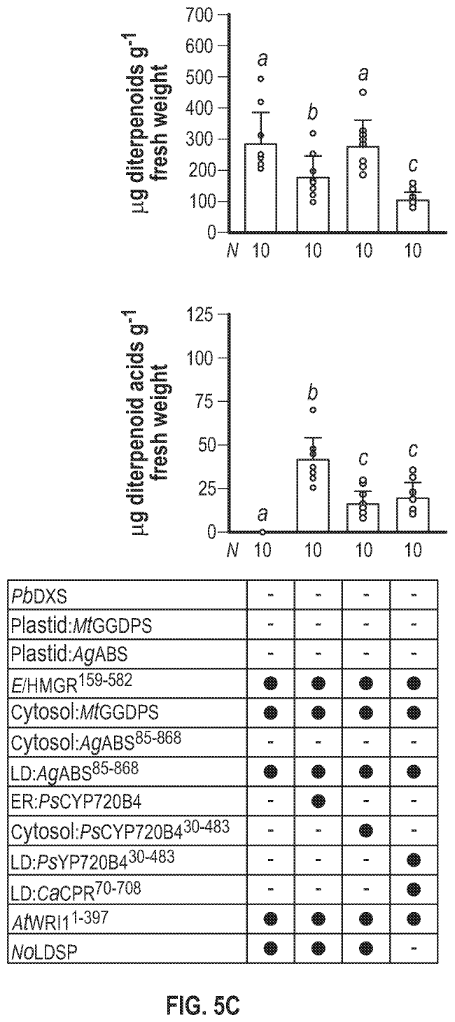

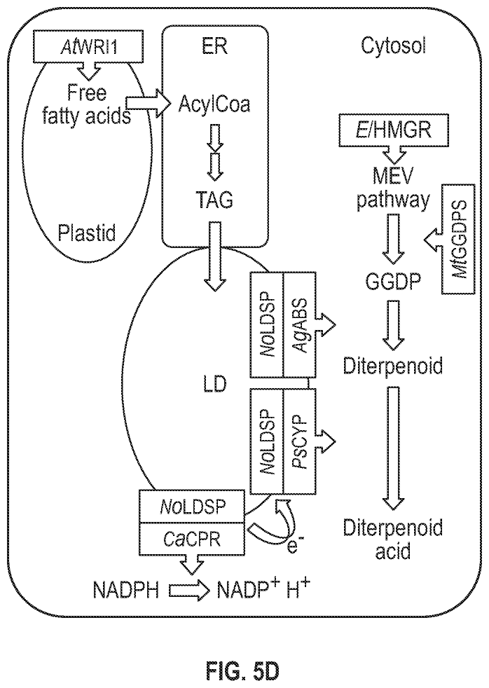

Highest accumulations of an example target sesquiterpenoid was achieved through compartmentation of the biosynthetic pathway in the plastid instead of the cytosol (FIG. 1C). Diterpenoid pathways were engineered in the plastid (PbDXS+plastid:MtGGDPS+ plastid:AgABS) or in the cytosol/lipid droplets (ElHMGR159-582+cytosol:MtGGDPS+ LD:AgABS85-868) with equal success yielding a high content of target diterpenoids in vegetative tissue and demonstrating the practicability of the chosen approaches (FIGS. 2 and 5).

Sequences of some of the enzymes useful for making precursors for terpene/terpenoid synthesis and other useful products are provided herein.

For example, a 1-deoxy-D-xylulose-5-phosphate synthase (EC 2.2.1.7; DXS) can facilitate synthesis of precursors for a variety of terpenes. Such a DXS enzyme can catalyze the following reaction:

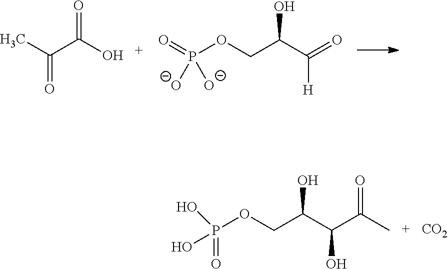

pyruvate+D-glyceraldehyde 3-phosphate [symbol] 1-deoxy-D-xylulose 5-phosphate+CO₂

One example of a useful DXS enzyme is a Plectranthus barbatus (Coleus forskohlii) 1-deoxy-D-xylulose 5-phosphate synthase (PbDXS; accession MH363713), which can have the following amino acid sequence (SEQ ID NO:3),

MASCGAIGSS FLPLLHSDES SFLSRHTAAL KIKKQKFSVG
AALYQDNTND VVPSGEGLTR QKPRTLSFTG EKPSTPILDT
INYPIHMKNL SVEELERLAD ELREEIVYTV SKTGGHLSSS
LGVSELTVAL HHVFNTPDDK IIWDVGHQAY PHKILTGRRS
RMHTIRQTFG LAGFPKRDES PHDAFGAGHS STSISAGLGM
AVGRDLLQKN NHVISVIGDG AMTAGQAYEA LNNAGFLDSN
LIIVLNDNKQ VSLPTATVDG PAPPVGALSK ALTKLQASRK
FRQLREAAKG MTKQMGNQAH EIASKVDTYV KGMMGKPGAS
LFEELGIYYI GPVDGHNIED LVYIFKKVKE MPAPGPVLIH
IITEKGKGYP PAEVAADKMH GVVKFDPTTG KQMKVKAKTQ
SYTQYFAESL VAEAEQDEKV VAIHAAMGGG TGLNIFQKRF
PDRCFDVGIA EQHAVTFAAG LATEGLKPGC TIYSSFLQRG
YDQVVHDVDL QKLPVRFMMD RAGLVGADGP THCGAFDTTY
MACLPNMVVM APSDEAELMH MVATAAVIDD RPSCVRYPRG
MGIGVPLPPN NKGIPLEVGK GRILKEGNRV AILGFGTIVQ
NCLAAAQLLQ EHGISVSVAD ARFCKPLDGD LIKNLVKEHE
VLITVEEGSI GGFSAHVSHF LSLNGLLDGN LKWRPMVLPD
RIYDHGAYPD QIEEAGLSSK HIAGTVLSLI GGGKDSLHLI
NM

An example of a nucleotide sequence that encodes the Plectranthus barbatus (Coleus forskohlii) 1-deoxy-D-xylulose 5-phosphate synthase (PbDXS) enzyme with SEQ ID NO:3 is shown below as SEQ ID NO:4:

ATGGCGTCTT GTGGAGCTAT CGGGAGTAGT TTCTTGCCAC
TGCTCCATTC CGACGAGTCA AGCTTCTTAT CTCGGCACAC
TGCTGCTCTT CACATCAAGA AGCAGAAGTT TTCTGTGGGA
GCTGCTCTGT ACCAGGATAA CACGAACGAT GTCGTTCCGA
GTGGAGAGGG TCTGACGAGG CAGAAACCAA GAACTCTGAG
TTTCACGGGA GAGAAGCCTT CAACTCCAAT TTTGGATACC
ATCAACTATC CAATCCACAT GAAGAATCTG TCCGTGGAGG
AACTGGAGAG ATTGGCCGAT GAACTGAGGG AGGAGATAGT
TTACAQCGGTG TCGAAACGG GAGGGCATTT GAGCTCAAGC
TTGGGTGTAT CAGAGCTCAC CGTTGCACTG CATCATGTAT
TCAACACACC CGATGACAAA ATCATCTGGG ATGTTGGACA
TCAGGCGTAT CCACACAAAA TCTTGACAGG GAGGAGGTCC
AGAATGCACA CCATCCGACA GACTTTCGGG CTTGCAGGGT
TCCCCAAGAG GGATGAGAGC CCGCACGACG CCTTCGGAGC
TGGTCACAGC TCCACTAGTA TTTCAGCTGG TCTAGGGATG
GCGGTGGGGA GGGACTTGCT GCAGAAGAAC AACCACGTGA
TCTCGGTGAT CGGCGACGGG GCCATGACAG CGGGGCAGGC
ATACGAGGCC TTGAACAATG CAGGATTTCT TGATTCCAAT
CTGATCATCG TGTTGAACGA CAACAAACAA GTGTCCCTGC
CTACAGCCAC AGTCGACGGC CCTGCTCCTC CCGTCGGAGC
CTTGAGCAAA GCCCTCACCA AGCTGCAAGC AAGCAGGAAG
TTCCGGCAGC TACGAGAAGC AGCAAAAGGC ATGACTAAGC
AGATGGGAAA CCAAGCACAC GAAATTGCAT CCAAGGTAGA
CACTTACGTT AAAGGAATGA TGGGGAAACC AGGCGCCTCC
CTCTTCGAGG AGCTCGGGAT TTATTACATC GGCCCTGTAG
ATGGACATAA CATCGAAGAT CTTGTCTATA TTTTCAAGAA
AGTTAAGGAG ATGCCTGCGC CCGGCCCTGT TCTTATTCAC
ATCATCACCG AGAAGGGCAA AGGCTACCCT CCAGCTGAAG
TTGCTGCTGA CAAAATGCAT GGTGTGGTGA AGTTTGATCC
AACAACGGGG AAACAGATGA AGGTGAAAGC GAAGACTCAA
TCATACACCC AATACTTCGC GGAGTCTCTG GTTGCAGAAG
CAGAGCAGGA CGAGAAAGTG GTGGCGATCC ACGCGGCCAT
GGGAGGCGGA ACGGGGCTGA ACATCTTCCA GAAACGGTTT
CCCGACCGAT GTTTCGATGT CGGGATAGCC GAGCAGCATG
CAGTCACCTT CGCCGCGGGT CTTGCAACGG AAGGCCTCAA
GCCCTTCTGC ACAATCTACT CTTCCTTCCT GCAGCGAGGC
TATGATCAGG TGGTGCACGA TGTGGATCTT CAGAAACTCC
CGGTGAGATT CATGATGGAC AGAGCTGGAC TGGTGGGAGC
TGACGGCCCA ACCCATTGCG GCGCCTTCGA CACCACCTAC
ATGGCCTGCC TGCCCAACAT GGTGGTCATG GCTCCCTCAG
ATGAGGCTGA GCTCATGCAC ATGGTCGCCA CCGCCGCCGT
CATTGATGAT CGCCCTAGCT GCGTTAGGTA CCCTAGAGGA
AACGGTATAG GGGTGCCCCT CCCTCCAAAC AACAAAGGAA
TTCCATTAGA GGTTGGGAAG GGAAGGATTT TGAAAGAGGG
TAACCGAGTT GCCATTCTAG GCTTCGGAAC TATCGTGCAA
AACTGTCTAG CAGCAGCCCA ACTTCTTCAA GAACACGGCA
TATCCGTGAG CGTAGCCGAT GCGAGATTCT GCAAGCCTCT
GGATGGAGAT CTGATCAAGA ATCTTGTGAA GGAGCACGAA
GTTCTCATCA CTGTGGAAGA GGGATCCATT GGAGGATTCA
GTGCACATGT CTCTCATTTC TTGTCCCTCA ATGGACTCCT
CGACGGCAAT CTTAAGTGGA GGCCTATGGT GCTCCCAGAT
AGGTACATTG ATCATGGAGC ATACCCTGAT CAGATTGAGG
AAGCAGGGCT GAGCTCAAAG CATATTGCAG GAACTGTTTT
GTCACTTATT GGTGGAGGGA AAGACAGTCT TCATTTGATC
AACATG

A Plectranthus barbatus 1-deoxy-D-xylulose 5-phosphate synthase (PbDXS) protein with SEQ ID NO:3 was used in experiments described in the Examples. The PbDXS nucleotide sequence used in the experiments (SEQ ID NO:3) described herein significantly differed from the previously published sequence (Gnanasekaran et al. J. Biol., Eng. 9, 24 (2015)).

DXS enzymes with sequences that are not identical to SEQ ID NO:3 can also be used. For example, a variant Plectranthus barbatus 1-deoxy-D-xylulose 5-phosphate synthase (PbDXS) protein (NCBI accession number KP889115.1) is shown below as SEQ ID NO:5.

| | |
|---|---|
| 1 | MASCGAIGSS FLPLLHSDES SLLSRPTAAL HIKKQKFSVG |
| 41 | AALYQDNTND VVPSGEGLTR QKPRTLSFTG EKPSTPILDT |
| 81 | INYPHIMKNL SVEELEILAD ELREEIVYTV SKTGGHLSSS |
| 121 | LGVSELTVAL HHVFNTPDDK IIWDVGHQAY PHKILTGRRS |
| 161 | RMHTIRQTFG LAGFPKRDES PHDAFGAGHS STSISAGLGM |
| 201 | AVGRDLLQKN NHVISVIGDG AMTAGQAYEA MNNAGFLDSN |
| 241 | LIIVLNDNKQ VSLPTATVDG PAPPVGALSK ALTKLQASRK |
| 281 | FRQLREAAKG MTKQMGNQAH EIASKVDTYV KGMMGKPGAS |
| 321 | LFEELGIYYI GPVDGHNIED LVYIFKKVKE MPAPGPVLIH |
| 361 | IITEKGKGPY PAEVAADKMH GVVKFDPTTG KQMKVKTKTQ |
| 401 | SYTQYFAESL VAEAEQDEKV VAIHAAMGGG TGLNIFQKRF |
| 441 | PDRCFDVGIA EQHAVTFAAG LATEGLKPFC TIYSSFLQRG |
| 481 | YDQVVHDVDL QKLPVRFMMD RAGLVGADGP THCGAFDTTY |
| 521 | MACLPNMVVM APSDEAELMH MVATAAVIDD RPSCVRYPRG |
| 561 | NGIGVPLPPN NKGIPLEVGK GRILKEGNRV AILGFGTIVQ |
| 601 | NCLAAAQLLQ EHGISVSVAD ARFCKPLDGD LIKNLVKEHE |
| 641 | VLITVEEGSI GGFSAHVSHF LSLNGLLDGN LKWRPMVLPD |
| 681 | RYIDHGAYPD QIEEAGLSSK HIAVTVLSLI GGGKDSLHLI |
| 721 | NM |

A cDNA sequence for Plectranthus barbatus 1-deoxy-D-xylulose 5-phosphate synthase (PbDXS) with SEQ ID NO:5 is shown below as SEQ ID NO:6.

| | |
|---|---|
| 1 | ATCGCGTCTT GTGGACCTAT CGGGAGTAGT TTCTTGCCAC |
| 41 | TGCTCCATTC CGACGAGTCA AGCTTGTTAT CTCGGCCCAC |
| 81 | TGCTGCTCTT CACATCAAGA AGCAGAAGTT TTCTGTGGGA |
| 121 | GCTGCTCTGT ACCAGGATAA CACGAACGAT GTCGTTCCGA |
| 161 | GTGGAGAGGG TCTGACGAGG CAGAAACCAA GAACTCTGAG |
| 201 | TTTCACGGGA GAGAAGCCTT CAACTCCAAT TTTGGATACC |
| 241 | ATCAACTATC CAATCCACAT GAAGAATCTG TCCGTGGAGG |
| 281 | AACTGGAGAT ATTGGCCGAT GAACTGAGGG AGGAGATAGT |
| 321 | TTACACGGTG TCGAAAACGG GAGGGCATTT GAGCTCAAGC |
| 361 | TTGGGTGTAT CAGAGCTCAC CGTTGCACTG CATCATGTAT |
| 401 | TCAACACACC CGATGACAAA ATCATCTGGG ATGTTGGACA |
| 441 | TCAGGCGTAT CCACACAAAA TCTTGACAGG GAGGAGGTCC |
| 481 | AGAATGCACA CCATCCGACA GACTTTCGGG CTTGCAGGGT |
| 521 | TCCCCAAGAG GGATGAGAGC CCGCACGACG CGTTCGGAGC |
| 561 | TGGTCACAGC TCCACTAGTA TTTCAGCTGG TCTAGGGATG |
| 601 | GCGGTGGGGA GGGACTTGCT ACAGAAGAAC AACCACGTGA |
| 641 | TCTCGGTGAT CGGAGACGGA GCCATGACAG CGGGGCAGGC |
| 681 | ATACGAGGCC ATGAACAATG CAGGATTTCT TGATTCCAAT |
| 721 | CTGATCATCG TGTTGAACGA CAACAAACAA GTGTCCCTGC |
| 761 | CTACAGCCAC CGTCGACGGC CCTGCTCCTC CCGTCGGAGC |
| 301 | CTTGAGCAAA GCCCTCACCA AGCTGCAAGC AAGCAGGAAG |
| 841 | TTCCGGCAGC TACGAGAAGC AGCAAAAGGC ATGACTAAGC |
| 381 | AGATGGGAAA CCAAGCACAC GAAATTGCAT CCAAGGTAGA |
| 921 | CACTTACGTT AAAGGAATGA TGGGGAAACC AGGCGCCTCC |
| 961 | CTCTTCGAGG AGCTCGGGAT TTATTACATC GGCCCTGTAG |
| 1001 | ATGGACATAA CATCGAAGAT CTTGTCTATA TTTTCAAGAA |
| 1041 | AGTTAAGGAG ATGCCTGCGC CCGGCCCTGT TCTTATTCAC |
| 1081 | ATCATCACCG AGAAGGGCAA AGGCTACCCT CCAGCTGAAG |
| 1121 | TTGCTGCTGA CAAAATGCAT GGTGTGGTGA AGTTTGATCC |
| 1161 | AACAACGGGG AAACAGATGA AGGTGAAAAC GAAGACTCAA |
| 1201 | TCATACACCC AATACTTCGC GGAGTCTCTG GTTGCAGAAG |
| 1241 | CAGAGCAGGA CGAGAAAGTG GTGGCGATCC ACGCGGCGAT |
| 1281 | GGGAGGCGGA ACGGGGCTGA ACATCTTCCA GAAACGGTTT |
| 1321 | CCCGACCGAT GTTTCGATGT CGGGATAGCC GAGCAGCATG |
| 1361 | CAGTCACCTT CGCCGCGGGT CTTGCAACGG AAGGCCTCAA |
| 1401 | GCCCTTCTGC ACAATCTACT CTTCCTTCCT GCAGCGAGGT |
| 1441 | TATGATCAGG TGGTGCACGA TGTGGATCTT CAGAAACTCC |
| 1481 | CGGTGAGATT CATGATGGAG AGAGCTGGAC TTGTGGGAGC |
| 1521 | TGACGGCCCA ACCCATTGCG GCGCCTTCGA CACCACCTAC |
| 1561 | ATGGCCTGCC TGCCCAACAT GGTCGTCATG GCTCCCTCCG |
| 1601 | ATGAGGCTGA GCTCATGCAC ATGGTCGCCA CTGCCGCTGT |
| 1641 | CATTGATGAT CGCCCTAGCT GCGTTAGGTA CCCTAGAGGA |
| 1681 | AACGGTATAG GGGTGCCCCT CCCTCCAAAC AATAAAGGAA |
| 1721 | TTCCATTAGA GGTTGGGAAG GGAAGGATTT TGAAAGAGGG |
| 1761 | TAACCGAGTT GCCATTCTAG GCTTCGGAAC TATCGTGCAA |
| 1801 | AACTGTCTAG CAGCAGCCCA ACTTCTTCAA GAACACGGCA |
| 1341 | TATCCGTGAG CGTAGCCGAT GCGAGATTCT GCAAGCCTCT |
| 1881 | GGATGGAGAT CTGATCAAGA ATCTTGTGAA GGAGCACGAA |
| 1921 | GTTCTCATCA CTGTGGAAGA GGGATCCATT GGAGGATTCA |
| 1961 | GTGCACATGT CTCTCATTTC TTGTCCCTCA ATGGACTCCT |
| 2041 | CGACGGCAAT CTTAAGTGGA GGCCTATGGT GCTCCCAGAT |
| 2081 | AGGTACATTG ATCATGGAGC ATACCCTGAT CAGATTGAGG |
| 2121 | AAGCAGGGCT GAGCTCAAAG CATATTGCAG GAACTGTTTT |
| 2161 | GTCACTTATT GGTGGAGGGA AAGACAGTCT TCATTTGATC |
| 2201 | AACATGTAA |

A comparison of the SEQ ID NO:3 and SEQ ID NO:5 Plectranthus barbatus 1-deoxy-D-xylulose 5-phosphate synthase (PbDXS) proteins is shown below, illustrating that these two DXS proteins have at least 99.3% sequence identity.

| | | |
|---|---|---|
| Sq3 | 1 | MASCGAIGSSFLPLLHSDESSFLSRHTAAIHIKKQKFSVGAAIYQDNTNDVVPSGEGLTR |
| Sq5 | 1 | MASCGAIGSSFLPLLHSDESSLLSRPTAALHIKKQKFSVGAALYQDNTNDVVPSGEGLTR |
| | | ********************* *** ********************************** |
| Sq3 | 61 | QKPRTLSFTGEKPSTPILDTINYPIHMKNLSVEELERLADELREEIVYTVSKTGGHLSSS |
| Sq5 | 61 | QKPRTLSFTGEKPSTPILDTINYPIHMKNLSVEELEILADELREEIVYTVSKTGGHLSSS |
| | | ************************************ *********************** |
| Sq3 | 121 | LGVSELTVALHHVFNTPDDKIIWDVGHQAYPHKILTGRRSRMHTIRQTFGLAGFPKRDES |
| Sq5 | 121 | LGVSELTVALHHVFNTPDDKIIWDVGHQAYPHKILTGRRSRMHTIRQTFGLAGFPKRDES |
| | | ************************************************************ |
| Sq3 | 181 | PHDAFGAGHSSTSISAGLGMAVGRDLLQKNNHVISVIGDGAMTAGQAYEALNNAGFLDSN |
| Sq5 | 181 | PHDAFGAGHSSTSISAGLGMAVGRDLLQKNNHVISVIGDGAMTAGQAYEAMNNAGFLDSN |
| | | ************************************************** ********* |
| Sq3 | 241 | LIIVLNDNIQVSLPTATVDGPAPPVGALSKALTKLQASRKFRQLREAAKGMTKQMGNQAH |
| Sq5 | 241 | LIIVLNDNKQVSLPTATVDGPAPPVGALSKALTKLQASRKFRQLREAAKGMTKQMGNQAH |
| | | ************************************************************ |
| Sq3 | 301 | EIASKVDTYVKGMMGKPGASLFEELGIYYIGPVDGHNIEDLVYIFKKVKEMPAPGPVLIH |
| Sq5 | 301 | EIASKVDTYVKGMMGKPGASLFEELGIYYIGPVDGHNIEDLVYIFKKVKEMPAPGPVLIH |
| | | ************************************************************ |
| Sq3 | 361 | IITEKGKGYPPAEVAADKMHGVVKFDPTTGKQMKVKAKTQSYTQYFAESLVAEAEQDEKV |
| Sq5 | 361 | IITEKGKGYPPAEVAADKMHGVVKFDPTTGKQMKVKTKTQSYTQYFAESLVAEAEQDEKV |
| | | ************************************ *********************** |
| Sq3 | 421 | VAIHAAMGGGTGLNIFQKRFPDRCFDVGIAEQHAVTFAAGLATEGLKPFCTIYSSFLQRG |
| Sq5 | 421 | VAIHAAMGGGTGLNIFQKRFPDRCFDVGIAEQHAVTFAAGLATEGLKPFCTIYSSFLQRG |
| | | ************************************************************ |
| Sq3 | 481 | YDQVVHDVDLQKLPVRFMMDRAGLVGADGPTHCGAFDTTYMACLPNMVVMAPSDEAELMH |
| Sq5 | 481 | YDQVVHDVDLQKLPVRFMMDRAGLVGADGPTHCGAFDTTYMACLPNMVVMAPSDEAELMH |
| | | ************************************************************ |
| Sq3 | 541 | MVATAAVIDDRPSCVRYPRGNGIGVPLPPNNKGIPLEVGKGRILKEGNRVAILGFGTIVQ |
| 3q5 | 541 | MVATAAVIDDRPSCVRYPRGNGIGVPLPPNNKGIPLEVGKGRILKEGNRVAILGFGTIVQ |
| | | ************************************************************ |
| Sq3 | 601 | NCLAAAQLLQEHGISVSVADARFCKPLDGDLIKNLVKEHEVLIIVEEGSIGGFSAHVSHF |
| Sq5 | 601 | NCLAAAQLLQEHGISVSVADARFCKPLDGDLIKNLVKEHEVLITVEEGSIGGFSAHVSHF |
| | | ************************************************************ |
| Sq3 | 661 | LSLNGLLDGNLKWRPMVLPDRYIDHGAYPDQIEEAGLSSKHIAGTVLSLIGGGKDSLHLI |
| Sq5 | 661 | LSLNGLLDGNLKWRPMVLPDRYIDHGAYPDQIEEAGLSSKHIAGTVLSLIGGGKDSLHLI |
| | | ************************************************************ |
| Sq3 | 721 | NM |
| Sq5 | 721 | NM |
| | | ** |

Another 1-deoxy-D-xylulose 5-phosphate synthase enzyme from Isodon rubescens can be used as a fusion partner with LDSP is the Isodon rubescens DXS protein (NCBI accession number AMM72794.1) shown below as SEQ ID NO:7.

| | |
|---|---|
| 1 | MASCGAIRSS FLPLLHSDDS SLLSRTAAAL PIKKQKFSVG |
| 41 | AALQQDNSND VAANGESLTR QKPRALSFTG EKPSTPILDT |
| 81 | INYPNHMKNL SVEELERLAD ELREEIVYSV SKTGGHLSSS |
| 121 | LGVSELTVAL HHVFNTPDDK IIWDVGHQAY PHKILTGRRS |
| 161 | RMNTIRQTFG LAGFPKRDES AHDAFGAGHS STSISAGLGM |
| 201 | AVGRDLLKKN NHVISVIGDG AMTAGQAYEA LNNAGFLDSN |
| 241 | LIVVLNDNKQ VSLPTATVDG PAPPVGALSK ALTRLQASRK |
| 281 | FRQLREAAKG MTKQMGNQAH EVASKVDTYV KGMMGKPGAS |
| 321 | LFEELGIYYI GPVDGHSMED LVYIFQKVKE MPAPGPVLIH |
| 361 | IITEKGKGYP PAEVAADKMH GVVKFDPTTG KQMKTKTKTQ |
| 401 | SYTQYFAESL VAEAEQDEKV VAIHAAMGGG TGLNIFQKRF |
| 441 | PERCFDVGIA EQHAVTFAAG LATEGLKPFC TIYSSFLQRG |
| 481 | YDQVVHDVDL QKLPVRFMMD RAGLVGADGP THCGAFDTTY |
| 521 | MACLPNMVVM APSDEAELMH MVATAGVIDD RPSCVPYPRG |
| 561 | NGIGVPLPPN NKGNPLEIGK GRILKEGSRV AILGFGTIVQ |
| 601 | NCLAAAQLLQ EHGISVSVAD ARFCKPLDGD LIKKLVKEHE |
| 641 | VLITVEEGSI GGFSAHVSHF LSLNGLLDGN LKWRPMVLPD |
| 681 | RYIDHGAYPD QIEEAGLSSK HIAGTVLSLI GGGKDSLHLI |
| 721 | NM |

A cDNA sequence that encodes the Isodon rubescens DXS protein SEQ ID NO:7 is available as NCBI accession number KT831764.1, shown below as SEQ ID NO:8.

| | |
|---|---|
| 1 | ATGCCATCTT GTGGACCTAT CAGGAGCAGT TTCCTGCCAT |
| 41 | TCCICCATIC TGACCATTCT ACCTTGTTAT CCCGCACTCC |
| 81 | TGCTGCTCTT CCCATCAAAA AGCAAAAGTT CTCTUFGGGA |
| 121 | GCAGCTCTTC AACAGGATAA CACCAACGAT GTGGCGGCGA |
| 161 | ATGGAGAGAG TCTCACGAGG CAGAAGCCAA GAGCTCTCAG |
| 201 | TTTTACGGGA GAAAAGCCTT CAACTCCAAT TTTGGATACT |
| 241 | ATTAACTATC CAAACCACAT GAAAAATCTT TCCGTCGAGG |
| 231 | AACTAGAGAG ATTGGCTGAT GAATTGAGGG AAGAGATAGT |
| 321 | TTACTCGGTG TCCAAAACGG GAGGGCATTT AAGTTCAAGC |
| 361 | CTAGGTGTAT CAGAGCTCAC AGTTGCACTT CATCATGTAT |
| 401 | TCAACACACC TGATGATAAA ATCATTTGGG ATGTCGGACA |
| 441 | TCAGGCGTAT CCACACAAAA TCTTGACGGG GAGGAGGTCA |
| 481 | AGAATGAACA CGATTCGACA CACTTTCGGG TTAGCCGGGT |
| 521 | TCCCCAAGAG GGATGAGAGC GCGCACGATG CGTTTGGAGC |
| 561 | TGGTCACAGT TCAACTAGCA TTTCAGCTGG TCTAGGGATG |
| 601 | GCGGTGGGGA GGGACTTGCT AAAGAAGAAC AACCACGTCA |
| 641 | TATCAGTGAT CGGAGATGGG GCCATGACAG CCGGACAGGC |
| 681 | ATATGAGGCT TTGAACAATG CAGGATTCCT GGACTCCAAT |
| 721 | CTCATCGTCG TCTTGAACGA CAACAAGCAA GTGTCCCTGC |
| 761 | CCACTGCCAC CGTCGACGGC CCTGCTCCCC CCGTTGGAGC |
| 801 | CCTCAGCAAA GCCCTCACCA GACTGCAAGC CAGCAGAAAA |
| 341 | TTCCGCCAGC TCCGTGAAGC AGCTAAAGGC ATGACTAAGC |
| 831 | AGATGGGAAA CCAAGCCCAC GAAGTTGCAT CAAAGGTGGA |
| 921 | CACTTATGTG AAGGGAATGA TGGGGAAACC CGGCGCCTCC |
| 961 | CTCTTCGAGG AGCTTGGGAT TTATTACATC CGCCCTGTAG |
| 1001 | ATGGCCACAG TATGGAAGAT CTTGTCTATA TTTTCCAGAA |
| 1041 | AGTTAAGGAG ATGCCGGCGC CTGGACCTGT TCTCATTCAC |
| 1081 | ATCATAACCG AGAAGGGCAA AGGCTATCCT CCTGCTGAAG |
| 1121 | TTGCTGCGGA TAAAATGCAT GGTGTGGTGA AGTTTGATCC |
| 1161 | AACGACAGGG AAACAGATGA AGACTAAAAC GAAGACACAA |
| 1201 | TCATACACTC AATACTTCGC GGAGTCCCTA GTTGCAGAAG |
| 1241 | CAGAGCAGGA CGAGAAGGTG GTGGCGATCC ACGCGGCAAT |
| 1281 | GGGAGGCGGG ACGGGCCTCA ACATCTTCCA GAAGCGGTTT |
| 1321 | CCTGAGCGAT GTTTTGATGT TGGGATTGCA GAGCAGCACG |
| 1361 | CAGTCACCTT TGCCGCGGGT CTTGCAACTG AAGGCCTCAA |
| 1401 | GCCTTTCTGC ACAATCTACT CTTCCTTCCT GCAGAGAGGC |
| 1441 | TACGATCAGG TGGTTCACGA TGTAGACCTT CAGAAGCTCC |
| 1481 | CCGTGAGATT CATGATGGAC AGAGCTGGAC TGGTGGGAGC |
| 1521 | AGACGGCCCC ACCCATTGCG GCGCCTTCGA CACCACCTAC |
| 1561 | ATGGCCTGCC TCCCCAACAT GGTGGTCATG GCTCCCTCCG |
| 1601 | ACGAGGCCGA GCTCATGCAC ATGGTCGCCA CCGCTGGAGT |
| 1641 | CATTGATGAC CGCCCCAGTT GCGTCAGATA CCCTAGAGGA |
| 1681 | AACGGTATAG GGGTACCTCT TCCACCAAAC AACAAAGGAA |
| 1721 | ATCCATTGGA GATTGGGAAG GGAAGGATCT TAAAAGAGGG |
| 1761 | GAGTAGAGTT GCCATTTTAG GCTTCGGGAC TATCGTTCAA |
| 1801 | AACTGTTTGG CAGCAGCCCA ACTTCTTCAA GAACACGGCA |
| 1841 | TATCTGTGAG CGTGGCTGAT GCAAGATTCT GCAAGCCCCT |
| 1881 | GGATGGAGAT CTGATCAAGA AACTGGTTAA GGAGCATGAA |
| 1921 | GTTCTAATCA CTGTGGAAGA GGGATCCATT GGCGGATTCA |
| 1961 | GTGCACATGT TTCTCATTTC TTGTCCCTCA ATGGACTGCT |
| 2001 | GGATCGGAAT CTTAAGTGGA GGCCGATGGT GCTCCCTGAT |
| 2041 | AGGTATATTG ATCATGGAGC ATACCCTGAT CAGATTGAAG |
| 2081 | AAGCAGGGCT GAGTTCAAAG CATATTGCAG GCACTGTTTT |
| 2121 | GTCACTGATT GGTGGAGGAA AAGACAGTCT TCATTTGATC |
| 2161 | AACATGTAA |

A comparison of the SEQ ID NO:3 and SEQ ID NO:7 Isodon rubescens DXS proteins is shown below, illustrating that these two DXS proteins have at least 95% sequence identity.

| | | |
|---|---|---|
| Sq3 | 1 | MASCGAIGSSFLPLLHSDESSFLSRHTAALHIKKQKFSVGAALYQDNTNDVVPSGEGLTR |
| Sq7 | 1 | MASCGAIRSSFLPLLHSDDSSLLSRTAAALPIKKQKFSVGAALQQDNSNDVAANGESLTR |
| | | ******* ********** ** *** *** ************ *** *** ** *** |
| Sq3 | 61 | QKPRTLSFTGEKPSTPILDTINYPTHMKNLSVEELERLADELREEIVYTVSKTGGHLSSS |
| Sq7 | 61 | QKPRALSFTGEKPSTPILDTINYPNHMKNLSVEELERLADELREEIVYSVSKTGGHLSSS |
| | | **** ******************* *********************** *********** |
| Sq3 | 121 | LGVSELTVALHHVFNTPDDKIIWDVGHQAYPHKILTGRRSRMHTIRQTFGLAGFPKRDES |
| Sq7 | 121 | LGVSELTVALHHVFNTPDPKIIWDVGHQAYPHKILTGRRSRMNTIRQTFGLAGFPKRDES |
| | | ****************************************** ***************** |
| Sq3 | 181 | PHDAFGAGHSSTSISAGLGMAVGRDLLQKNNHVISVIGDGAMTAGQAYEALNNAGFLDSN |
| Sq7 | 181 | AHDAFGAGHSSTSISAGLGMAVGRDLLKKNNHVISVIGDGAMTAGQAYEALNNAGFLDSN |
| | | ************************** ******************************** |
| Sq3 | 241 | LIIVLNDNKQVSLPTATVDGPAPPVGALSKALTKLQASRKFRQLREAAKGMTKQMGNQAH |
| Sq7 | 241 | LIVVLNDNKQVSLPTATVDGPAPPVGALSKALTRLQASRKFRQLREAAKGMTKQMGNQAH |
| | | ** ****************************** ************************** |
| Sq3 | 301 | EIASKVDTYVKGMMGKPGASLFEELGIYYIGPVDGHNIEDLVYIFKKVKEMPAPGPVLIH |
| Sq7 | 301 | EVASKVDTYVKGMMGKPGASLFFELGIYYIGPVDGHSMEDLVYIFQKVKEMPAPGPVLIH |
| | | * ********************************** ******* ************** |
| Sq3 | 361 | IITEKGKGYPPAEVAADKMHGVVKFDPTTGKQMKVKAKTQSYTQYFAESLVAEAEQDEKV |
| Sq7 | 361 | IITEKGKGYPPAEVAADKMHGVVKFDPTTGKQMKTKTKTQSYTQYFAESLVAEAEQDEKV |
| | | ********************************** * *********************** |
| Sq3 | 421 | VAIHAAMGGGTGLNIFQKRFPDRCFDVGIAEQHAVTFAAGLATEGLKPFCTIYSSFLQRG |
| Sq7 | 421 | VAIHAAMGGGTGLNIFQKRFPERCFDVGIAEQHAVTFAAGLATEGLKPFCTIYSSFLQRG |
| | | ********************* ************************************** |
| Sq3 | 481 | YDQVVHDVDLQKLPVRFMMDRAGLVGADGPTHCGAFDTTYMACLPNMVVMAPSDEAELMH |
| Sq7 | 481 | YDQVVHDVDLQKLPVRFMMDRAGLVGADGPTHCGAFDTTYMACLPNMVVMAPSDEAELMH |
| | | ************************************************************ |
| Sq3 | 541 | MVATAAVIDDRPSCVRYPRGNGIGVPLPPNNKGIPLEVGKGRILKEGNRVAILGFGIIVQ |
| Sq7 | 541 | MVATAGVIDDRPSCVRYPRGNGIGVPLPPNNKGNPLEIGKGRILKEGSRVAILGFGTIVQ |
| | | ***** *************************** *** ********* ************ |
| Sq3 | 601 | NCLAAAQLLQEHGISVSVADARFCKPLDGDLIKNLVKEHEVLITVEEGSIGGFSAHVSHF |
| Sq7 | 601 | NCLAAAQLLQEHGISVSVADARFCKPLDGDLIKKLVKEHEVLITVEEGSIGGFSAHVSHF |
| | | ********************************* ************************** |
| Sq3 | 661 | LSLNGLLDGNLKWRPMVLPDRYIDHGAYPDQIEEAGLSSKHIAGTVLSLIGGGKDSLHLI |
| Sq7 | 661 | LSLNGLLDGNLKWRPMVLPDRYIDHGAYPDQIEEAGLSSKHIAGTVLSLIGGGKDSLHLI |
| | | ************************************************************ |
| Sq3 | 721 | NM |
| Sq7 | 721 | NM |
| | | ** |

Another enzyme that is useful for making precursors for terpene/terpenoid production is a geranylgeranyl diphosphate synthase (GGDPS; EC 2.5.1.29). This enzyme is at a branch point in the mevalonate pathway, and catalyzes the synthesis of geranylgeranyl diphosphate (GGPP, shown below) from dimethylallyl diphosphate and isopentenyl diphosphate.

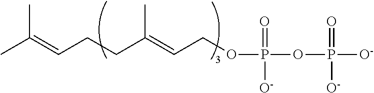

### Geranylgeranyl Diphosphate (GGPP)

A variety of different GGDPS enzymes can be used in the methods and expression systems described herein. One example of such a GGDPS enzyme is a Methanothermobacter thermautotrophicus (MtGGDPS) enzyme, which is a cytosolic protein. The Methanothermobacter thermautotrophicus (MtGGDPS) enzyme with the following sequence SEQ ID NO:9.

| | |
|---|---|
| 1 | MMEVMDILRK YSEMADERIR ESISDITPET LLRASEHLIT |
| 41 | AGGKKIRPSL ALLSSEAVGG DPGDAAGVAA AIELIHTFSL |
| 81 | IHDDIMDDDE IRRGEPAVHV LWGEPMAILA GDVLFSKAFE |
| 121 | AVIRNGDSEM VKEALAVVVD SCVKICEGQA LDMGFEERLD |
| 161 | VTEEEYMEMI YKKTAALIAA ATKAGAIMGG GSPQEIAALE |
| 201 | DYGRCIGLAF QIHDDYLDVV SDEESLGKPV GSDIAEGKMT |
| 241 | LMVVKALERA SEKDRERLIS ILGSGDEKLV AEAIEIFERY |
| 281 | GATEYAHAVA LDHVRMAKER LEVLEESDAR EALAMIADFV |
| 321 | LEREH |

An optimized cDNA sequence for this Methanothermobacter thermautotrophicus (MtGGDPS) with SEQ ID NO:9 is shown below as SEQ ID NO:10.

ATGATGGAGG TAATGGACAT ACTCCGAAAG TATTCAGAAA
TGGCAGATGA GAGGATCCGA GAGTCTATAA GTGATATTAC
TCCTGAAACG CTGCTTAGAG CATCAGAGCA CCTGATAACA
GCCGGAGGCA AGAAAATCAG GCCGAGCCTT GCTCTCTTAT
CCAGCGAAGC TGTGGGCGGG GACCCCGGAG ACGCTGCTGG
AGTCGCCGCC GCAATAGAGT TGATACATAC ATTCTCCTTA
ATACATGATG ATATCATGGA CGATCACGAG ATCAGGAGGG
GTGAGCCAGC CGTCCATGTC TTGTGGGGTG AGCCGATGGC
TATTCTCGCA GGTGACGTCT TGTTTAGTAA GGCTTTTGAG
GCCGTAATTA GAAATGGGGA TTCAGAGATG GTCAAAGAAG
CCCTTGCTGT TGTGGTGGAT TCATGTGTCA AGATATGCGA
GGGTCAAGCT CTTGACATGG GTTTCGAAGA GCGACTGGAC
GTAACCCAGG AAGAGTATAT GGAGATGATA TATAAAAAAA
CTGCAGCATT GATTGCTGCT GCTACAAAGG CAGGAGCCAT
CATGGGTGGC GGATCACCCC AGGAAATCGC AGCTCTTGAA
GACTATGGGA GATGTATTGG GTTGGCATTT CAAATCCACG
ACGACTATTT AGATGTAGTT TCTGATGAGG AAAGTCTGGG
AAAGCCCGTT GGGTCTGACA TAGCAGAAGG CAAGATGACA
CTGATGGTCG TCAAAGCCTT AGAGAGAGCT TCTGAAAAAG
ATAGGGAGAG GTTGATCTCT ATACTCGGGA GTGGCGACCA
GAAGCTTGTG GCCGAAGCCA TCGAAATTTT CGAACGATAC
GGAGCAACTG AATATGCTCA CGCCGTGGCC CTGGATCATG
TGCGTATGGC TAAGGAGCGT TTGGAAGTCC TCGAAGAGTC
CGATGCCAGG GAAGCTTTAG CCATGATTGC AGATTTTGTG
TTAGAGCGTG AACACTAA

Another example of a GGDPS enzyme that can be used is an Euphorbia peplus GGDPS1 (EpGGDPS1; accession no. MH363711) enzyme, which can increase precursor availability for diterpenoid synthesis. Such an Euphorbia peplus GGDPS1 (EpGGDPS1) enzyme can have the following amino acid sequence (SEQ ID NO:11).

MAFSATFSSC DYSLLLKKSS VNGLKNHPKV PFSGQHFKLM
KANFTTRALT VSKSSAVQQP PLTAADSQGS NSNTIPLPPF
AFDEYMKTKA KSVNKALDDA IPIQHPIKIH ESMRYSLLAG
GKRVRPVLCI AACELVGGDE AAAMPSACAM EMIHTMSLIH
DDLPCMDNDD LRRGKPTNHI KYCEETAILA GDALLSFSFE
HVARATKNVS PDRMIRVIGE LGSAVGSEGL VAGQIVDIDS
EGKEVSLSDL EYIHIHKTAK LLEAAVVCGA IVGGADDESV
ERMRKYARCI GLLFQVVDDI LDVTKSSEEL GKTACKDLAT
DKATYPKLLG IDEARKLAAK LVEQANQELA YFDAAKAAPL
YHFANYIASR QN

A nucleotide sequence encoding the Euphorbia peplus GGDPS1 enzyme with SEQ ID NO:11 is shown below as SEQ ID NO:12.

ATGGCCTTCT CCGCGACATT TTCCAGCTGC GACTACTCAC
TTCTTTTAAA AAAATCATCC GTCAATGGCC TCAAAAACCA
CCCGAAAGTT CCATTTTCTG GTCAACACTT CAAGTTAATG
AAAGCCAACT TCACCACCCG TGCCCTGACC GTTTCCAAAT
CCTCCGCGGT GCAGCAACCA CCGCTCACTG CGGCGGATTC
TCAAGGATCA AATTCCAATA CTATCCCTCT TCCTCCATTC
GCATTCGACG AATACATGAA AACCAAGGCT AAAAGGGTCA
ACAAAGCATT AGACGACGCT ATTCCGATTC AACATCCGAT
CAAAATCCAT GAATCCATGA GATACTCTCT CCTCGCCGGC
GGCAAGCGTG TCCGGCCAGT TTTATGTATA GCTGCTTGTG
AACTAGTCGG AGGAGAGGAA GCAGCAGCTA TGCCGTCAGC
ATGTGCTATG GAAATGATCC ATACCATGTC ATTAATCCAC
GACGATCTTC CTTGTATGGA CAACGACGAT CTTCGTCGCG
GAAAACCAAC AAACCACATA AAATACGGGG AAGAAACCGC
CATTCTTGCC GGCGATGCAC TCCTTTCATT TTCCTTTGAA
CACGTAGCTA GGGCAACAAA AAACGTTTCC CCGGACCGGA
TGATCCGAGT CATAGGGGAG CTAGGTTCAG CTGTGGGTTC
GGAAGGTTTA GTCGCGGGAC AAATCGTGGA CATCGATAGC
GAGGGGAAGG AAGTGAGTTT AAGTGATTTG GAGTATATTC
ATATTCATAA GACGGCTAAG CTTTTGGAAG CAGCCGTCGT
GTGTGGTGCG ATAGTCGGTG GCGCCGACGA TGAAAGTGTG
GAGAGAATGA GGAAATATGC TAGATGTATA GGCCTATTGT
TCCAAGTTGT GGATGATATA TTAGATGTGA CAAAGTCATC
GGAGGAGCTC GGGAAGACCG CGGGGAAAGA TTTAGCGACG
GATAAAGCGA CGTATCCGAA GTTGTTGGGG ATTGACGAGG
CGAGGAAACT TGCAGCTAAA TTGGTGGAGC AAGCTAATCA
AGAACTTGCT TATTTTGATG CTGCTAAGGC TGCTCCGTTA
TATCATTTTG CTAATTATAT TGCTAGTAGG CAAAATTGA

Another example of a GGDPS enzyme that can be used is an Euphorbia peplus GGDPS2 (EpGGDPS2; accession no. MH363712) enzyme, which can have the following amino acid sequence (SEQ ID NO:13).

MNSMNLGSWL NTSSIFNQST RSRSPPLKSF SIRLPRHKPR
FISSIMTKEE ETLTQKPQFD FKSYMLQKAA SIHQALDAAV
SIKEPAKIHE SMRYSLLAGG KRVRPALCLA ACELVGGNDS
QAMPAACAVE MVHTMSLIHD DLPCMDNDDL RRGKPTNHIV
FGEDVAVLAG DALLSFAFEH IAVATVNVSP ERIVRAIGEL
ASAIGAEGLV AGQVVDIACE KACDVGLETL EFIHVHKTAK
LLECAVVLGA ILGGGKDDEI EKLRKYARGI GLLFQVVDDI
LDVTKSSEEL GKTAGKDLVA DKVTYPKLLG IEKSREFAEK
LNREAQQQLS EFDVEKAAPL IALANYIAYR QN

A nucleotide sequence encoding the Euphorbia peplus GGDPS2 enzyme with SEQ ID NO:13 is shown below as SEQ ID NO:14.

ATGAACTCCA TGAATTTGGG TTCATGGCTC AACACTTCTT
CAATCTTCAA CCAATCTACC AGATCCAGAT CCCCGCCATT
AAAATCCTTC TCAATTCGTC TTCCCCGTCA CAAACCCAGA
TTCATTTCTT CAATTATGAC CAAAGAAGAA GAAACCCTAA
CCCAAAAACC CCAATTTGAT TTCAAATCTT ACATGCTCCA
AAAAGCTGCT TCCATTCATC AAGCTCTAGA CGCCGCCGTT
TCGATCAAAG AACCCGCTAA AATCCATGAA TCCATGCGGT
ATTCCCTCTT AGCCGGCGGG AAAAGAGTCC GGCCAGCGTT
ATGTTTAGCC GCGTGTGAGC TCGTCGGCGG GAACGATTCT
CAGGCGATGC CGGCGGCTTG CGCGGTGGAA ATGGTCCACA
CGATGTCTCT TATTCACGAT GATCTCCCCT GTATGGATAA
CGATGATCTA CGCCGCGGAA AACCCACGAA CCATATCGTG
TTCGGGGAAG ACGTGGCGGT TCTCGCTGGG GATGCGTTGC
TCTCGTTCGC ATTCGAGCAC ATTGCGGTTG CTACGGTGAA
TGTGTCACCG GAGAGGATTG TCCGGGCCAT CGGGGAATTA
GCCAGCGCGA TTGGGGCAGA AGGGTTAGTT GCTGGACAAG
TGGTTGATAT AGCTTGTGAG AAAGCTTGTG ATGTGGGATT
AGAAACGTTG GAGTTCATTC ATGTTCACAA AACGGCGAAA
TTCCTGGAAT GCGCTGTCGT ATTCGGGGCA ATATTAGGGG
GAGGAAAGGA TGATGAGATT GAGAAGTTGA GGAAATATGC
AAGAGGAATA GGGTTGTTGT TTCAAGTAGT GGATGATATT
TTAGATGTCA CAAAATCATC GGAAGAGTTG GGGAAAACTG
CAGGGAAAGA TTTGGTGGCG GATAAGGTAA CATACCCTAA
ACTTTTAGGG ATTGAAAAAT CAAGGGAATT TGCTGAGAAA
TTGAATAGGG AAGCTCAACA ACAGTTGAGT GAGTTTGATG
TGGAAAAGGC AGCTCCTTTG ATTGCTTTGG CTAATTATAT
TGCTTATAGG CAGAATTGA

Another example of a GGDPS enzyme that can be used is an Sulfolobus acidocaldarius GGDPS enzyme, which is a cytosolic protein. The Sulfolobus acidocaldarius GGDPS enzyme can have the following amino acid sequence (SEQ ID NO:15).

MSYFDNYFNE IVNSVNDIIK SYISGDVPKL YEASYHLFTS
GGKRLRPLIL TISSDLFGGQ RERAYYAGAA IEVLHTFTLV
HDDIMDQDNI RRGLPTVHVK YGLPLAILAG DLLHAKAFQL
LTQALRGLPS ETIIKAFDIF TRSIIIISEG QAVDMEFEDR
IDIKFQEYLD MISRYTAALF SASSSIGALI AGANDNDVRL
MSDFGTNLGI AFQIVDDILG LTADEKELGK PVFSDIREGK
KTILVIKTLE LCKEDEKKIV LKALGNKSAS KEELMSSADI
IKKYSLDYAY NLAEKYYNNA IDSLNQVSSK SDIPGKALKY
LAEFTIRRRK

A codon optimized nucleotide sequence encoding the Sulfolobus acidocaldarius GGDPS (SaGGDPS) enzyme with SEQ ID NO:15 is shown below as SEQ ID NO:16.

ATGAGTTATT TTGACAACTA CTTCAATGAA ATAGTCAACA
GCGTCAATGA TATAATCAAA TCCTACATCA GTGGAGACGT
GCCAAAACTC TACGAAGCAT CATACCACCT GTTCACATCT
GGAGGAAAAC GATTGAGACC CTTGATATTA ACCATAAGTA
GCGACCTCTT TGGGGGCCAG AGAGAAAGAG CATATTACGC
TGGAGCAGCT ATCGAGGTGT TACATACATT CACCTTGGTG
CATGATGACA TTATGGATCA GGACAATATA AGGCGAGGTT
TACCGACTGT GCATGTGAAA TACGGTCTGC CGCTGGCTAT
TCTGGCCGGC GATTTACTCC ATGCCAAGGC CTTCCAGTTG
CTCACCCAGG CACTCCGTGG ACTGCCCAGC GAGACAATTA
TCAAAGCCTT TGACATTTTC ACGAGATCCA TAATAATTAT
TTCCGAGGGC CAAGCTGTCG ATATGGAATT TGAAGATAGG
ATAGATATTA AAGAGCAGGA ATATCTCGAC ATGATTAGCC
GAAAAACCGC TGCTCTCTTC ACTGCCTCTA GCTCCATCGG
CGCTTTAATC GCCGGCGCAA ACGATAATGA CGTCAGACTT
ATGTCTGATT TCGGGACTAA TCTCGGCATC GCCTTTCAGA
TCGTAGACGA TATTCTTGGT CTGACTGCAG ATGAAAAGGA
GCTTGGGAAG CCGGTGTTCT CCGACATCCG TGAAGGTAAA
AAGACGATCT TGGTCATCAA GACGCTGGAA CTTTGCAAAG
AAGATGAGAA GAAGATCGTG CTCAAGGCCT TAGGCAACAA
GAGCGCCAGT AAGGAGGAGC TCATGTCTAG TGCTGATATC
ATTAAAAAGT ACAGCCTTGA CTACGCCTAT AACCTCGCAG
AGAAATACTA TAAGAACGCT ATCGATTCTT TAAACCAAGT
CAGCTCTAAG AGCGATATCC CTGGTAAACC ACTGAAGTAT
CTCGCTGAAT TTACAATAAG GAGACGTAAG TAA

Another example of a GGDPS enzyme that can be used is a Mortierella elongate GGDPS (MeGGDPS), which is a cytosolic protein. The Mortierella elongate GGDPS enzyme can have the following amino acid sequence (SEQ ID NO:17).

MAIPSIYPTD HDEAALLEPY TYICSNPGKE MRTELIEAFN
IWIKVPPQEL AIITKVVKML HTSSLLVDDI EDDSTLRRGE
PVAHKIFGVP ATINCANYVY FLALAELSKI SNPKMLTIFT
EELLCLHRGQ GMELLWRDSL TCPTEEEYIA MVNDKTGGLL
RLAVKLMQAA SDSTVDYVPM VELIGIHFQI RDDYLNIQSS
QYSANKGFCE DLTEGKFSYP IIHSIRAAPN SRKLLNILKQ
KPKDHELKVY AVSLMNATKT FEYCRQQLTL YEERARAEVR
RLGGNARLEK IIDRLSIPDP DSADAEKDVV PMFVATSTAG
GAAK

A codon optimized nucleotide sequence encoding the Mortierelia elongate GGDPS enzyme with SEQ ID NO:17 is shown below as SEQ ID NO:18.

ATGGCTATAC CTTCTATTTA CCCTACGGAT CACGATGAAG
CTGCCCTTCT GGAGCCGTAC ACGTATATAT GCAGTAATCC
GGGAAAGGAG ATGAGGACCG AGTTAATAGA AGCCTTTAAT
ATCTGGATCA AAGTGCCCCC TCAGGAGTTG GCAATCATCA
CAAAGGTCGT TAAGATGTTA CATACAAGCT CACTCTTGGT
AGATGACATT GAAGATGATA GTATTCTCCG TCGAGGCGAG
CCAGTTGCAC ACAAAATATT CGGTGTTCCG GCAACTATAA
ACTGTGCTAA TTATGTTTAC TTCCTCGCCT TAGCTGAATT
GTCTAAGATA TCTAATCCAA AAATGCTTAC CATATTTACC
GAAGAGCTTC TTTGCCTTCA TAGGGGACAA GGCATGGAGC
TCCTTTGGCC TGATAGCTTA ACCTGCCCGA CCGAGGAACA
GTATATAGCT ATGGTGAACG ATAAAACTGG AGGCCTTCTT
AGACTGGCCG TTAAGCTCAT GCAGGCAGCT AGTGACTCTA
CCGTAGACTA CGTCCCAATG GTGGAACTCA TTGGCATTCA
TTTTCAAATA AGGGACGATT ACTTAAACCT TCAGAGTTCT
CAGTACAGTG CAAACAAAGG TTTTTGCGAG GACCTGACTG
AGGGCAAGTT TTCCTATCCG ATTATTCACT CCATAAGGGC
AGCACCTAAT AGTCGAAAGT TGTTGAACAT CTTGAAGCAG
AAACCTAAAG ATCATGAACT CAAGGTTTAT GCCGTGTCAT
TAATGAACGC TACGAAAACA TTTGAGTATT GTAGGCAGCA
GCTGACCCTT TACGAGGAAC GTGCCCGAGC AGAAGTGAGG
CGTTTGGGAG GGAATGCTAG GCTCGAAAAA ATCATCGACA
GACTCTCTAT TCCACACCCC CACAGCGCAG ATCCAGAGAA
GGACGTGGTT CCTATGTTCG TTGCAACGTC AACTGCTGGT
GGAGCTGCAA AGTAA

Some tests indicated that a plastid-targeted form of Mortierelia elongate GGDPS was not particularly active for terpenoid synthesis. Hence, in some cases the GGDPS enzyme is not a plastid-targeted form of Mortierella elongate GGDPS.

Another example of a GGDPS enzyme that can be used is a Tolypothrix sp. PCC 7601 geranylgeranyl diphosphate synthase genomic (TsGGDPS). The Tolypothrix sp. PCC 7601 GGDPS enzyme can have the following amino acid sequence (SEQ ID NO:19).

MVATDKFKKM PETATFNLSA YLKERQQLCE TALDQALPVS
YPEKIYESMR YSLLAGGKRV RPILCLATSE MMGCTIEMAM
PTACAVEMIH TMSLIHDDLP AMDNDDYRRG KLTNHKVYGE
DIAILAGDGL LAYAFEEVAI ATPLTVPRDR VLQVVARLAR
ALGAAGLVGG QVVDLESEGK TDTSLETLNY IHNHKTAALL
EACVVCGGIL AGASVEDVQR LTRYAQNIGL AFQIVDDILD
ITATQEQLGK TAGKDLEAQK VTYPSLWGIE ESRVKAEQLI
EAACARLDVF GEKAQPLKAI AHFIISRNH

A genomic nucleotide sequence encoding the Tolypothrix sp. PCC 7601 GGDPS enzyme with SEQ ID NO:19 is shown below as SEQ ID NO:20.

ATGGTAGCAA CTGATAAGTT TAAAAAGATG CCAGAGACAG
CCACGTTTAA CCTATCAGCG TATCTCAAAG AGCGTCAACA
GCTTTGTGAA ACTGCTTTGG ATCAAGCGCT TCCCGTTTCC
TATCCAGAGA AGATTTACGA GTCGATGCGC TATTCTCTCT
TAGCTGGTGG CAAACGTGTG CGTCCTATCC TGTGCCTTGC
TACCAGTGAA ATGATGGGCG GCACAATCGA AATGGCAATG
CCAACAGCTT GTGCGGTGGA AATGATCCAC ACAATGTCAT
TAATTCATGA TGATTTGCCA GCGATGGATA ATGACGATTA
CCGTCGGGGT AAGCTGACAA ACCACAAGGT TTATGGCGAA
GATATCGCGA TTTTAGCTGG CGATGGTTTG TTGGCCTATG
CTTTTGAATT TGTTGCGATC GCCACCCCTT TAACTGTCCC
TAGAGATAGA GTATTGCAGG TAGTAGCGCG TCTTGCTCGG
GCATTAGGGG CTGCTGGCTT GGTTGGGGGC CAAGTAGTGG
ATCTAGAATC AGAAGGTAAA ACAGATACTT CCCTAGAGAC
TCTGAATTAC ATTCATAACC ACAAAACAGC TGCCCTTTTG
GAAGCTTGTG TTGTTTGTGG TGGTATTTTA GCGGGAGCAT
CTGTTGAAGA TGTACAAAGA CTAACTCGGT ATGCTCAGAA
TATTGGTCTG GCATTCCAAA TTGTTGATGA TATTTTAGAT
ATCACCGCTA CTCAAGAACA ATTAGGCAAA ACTGCTGGCA
AGGATTTGAA AGCGCAGAAA GTTACTTATC CCAGCCTGTG
GGGAATTGAA GAATCTCGCG TTAAAGCCGA ACAACTCATT
GAAGCAGCAT GTGCGGAATT AGACGTATTT GGAGAAAAAG
CACAACCTTT AAAACCGATC GCTCATTTTA TTATCAGCCG
CAATCACTAA

Another enzyme that can be used in the methods described herein is 3-hydroxy-3-methyl-glutaryl-coenzyme A reductase (HMG-CoA reductase or HMGR) is an NADH-dependent enzyme (EC 1.1.1.88) or in some cases an NADPH-dependent enzyme (EC 1.1.1.34) enzyme that is rate-controlling in the mevalonate pathway, which is the metabolic pathway that produces cholesterol and other isoprenoids. HMG-CoA reductase converts HMG-CoA to rad/atonic acid.

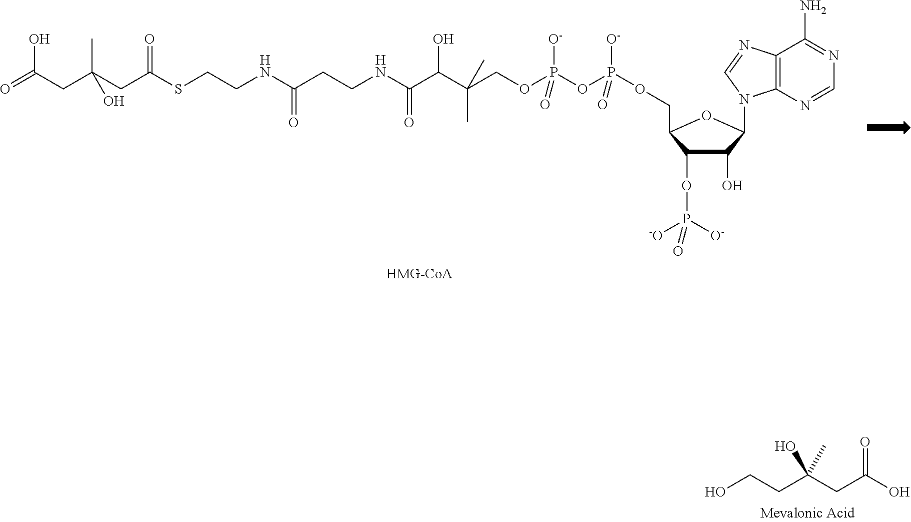

Such HMG-CoA reductase enzymes are useful for sesquiterpenoid synthesis.

One example of an HMG-CoA reductase that can be used is an Euphorbia lathyris hydroxymethylglutaryl coenzyme A reductase ((ElHMGR), for example, with accession number JQ694150.1, and with the sequence shown below (SEQ ID NO:21.

| | |
|---|---|
| 1 | MDSTRPESKL PRPIRRISDE VDHHGRCLSP PPKASDALPL |
| 41 | PLYLTNAVFF TLFFSVAYYL LHRWRDKIRN STPLHVVTLS |
| 81 | EIAAIVSLIA SFIYLLGEFG IDFVQSFIAR ASHDTWDLDD |
| 121 | ADRNYLIDGD HRLVTCSPAK ISPINSLPPK MSSPPEPIIS |
| 161 | PLASEEDEEI VKSVVNGTIP SYSLESKLGD CKRAAEIRRE |
| 201 | ALQRMMGRSL EGLPVEGFDY ESILGQCCEM PVGYVQIPVG |
| 241 | IAGPLLLDGQ EYSVPMATTE GCLVASTNRG CKAIHLSGGA |
| 281 | SSVLLKDGMT RAPVVRFASA MRAADLKFFL ENPENFDSLS |
| 321 | IAFNRSSRFA KLQSIQCSIA GKNLYMRFTC STGDAMGMNM |
| 361 | VSKGVQNVLD FLQSDFPDMD VIGISGNFCS DKKPAAVNWI |
| 401 | QGRGKSVVCE AIIKEEVVKK VLKSSVASLV ELNMLKNLTG |
| 441 | SAIAGALGGF NAHAGNIVSA IFIATGQDPA QNVESSHCIT |
| 481 | MMEAVNDGKD LHISVTMPSI EVGTVGGGTQ LASQSACLNL |
| 521 | LGVKGASKES PGANSRLLAT IVAGSVLAGE LSLMSAIAAG |
| 561 | QLVRSHMKYN RSSKDVTKFA SS |

A nucleic acid sequence for a full-length E. lathyris HMGR (ElHMGR159-582 JQ694150.1; SEQ ID NO:21) is shown below as SEQ ID NO:22.

| | |
|---|---|
| 1 | ACGCATAAAC ACATTCAAAC AGCTACTCTT CCAGCTCTTC |
| 41 | CTTTTTTCCC CCATTTCCAC TTCCATTATT TTATCCCCCC |
| 81 | TTTTTTCTCT CTTCTTCTCG ATTCATCCAT GGATTCCACT |
| 121 | CGGCCGGAAT CCAAACTCCG GCGACCGATC CGCCGCATCT |
| 161 | CGGACGAGGT TGACCACCAC GGCCGCTGTC TCTCTCCGCC |
| 201 | TCCTAAAGCC TCCGATGCTC TCCCTCTCCC GTTGTATTTA |
| 241 | ACCAATGCGG TTTTCTTTAC TCTCTTTTTC TCCGTCGCGT |
| 281 | ACTATCTTCT CCACCGGTGG AGAGATAAGA TCCGTAATTC |
| 321 | TACTCCTCTT CATCTCGTTA CTCTCTCTGA AATTGCCGCC |
| 361 | ATTGTTTCTC TCATTGCGTC TTTCATCTAC CTGCTTGGAT |
| 401 | TCTTCGGGAT TGATTTCGTT CAGTCTTTCA TTGCACGCGC |
| 441 | TTCTCATGAC ACGTGGGACC TTGATGATGC GGATCGTAAC |
| 481 | TACCTCATTG ATGGAGATCA CCGTCTCGTT ACTTGCTCTC |
| 521 | CTGCGAAGAT TTCTCCGATT AATTCTCTTC CTCCTAAAAT |
| 561 | GTCTTCCCCG CCGGAACCGA TTATTTCGCC TCTGGCATCC |
| 601 | GAGGAGGATG AGGAAATTGT TAAATCTGTT GTTAATGGAA |
| 641 | CGATTCCTTC GTATTCGTTG GAATCGAAGC TTGGGGATTG |
| 681 | TAAAAGAGCG GCTGAGATTC GACGGGAGGC TTTGCAGAGA |
| 721 | ATGATGGGGA GGTCGTTGGA GGGTTTACCT GTTGAAGGAT |
| 761 | TCGATTATGA GTCGATTTTA GGTCAGTGCT GTGAAATGCC |
| 801 | TGTTGGTTAT GTGCAGATTC CGGTTGGAAT TGCTGGGCCG |
| 841 | TTGCTGCTAG ACGGGCAAGA GTACTCTGTT CCGATGGCGA |
| 881 | CCACCGAGGG TTGTTTGGTT GCTAGCACTA ATAGAGGGTG |
| 921 | TAAAGCGATC CATTTGTCAG GTGGTGCTAG TAGTGTCTTG |
| 961 | TTGAAGGATG GCATGACTAG AGCTCCCGTT GTTCGATTCG |
| 1001 | CCTCGGCCAT CAGGGCCGCG GATTTGAAGT TTTTCTTAGA |
| 1041 | GAATCCTGAG AATTTCGATA GCTTGTCCAT CGCTTTCAAT |
| 1081 | AGGTCCAGTA GATTTGCAAA GCTCCAAAGC ATACAATGTT |
| 1121 | CTATTGCTGG AAAGAATCTA TATATGAGAT TCACCTGCAG |
| 1161 | CACTGGTGAT GCAATGGGGA TGAACATGGT TTCCAAAGGG |
| 1201 | GTTCAAAACG TTCTTGACTT CCTTCAAAGT GATTTCCCTG |
| 1241 | ACATGGATGT TATTGGCATC TCAGGAAATT TTTGTTCGGA |
| 1281 | CAAGAAGCCA GCTGCTGTGA ACTGGATTCA AGGGCGAGGC |
| 1321 | AAATCGGTTG TTTGCGAGGC AATTATCAAG GAAGAGGTGG |
| 1361 | TGAAGAAGGT ATTGAAATCA AGTGTTGCTT CACTAGTAGA |
| 1401 | GCTGAACATG CTCAAGAATC TTACTGGTTC AGCTATTGCT |
| 1441 | GGAGCTCTTG GTGGATTCAA TGCACATGCT GGCAACATAG |
| 1481 | TCTCTGCAAT TTTCATTGCC ACTGGCCAGG ATCCAGCCCA |
| 1521 | GAATGTTGAG AGTTCTCATT GCATCACCAT GATGGAAGCT |
| 1561 | GTCAATGATG GAAAAGATCT CCACATCTCT GTAACCATGC |
| 1601 | CTTCAATCGA GGTAGGAACA GTTGGAGGAG GGACACAACT |
| 1641 | AGCATCCCAA TCAGCATGTC TGAACCTACT CGGTGTAAAA |
| 1681 | GGAGCAAGTA AAGAATCACC AGGAGCAAAC TCAAGGCTCC |
| 1721 | TAGCCACAAT AGTAGCTGGT TCAGTCCTAG CTGGTGAACT |
| 1761 | CTCCCTAATG TCAGCCATAG CAGCAGGACA ACTAGTCCGG |
| 1801 | AGCCAGATGA AGTACAACAG ATCCAGCAAA GATGTAACCA |
| 1841 | AATTTGCATC ATCTTAATCA AAACTGGTTC ACAATAATAA |
| 1881 | AAGCGTCCGA ACCAAACCTC ATAGACAGAG AGCCAGATAG |
| 1921 | ACAGAGCCAG AAAGAGAAAG GGGAAGAAAA TGGAAGAAGA |
| 1961 | AGACTGTACT GTAGGGTACC TACCCCATGT GAGTTTTTTT |
| 2001 | ATTTTTTTTC AAAGCTTTTA ATAGCTGTAA AGTTGCTTAA |
| 2041 | TCATATGGAG AGAAGAAAGA AGAATTAGGT ACACAAAACT |
| 2081 | TTTGAAAATC TCCATTTTCT TACCCCAAAT TTGAGAAGTG |
| 2121 | GGTGTACTGT ATTAGTATGT TGGTGAGCAC ATGTGAGCAA |
| 2161 | AAAAGGTCCC CACTATCTAC TACCTAGTGT TTTTTGTGTA |
| 2201 | TGTTTGTGTC CTAATTTATT TGTTAATGTT TAGTTGCTTT |
| 2241 | CTTTCTTCTA TTTTTTGCAT ACATATGTTG TGTACACTTG |
| 2281 | TTTTTGTGTT TGAACTTACC TGGGGCTGAC ATGTGACACG |
| 2321 | TGGCGTGATA TTGTTTGTTG TTGATTTCCT TTTTTTTT |

A truncated ElHMGR159-582 polypeptide can also be used and is particularly useful because it is a feedback-insensitive form of ElHMGR. Such a truncated ElHMGR159-582 enzyme is shown below as SEQ ID NO:23.

MISPLASEED EEIVKSVVNG TIPSYSLESK LGDCKRAAEI
RREALQRMMG RSLEGLPVEG FDYESILGQC CEMPVGYVQI
PVGIAGPLLL DGQEYSVPMA TTEGCLVAST NRGCKAIHLS
GGASSVLLKD GMTRAPVVRF ASAMRAADLK FFLENPENFD
SLSIAFNRSS RFAKLQSIQC SIAGKNLYMR FTCSTGDAMG
MNMVSKGVQN VLDFLQSDFP DMDVIGISGN FCSDKKPAAV
NWIQGRCKSV VCEAIIKEEV VKKVLKSSVA SLVELNMLKN
LTGSAIAGAL GGFNAHAGNI VSAIFIATCQ DPAQNVESSH
CITMMEAVND GKDLHISVTM PSIEVGTVGG GTQLASQSAC
LNLLGVKGAS KESPGANSRL LATIVAGSVL AGELSLMSAI
AAGQLVRSHM KYNRSSKDVT KFASS

Note that a methionine was added to the N-terminus of this ElHMGR159-582 polypeptide to facilitate expression. A nucleotide sequence for the ElHMGR159-582 polypeptide with SEQ ID NO:23 is shown below with the added ATG (SEQ ID NO:24).

| | |
|---|---|
| 1 | ATGATTTCGC CTCTGGCATC CGAGGAGGAT GAGGAAATTG |
| 41 | TTAAATCTGT TGTTAATGGA ACGATTCCTT CGTATTCGTT |
| 81 | GGAATCGAAG CTTGGGGATT GTAAAAGAGC GGCTGAGATT |
| 121 | CGACGGGAGG CTTTGCAGAG AATGATGGGG AGGTCGTTGG |
| 161 | AGGGTTTACC TGTTGAAGGA TTCGATTATG AGTCGATTTT |
| 201 | AGGTCAGTGC TGTGAAATGC CTGTTGGTTA TGTGCAGATT |
| 241 | CCGGTTGGAA TTGCTGGGCC GTTGCTGCTA GACGGGCAAG |
| 281 | AGTACTCTGT TCCGATGGCG ACCACCGAGG GTTGTTTGGT |
| 321 | TGCTAGCACT AATAGAGGGT GTAAAGCGAT CCATTTGTCA |
| 361 | GGTGGTGCTA GTAGTGTCTT GTTGAAGGAT GGCATGACTA |
| 401 | GAGCTCCCGT TGTTCGATTC GCCTCGGCCA TGAGGGCCGC |
| 441 | GGATTTGAAG TTTTTCTTAG AGAATCCTGA GAATTTCGAT |
| 481 | AGCTTGTCCA TCGCTTTCAA TAGGTCCAGT AGATTTGCAA |
| 521 | AGCTCCAAAG CATACAATGT TCTATTGCTG GAAAGAATCT |
| 561 | ATATATGAGA TTCACCTGCA GCACTGGTGA TGCAATGGGG |
| 601 | ATGAACATGG TTTCCAAAGG GGTTCAAAAC GTTCTTGACT |
| 641 | TCCTTCAAAG TGATTTCCCT GACATGGATG TTATTGGCAT |
| 681 | CTCAGGAAAT TTTTGTTCGG ACAAGAAGCC AGCTGCTGTG |
| 721 | AACTGGATTC AAGGGCGAGG CAAATCGGTT GTTTGCGAGG |
| 761 | CAATTATCAA GGAAGAGGTG GTGAAGAAGG TATTGAAATC |
| 801 | AAGTGTTGCT TCACTAGTAG AGCTGAACAT GCTCAAGAAT |
| 841 | CTTACTGGTT CAGCTATTGC TGGAGCTCTT GGTGGATTCA |
| 881 | ATGCACATGC TGGCAACATA GTCTCTGCAA TTTTCATTGC |
| 921 | CACTGGCCAG GATCCAGCCC AGAATGTTGA GAGTTCTCAT |
| 961 | TGCATCACCA TGATGGAAGC TGTCAATGAT GGAAAAGATC |
| 1001 | TCCACATCTC TGTAACCATG CCTTCAATCG AGGTAGGAAC |
| 1041 | AGTTGGAGGA GGGACACAAC TAGCATCCCA ATCAGCATGT |
| 1081 | CTGAACCTAC TCGGTGTAAA AGGAGCAAGT AAAGAATCAC |
| 1121 | CAGGAGCAAA CTCAAGGCTC CTAGCCACAA TAGTAGCTGG |
| 1161 | TTCAGTCCTA GCTGGTGAAC TCTCCCTAAT GTCAGCCATA |
| 1201 | GCAGCAGGAC AACTAGTCCG GAGCCACATG AAGTACAACA |
| 1241 | GATCCAGCAA AGATGTAACC AAATTTGCAT CATCTTAA |

Another enzyme that is useful for making precursors for terpene/terpenoid production is a farnesyl diphosphate synthase, which makes precursors for the biosynthesis of essential isoprenoids like carotenoids, withanolides, ubiquinones, dolichols, sterols, among others. Farnesyl diphosphate synthase makes farnesyl diphosphate, shown below.

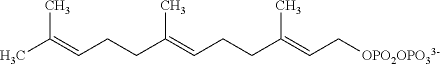

One example of a farnesyl diphosphate synthase that can be used is from Arabidopsis thaliana. An example of an Arabidopsis thaliana farnesyl diphosphate synthase sequence is shown below (accession AAB49290.1, SEQ ID NO:25).

| | |
|---|---|
| 1 | MSVSCCCRNL GKTIKKAIPS HHLHLRSLGG SLYRRRIQSS |
| 41 | SMETDLKSTF LNVYSVLKSD LLHDPSFEFT NESRLWVDRM |
| 81 | LDYNVRGGKL NRGLSVVDSF KLLKQGNDLT EQEVFLSCAL |
| 121 | GWCIEWLQAY FLVLDDIMDN SVTRRGQPCW FRVPQVGMVA |
| 161 | INDGILLRNH IHRILKKHFR DKPYYVDLVD LFNEVELQTA |
| 201 | CGQMIDLITT FEGEKDLAKY SLSIHRRIVQ YKTAYYSFYL |
| 241 | PVACALLMAG ENLENHIDVK NVLVDMGIYF QVQDDYLDCF |
| 281 | ADPETLGKIG TDIEDFKCSW LVVKATERCS EEQTKILYEN |
| 321 | YGKPDPSNVA KVKDLYKELD LEGVFMEYES KSYEKLTGAI |
| 361 | EGHQSKAIQA VLKSFLAKIY KRQK |

A nucleotide sequence encoding the Arabidopsis thaliana farnesyl diphosphate synthase with SEQ ID NO:25 is shown below as SEQ ID NO:26.

| | |
|---|---|
| 1 | GGCGTTTTCG GGAGAAGAAG GAGGAATATG AGTGTGAGTT |
| 41 | GTTGTTGTAG GAATCTGGGC AAGACAATAA AAAAGGCAAT |
| 81 | ACCTTCACAT CATTTGCATC TGAGAAGTCT TGGTGGGAGT |
| 121 | CTCTATCGTC GTCGTATCCA AAGCTCTTCA ATGGAGACCG |
| 161 | ATCTCAAGTC AACCTTTCTC AACGTTTATT CTGTTCTCAA |
| 201 | GTCTGACCTT CTTCATGACC CTTCCTTCGA ATTCACCAAT |
| 241 | GAATCTCGTC TCTGGGTTGA TCGGATGCTG GACTACAATG |
| 281 | TACGTGGAGG GAAACTCAAT CGGGGTCTCT CTGTTGTTGA |
| 321 | CAGTTTCAAA CTTTTGAAGC AAGGCAATGA TTTGACTGAG |
| 361 | CAAGAGGTTT TCCTCTCTTG TGCTCTCGGT TGGTGCATTG |
| 401 | AATGGCTCCA AGCTTATTTC CTTGTGCTTG ATGATATTAT |
| 441 | GGATAACTCT GTCACTCGCC GTGGTCAACC TTGCTGGTTC |
| 481 | AGAGTTCCTC AGGTTGGTAT GGTTGCCATC AATGATGGGA |
| 521 | TTCTACTTCG CAATCACATC CACAGGATTC TCAAAAAGCA |
| 561 | TTTCCGTGAT AAGCCTTACT ATGTTGACCT TGTTGATTTG |
| 601 | TTTAATGAGG TTGAGTTGCA AACAGCTTGT GGCCAGATGA |
| 641 | TAGATTTGAT CACCACCTTT GAAGGAGAAA AGGATTTGGC |
| 681 | CAAGTACTCA TTGTCAATCC ACCGTCGTAT TGTCCAGTAC |
| 721 | AAAACGGCTT ATTACTCATT TTATCTCCCT GTTGCTTGTG |
| 761 | CGTTGCTTAT GGCGGGCGAA AATTTGGAAA ACCATATTGA |
| 801 | CGTGAAAAAT GTTCTTGTTG ACATGGGAAT CTACTTCCAA |
| 841 | GTGCAGGATG ATTATCTGGA TTGTTTTGCT GATCCCGAGA |
| 881 | CGCTTGGCAA GATAGGAACA GATATAGAAG ATTTCAAATG |
| 921 | CTCGTGGTTG GTGGTTAAGG CATTAGAGCG CTGCAGCGAA |
| 961 | GAACAAACTA AGATATTATA TGAGAACTAT GGTAAACCCG |
| 1001 | ACCCATCGAA CGTTGCTAAA GTGAAGGATC TCTACAAAGA |
| 1041 | GCTGGATCTT GAGGGAGTTT TCATGGAGTA TGAGAGCAAA |
| 1081 | AGCTACGAGA AGCTGACTGG AGCGATTGAG GGACACCAAA |
| 1121 | GTAAAGCAAT CCAAGCAGTG CTAAAATCCT TCTTGGCTAA |
| 1161 | GATCTACAAG AGGCAGAAGT AGTAGAGACA GACAAACATA |
| 1201 | AGTCTCAGCC CTCAAAAATT TCCTGTTATG TCTTTGATTC |
| 1241 | TTGGTTGGTG ATTTGTGTAA TTCTGTTAAG TGCTCTGATT |
| 1281 | TTCAGGGGGA ATAATAAACC TGCCTCACTT TTATTCTTGT |
| 1321 | GTTACAATTG TATTTGTITC ATGACTATGA TCTTCTTCTT |
| 1361 | TCATCAGTTA TATGAATTTG AGATTCTTGT TGGTTG |

Another amino acid sequence for a full length cytosolic A. thaliana farnesyl diphosphate synthase (cytosol:AtFDPS, NM_117823.4); SEQ ID NO:27) is shown below.

| | |
|---|---|
| 1 | MADLKSTFLD VYSVLKSDLL QDPSFEFTHE SRQWLERMLD |
| 41 | YNVRGGKLNR GLSVVDSYKL LKQGQDLTEK ETFLSCALGW |
| 81 | CIEWLQAYFL VLDDIMDNSV TRRGQPCWFR KPKVGMIAIN |
| 121 | DGILLRNHIH RILKKHFREM PYYVDLVDLF NEVEFQTACG |
| 161 | QMIDLITTFD GEKDLSKYSL QIHRRIVEYK TAYYSFYLPV |
| 201 | ACALLMAGEN LENHTDVKTV LVDMGIYFQV QDDYLDCFAD |
| 241 | PETLGKIGTD IEDFKCSWLV VKALERCSEE QTKILYENYG |
| 281 | KAEPSNVAKV KALYKELDLE GAFMEYEKES YEKLTKLIEA |
| 321 | HQSKAIQAVL KSFLAKIYKR QK |

A nucleic acid sequence for a full-length cytosolic A. thaliana FDPS (cytosol:AtFDPS, NM_117823.4; SEQ ID NO:28) is shown below.

| | |
|---|---|
| 1 | CAATCAGGTT CCACATTTGG CTTTGCACAC CTTCCTTGAT |
| 41 | CCTATCAATG GCGGATCTGA AATCAACCTT CCTCGACGTT |
| 81 | TACTCTGTTC TCAAGTCTGA TCTGCTTCAA GATCCTTCCT |
| 121 | TTGAATTCAC CCACGAATCT CGTCAATGGC TTGAACGGAT |
| 161 | GCTTGACTAC AATGTACGCG GAGGGAAGCT AAATCGTGGT |
| 201 | CTCTCTGTGG TTGATAGCTA CAAGCTGTTG AAGCAAGGTC |
| 241 | AAGACTTGAC GGAGAAAGAG ACTTTCCTCT CATGTGCTCT |
| 281 | TGGTTGGTGC ATTGAATGGC TTCAAGCTTA TTTCCTTGTG |
| 321 | CTTGATGACA TCATGGACAA CTCTGTCACA CGCCGTGGCC |
| 361 | AGCCTTGTTG GTTTAGAAAG CCAAAGGTTG GTATGATTGC |
| 401 | CATTAACGAT GGGATTCTAC TTCGCAATCA TATCCACAGG |
| 441 | ATTCTCAAAA AGCACTTCAG GGAAATGCCT TACTATGTTG |
| 481 | ACCTCGTTGA TTTGTTTAAC GAGGTAGAGT TTCAAACAGC |
| 521 | TTGCGGCCAG ATGATTGATT TGATCACCAC CTTTGATGGA |
| 561 | GAAAAAGATT TGTCTAAGTA CTCCTTGCAA ATCCATCGGC |
| 601 | GTATTGTTGA GTACAAAACA GCTTATTACT CATTTTATCT |
| 641 | TCCTGTTGCT TGCGCATTGC TCATGGCGGG AGAAAATTTG |
| 681 | GAAAACCATA CTGATGTGAA GACTGTTCTT GTTGACATGG |
| 721 | GAATTTACTT TCAAGTACAG GATGATTATC TGGACTGTTT |
| 761 | TGCTGATCCT GAGACACTTG GCAAGATAGG GACAGACATA |
| 801 | GAAGATTTCA AATGCTCCTG GTTGGTAGTT AAGGCATTGG |
| 841 | AACGCTGCAG TGAAGAACAA ACTAAGATAC TATACGAGAA |
| 881 | CTATGGTAAA GCCGAACCAT CAAACGTTGC TAAGGTGAAA |
| 921 | GCTCTCTACA AAGAGCTTGA TCTCGAGGGA GCGTTCATGG |
| 961 | AATATGAGAA GGAAAGCTAT GAGAAGCTGA CAAAGTTGAT |
| 1001 | CGAAGCTCAC CAGAGTAAAG CAATTCAAGC AGTGCTAAAA |
| 1041 | TCTTTCTTGG CTAAGATCTA CAAGAGGCAG AAGTAGAGAC |
| 1081 | ATACTCGGGC CTCTCTCCGT TTTATTCTTC TGACATTTAT |
| 1121 | GTATTGGTGC ATGACTTCTT TTGCCTTAGA TCTTATGTTC |
| 1161 | CCTTCCGAAA ATAGAATTTG AGATTCTTGT TCATGCTTAT |
| 1201 | ACTATAGAGA CTTAGAAAAT GTCTATGTTT CTTTTAATTT |
| 1241 | CTGAATAAAA AATGTGCAAT CAGTGATAAA TTGATACTTG |
| 1281 | TTAATGTGGC AAAAATTTTG TGTCACATGA GGGTGCAACA |
| 1321 | GAAATTTGGA AGGACCTGAG GCTGTTTGAG CT |

A variety of enzymes can be used in the methods described herein including enzymes that can synthesize terpene precursors, monoterpenes, diterpenes, triterpenes, sesquiterpenes, and combinations thereof. The terpene synthases can be monoterpene synthases, diterpene synthases, sesquiterpene synthases, sesterterpene synthases, triterpene synthases, tetraterpene synthases, polyterpene synthases, or combinations thereof. Such terpene synthases can be fused to LDSP polypeptides.

For example, one enzyme that can be fused LDSP is an Abies grandis abietadiene synthase enzyme (EC 4.2.3.18), which is an enzyme that catalyzes the conversion of GGDP via CPP, a carbocation, and tertiary allylic alcohol to form a mixture of four products, where abietadiene is the main product.

An amino acid sequence for an A. grandis abietadiene synthase (U50768.1) is shown below as SEQ ID NO:31.

| | |
|---|---|
| 1 | MAMPSSSLSS QIPTAAHHLT ANAQSIPHFS TTLNAGSSAS |
| 41 | KRRSLYLRWG KGSNKIIACV GEGGATSVPY QSAEKNDSLS |
| 81 | SSTLVKREFP PGFWKDDLID SLTSSHKVAA SDEKRIETLI |
| 121 | SEIKNMFRCM GYGETNPSAY DTAWVARIPA VDGSDNPHFP |
| 161 | ETVEWILQNQ LKDGSWGEGF YFLAYDRILA TLACIITLTL |
| 201 | WRTGETQVQK GIEFFRTQAG KMEDFADSHR PSGFEIVFPA |
| 241 | MLKEAKILGL DLPYDLPFLK QIIEKREAKL KRIPTDVLYA |
| 281 | LPTTLLYSLE GLQEIVDWQR IMKLQSKDGS FLSSPASTAA |
| 321 | VFMRTGNKKC LDFLNFVLKK FGNHVPCHYP LDLFERLWAV |
| 361 | DTVERLGIDR HFKEEIKEAL DYVYSHWDER GIGWARENPV |
| 401 | PDIDDTAMGL RILRLHGYHV SSDVLKTFRD ENGEFFCFLG |
| 441 | QTQRGVTDML NVNRCSHVSF PGETIMEEAK LCTERYLRNA |
| 481 | LENVDAFDKW AFKKNIRGEV EYALKYPWHK SMPRLEARSY |
| 521 | IENYGPDDVW LGKTVYMMPY ISNEKYLELA KLDFNKVQSI |
| 561 | HQTELQDLRR WWKSSGFTDL NFTRERVTEI YFSPASFIFE |
| 601 | PEFSKCREVY TKTSNFTVIL DDLYDAHGSL DDLKLFTESV |
| 641 | KRWDLSLVDQ MPQQMKICFV GFYNTFNDIA KEGRERQGRD |
| 681 | VLGYIQNVWK VQLEAYTKEA EWSEAKYVPS FNEYIENASV |
| 721 | SIALGTVVLI SALFTGEVLT DEVLSKIDRE SRFLQLMGLT |
| 761 | GRLVNDTKTY QAERGQGEVA SAIQCYMKDH PKISEEEALQ |
| 801 | HVYSVMENAL EELNREFVNN KIPDIYKRLV FETARIMQLF |
| 841 | YMQGDGLTLS HDMEIKEHVK NCLFQPVA |

A nucleic acid sequence for the A. grandis abietadiene synthase (U50768.1; SEQ ID NO:31) is shown below as SEQ ID NO:32.

| | |
|---|---|
| 1 | AGATGGGCAT GCCTTCCTCT TCATTGTCAT CACAGATTCC |
| 41 | CACTGCTGCT CATCATCTAA CTGCTAACGC ACAATCCATT |
| 81 | CCGCATTTCT CCACGACGCT GAATGCTGGA AGCAGTGCTA |
| 121 | GCAAACGGAG AAGCTTGTAC CTACGATGGG GTAAAGGTTC |
| 161 | AAACAAGATC ATTGCCTGTG TTGGAGAAGG TGGTGCAACC |
| 201 | TCTGTTCCTT ATCAGTCTGC TGAAAAGAAT GATTCGCTTT |
| 241 | CTTCTTCTAC ATTGGTGAAA CGAGAATTTC CTCCAGGATT |
| 281 | TTGGAAGGAT GATCTTATCG ATTCTCTAAC GTCATCTCAC |
| 321 | AAGGTTGCAG CATCAGACGA GAAGCGTATC GAGACATTAA |
| 361 | TATCCGAGAT TAAGAATATG TTTAGATGTA TGGGCTATGG |
| 401 | CGAAACGAAT CCCTCTGCAT ATGACACTGC TTGGGTAGCA |
| 441 | AGGATTCCAG CAGTTGATGG CTCTGACAAC CCTCACTTTC |
| 481 | CTGAGACGGT TGAATGGATT CTTCAAAATC AGTTGAAAGA |
| 521 | TGGGTCTTGG GGTGAAGGAT TCTACTTCTT GGCATATGAC |
| 561 | AGAATACTGG CTACACTTGC ATGTATTATT ACCCTTACCC |
| 601 | TCTCGCGTAC TGGGGAGACA CAAGTACAGA AAGGTATTGA |
| 641 | ATTCTTCAGG ACACAAGCTG GAAAGATGGA AGATGAAGCT |
| 681 | GATAGTCATA GGCCAAGTGG ATTTGAAATA GTATTTCCTG |
| 721 | CAATGCTAAA GGAAGCTAAA ATCTTAGGCT TGGATCTGCC |
| 761 | TTACGATTTG CCATTCCTGA AACAAATCAT CGAAAAGCGG |
| 801 | GAGGCTAAGC TTAAAAGGAT TCCCACTGAT GTTCTCTATG |
| 841 | CCCTTCCAAC AACGTTATTG TATTCTTTGG AAGGTTTACA |
| 881 | AGAAATAGTA GACTGGCAGA AAATAATGAA ACTTCAATCC |
| 921 | AAGGATGGAT CATTTCTCAG CTCTCCGGCA TCTACAGCGG |
| 961 | CTGTATTCAT GCGTACAGGG AACAAAAAGT GCTTGGATTT |
| 1001 | CTTGAACTTT GTCTTGAAGA AATTCGGAAA CCATGTGCCT |
| 1041 | TGTCACTATC CGCTTGATCT ATTTGAACGT TTGTGGGCGG |
| 1081 | TTGATACAGT TGAGCGGCTA GGTATCGATC GTCATTTCAA |
| 1121 | AGAGGAGATC AAGGAAGCAT TGGATTATGT TTACAGCCAT |
| 1161 | TGGGACGAAA GAGGCATTGG ATGGGCGAGA GAGAATCCTG |
| 1201 | TTCCTGATAT TGATGATACA GCCATGGGCC TTCGAATCTT |
| 1241 | GAGATTACAT GGATACAATG TATCCTCAGA TGTTTTAAAA |
| 1281 | ACATTTAGAG ATGAGAATGG GGAGTTCTTT TGCTTCTTGG |
| 1321 | GTCAAACACA GAGAGGAGTT ACAGACATGT TAAACGTCAA |
| 1361 | TCGTTGTTCA CATGTTTCAT TTCCGGGAGA AACGATCATG |
| 1401 | GAAGAAGCAA AACTCTGTAC CGAAAGGTAT CTGAGGAATG |
| 1441 | CTCTGGAAAA TGTGGATGCC TTTGACAAAT GGGCTTTTAA |
| 1481 | AAAGAATATT CGGGGAGAGG TAGAGTATGC ACTCAAATAT |
| 1521 | CCCTGGCATA AGAGTATGCC AAGGTTGGAG GCTAGAAGCT |
| 1561 | ATATTGAAAA CTATGGGCCA GATGATGTGT GGCTTGGAAA |
| 1601 | AACTGTATAT ATGATGCCAT ACATTTCGAA TGAAAAGTAT |
| 1641 | TTAGAACTAG CGAAACTGGA CTTCAATAAG GTGCAGTCTA |
| 1681 | TACACCAAAC AGAGCTTCAA GATCTTCGAA GGTGGTGGAA |
| 1721 | ATCATCCGGT TTCACGGATC TGAATTTCAC TCGTGAGCGT |
| 1761 | GTGACGGAAA TATATTTCTC ACCGGCATCC TTTATCTTTG |
| 1801 | AGCCCGAGTT TTCTAAGTGC AGAGAGGTTT ATACAAAAAC |
| 1841 | TTCCAATTTC ACTGTTATTT TAGATGATCT TTATGACGCC |
| 1881 | CATGGATCTT TAGACGATCT TAAGTTGTTC ACAGAATCAG |
| 1921 | TCAAAAGATG GGATCTATCA CTAGTGGACC AAATGCCACA |
| 1961 | ACAAATGAAA ATATGTTTTG TGGGTTTCTA CAATACTTTT |
| 2001 | AATGATATAG CAAAAGAAGG ACGTGAGAGG CAAGGGCGCG |
| 2041 | ATGTGCTAGG CTACATTCAA AATGTTTGGA AAGTCCAACT |
| 2081 | TGAAGCTTAC ACGAAAGAAG CAGAATGGTC TGAAGCTAAA |
| 2121 | TATGTGCCAT CCTTCAATGA ATACATAGAG AATGCGAGTC |
| 2161 | TGTCAATAGC ATTGGGAACA GTCGTTCTCA TTAGTGCTCT |
| 2201 | TTTCACTGGG GAGGTTCTTA CAGATGAAGT ACTCTCCAAA |
| 2241 | ATTGATCGCG AATCTAGATT TCTTCAACTC ATGGGCTTAA |
| 2281 | CAGGGCGTTT GGTGAATGAC ACCAAAACTT ATCAGGCAGA |
| 2321 | GAGAGGTCAA GGTGAGGTGG CTTCTGCCAT ACAATGTTAT |
| 2361 | ATGAAGGACC ATCCTAAAAT CTCTGAAGAA GAAGCTCTAC |
| 2401 | AACATGTCTA TAGTGTCATG GAAAATGCCC TCGAAGAGTT |
| 2441 | GAATAGGGAG TTTGTGAATA ACAAAATACC GGATATTTAC |
| 2481 | AAAAGACTGG TTTTTGAAAC TGCAAGAATA ATGCAACTCT |
| 2521 | TTTATATGCA AGGGGATGGT TTGACACTAT CACATGATAT |
| 2561 | GGAAATTAAA GAGCATGTCA AAAATTGCCT CTTCCAACCA |
| 2601 | GTTGCCTAGA TTAAATTATT CAGTTAAAGG CCCTCATGGT |
| 2641 | ATTGTGTTAA CATTATAATA ACAGATGCTC AAAAGCTTTG |
| 2681 | AGCGGTATTT GTTAAGGCTA TCTTTGTTTG TTTGTTTGTT |
| 2721 | TACTGCCAAC CAAAAAGCGT TCCTAAACCT TTGAAGACAT |
| 2761 | TTCCATCCAA GAGATGGAGT CTACATTTTA TTTATGAGAT |
| 2801 | TGAATTATTT CAAGAGAATA TACTACATAT ATTTAAAAGT |
| 2841 | AAAAAAAAAA AAAAAAAAAA A |

However, a truncated Abies grandis abietadiene synthase enzyme that is missing the first 84 amino acids (AgABS⁸⁵⁻⁸⁶⁸) can be used for cytosolic expression of the enzyme (cytosol:AgABS⁸⁵⁻⁸⁶⁸). A sequence for this cytosol:AgABS⁸⁵⁻⁸⁶⁸ enzyme is shown below as SEQ ID NO:33.

VKREFPPGFW KDDLIDSLTS SHKVAASDEK RIETLISEIK
NMFRCMGYGE TNPSAYDTAW VARIPAVDGS DNPHFPETVE
WILQNQLKDG SWGEGFYFLA YDRILATLAC IITLTLWRTG
ETQVQKGIEF FRTQAGKMED EADSHRPSGF EIVFPAMLKE
AKILGLDLPY DLPFLKQIIE KREAKLKRIP TDVLYALPTT
LLYSLEGLQE IVDWQKIMKL QSKDGSFLSS PASTAAVFMR
TGNKKCLDFL NFVLKKFGNH VPCHYPLDLF ERLWAVDTVE
RLGIDRHFKE EIKEALDYVY SHWDERGIGW ARENPVPDID
DTAMGLRILR LHGYNVSSDV LKTFRDENGE FFCFLGQTQR
GVTDMLNVNR CSHVSFPGET IMEEAKICTE RYLRNALENV
DAFDKWAFKK NIRGEVEYAL KYPWHKSMPR LEARSYIENY
GPDDVWLGKT VYMMPYISNE KYLELAKLDF NKVQSIHQTE
LQDLRRWWKS SGFTDLNFTR ERVTEIYFSP ASFIFEPEFS
KCREVYTKTS NFTVILDDLY DAHGSLDDLK LFTESVKRWD
LSLVDQMPQQ MKICFVGFYN TFNDIAKEGR ERQGRDVLGY
IQNVWKVQLE AYTKEAEWSE AKYVPSFNEY IENASVSIAL
GTVVLISALF TGEVLTDEVL SKIDRESRFL QLMGLTGRLV
NDTKTYQAER GQGEVASAIQ CYMKDHPKIS EEEALQHVYS
VMENALEELN REFVNNKIPD IYKRIVFETA RIMQLFYMQG
DGLTLSHDME IKEHVKNCLF QPVA

A nucleotide sequence for this cytosol:AgABS⁸⁵⁻⁸⁶⁸ enzyme with SEQ ID NO:33 is shown below as SEQ ID NO:34.

GTGAAACGAG AATTTCCTCC AGGATTTTGG AAGGATGATC
TTATCGATTC TCTAACGTCA TCTCACAAGG TTGCAGCATC
AGACGAGAAG CGTATCGAGA CATTAATATC CGAGATTAAG
AATATGTTTA GATGTATGGG CTATGGCGAA ACGAATCCCT
CTGCATATGA CACTGCTTGG GTAGCAAGGA TTCCAGCAGT
TGATGGCTCT GACAACCCTC ACTTTCCTGA GACGGTTGAA
TGGATTCTTC AAAATCAGTT GAAAGATGGG TCTTGGGGTG
AAGGATTCTA CTTCTTGGCA TATGACAGAA TACTGGCTAC
ACTTGCATGT ATTATTACCC TTACCCTCTG GCGTACTGGG
GAGACACAAG TACAGAAAGG TATTGAATTC TTCAGGACAC
AAGCTGGAAA GATGGAAGAT GAAGCTGATA GTCATAGGCC
AAGTGGATTT GAAATAGTAT TTCCTGCAAT GCTAAAGGAA
GCTAAAATCT TAGGCTTGGA TCTGCCTTAC GATTTGCCAT
TCCTGAAACA AATCATCGAA AAGCGGGAGG CTAAGCTTAA
AAGGATTCCC ACTGATGTTC TCTATGCCCT TCCAACAACG
TTATTGTATT CTTTGGAAGG TTTACAAGAA ATAGTAGACT
GGCAGAAAAT AATGAAACTT CAATCCAAGG ATGGATCATT
TCTCAGCTCT CCGGCATCTA CAGCGGCTGT ATTCATGCGT
ACAGGGAACA AAAAGTGCTT GGATTTCTTG AACTTTGTCT
TGAAGAAATT CGGAAACCAT GTGCCTTGTC ACTATCCGCT
TGATCTATTT GAACGTTTGT GGGCGGTTGA TACAGTTGAG
CGGCTAGGTA TCGATCGTCA TTTCAAAGAG GAGATCAAGG
AAGCATTGGA TTATGTTTAC AGCCATTGGG ACGAAAGAGG
CATTGGATGG GCGAGAGAGA ATCCTGTTCC TGATATTGAT
GATACAGCCA TGGGCCTTCG AATCTTGAGA TTACATGGAT
ACAATGTATC CTCAGATGTT TTAAAAACAT TTAGAGATGA
GAATGGGGAG TTCTTTTGCT TCTTGGGTCA AACACAGAGA
GGAGTTACAG ACATGTTAAA CGTCAATCGT TGTTCACATG
TTTCATTTCC GGGAGAAACG ATCATGGAAG AAGCAAAACT
CTGTACCGAA AGGTATCTGA GGAATGCTCT GGAAAATGTG
GATGCCTTTG ACAAATGGGC TTTTAAAAAG AATATTCGGG
GAGAGGTAGA GTATGCACTC AAATATCCCT GGCATAAGAG
TATGCCAAGG TTGGAGGCTA GAAGCTATAT TGAAAACTAT
GGGCCAGATG ATGTGTGGCT TGGAAAAACT GTATATATGA
TGCCATACAT TTCGAATGAA AAGTATTTAG AACTAGCGAA
ACTGGACTTC AATAAGGTGC AGTCTATACA CCAAACAGAG
CTTCAAGATC TTCGAAGGTG GTGGAAATCA TCCGGTTTCA
CGGATCTGAA TTTCACTCGT GAGCGTGTGA CGGAAATATA
TTTCTCACCG GCATCCTTTA TCTTTGAGCC CGACTTTTCT
AAGTGCAGAG AGGTTTATAC AAAAACTTCC AATTTCACTG
TTATTTTAGA TGATCTTTAT GACGCCCATG GATCTTTAGA
CGATCTTAAG TTGTTCACAG AATCAGTCAA AAGATGGGAT
CTATCACTAG TGGACCAAAT GCCACAACAA ATGAAAATAT
GTTTTGTGGG TTTCTACAAT ACTTTTAATG ATATAGCAAA
AGAAGGACGT GAGAGGCAAG GGCGCGATGT GCTAGGCTAC
ATTCAAAATG TTTGGAAAGT CCAACTTGAA GCTTACACGA
AAGAAGCAGA ATGGTCTGAA GCTAAATATG TGCCATCCTT
CAATGAATAC ATAGAGAATG CGAGTGTGTC AATAGCATTG
GGAACAGTCG TTCTCATTAG TGCTCTTTTC ACTGGGGAGG
TTCTTACAGA TGAAGTACTC TCCAAAATTG ATCGCGAATC
TAGATTTCTT CAACTCATGG GCTTAACAGG GCGTTTGGTG
AATGACACCA AAACTTATCA GGCAGAGAGA GGTCAAGGTG
AGGTGGCTTC TGCCATACAA TGTTATATGA AGGACCATCC
TAAAATCTCT CAAGAAGAAG CTCTACAACA TGTCTATAGT
GTCATGGAAA ATGCCCTCGA AGAGTTGAAT AGGGAGTTTG
TGAATAACAA AATACCGGAT ATTTACAAAA GACTGGTTTT
TGAAACTGCA AGAATAATGC AACTCTTTTA TATGCAAGGG
GATGGTTTGA CACTATCACA TGATATGGAA ATTAAAGAGC
ATGTCAAAAA TTGCCTCTTC CAACCAGTTG CC

Another enzyme that can be used in the methods is a cytochrome P450 (CYP720B4) enzyme, which can convert abietadiene and several isomers to the corresponding diterpene resin acids. One example of a cytochrome P450 that can be used is a Picea sitchensis CYP720B4, which is expressed in the endoplasmic reticulum (ER:PsCYP720B4). Such a Picea sitchensis CYP720B4, for example, can have accession number HM245403.1 and the following amino acid sequence SEQ ID NO:35.

| | |
|---|---|
| 1 | MAPMADQISL LLVVFTVAVA LLHLIHRWWN IQRGPKMSNK |
| 41 | EVHLPPGSTG WPLIGETFSY YRSMTSNHPR KFIDDREKRY |
| 81 | DSDIFISHLF GGRTVVSADP QFNKFVLQNE GRFFQAQYPK |
| 121 | ALKALIGNYG LLSVHGDLQR KLHGIAVNLL RFERLKVDFM |
| 161 | EEIQNLVHST LDRWADMKEI SLQNECHQMV LNLMAKQLLD |
| 201 | LSPSKETSDI CELFVDYTNA VIAIPIKIPG STYAKGLKAR |
| 241 | ELLIKKISEM IKERRNHPEV VHNDLLTKLV EEGLISDEII |
| 281 | CDFILFLLFA GHETSSRAMT FAIKFLTYCP KALKQMKEEH |
| 321 | DAILKSKGGH KKLNWDDYKS MAFTQCVINE TLRLGNFGPG |
| 361 | VFREAKEDTK VKDCLIPKGW VVFAFLTATH LHEKEHNEAL |
| 401 | TFNPWRWQLD KDVPDDSLFS PFGGGARLCP GSHLAKLELS |
| 441 | LELHIFITRF SWEARADDRT SYFPLPYLTK GFPISLHGRV |
| 481 | ENE |

This endoplasmic Picea sitchensis CYP720B4 (PsCYP720B4, HM245403.1; SEQ ID NO:35) can be encoded by the following cDNA sequence (SEQ ID NO:36).

| | |
|---|---|
| 1 | ATGGCGCCCA TGGCAGACCA |
| | AATATCATTA CTGTTGGTGG |
| 41 | TGTTCACGGT AGCGGTGGCG |
| | CTCCTCCACC TTATTCACAG |
| 81 | GTGGTGGAAT ATCCAGAGAG |
| | GCCCAAAAAT GAGTAATAAG |
| 121 | GAGGTTCATC TGCCTCCTGG |
| | GTCGACTGGA TGGCCGCTTA |
| 161 | TTGGCGAAAC CTTCAGTTAT |
| | TATCGCTCCA TGACCAGCAA |
| 201 | TCATCCCAGG AAATTCATCG |
| | ACGACAGAGA GAAAAGATAT |
| 241 | GATTCCGACA TTTTCATATC |
| | TCATCTATTT CGAGGCCGCA |
| 281 | CGGTTGTATC AGCGGATCCC |
| | CAGTTCAACA AGTTTGTTCT |
| 321 | ACAAAACCAC GGGAGATTCT |
| | TTCAAGCCCA ATACCCAAAC |
| 361 | GCACTGAAGG CTTTCATAGG |
| | CAACTACCGG CTCCTCTCTC |
| 401 | TGCATCGAGA TCTCCAGAGA |
| | AACCTCCACG CAATACCTCT |
| 441 | GAATTTCCTG AGGTTTGAGA |
| | GACTGAAAGT CGATTTCATG |
| 481 | CACGAGATAC AGAATCTCGT |
| | GCACTCCACG TTGGATAGAT |
| 521 | GCCCAGATAT CAAGGAAATT |
| | TCTCTGCAGA ATGAATGTCA |
| 561 | CCAGATGGTT CTCAACTTGA |
| | TGGCCAAACA ACTGCTGGAT |
| 601 | TTATCTCCTT CCAAAGAGAC |
| | GAGTGATATT TGCGAGCTAT |
| 641 | TCGTTGACTA TACCAATGCA |
| | GTGATTGCCA TTCCCATCAA |
| 681 | AATCCCAGGT TCCACCTATG |
| | CAAAGGGGCT TAAGGCAAGG |
| 721 | GAGCTTCTCA TAAAAAAGAT |
| | TTCAGAAATG ATAAAAGAGA |
| 761 | GAAGGAATCA TCCTGAAGTT |
| | GTTCATAATG ATTTGTTAAC |
| 801 | TAAACTTCTC GAAGAGGGCC |
| | TCATTTCAGA TGAAATTATT |
| 841 | TGTGATTTTA TTTTATTTTT |
| | ACTTTTTGCT GGACATGAGA |
| 881 | CTTCCTCTAG AGCCATGACA |
| | TTTGCTATCA AGTTTCTTAC |
| 921 | CTATTGCCCC AAGGCATTGA |
| | AGCAAATCAA GGAAGACCAT |
| 961 | GATGCTATAT TAAAATCAAA |
| | GGGAGGTCAT AAGAAACTTA |
| 1001 | ATTGGGATGA CTACAAATCA |
| | ATGGCATTCA CTCAATGTGT |
| 1041 | TATAAATGAA ACACTTCGAT |
| | TAGGTAACTT TGGTCCAGGG |
| 1081 | GTGTTTAGAG AAGCTAAAGA |
| | AGACACTAAA GTAAAAGATT |
| 1121 | GTCTCATTCC AAAAGGATGG |
| | GTGGTATTTG CTTTTCTGAC |
| 1161 | TGCAACACAT CTACATGAAA |
| | AGTTTCATAA TGAAGCTCTT |
| 1201 | ACTTTTAACC CATGGCGATG |
| | GCAATTGGAT AAAGATGTAC |
| 1241 | CAGATGATAG TTTGTTTTCA |
| | CCTTTTGGAG GTGGAGCTAG |
| 1281 | GCTTTGTCCA GGATCTCATC |
| | TAGCTAAACT TGAATTGTCA |
| 1321 | CTTTTTCTTC ACATATTTAT |
| | CACAAGATTC AGTTGGGAAG |
| 1361 | CGCCTGCAGA TGATCGTACC |
| | TCATATTTTC CATTACCTTA |
| 1401 | TTTAACTAAA GGCTTTCCCA |
| | TTAGCCTTCA TGCTAGAGTA |
| 1441 | GAGAATGAAT AA |

To target terpenoid synthesis to the lipid droplets, a truncated CYP720B4 lacking the membrane-binding domain was produced that is missing amino acids 1-29 and that is expressed in the cytosol (cytosol:CYP720B4(30-483)). This truncated CYP720B4 can be a fusion partner with LDSP. A sequence for such a truncated Picea sitchensis CYP720B4 is shown below as SEQ ID NO:37.

NIQRGPKMSN KEVHLPPGST GWPLIGETFS YYRSMTSNHP
RKFIDDREKR YDSDIFISHL FGGRTVVSAD PQFNKFVLQN
EGRFFQAQYP KALKALIGNY GLLSVHGDLQ RKLHGIAVNL
LRFERLKVDF MEEIQNLVHS TLDRWADMKE ISLQNECHQM
VLNLMAKQLL DLSPSKETSD ICELFVDYTN AVIAIPIKIP
GSTYAKGLKA RELLIKKISE MIKERRNHPE VVHNDLLTKL
VEEGLISDEI ICDFILFLLF AGHETSSRAM TFAIKFLTYC
PKALKQMKEE HDAILKSKGG HKKLNWDDYK SMAFTQCVIN
ETLRLGNFGP GVFREAKEDT KVKDCLIPKG WVVFAFLTAT
HLHEKFHNEA LTFNPWRWQL DKDVPDDSLF SPFGGGARLC
PGSHLAKLEL SLFLHIFITR FSWEARADDR TSYFPLPYLT
KCFPISLHCR VENE

This truncated PsCYP720B4(30-483) polypeptide can have a methionine at its N-terminus. This truncated cytosolic Picea sitchensis CYP720B4 (PsCYP720B4) can be encoded by the following cDNA sequence (SEQ ID NO:38).

AATATCCAGA GAGGCCCAAA AATGACTAAT AACCAGGTTC
ATCTGCCTCC TGGGTCGACT GGATGGCCGC TTATTGCCGA
AACCTTCAGT TATTATCGCT CCATGACCAG CAATCATCCC
AGGAAATTCA TCGACGACAG AGAGAAAAGA TATGATTCGG
ACATTTTCAT ATCTCATCTA TTTGGAGGCC GGACGGTTGT
ATCAGCGGAT CCCCAGTTCA ACAAGTTTGT TCTACAAAAC
GAGGGGAGAT TCTTTCAAGC CCAATACCCA AAGGCACTGA
AGGCTTTGAT AGGCAACTAC GGGCTGCTCT CTGTGCATGG
AGATCTCCAG AGAAAGCTCC ACGGAATAGC TGTGAATTTG
CTGAGGTTTG AGAGACTGAA AGTCGATTTC ATGGAGGAGA
TACAGAATCT CGTGCACTCC ACGTTGGATA GATGGGCAGA
TATGAAGGAA ATTTCTCTGC AGAATGAATG TCACCAGATG
GTTCTCAACT TGATGGCCAA ACAACTGCTG GATTTATCTC
CTTCCAAAGA GACGAGTGAT ATTTGCGAGC TATTCGTTGA
CTATACCAAT GCAGTGATTG CCATTCCCAT CAAAATCCCA
GGTTCCACCT ATGCAAAGGG GCTTAAGGCA AGGGAGCTTC
TCATAAAAAA GATTTCAGAA ATGATAAAAG AGAGAAGGAA
TCATCCTGAA GTTGTTCATA ATGATTTGTT AACTAAACTT
GTGGAAGAGG GGCTCATTTC AGATGAAATT ATTTGTGATT
TTATTTTATT TTTACTTTTT GCTGGACATG AGACTTCCTC
TAGAGCCATG ACATTTGCTA TCAAGTTTCT TACCTATTGC
CCCAAGGCAT TGAAGCAAAT CAAGCAACAG CATGATGCTA
TATTAAAATC AAAGGGAGGT CATAAGAAAC TTAATTGGGA
TGACTACAAA TCAATGGCAT TCACTCAATG TGTTATAAAT
GAAACACTTC GATTAGGTAA CTTTGGTCCA GGGGTGTTTA
GAGAAGCTAA AGAAGACACT AAAGTAAAAG ATTGTCTCAT
TCCAAAAGGA TGGGTGGTAT TTGCTTTTCT GACTGCAACA
CATCTACATG AAAAGTTTCA TAATGAAGCT CTTACTTTTA
ACCCATGGCG ATGGCAATTG GATAAAGATG TACCAGATGA
TAGTTTCTTT TCACCTTTTG GAGGTGGAGC TAGGCTTTGT
CCAGGATCTC ATCTAGCTAA ACTTGAATTG TCACTTTTTC
TTCACATATT TATCACAAGA TTCAGTTGGG AAGCGCGTGC
AGATGATCGT ACCTCATATT TTCCATTACC TTATTTAACT
AAAGGCTTTC CCATTAGCCT TCATGGTAGA GTAGAGAATG
AATAA

This cDNA with SEQ ID NO:38, which encodes a truncated Picea sitchensis CYP720B4 (PsCYP720B4), can have an ATG at the 5′ end.

To facilitate the catalytic activity of the cytochrome P450, a cytochrome P450 reductase can also be expressed. One example of a cytochrome P450 reductase that can be used is a Camptotheca acuminata cytochrome P450 reductase (CaCPR), for example with accession number KP162177.1 and the following amino acid sequence (SEQ ID NO:39.

| | |
|---|---|
| 1 | MQSSSVKVST FDLMSAILRG |
| | RSMDQTNVSF ESGESPALAM |
| 41 | LIENRELVMI LTTSVAVLIG |
| | CFVVLLWRRS SGKSGKVTEP |
| 81 | PKPLHVKTEP EPEVDDGKKK |
| | VSIFYGTQTG TAEGFAKALA |
| 121 | EEAKVRYEKA SFKVIDLDDY |
| | AADDEEYEEK LKKETLTFFF |
| 161 | LATYGDGEPT DNAARFYKWF |
| | MEGKERGDWL KNLHYGVFGL |
| 201 | GNRQYEHFNR IAKVVDDTIA |
| | EQGGKRLIPV GLGDDDQCIE |
| 241 | DDFAAWRELL WPELDQLLQD |
| | EDGTTVATPY TAAVLEYRVV |
| 281 | FHDSPDASLL DKSFSKSNGH |
| | AVHDAQHPCR ANVAVRRELH |
| 321 | TPASDRSCTH LEFDISGTGL |
| | VYETGDHVGV YCENLIEVVE |
| 361 | EAEMLLGLSP DTFFSIHTDK |
| | EDGTPLSGSS LPPPFPPCTL |
| 401 | RRALTQYADL LSSPKKSSLL |
| | ALAAHCSDPS EADRLRHLAS |
| 441 | PSGKDEYAQW VVASQRSLLE |
| | VMAEFPSAKP PIGAFFAGVA |
| 481 | PRLQPRYYSI SSSPRKAPSR |
| | IHVTCALVFE KTPVGRIHKG |
| 521 | VCSTWMKNAV PLDESRDCSW |
| | APIFVRQSNF KLPADTKVPV |
| 561 | LKIGPGTGLA PFRGFLQERL |
| | ALKEAGAELG PAILFFGCRN |
| 601 | RQMDYIYEDE LNNFVETGAL |
| | SELIVAFSRE GPKKEYVQHK |
| 641 | MMEKASDIWN MISQEGYIYV |
| | CGDAKGMARD VHRTLHTIVQ |
| 681 | EQGSLDSSKT ESMVKNLQMN |
| | GRYLRDVW |

A nucleotide sequence that encodes the Camptotheca acuminata cytochrome P450 reductase with SEQ ID NO:39 is shown below as SEQ ID NO:40.

| | |
|---|---|
| 1 | AGTCTCTGCA ACCATAACCA |
| | TAACCAGAAC CAGAACCAGG |
| 41 | AAGCCAGAGG CTCTCTTTTC |
| | TTTCTCTCTC TCTCATTACC |
| 81 | AATTCTCCGG TAATTTTCTA |
| | GCCGGCCACA GGACCTTTAT |
| 121 | TTTTTTCCCG GTAACATGCA |
| | ATCCACTTCG GTTAACCTCT |
| 161 | CGACGTTTGA TTTGATGTCA |
| | GCGATTTTGA GGCCGAGGAG |
| 201 | TATGGATCAC ACCAACCTCT |
| | CGTTCGAATC CGGCGAGTCT |
| 241 | CCCGCGTTGC CCATGTTCAT |
| | CCAGAATCCG GACCTGGTGA |
| 281 | TGATCCTGAC GACGTCTGTG |
| | GCGGTGTTGA TAGGGTGTTT |
| 321 | TGTAGTGTTG TTCTGGCGGA |
| | GATCGTCAGG AAAGTCCGGG |
| 361 | AAACTGACAC AACCTCCGAA |
| | GCCGCTGATC CTGAAGACTG |
| 401 | AGCCGGAGCC CGAAGTTGAT |
| | GACCGCAAGA AGAAGGTTTC |
| 441 | TATCTTCTAT GGCACGCAGA |
| | CCGGTACCGC CGAAGGTTTC |
| 481 | GCAAAGGCAC TCGCCGAGGA |
| | AGCAAAAGTG AGATACGAAA |
| 521 | AGGCGTCATT TAAAGTGATA |
| | GATTTGGATG ATTATGCCGC |
| 561 | CGACGATGAA GAATACGAAG |
| | AGAAATTGAA GAAAGAAACT |
| 601 | TTAACATTTT TCTTCTTAGC |
| | TACATACGGA GATCGAGAAC |
| 641 | CAACTGACAA TGCCGCCAGA |
| | TTCTACAAAT GGTTTATGGA |
| 681 | CGCAAAACAC ACACGCGACT |
| | GCCTTAAGAA TCTCCATTAC |
| 721 | GGAGTATTTG GTCTCCGCAA |
| | CAGGCAGTAT GAGCATTTCA |
| 761 | ACAGCATTGC AAACGTGCTG |
| | GATGATACCA TTCCCGACCA |
| 801 | GCGTGGCAAG CGCCTCATTC |
| | CTCTGCGCCT TGGAGATGAT |
| 341 | CATCAATCCA TTGAACATGA |
| | TTTTCCTGCA TGCCCGGAGT |
| 881 | TATTGTGGCC CGAGTTGGAT |
| | CAGTTGCTTC AAGATGAAGA |
| 921 | TGGCACAACT GTTGCTACTC |
| | CTTACACTGC CGCTGTATTG |
| 961 | GAATATCGTG TTGTATTCCA |
| | TGACAGCCCA GATGCATCAT |
| 1001 | TACTGGACAA GAGCTTCAGT |
| | AAGTCAAATG GTCATGCTGT |
| 1041 | TCATGATGCT CAACATCCAT |
| | GCAGAGCTAA CGTGGCTGTG |
| 1081 | AGAAGGGAGC TTCACACTCC |
| | CGCATCTGAT CGTTCTTGCA |
| 1121 | CTCATCTGGA ATTTGATATT |
| | TCTGGCACTG GACTTGTATA |
| 1161 | TGAAACTGGG GACCATGTTG |
| | GTGTGTATTG TGAGAATTTA |
| 1201 | ATTGAAGTTG TGGAGGAGGC |
| | AGAAATGTTA TTAGGTTTAT |
| 1241 | CACCAGATAC CTTTTTCTCC |
| | ATTCACACTG ATAAGGAGGA |
| 1281 | TGGCACACCA CTTAGTGGAA |
| | GCTCCTTGCC ACCTCCTTTC |
| 1321 | CCCCCCTCTA CTTTAAGAAG |
| | ACCGCTGACT CAATATGCAC |
| 1361 | ATCTTTTGAG TTCTCCCAAA |
| | AAGTCCTCTT TGCTTGCTCT |
| 1401 | AGCAGCTCAT TGTTCTGATC |
| | CAAGTGAAGC TGATCGATTA |
| 1441 | ACACACCTTG CATCTCCTTC |
| | TGGAAAGGAT GAATATCCAC |
| 1481 | AGTGGGTAGT TGCAAGTCAG |
| | AGAAGTCTCC TTGAGGTCAT |
| 1521 | GGCAGAATTT CCATCAGCAA |
| | AGCCCCCGAT TGGAGCTTTC |
| 1561 | TTTGCCGGAG TTGCCCCACG |
| | TCTGCAACCC AGATACTATT |
| 1601 | CAATTTCATC CTCCCCAAGG |
| | ATGGCACCAT CTAGAATCCA |
| 1641 | CGTTACTTGT GCATTAGTTT |
| | TTGAGAAAAC ACCTGTAGGA |
| 1681 | CGGATTCACA AGGGTGTGTG |
| | TTCAACTTGG ATGAAGAATG |
| 1721 | CTGTGCCACT AGATGAGAGC |
| | CGTGATTGCA GCTGGGCACC |
| 1761 | TATTTTTGTT AGGCAATCTA |
| | ACTTCAAACT TCCTGCTGAT |
| 1801 | ACTAAAGTAC CTGTTTTAAT |
| | GATTGGACCT GGCACAGGAT |
| 1841 | TGGCTCCTTT TAGGGGTTTC |
| | CTGCAGGAAA GATTGGCTCT |
| 1881 | GAAAGAACCT CGAGGAGAAC |
| | TTGGACCTGC CATACTATTT |
| 1921 | TTTGGATCCA GGAATCGTCA |
| | AATGGATTAC ATTTATGAGG |
| 1961 | ATGACCTGAA CAACTTTCTT |
| | CAAACTGGTG CACTCTCTCA |
| 2001 | GCTTATTGTC GCTTTCTCAC |
| | GCGAGGGACC CAAAAAGGAA |
| 2041 | TATGTGCAAC ATAACATGAT |
| | CGAGAAACCG TCGGLTATCT |
| 2081 | GGAACATGAT TTCTCAGGAA |
| | GGATATATAT ATGTATGTGG |
| 2121 | TGACGCCAAA GGCATGGCGA |
| | GGGATCTCCA CAGAACACTA |
| 2161 | CACACTATTG TGCAAGAGCA |
| | GGGATCTCTA GACAGCTCCA |
| 2201 | AGACTGAAAG CATGGTGAAG |
| | AATCTGCAAA TGAATGGAAG |
| 2241 | GTATTTGCGT GATGTGTGGT |
| | GATTAGTACC CTCAAGTTAA |
| 2281 | CCCATCATAA AGTTGGGGCA |
| | AATGAAAGAA AATTATGTAA |
| 2321 | TTTATACTGG CCGAGGCCAA |
| | ATTGCCGGGG ATAAAAGAAA |
| 2361 | GCATGCAGCA AGGCAAAGTG |
| | AGAAGATTAC TCACCTTCGC |
| 2401 | TGCCAATTCT TAATAGTGAT |
| | CAGTTCTGTG ATTCTTTTTA |
| 2441 | CTCTTCTTGT GCGAAGGATT |
| | TTTTGGTTCA TGTAATTTAT |
| 2481 | ATATATATAC ACACAATATG |
| | TTGTAGTTAT AATACCAGTA |
| 2521 | ATTGGGAGGC ATTTTTACTG |
| | GACTTTCTCT CTCTAATTTT |
| 2561 | ACTCTAATGA CCAGATAAGT |
| | TAATTGATTC TGGACAAAAA |
| 2601 | AAAAAA |

A truncated Camptotheca acuminate cytochrome P450 reductase, which is expressed in the cytosol, can be used. Such a truncated cytochrome P450 reductase can have the N-terminal 1-69 amino acids missing and, for example, can be referred to as CaCPR⁷⁰⁻⁷⁰⁸ when the cytochrome P450 reductase is from Camptotheca acuminate. A sequence for this truncated Camptotheca acuminate cytochrome P450 reductase (CaCPR⁷⁰⁻⁷⁰⁸) is shown below as SEQ ID NO:41.

SSGKSGRVTE PPKPLMVKTE PEPEVDDGKK KVSIFYGTQT
GTAEGFAKAL AEEAKVRYEK ASFKVIDLDD YAADDEEYEE
KLKKETLTFF FLATYGDGEP TDNAARFYKW FMEGKERGDW
LKNLHYGVFG LGNRQYEHFN RIAKVVDDTI AEQGGKRLIP
VGLGDDDQCI EDDFAAWREL LWPELDQLLQ DEDGTTVATP
YTAAVLEYRV VFHDSPDASL LDKSFSKSNG HAVHDAQHPC
RANVAVRREL HTPASDRSCT HLEFDISGTG LVYETGDHVG
VYCENLIEVV EEAEMLLGLS PDTFFSIHTD KEDGTPLSGS
SLPPPFPPCT LRRALTQYAD LLSSPKKSSL LALAAHCSDP
SEADRLRHLA SPSGKDEYAQ WVVASQRSLL EVMAEFPSAK
PPIGAFFAGV APRLQPRYYS ISSSPRMAPS RIHVTCALVF
EKTPVGRIHK GVCSTWMKNA VPLDESRDCS WAPIFVRQSN
FKLPADTKVP VLMIGPGTGL APFRGFLQER LALKEAGAEL
GPAILFFGCR NRQMDYIYED ELNNFVETGA LSELIVAFSR
EGPKKEYVQH KMMEKASDIW NMISQEGYIY VCGDAKGMAR
DVHRTLHTIV QEQGSLDSSK TESMVKNLQM NGRYLRDVW

This truncated Camptotheca acuminate cytochrome P450 reductase (CaCPR⁷⁰⁻⁷⁰⁸) polypeptide can have a methionine at its N-terminus, and it can be encoded by the following cDNA sequence (SEQ ID NO:42).

TCGTCAGGAA AGTCGGGGAA AGTGACAGAA CCTCCGAAGC
CGCTGATGGT GAAGACTGAG CCGGAGCCGG AAGTTGATGA
CGGCAAGAAG AAGGTTTCTA TCTTCTATGG CACGCAGACC
GGTACCGCCG AAGGTTTCGC AAAGGCACTC GCCGAGGAAG
CAAAAGTGAG ATACGAAAAG GCGTCATTTA AAGTGATAGA
TTTGGATGAT TATGCCGCCG ACGATGAAGA ATACGAAGAG
AAATTGAAGA AAGAAACTTT AACATTTTTC TTCTTAGCTA
CATACGGAGA TGGAGAACCA ACTGACAATG CCGCCAGATT
CTACAAATGG TTTATGCAGG GAAAAGAGAG AGGGGACTGG
CTTAAGAATC TCCATTACGG AGTATTTGGT CTCGGCAACA
GGCAGTATGA GCATTTCAAC AGGATTGCAA AGGTGGTGGA
TGATACCATT GCCGAGCAGG GTGGGAAGCG CCTCATTCCT
GTGGGCCTTG GAGATGATGA TCAATGCATT GAAGATGATT
TTGCTGCATG GCGGGAGTTA TTGTGGCCCG AGTTGGATCA
GTTGCTTCAA GATGAAGATG GCACAACTGT TGCTACTCCT
TACACTGCCG CTGTATTGGA ATATCGTGTT GTATTCCATG
ACAGCCCAGA TGCATCATTA CTGGACAAGA GCTTCAGTAA
GTCAAATGGT CATGCTGTTC ATGATGCTCA ACATCCATGC
AGAGCTAACG TGGCTGTGAG AAGGGAGCTT CACACTCCCG
CATCTGATCG TTCTTGCACT CATCTGGAAT TTGATATTTC
TGGCACTGGA CTTGTATATG AAACTCGGGA CCATGTTGCT
GTGTATTGTG AGAATTTAAT TGAAGTTGTG GAGGAGGCAG
AAATGTTATT AGGTTTATCA CCAGATACCT TTTTCTCCAT
TCACACTGAT AAGCAGGATG GCACACCACT TAGTGCAAGC
TCCTTGCCAC CTCCTTTCCC CCCCTGTACT TTAAGAAGAG
CGCTGACTCA ATATGCAGAT CTTTTGAGTT CTCCCAAAAA
GTCCTCTTTG CTTGCTCTAG CAGCTCATTG TTCTGATCCA
AGTGAAGCTG ATCGATTAAG ACACCTTGCA TCTCCTTCTG
GAAAGGATGA ATATGCACAG TGGGTAGTTG CAAGTCAGAG
AAGTCTCCTT GAGGTCATGG CAGAATTTCC ATCAGCAAAG
CCCCCGATTG GAGCTTTCTT TGCCGGAGTT GCCCCACGTC
TGCAACCCAG ATACTATTCA ATTTCATCCT CCCCAAGGAT
GGCACCATCT AGAATCCACG TTACTTGTGC ATTAGTTTTT
GAGAAAACAC CTGTAGGACG GATTCACAAG GGTGTGTGTT
CAACTTGGAT GAAGAATGCT GTGCCACTAG ATGAGAGCCG
TGATTGCAGC TGGGCACCTA TTTTTGTTAG GCAATCTAAC
TTCAAACTTC CTGCTGATAC TAAAGTACCT GTTTTAATGA
TTGGACCTGG CACAGGATTG GCTCCTTTTA GGGGTTTCCT
GCAGGAAAGA TTGGCTCTGA AAGAAGCTGG AGCAGAACTT
GGACCTGCCA TACTATTTTT TGGATGCAGG AATCGTCAAA
TGGATTACAT TTATGAGGAT GAGCTGAACA ACTTTGTTGA
AACTGGTGCA CTCTCTGAGC TTATTGTCGC TTTCTCACGC
GAGGGACCCA AAAAGGAATA TGTGCAACAT AAGATGATGG
AGAAAGCGTC GGATATCTGG AACATGATTT CTCAGGAAGG
ATATATATAT GTATGTGGTG ACGCCAAAGG CATGGCGAGG
GATGTCCACA GAACACTACA CACTATTGTG CAAGAGCAGG
GATCTCTAGA CAGCTCCAAG ACTGAAAGCA TGGTGAAGAA
TCTGCAAATG AATGGAAGGT ATTTGCGTGA TGTGTGGTGA

An amino acid sequence for a cytosolic P. cablin patchoulol synthase (cytosol:PcPAS, AY508730; SEQ ID NO:43) is shown below.

| | |
|---|---|
| 1 | MELYAQSVGV GAASRPLANF |
| | HPCVWGDKFI VYNPQSCQAG |
| 41 | EREEAEELKV ELKRELKEAS |
| | DNYMRQLKMV DAIQRLGIDY |
| 81 | LFVEDVDEAL KNLFEMFDAF |
| | CKNNHDMHAT ALSFRLLRQH |
| 121 | GYRVSCEVFE KFKDGKDGFK |
| | VPNEDGAVAV LEFFEATHLR |
| 161 | VHGEDVLDNA FDFTRNYLES |
| | VYATLNDPTA KQVHNALNEF |
| 2C1 | SFRRGLPRVE ARKYISIYEQ |
| | YASHHKGLLK LAKLDFNLVQ |
| 241 | ALHRRELSED SRWWKTLQVP |
| | TKLSFVRDRL VESYFWASGS |
| 281 | YFEPNYSVAR MILAKGLAVL |
| | SLMDDVYDAY GTFEELQMFT |
| 321 | DAIERWDASC LDKLPDYMKI |
| | VYKALLDVFE EVDEELIKLG |
| 361 | APYRAYYGKE AMKYAARAYM |
| | EEAQWREQKH KPTTKEYMKL |
| 401 | ATKTCGYITL IILSCLGVEE |
| | GIVTKEAFDW VFSRPPFIEA |
| 441 | TLIIARLVND ITGHEFEKKR |
| | EHVRTAVECY MEEHKVGKQE |
| 481 | VVSEFYNQME SAVVKDINEGF |
| | LRPVEFPIPL LYLILNSVRT |
| 521 | LEVIYKEGDS YTHVGPAMQN |
| | IIKQLYLHPV PY |

A nucleic acid sequence for a cytosolic P. cablin patchoulol synthase (cytosol:PcPAS, AY508730; SEQ ID NO:44) is shown below.

| | |
|---|---|
| 1 | ATGGAGTTGT ATGCCCAAAG |
| | TGTTGGAGTG GGTGCTGCTT |
| 41 | CTCGTCCTCT TGCGAATTTT |
| | CATCCATGTG TGTGGGGAGA |
| 81 | CAAATTCATT GTCTACAACC |
| | CACAATCATG CCAGGCTGGA |
| 121 | GAGAGAGAAG AGGCTGAGGA |
| | GCTGAAAGTG GAGCTGAAAA |
| 161 | GAGAGCTGAA GGAAGCATCA |
| | GACAACTACA TGCGGCAACT |
| 201 | GAAAATGGTG GATGCAATAC |
| | AACGATTAGG CATTGACTAT |
| 241 | CTTTTTGTGG AAGATCTTGA |
| | TCAAGCTTTG AAGAATCTGT |
| 281 | TTGAAATGTT TGATGCTTTC |
| | TGCAAGAATA ATCATGACAT |
| 321 | GCACGCCACT GCTCTCAGCT |
| | TTCGCCTTCT CAGACAACAT |
| 361 | GGATACAGAG TTTCATGTGA |
| | AGTTTTTGAA AAGTTTAAGG |
| 401 | ATGGCAAAGA TGGATTTAAG |
| | GTTCCAAATG AGGATGGAGC |
| 441 | GGTTGCAGTC CTTGAATTCT |
| | TCGAAGCCAC GCATCTCAGA |
| 481 | GTCCATGGAG AAGACGTCCT |
| | TGATAATGCT TTTGACTTCA |
| 521 | CTAGGAACTA CTTGGAATCA |
| | GTCTATGCAA CTTTGAACGA |
| 561 | TCCAACCGCG AAACAAGTCC |
| | ACAACGCATT GAATGAGTTC |
| 601 | TCTTTTCGAA GAGGATTGCC |
| | ACGCGTGGAA GCAAGGAAGT |
| 641 | ACATATCAAT CTACGAGCAA |
| | TACGCATCTC ATCACAAAGG |
| 681 | CTTGCTCAAA CTTGCTAAGC |
| | TGGATTTCAA CTTGGTACAA |
| 721 | GCTTTGCACA GAAGGGAGCT |
| | GAGTGAAGAT TCTAGGTGGT |
| 761 | GGAAGACTTT ACAAGTGCCC |
| | ACAAAGCTAT CATTCGTTAG |
| 301 | AGATCGATTG GTGGAGTCCT |
| | ACTTCTGGGC TTCGGGATCT |
| 841 | TATTTCGAAC CGAATTATTC |
| | GGTAGCTAGG ATGATTTTAG |
| 881 | CAAAAGGGCT GGCTGTATTA |
| | TCTCTTATGG ATGATGTGTA |
| 921 | TGATGCATAT GGTACTTTTG |
| | AGGAATTACA AATGTTCACA |
| 961 | GATGCAATCG AAAGGTGGGA |
| | TGCTTCATGT TTAGATAAAC |
| 1001 | TTCCAGATTA CATGAAAATA |
| | GTATACAAGG CCCTTTTGGA |
| 1041 | TGTGTTTGAG GAAGTTGACG |
| | AGGAGTTGAT CAAGCTAGGC |
| 1081 | GCACCATATC GAGCCTACTA |
| | TGGAAAAGAA GCCATGAAAT |
| 1121 | ACGCCGCGAG AGCTTACATG |
| | GAAGAGGCCC AATGGAGGGA |
| 1161 | GCAAAAGCAC AAACCCACAA |
| | CCAAGGAGTA TATGAAGCTG |
| 1201 | GCAACCAAGA CATGTGGCTA |
| | CATAACTCTA ATAATATTAT |
| 1241 | CATGTCTTGG AGTGGAAGAG |
| | GGCATTGTGA CCAAAGAAGC |
| 1281 | CTTCGATTGG GTGTTCTCCC |
| | GACCTCCTTT CATCGAGGCT |
| 1321 | ACATTAATCA TTGCCAGGCT |
| | CGTCAATGAT ATTACAGGAC |
| 1361 | ACGAGTTTGA GAAAAAACGA |
| | GAGCACGTTC GCACTGCAGT |
| 1401 | AGAATGCTAC ATGGAAGAGC |
| | ACAAAGTGGG GAAGCAAGAG |
| 1441 | GTGGTGTCTG AATTCTACAA |
| | CCAAATGGAG TCAGCATGGA |
| 1481 | AGGACATTAA TGAGGGGTTC |
| | CTCAGACCAG TTGAATTTCC |
| 1521 | AATCCCTCTA CTTTATCTTA |
| | TTCTCAATTC AGTCCGAACA |
| 1561 | CTTGAGGTTA TTTACAAAGA |
| | GGGCGATTCG TATACACACG |
| 1601 | TGGGTCCTGC AATGCAAAAC |
| | ATCATCAAGC AGTTGTACCT |
| 1641 | TCACCCTGTT CCATATTAA |

An example of a Picea abies FPPS (PaFPPS) sequence is shown below as SEQ ID NO:45 (NCBI accession no. ACΔ21460.1).

| | |
|---|---|
| 1 | MASNGIVDVK TKFEEIYLEL |
| | KAQILNDPAF DYTEDARQWV |
| 41 | EKMLDYTVPG GKLNRGLSVI |
| | DSYRLLKAGK EISEDEVFLG |
| 81 | CVLGWCIEWL QAYFLILDDI |
| | MDSSHTRRGQ PCWFRLPKVG |
| 121 | LIAVNDGILL RNHICRILKK |
| | HFRTKPYYVD LLDLFNEVEF |
| 161 | QTASGQLLDL ITTHECATDL |
| | SKYKMPTYVR IVQYKTAYYS |
| 201 | FYLPVACALV MAGENLDNHV |
| | DVKNILVEMG TYFQVQDDYL |
| 241 | DCFGDPEVIG KIGTDIEDFK |
| | CSWLVVQALE RANESQLQRL |
| 281 | YANYGKKDPS CVAEVKAVYR |
| | DLGLQDVFLE YERTSHKELI |
| 321 | SSIEAQENES LQLVLKSFLG |
| | KIYKRQK |

A cDNA encoding the Picea abies FPPS (PaFPPS) with SEQ ID NO:45 is shown below as SEQ ID NO:46.

| | |
|---|---|
| 1 | ATGGCTTCAA ACGGCATCGT |
| | CGACGTGAAA ACCAAGTTTG |
| 41 | AGGAAATCTA TCTTGAGCTT |
| | AAGGCTCAGA TTCTGAACGA |
| 81 | TCCTGCCTTC GATTACACCG |
| | AAGACGCCCG TCAATGGGTC |
| 121 | GAGAAGATGC TGGACTACAC |
| | GGTGCCCGGA GGAAAGCTGA |
| 161 | ACCGCGGTCT GTCTGTAATA |
| | GACAGCTACA GGCTATTGAA |
| 201 | AGCAGGAAAG GAAATATCAG |
| | AAGATGAAGT CTTTCTTGGA |
| 241 | TGTGTGCTTG GCTGGTGTAT |
| | TGAATGGCTT CAAGCATATT |
| 281 | TCCTCATATT AGATGACATC |
| | ATGGACAGCT CTCACACTAG |
| 321 | GCGTGGACAA CCTTGTTGGT |
| | TCAGATTACC TAAGGTTGGC |
| 361 | TTAATTGCTG TTAATGATGG |
| | AATATTGCTT CGTAACCACA |
| 401 | TATGCAGAAT TCTGAAAAAG |
| | CATTTTCGCA CTAAGCCTTA |
| 441 | CTATGTGGAT CTCCTTGATT |
| | TATTCAATGA GGTTGAGTTT |
| 481 | CAAACAGCTA GTGGACAGTT |
| | GCTGGACCTT ATCACTACTC |
| 521 | ATGAAGGAGC AACTGACCTT |
| | TCAAAGTACA AAATGCCAAC |
| 561 | TTATGTTCGT ATAGTTCAAT |
| | ACAAGACTGC CTACTATTCA |
| 601 | TTCTATCTGC CGGTTGCCTG |
| | TGCACTGGTA ATGGCAGGGG |
| 641 | AAAATTTAGA TAATCACGTA |
| | GATGTCAAGA ATATTTTAGT |
| 681 | CGAAATGGGA ACCTATTTTC |
| | AAGTACAGGA TGATTATCTT |
| 721 | GATTGCTTTG GTGATCCAGA |
| | AGTGATTGGG AAGATTGGAA |
| 761 | CTGATATCGA AGACTTCAAG |
| | TGCTCTTGGT TGGTGGTGCA |
| 301 | AGCCCTTGAA CGGGCAAATG |
| | AGAGCCAACT TCAACGATTA |
| 841 | TATGCCAATT ATGGAAAGAA |
| | AGATCCTTCT TGTGTTGCAG |
| 381 | AAGTGAAGGC TGTATATAGG |
| | GATCTTGGAC TTCAGGATGT |
| 921 | TTTTCTGGAA TACGAGCGTA |
| | CTAGTCACAA GGAGCTCATT |
| 961 | TCTTCCATCG AGGCTCAGGA |
| | GAATGAATCT TTGCAGCTTG |
| 1001 | TTCTGAAGTC CTTCCTAGGG |
| | AAGATATACA AGCGACAGAA |
| 1041 | GTAA |

An example of a Gallus gallus FPPS (GgFPPS) polypeptide sequence is shown below as SEQ ID NO:47 (NCBI accession no. XP_015154133.1).

| | |
|---|---|
| 1 | MSADGAKRTA AEREREEFVG |
| | FFPQIVRDLT EDGIGHPEVG |
| 41 | DAVARLKEVL QYNAPGGKCN |
| | RGLTVVAAYR ELSGPGQKDA |
| 81 | ESLRCALAVG WCIELFQAFF |
| | LVADDIMDQS LTRRGQLCWY |
| 121 | KKEGVGLDAI NDSFLLESSV |
| | YRVLKKYCGQ RPYYVHLLEL |
| 161 | FLQTAYQTEL GQMLDLITAP |
| | VSKVDLSHFS EERYKAIVKY |
| 201 | KTAFYSFYLP VAAAMYKVGI |
| | DSKEEHENAK AILLEMGEYF |
| 241 | QIQDDYLDCF GDPALTGKVG |
| | TDIQDNKCSW LVVQCLQRVT |
| 281 | PEQRQLLEDN YGRKEPEKVA |
| | KVKELYEAVG MRAAFQQYEE |
| 321 | SSYRRLQELI EKHSNRLPKE |
| | IFLGLAQKIY KRQK |

A cDNA encoding the Gallus gallus FPPS (GgFPPS) with SEQ ID NO:47 is shown below as SEQ ID NO:48.

| | |
|---|---|
| 1 | ACAATGCCCC GCGCGGCGCC |
| | GGGCGGAGCG CACGGAAAGG |
| 41 | TCGCGGGGCA AAAAGCGGCG |
| | CTGAGCGGAC GGGGCCGAAC |
| 81 | GCGTCGGGGT CGCCATGAGC |
| | GCGGATGGGG CGAAGCGGAC |
| 121 | GCCGGCCGAG ACCGAGAGGG |
| | AGGACTTCCT GGGCTTCTTC |
| 161 | CCGCAGATCG TCCGCGATCT |
| | GACCGAGGAC GGCATCGGAC |
| 201 | ACCCGGAGGT GGGCGACGCT |
| | GTGGCGCGGC TGAAGGAGGT |
| 241 | GCTGCAATAC AACGCTCCCG |
| | GTGGGAAATG CAACCGTGGG |
| 281 | CTGACGGTGG TGGCTGCGTA |
| | CCGGGAGCTG TCGGGGCCGG |
| 321 | GGCAGAAGGA TGCTGAGAGC |
| | CTGCGGTGCG CGCTGGCCGT |
| 361 | GGGTTGGTGC ATCGAGTTGT |
| | TCCAGGCCTT CTTCCTGGTG |
| 401 | GCTGATGATA TCATGGATCA |
| | GTCCCTCACG CGCCGGGGGC |
| 441 | AGCTGTGTTG GTATAAGAAG |
| | GAGGGGGTCG GTTTGGATGC |
| 481 | CATCAACCAC TCCTTCCTCC |
| | TCGAGTCCTC TGTGTACAGA |
| 521 | GTGCTGAAGA AGTACTGCGG |
| | GCAGCGGCCG TATTACGTGC |
| 561 | ATCTGTTGGA GCTCTTCCTG |
| | CAGACCGCCT ACCAGACTGA |
| 601 | GCTCGGGCAG ATGCTGGACC |
| | TCATCACAGC TCCCGTCTCC |
| 641 | AAAGTGGATT TGAGTCACTT |
| | CAGCGAGGAG AGGTACAAAG |
| 681 | CCATCGTTAA GTACAAGACT |
| | GCCTTCTACT CCTTCTACCT |
| 721 | ACCCGTGGCT GCTGCCATGT |
| | ATATGGTTGG GATCGACAGT |
| 761 | AAGGAAGAAC ACGAGAATGC |
| | CAAAGCCATC CTGCTGGAGA |
| 801 | TGGGGGAATA CTTCCAGATC |
| | CAGGATGATT ACCTGGACTG |
| 841 | CTTTGGGGAC CCGGCGCTCA |
| | CGGGGAAGGT GGGCACCGAC |
| 881 | ATCCAGGACA ATAAATGCAG |
| | CTGGCTCGTG GTGCAGTGCC |
| 921 | TGCAGCGCGT CACGCCGGAG |
| | CAGCGGCAGC TCCTGGAGGA |
| 961 | CAACTACGGC CGTAAGGAGC |
| | CCGAGAAGGT GGCGAAGGTG |
| 1001 | AAGGAGCTGT ATGAGGCCGT |
| | GGGGATGAGG GCTGCGTTCC |
| 1041 | AGCAGTACGA GGAGAGCAGC |
| | TACCGGCGCC TGCAGGAACT |
| 1081 | GATAGAGAAG CACTCGAACC |
| | GCCTCCCGAA GGAGATCTTC |
| 1121 | CTCGGCCTGG CACAGAAGAT |
| | CTACAAACGC CAGAAATGAG |
| 1161 | GGGTGGGGGC GGCAGCGGCT |
| | CTGTGCTTCG CGCTGTGTTG |
| 1201 | GGTGGCTTCG CAGCCCCGGA |
| | CCCGGTGCTC CCCCCACCCG |
| 1241 | TTATCCCCGG AGATGCGGGG |
| | GGGGGGCGGT GCGGGGCGCG |
| 1281 | CATCCATCGG TGCCGTCAGA |
| | CTGTGTGTCA ATAAACGTTA |
| 1321 | ATTTATTGCC |

An Arabidopsis thaliana ribulose bisphosphate carboxylase small chain 1A protein encoded shown below as SEQ ID NO:49.

| | |
|---|---|
| 1 | MASSMLSSAT MVASPAQATM |
| | VAPFNGLKSS AAFPATRKAN |
| 41 | NDITSITSNG GRVNCKQVWP |
| | PIGKKKFETL SYLPDLTDSE |
| 81 | LAKEVDYLIR NKWIPCVEFE |
| | LEHGFVYREH GNSPGYYDGR |
| 121 | YWTKWKLPLF GCTDSAQVLK |
| | EVEECKKEYP NAFIRIIGFD |
| 161 | NTRQVQCISF IAYKPPSFTG |

A nucleotide sequence for the Arabidopsis thaliana ribulose bisphosphate carboxylase small chain 1A (NM_105379.4) is shown below as SEQ ID NO:50.

| | |
|---|---|
| 1 | CCAAGGTAAA AAAAAGGTAT |
| | GAAAGCTCTA TAGTAAGTAA |
| 41 | AATATAAATT CCCCATAAGG |
| | AAAGGGCCAA GTCCACCAGG |
| 81 | CAAGTAAAAT GAGCAAGCAC |
| | CACTCCACCA TCACACAATT |
| 121 | TCACTCATAG ATAACGATAA |
| | GATTCATGGA ATTATCTTCC |
| 161 | ACGTGGCATT ATTCCAGCGG |
| | TTCAAGCCGA TAAGGGTCTC |
| 201 | AACACCTCTC CTTAGGCCTT |
| | TGTGGCCGTT ACCAAGTAAA |
| 241 | ATTAACCTCA CACATATCCA |
| | CACTCAAAAT CCAACGGTGT |
| 281 | AGATCCTAGT CCACTTGAAT |
| | CTCATGTATC CTAGACCCTC |
| 321 | CGATCACTCC AAAGCTTGTT |
| | CTCATTGTTG TTATCATTAT |
| 361 | ATATAGATGA CCAAAGCACT |
| | AGACCAAACC TCAGTCACAC |
| 401 | AAAGAGTAAA GAAGAACAAT |
| | GGCTTCCTCT ATGCTCTCTT |
| 441 | CCGCTACTAT GGTTGCCTCT |
| | CCGGCTCAGG CCACTATGGT |
| 481 | CGCTCCTTTC AACGGACTTA |
| | AGTCCTCCGC TGCCTTCCCA |
| 521 | GCCACCCGCA AGGCTAACAA |
| | CGACATTACT TCCATCACAA |
| 561 | GCAACGGCGG AAGAGTTAAC |
| | TGCATGCAGG TGTGGCCTCC |
| 601 | GATTGGAAAG AAGAAGTTTG |
| | AGACTCTCTC TTACCTTCCT |
| 641 | GACCTTACCG ATTCCGAATT |
| | GGCTAAGGAA GTTGACTACC |
| 681 | TTATCCGCAA CAAGTGGATT |
| | CCTTGTGTTG AATTCGAGTT |
| 721 | GGAGCACGGA TTTGTGTACC |
| | GTGAGCACGG TAACTCACCC |
| 761 | GGATACTATG ATGGACGGTA |
| | CTGGACAATG TGGAAGCTTC |
| 301 | CCTTGTTCGG TTGCACCGAC |
| | TCCGCTCAAG TGTTGAAGGA |
| 841 | AGTGGAAGAG TGCAAGAAGG |
| | AGTACCCCAA TGCCTTCATT |
| 881 | AGGATCATCG GATTCGACAA |
| | CACCCGTCAA GTCCAGTGCA |
| 921 | TCAGTTTCAT TGCCTACAAG |
| | CCACCAAGCT TCACCGGTTA |
| 961 | ATTTCCCTTT GCTTTTGTGT |
| | AAACCTCAAA ACTTTATCCC |
| 1001 | CCATCTTTGA TTTTATCCCT |
| | TGTTTTTCTG CTTTTTTCTT |
| 1041 | CTTTCTTGGG TTTTAATTTC |
| | CGGACTTAAC GTTTGTTTTC |
| 1081 | CGGTTTGCGA GACATATTCT |
| | ATCGGATTCT CAACTGTCTG |
| 1121 | ATGAAATAAA TATGTAATGT |
| | TCTATAAGTC TTTCAATTTG |
| 1161 | ATATGCATAT CAACAAAAAG |
| | AAAATAGGAC AATGCGGCTA |
| 1201 | CAAATATGAA ATTTACAAGT |
| | TTAAGAACCA TGAGTCGCTA |
| 1241 | AAGAAATCAT TAAGAAAATT |
| | AGTTTCAC |

In some cases, a portion of the Arabidopsis thaliana ribulose bisphosphate carboxylase small chain 1A protein can be used as a chloroplast transit peptide to re-localize cytosolic proteins to the chloroplast, for example, an Arabidopsis thaliana ribulose bisphosphate carboxylase small chain 1A peptide with SEQ ID NO:101 (shown below).

```
  1 MASSMLSSAT MVASPAQATM
      VAPFNGLKSS AAPPAIRKAN
41 NDITSITSNG GRVN
```

A nucleic acid segment that encodes the Arabidopsis thaliana ribulose bisphosphate carboxylase small chain 1A peptide with SEQ ID NO:101 is shown below as SEQ ID NO:102.

| | |
|---|---|
| 1 | ATGGCTTCCT CTATGCTCTC |
| | TTCCGCTACT ATGGTTGCCT |
| 41 | CTCCGGCTCA GGCCACTATG |
| | GTCGCTCCTT TCAACGGACT |
| 81 | TAAGTCCTCC GCTGCCTTCC |
| | CAGCCACCCG CAAGGCTAAC |
| 121 | AACGACATTA CTTCCATCAC |
| | AAGCAACGGC GGAAGAGTTA |
| 161 | AC |

The enzyme and protein sequences shown herein can have one or more deletions, insertions, replacements, or substitutions without loss of their enzymatic activities. Such enzymatic activities include the synthesis of terpenes/terpenoids. The terpene synthase enzymes can have, for example, at least 60%, or at least 70%, or at least 80%, or at least 90%, or at least 95%, or at least 97%, or at least 98%, or at least 99% sequence identity to a sequence described herein.

In some cases, the enzymes and proteins described herein are naturally expressed in the cytosol, but it can be desirable to express some of these enzymes and/or proteins in plastids or other subcellular locations.

In some cases, it is useful to target enzymes and/or proteins to the plastid. To do this, a nucleic acid segment encoding the enzymes or proteins can be fused to sequences were fused at their N-terminus to the plastid targeting sequence. For example, a plastid targeting sequence of the Arabidopsis thaliana ribulose bisphosphate carboxylase small chain 1A (NM_105379.4; SEQ ID NO:49 or 101) can be used.

For example, wild type ElHMGR, AtWRI11-397 (transcription factor), NoLDSP (lipid droplet surface protein), SaGGDPS, MtGGDPS, TsGGDPS, MeGGDPS, AtFDPS and PcPAS are cytosolic proteins. However, in some cases it can be useful to target these enzymes and/or proteins to the plastid. Hence, SaGGDPS, MtGGDPS, TsGGDPS, MeGGDPS, AtFDPS and PcPAS can be targeted to plastids by fusing each of their N-termini to the plastid targeting sequence of the of the Arabidopsis thaliana ribulose bisphosphate carboxylase small chain 1A (NM_105379.4; SEQ ID NO:49 or 101).

Some proteins/enzymes are naturally targeted to plastids, but in some cases, it can be useful to target them to the cytosol. This can be some in some cases by removing a natural plastid targeting sequence. For example, native PbDXS (CfDXS) and AgABS (plastid:AgABS) each have a plastid targeting sequence in their N-terminus. To target AgABS to the cytosol, for example, the plastid targeting sequence can be removed (e.g., cytosol:AgABS⁸⁵⁻⁸⁶⁸, residues 1-84 were removed).

Similarly, native PsCYP720B4 and native CaCPR are naturally localized at the endoplasmic reticulum (ER; e.g., ER:PcCYP720B4 and ER:CaCPR, respectively). To target PcCYP720B4 to the cytosol, the hydrophobic region that including amino acids 1-29 was removed (cytosol:PsCYP720B4³⁰⁻⁴⁸³). To target PsCYP720B4 and CaCPR to lipid droplets, hydrophobic regions were removed, and the truncated proteins were fused to NoLDSP (LD:PsCYP720B4³⁰⁻⁴⁸³ and LD:CaCPR⁷⁰⁻⁷⁰⁸, respectively).

Hence, the enzymes and proteins described herein can have sequences that are modified (compared to wild type) to include a segment encoding a plastid targeting sequence, or a LDSP. In some cases, the enzymes and proteins described herein can have sequences that are modified (compared to wild type) by removal of plastid targeting segments or hydrophobic regions.

### Squalene Synthases

A variety of squalene synthase enzymes can be used in the methods described herein to synthesize squalene and compounds derived from squalene. Squalene is useful as a component in numerous formulations and it is a biochemical precursor to a family of steroids. Squalene synthases can be used in the expression systems and methods described herein in native or modified form. For examples, in some cases, the squalene synthases can be modified by removal of a plastidial targeting sequence or a hydrophobic region. In addition, the native or modified forms of squalene synthases can be fused to a lipid droplet surface protein (LDSP). For example, the LDSP protein can replace the truncated segments of a squalene synthase.

Examples of squalene synthases that can be used include those from Amaranthus hybridus, Botryococcus braunii, Euphorbia lathyrism, Ganoderma lucidum, and Mortierella alpine.

For example, an Amaranthus hybridus squalene synthase (AhSQS) with the following sequence is shown below as SEQ ID NO:51 (also as NCBI accession no. BAW27654.1).

| | |
|---|---|
| 1 | MGSLGAILKH PDEFYPLLKL |
| | KMAVKEAEKQ IPSESHWGFC |
| 41 | YSMLHKVSRS FALVTQQLGT |
| | ELRNAVCVFY LVLRALDTVE |
| 81 | DDISIATDVY LPILKAFYQH |
| | IYDREWHFSC GTKHYKVLMD |
| 121 | EFHQVSTAFL ELERGYQLAI |
| | EDITKRMGAG MAKFICOEVE |
| 161 | TVSDYDEYCH YVAGLVGLGL |
| | SKLFHNAGLE DLASDDLSNS |
| 201 | MGLFLQKTNI IRDYLEDINE |
| | IPKCRMEWPR EIWSKYVNKL |
| 241 | EDLKYEENSV KAVQCLNDMV |
| | TNALLHVEDC LKYMSALRDH |
| 281 | AIFRFCAIPQ IMAIGTLALC |
| | YNNVEVFRGV VKMRRGLTAR |
| 321 | VIDKTDSMPD VYGAFYDFAC |
| | MIKPKVDKND PNAMKTLSRI |
| 361 | DAIEKICRDS GTLNKRKLHI |
| | ISIKSAYIPI MVMVLFIVLA |
| 401 | IFFNRLSESN RMINN |

In some cases, the Amaranthus hybridus squalene synthase can have a C-terminal truncation of about 30-50 amino acids. For example, the Amaranthus hybridus squalene synthase sequence with SEQ ID NO:51 can have a 41-amino acid C-terminal truncation (AhSQS CΔ41), with a sequence such as that shown below (SEQ ID NO:52).

| | |
|---|---|
| 1 | MGSLGAILKH PDEFYPLLKL |
| | KMAVKEAEKQ IPSESHWGFC |
| 41 | YSMLHKVSRS FALVIQQLGT |
| | ELRNAVCVFY LVLRALDTVE |
| 81 | DDTSIATDVK LPILKAFYQH |
| | IYDREWHFSC GTKHYKVLMD |
| 121 | EFHQVSTAFL ELERGYQLAI |
| | EDITKRMGAG MAKFICQEVE |
| 161 | TVSDYDEYCH YVAGLVGLGL |
| | SKLFHNAGLE DLASDDLSNS |
| 201 | MGLFLQKTNI IRDYLEDINE |
| | IPKCRMFWPR EIWSKYVNKL |
| 241 | EDLKYEENSV KAVQCLNDMV |
| | TNALLHVEDC LKYMSALRDH |
| 281 | AIFRFCAIPQ IMAIGTLALC |
| | YNNVEVFRGV VKMRRGLTAR |
| 321 | VIDKTDSMPD VYGAFYDFAC |
| | MIKPKVDKND PNAMKTLSRI |
| 361 | DAIEKICRDS GTLN |

In another example, a Botryococcus braunii squalene synthase can be used, for example, with the following sequence (SEQ ID NO:53; NCBI accession no. AAF20201.1).

| | |
|---|---|
| 1 | MGMLRWGVES LQNPDELIPV |
| | LRMIYADKFG KIKPKDEDRG |
| 41 | FCYEILNLVS RSFAIVIQQL |
| | PAQLRDPVCI FYLVLRALDT |
| 81 | VEDDMKIAAT TKIPLLRDFY |
| | EKISDRSFRM TAGDQKDYIR |
| 121 | LLDQYPKVTS VFLKLTPREQ |
| | EIIADITKRM GNGMADFVHK |
| 161 | GVPDTVGDYD LYCHYVAGVV |
| | GLGLSQLFVA SGLQSPSLTR |
| 201 | SEDLSNHMGL FLQKTNIIRD |
| | YFEDINELPA PRMFWPREIW |
| 241 | GKYANNLAEF KDPANKAAAM |
| | CCLNEMVTDA LRHAVYCLQY |
| 281 | MSMIEDPQIF NFCAIPQTMA |
| | FGTLSLCYNN YTIFTGPKAA |
| 321 | VKLRRGTTAK LMYTSNNKFA |
| | MYRHFLNFAE KLEVRCNTET |
| 361 | SEDPSVTTTL EHLHKIKAAC |
| | KAGLARTKDD TFDELRSRLL |
| 401 | ALTGGSFYLA WTYNFLDLRG |
| | PGDLPTFLSV TQHWWSILIF |
| 441 | LISIAVFFIP SRPSPRPTLS |
| | A |

A nucleotide sequence encoding the Botryococcus braunii squalene synthase with SEQ ID NO:53 is shown below as SEQ ID NO:54 (NCBI accession no. AF205791.1).

| | |
|---|---|
| 1 | AACAGCAACA AGTCCTCTGC |
| | GTCAGGCAAA ACGTCCGTTT |
| 41 | GTATGGCTTG GCGCTTGAAA |
| | GCTGCTGGGG ATAAACGTCA |
| 31 | AAAGAAAGAA GCTCTGTTCG |
| | GGTTCACGGG TGTCGTTTAG |
| 121 | TACTTTCCCC TACGACATTG |
| | TCAGCCTTGG CTCATCGCAA |
| 161 | TCCAACCAAA TATGGGGATG |
| | CTTCGCTGGG GAGTGGAGTC |
| 201 | TTTGCAGAAT CCAGATGAAT |
| | TAATCCCGGT CTTGAGGATG |
| 241 | ATTTATGCTG ATAAGTTTGG |
| | AAAGATCAAG CCAAAGGACG |
| 281 | AAGACCGGGG CTTCTGCTAT |
| | GAAATTTTAA ACCTTGTTTC |
| 321 | AAGAAGTTTT GCAATCGTCA |
| | TCCAACAGCT CCCTGCACAG |
| 361 | CTGAGGGACC CAGTCTCCAT |
| | ATTTTACCTT CTACTACGCG |
| 401 | CCCTGGACAC AGTCGAAGAT |
| | GATATGAAAA TTGCAGCAAC |
| 441 | CACCAAGATT CCCTTGCTGC |
| | GTGACTTTTA TGAGAAAATT |
| 481 | TCTGACAGGT CATTCCGCAT |
| | GACGCCCGGA GATCAAAAAG |
| 521 | ACTACATCAG GCTGTTGGAT |
| | CAGTACCCCA AAGTGACAAG |
| 561 | CGTTTTCTTG AAATTGACCC |
| | CCCGTGAACA AGAGATAATT |
| 601 | GCAGACATTA CAAAGCGGAT |
| | GGGGAATGGA ATGGCTGACT |
| 641 | TCGTGCATAA GGGTGTTCCC |
| | GACACAGTGG GGGACTACGA |
| 681 | CCTTTACTGC CACTATGTTG |
| | CTGGGGTGGT GGGTCTCGGG |
| 721 | CTTTCCCAGT TGTTCGTTGC |
| | GAGTGGACTA CAGTCACCCT |
| 761 | CTTTGACCCG CAGTGAAGAC |
| | CTTTCCAATC ACATGGGCCT |
| 801 | CTTCCTTCAG AAGACCAACA |
| | TCATCCGCGA CTACTTTGAG |
| 841 | GACATCAATG AGCTGCCTGC |
| | CCCCCGGATG TTCTGGCCCA |
| 881 | GAGAGATCTG GGGCAAGTAT |
| | GCGAACAACC TCGCTGAGTT |
| 921 | CAAAGACCCG GCCAACAAGG |
| | CGGCTGCAAT GTGCTGCCTC |
| 961 | AACGAGATGG TCACAGATGC |
| | ATTGAGGCAC GCGGTGTACT |
| 1001 | GCCTGCAGTA CATGTCCATG |
| | ATTGAGGATC CGCAGATCTT |
| 1041 | CAACTTCTGT GCCATCCCTC |
| | AGACCATGGC CTTCGGCACC |
| 1081 | CTGTCTTTGT GTTACAACAA |
| | CTACACTATC TTCACAGGGC |
| 1121 | CCAAAGCGGC TGTGAAGCTG |
| | CGTAGGGGCA CCACTGCCAA |
| 1161 | GCTGATGTAC ACCTCTAACA |
| | ATATGTTTGC GATGTACCGT |
| 1201 | CATTTCCTCA ACTTCGCAGA |
| | GAAGCTGGAA GTCAGATGCA |
| 1241 | ACACCGAGAC CAGCGAGGAT |
| | CCCAGCGTGA CCACCACTCT |
| 1281 | GGAACACCTG CATAAGATCA |
| | AAGCTGCCTG CAAGGCTGGG |
| 1321 | CTGGCACGCA CAAAAGATGA |
| | CACCTTTGAC GAATTGAGGA |
| 1361 | GCACGTTGTT AGCGCTGACG |
| | GGAGGCAGCT TCTACCTCGC |
| 1401 | CTGGACCTAC AATTTCCTAG |
| | ACCTTCGAGG CCCGGGAGAC |
| 1441 | CTGCCCACCT TCTTATCTGT |
| | AACCCAACAT TGGTGGTCTA |
| 1481 | TTCTGATCTT CCTCATTTCG |
| | ATTGCCGTCT TCTTTATTCC |
| 1521 | GTCGAGGCCC TCACCTAGAC |
| | CCACACTCAG CGCCTAATCC |
| 1561 | TTTGGCTCTC GTCAATTCCG |
| | GAGTCCCCCA TTGTTGTCAG |
| 1601 | CACTTGGGGA ATTTCGTGGT |
| | CTTCTTGACC ACACTCTTGT |
| 1641 | CTCTGGCAGA GGTCAAGGAC |
| | ACTGTCAGGG ACAAGTGAGT |
| 1681 | ATTCTGACCC CCCCCCCCCC |
| | CCCCCTCTGC TCCTTTCACC |
| 1721 | ACCCCTCCCT ATCATCTGGG |
| | GCAAAGCTTG GGAATGGGCC |
| 1761 | CGTCCCCCTG TTGTCCCGCT |
| | CAGATGCAAA GTTTGGGTTA |
| 1801 | TGTAACTGGG TTGAACGGCT |
| | CGGGGCGGTT TGAAGCTGTC |
| 1841 | CCTTGTTGGA GATGGAAAAT |
| | TGCAGGGCCC GGGGGGGTTA |
| 1381 | ACTGGACACG CTCTTCCGTC |
| | CCGCAGTCTC CTTCTGGCTT |
| 1921 | TATTCTGCCG TGGATGCTGT |
| | GAACCCGCCC CCTCTCTGGG |
| 1961 | CCGGCTCAAT ATACAAGTAT |
| | TAGTTTCGGT GTTTGTGTCA |
| 2001 | ATCCTTTCTC ACAACTTCCC |
| | TGTTCGTTGG ACTGGACACG |
| 2041 | CACCCTTAGG TCCTTTGATT |
| | GGGAATGCGG CCCCTTTGGG |
| 2081 | TCTTTAGGCT CTCGGGTAGT |
| | CTAGTTTGCA ATTGTTGCAT |
| 2121 | GGGCGCGGCT TTGCACAGAC |
| | GCCTGGACCT TCATTGAGAC |
| 2161 | ACGTTTCGGA AAACTCGACA |
| | GTTTTGAGGT AACCTGCTCG |
| 2201 | TGGGCCTCGG TGTGTCTGGA |
| | GGTGTCAGGG GCCTGTGCTC |
| 2241 | CCTGCTGGGA TGTTCCCGCT |
| | TTGCTGTAAA AAGTCGGACG |
| 2281 | TTTGTTATCC TTTGCGGGGG |
| | TTCATCTTTG AGTGGGCCCT |
| 2321 | GCTTCTCTGC CCGTGTGATG |
| | TAATGGTTTG TATTGGATAG |
| 2361 | GTATGTTGCC TTATCTCGTG |
| | TATGGAATTC GTATGGTACT |
| 2401 | TGCAGTATTC AGGAGACTTG |
| | AGTAACGACA TCGAGGACAG |
| 2441 | GTAACAAGCG CTCCGATTAT |
| | GTGCTCTGTT ACACCCGACT |
| 2481 | TCCAAAGATT TATGCGAGGT |
| | CCTGCGGAAC GCAGATTTGA |
| 2521 | CATTGGAGAG CCCCAATTGG |
| | CCGTGGCAAT CTGTAGAATG |
| 2561 | TCAAAAGAGA AAACAGGAAA |
| | TCAGGTTTTA AAGTCCGTGC |
| 2601 | CTATCAGCAT CCTGTGAAAG |
| | CTGATGCGGT TACGGGATGA |
| 2641 | ATGTCAGGAA TACTCGCTCC |
| | AGTATTAACG TGCGCAGATT |
| 2681 | CCGACTGAAG CAAATCGATG |
| | AAATTTGGGG AGGTGTCGTT |
| 2721 | TTTAGACCTT GACAACGGCC |
| | ATGGGTCGTA CCTTTTTGCA |
| 2761 | AAGTATATAT TTATTTGCAC |
| | TAACTCATTA GGCACGTTGG |
| 2801 | TTTTTTTTGT CCCCCTCGGA |
| | ACGCCTTTTT AAGATAGTTA |
| 2841 | ACTAGTTTGG TCAGGGTATT |
| | CGTCAGAAGC ACGAAGCACA |
| 2881 | GAAGGTTTCT TTTGAGATGG |
| | CGGCGATTGT TTTCCACGAG |
| 2921 | AGCAGAGTCA ATCTCACGCG |
| | TACTCGAGCA AACATCGTTG |
| 2961 | GTCAGGACAT GGTGTTGTCT |
| | CTTGGCCGGC CCTGTAACTT |
| 3001 | TGATGCCCCC AAAAAAAAAA |
| | AAAAAAAAAA AAAAAAAAAA |
| 3041 | AAAAAAAAAA AAAAAAAAAA |
| | AAAAAAAAAA AAAAAA |

In some cases, the Botryococcus braunii squalene synthase can have a C-terminal truncation. for example, of about 40-85 amino acids. Such a C-terminal truncation of a Botryococcus braunii squalene synthase can have 40 amino acids truncated from the C-terminus, and the following sequence (SEQ ID NO:55) (also called BbSQS CΔ40).

| | |
|---|---|
| 1 | MGMLRWGVES LQNPDELIPV |
| | LRMIYADKFG KIKPKDEDRG |
| 41 | FCYEILNLVS RSFAIVIQQL |
| | PAQLRDPVCI FYLVLRALDT |
| 81 | VEDDMKIAAT TKIPLLRDFY |
| | EKISDRSFRM TAGDQKDYIR |
| 121 | LLDQYPKVTS VFLKLTPREQ |
| | EIIADITKRM GNGMADFVHK |
| 161 | GVPDTVGDYD LYCHYVAGVV |
| | GLGLSQLFVA SGLQSPSLTR |
| 201 | SEDLSNHMGL FLQKTNIIRD |
| | YFEDINELPA PRMFWPREIW |
| 241 | GKYANNLAEF KDPANKAAAM |
| | CCLNEMVTDA LRHAVYCLQY |
| 281 | MSMIEDPQIF NFCAIPQTMA |
| | FGTLSLCYNN YTIFTGPKAA |
| 321 | VKLRRGTTAK LMYTSNNMFA |
| | MYRHFLNFAE KLEVRCNTET |
| 361 | SEDPSVTTTL EHLHKIKAAC |
| | KAGLARTKDD TFDELRSRLL |
| 401 | ALTGGSFYLA WTYNFLDLRG |
| | P |

Another a C-terminal truncation of a Botryococcus braunii squalene synthase can have 83 amino acids truncated from the C-terminus, and the following sequence (SEQ ID NO:56) (also called BbSQS CΔ83).

| | |
|---|---|
| 1 | MGMLRWGVES LQNPDELIPV |
| | LRMIYADKFG KIKPKDEDRG |
| 41 | FCYEILNLVS RSFAIVIQQL |
| | PAQLRDPVCI FYLVLRALDT |
| 81 | VEDDKKIAAT TKIPLLRDFY |
| | EKISDRSFRM TAGDQKDYIR |
| 121 | LLDQYPKVTS VFLKLTPREQ |
| | EIIADITKRM GNGMADFVHK |
| 161 | GVPDTVGDYD LYCHYVAGVV |
| | GLGLSQLFVA SGLQSPSLTR |
| 201 | SEDLSNHMGL FLQKTNIIRD |
| | YFEDINELPA PRMFWPREIW |
| 241 | GKYANNLAEF KDPANKAAAM |
| | CCLNEMVTDA LRHAVYCLQY |
| 281 | MSKIEDPQIF NFCAIPQTMA |
| | FGTLSLCYNN YTIFTGPKAA |
| 321 | VKLRRGTTAK LMYTSNNMFA |
| | MYRHFLNFAE KLEVRCNTET |
| 361 | SEDPSVTTTL EHLHKIKA |

In another example, an Euphorbia lathyris is squalene synthase can be used, for example, with the following sequence (SEQ ID NO:57; UNIPROT accession no. A0A0A6ZA44_9ROSI).

| | |
|---|---|
| 1 | MGSLGAILKH PDDFYPLLKL |
| | KMAAKHAEKQ IPAQPHWGFC |
| 41 | YSMLHKVSRS FSLVIQQLGT |
| | ELRDAVGIFY LVLRALDTVE |
| 81 | DDTSIPTDVK VPILIAFHKH |
| | IYDPEWHFSC GTKEYKVLMD |
| 121 | QIHHLSTAFL ELGKSYQEAI |
| | EDITKKMGAG MAKFICKEVE |
| 161 | TVDDYDEYCH YVAGLVGLGL |
| | SKLFDASGFE DLAPDDLSNS |
| 201 | MGLFLQKTNI IRDYLEDINE |
| | IPKSRMFWPR QIWSKYVNKL |
| 241 | EDLKYEENSV KAVQCLNDMV |
| | TNALIHMDDC LKYKSALRDP |
| 281 | AIFRFCAIPQ IMAIGTLALC |
| | YNNVEVFRGV VKMRRGLTAK |
| 321 | VIDRTRTMAD VYRAFFDFSC |
| | MMKSKVDRND PNAEKTLNRL |
| 361 | EAVQKTCKES GLLHKRRSYI |
| | NESKPYNSTM VILLKIVLAI |
| 401 | ILAYLSKRAN |

A nucleotide sequence encoding the Euphorbia lathyris squalene synthase with SEQ ID NO:57 is shown below as SEQ ID NO:58 (NCBI accession no. JQ694152.1).

| | |
|---|---|
| 1 | GAACCTTGTG GCGTGCAGAG |
| | AGAGACAGAG AGAGACAGAG |
| 41 | ATTGTTGAAT CTCTATTTAA |
| | TTCATAGTAG CCTCATTGGA |
| 81 | CTCAATCCGT CGTTTTCGTT |
| | TCCATCTCCT TTAAAAACCA |
| 121 | GTCGATCGTT TCTCCTCAAT |
| | TTCGACTTCA ACTCTTTCTT |
| 161 | TCGCTTATTC ATTTGGTTTT |
| | TCAAGGGATC TGAGGATAAT |
| 201 | GGGGAGTTTG GGAGCAATTC |
| | TGAAGCATCC GGATGATTTT |
| 241 | TACCCGCTTT TGAAGCTGAA |
| | AATGGCTGCT AAACATGCTG |
| 281 | AGAAGCAGAT CCCAGCACAA |
| | CCTCACTGGG GTTTCTGTTA |
| 321 | CTCCATGCTT CATAAGGTCT |
| | CTCGTAGCTT TTCTCTTGTC |
| 361 | ATTCAACAGC TTGGCACTGA |
| | GCTCCGTGAC GCTGTTTGTA |
| 401 | TATTCTATTT GGTTCTTCGA |
| | GCCCTTGATA CTGTTGAGGA |
| 441 | TCATACAACC ATCCCTACAG |
| | ATGTGAAAGT GCCGATCTTG |
| 481 | ATAGCTTTTC ACAAGCACAT |
| | ATACGATCCT GAATGGCATT |
| 521 | TTTCTTGTGG TACTAAGGAA |
| | TATAAAGTTC TCATGGACCA |
| 561 | GATTCATCAT CTTTCAACTG |
| | CTTTTCTTGA GCTTGGGAAA |
| 601 | AGTTATCAGG AGGCAATCGA |
| | GGATATCACG AAAAAAATGG |
| 641 | GTGCAGGAAT GGCTAAATTC |
| | ATATGCAAAG AGGTGGAAAC |
| 681 | AGTTGATGAC TACGATGAAT |
| | ATTGCCATTA TGTTGCAGGA |
| 721 | CTTGTTGGAC TAGGTCTTTC |
| | CAAGCTTTTT GATGCCTCTG |
| 761 | GATTTGAAGA TTTGGCACCA |
| | GATGACCTTT CCAACTCGAT |
| 801 | GGGGTTATTT CTCCAGAAAA |
| | CAAACATTAT CCGGGATTAT |
| 841 | TTGGAGGATA TAAATGAGAT |
| | ACCTAAGTCA CGCATGTTTT |
| 381 | GGCCTCGCCA GATCTGGAGT |
| | AAATATGTTA ATAAACTTGA |
| 921 | GGACTTGAAA TATGAAGAAA |
| | ACTCAGTCAA GGCAGTGCAA |
| 961 | TGCTTGAATG ATATGGTTAC |
| | TAATGCTTTG ATACATATGG |
| 1001 | ATGATTGCTT GAAATACATG |
| | TCGGCACTAC GAGATCCTGC |
| 1041 | TATATTTCGT TTTTGTGCCA |
| | TCCCTCAGAT TATGGCAATT |
| 1081 | GGAACCCTAG CATTGTGCTA |
| | CAACAACGTT GAAGTATTTA |
| 1121 | GACCTGTACT GAAGATCAGG |
| | CGTGCTCTTA CTGCAAAGGT |
| 1161 | CATTGACAGA ACAAGGACCA |
| | TGGCAGATGT CTATCGGGCC |
| 1201 | TTCTTTGACT TCTCATGTAT |
| | GATGAAATCC AAGGTTGACA |
| 1241 | GGAATGATCC AAATGCAGAA |
| | AAGACATTGA ACAGGCTGGA |
| 1281 | AGCAGTGCAA AAAACTTGCA |
| | AGGAGTCTGG GCTGCTAAAC |
| 1321 | AAAAGGAGAT CTTAGATAAA |
| | TGAGAGCAAG CCATATAATT |
| 1361 | CTACTATGGT TATTCTACTG |
| | ATGATTGTAT TGGCAATCAT |
| 1401 | TTTGGCTTAT CTGAGCAAAC |
| | GGGCCAACTA ACTAGTGTAA |
| 1441 | CTTCTGTTAA GTAATCAGTT |
| | GAGGATTTGA ATCCGGTTAT |
| 1481 | CGTGAAACCG GGTTATTGCA |
| | GGATGTCTAC TTCTGTGAAC |
| 1521 | AATTTCTGCA GATGGATGGC |
| | TAGCTAGCAA TGAAGGTGCT |
| 1561 | TGCTGGACTT GTTCCAGGAG |
| | AGTTGTGAAT TTGATGTTTC |
| 1601 | AGTATATAGT GTAGTGCCAT |
| | AACAATGTTT GTGTCCAATG |
| 1641 | TGCCACTAAT GTGATCATAT |
| | TAGTGTTTTG TTCTCGTGGG |
| 1681 | TTGTTATTAT ACTCCTTAAT |
| | TATGGAATTG AAGCAATATC |
| 1721 | TTGAAGGATC TTCTGAATAT |
| | CTTGATTCAA GTCGCTGTTA |
| 1761 | TTCACATC |

In some cases, the Euphorbia lathyris squalene synthase can have a C-terminal truncation, for example, of about 20-50 amino acids. Such a C-terminal truncation of a Euphorbia lathyris squalene synthase can have 36 amino acids truncated from the C-terminus, and the following sequence (SEQ ID NO:59) (also called ElSQS CΔ36).

| | |
|---|---|
| 1 | MGSLGAILKH PDDFYPLLKL |
| | KMAAKHAEKQ IPAQPHWGFC |
| 41 | YSMLHKVSRS FSLVIQQLGT |
| | ELRDAVCIFY LVLRALDTVE |
| 81 | DDTSIPTDVK VPILIAFHKH |
| | IYDPEWHFSC GTKEYKVLMD |
| 121 | QIHHLSTAFL ELGKSYQEAI |
| | EDITKKMGAG MAKFICKEVE |
| 161 | TVDDYDEYCH YVAGLVGLGL |
| | SKLFDASGFE DLAPDDLSNS |
| 201 | MGLFLQKTNI IRDYLEDINE |
| | IPKSRMFWPR QIWSKYVNKL |
| 241 | EDLKYEENSV KAVQCLNDMV |
| | TNALIHMDDC LKYMSALRDP |
| 281 | AIFRFCAIPQ IMAIGTLALC |
| | YNNVEVFRGV VKMRRGLTAK |
| 321 | VIDRTRTMAD VYRAFFDFSC |
| | MMKSKVDRND PNAEKTLNRL |
| 361 | EAVQKTCKES GLLN |

In another example, a Ganoderma lucidum squalene synthase can be used, for example, with the following sequence (SEQ ID NO:61; NCBI accession no. ABF57213.1).

| | |
|---|---|
| 1 | MGATSMLTLL LTHPFEFRVL |
| | IQYKLWHEPK RDITQVSEHP |
| 41 | TSGWDRPTMR RCWEFLDQTS |
| | RSFSGVIKEV EGDLARVICL |
| 81 | FYLVLRGLDT IEDDMTLPDE |
| | KKQPILRQFH KLAVKPGWTF |
| 121 | DECGPKEKDR QLLVEWTVVS |
| | EELNRLDACY RDIIIDIAEK |
| 161 | MQTGMADYAH KAATTNSIYI |
| | GTVDEYNLYC HYVAGLVGEG |
| 201 | LTRFWAASGK EAEWLGDQLE |
| | LTNAMGLMLQ KTNIIRDFRE |
| 241 | DAEERRFFWP REIWGRDAYG |
| | KAVGRANGFR EMHELYERGN |
| 281 | EKQALWVQSG MVVDVLGHAT |
| | DSLDYLRLLT KQSIFCFCAI |
| 321 | PQTMAMATLS LCFMNYDKFH |
| | NHIKIRRAEA ASLIMRSTNP |
| 361 | RDVAYIFRDY ARKMHARALP |
| | EDPSFLRLSV ACGKIEQWCE |
| 401 | RHYPSFVRLQ QVSGGGIVFD |
| | PSDARTKVVE AAQARDNELA |
| 441 | REKRLAELRD KTGKLERKLR |
| | WSQAPSS |

A nucleotide sequence encoding the Ganoderma lucidum squalene synthase with SEQ ID NO:61 is shown below as SEQ ID NO:62 (NCBI accession no. DQ494674.1).

| | |
|---|---|
| 1 | ATGGGCGCGA CGTCTATGCT |
| | CACCCTCCTC CTCACACACC |
| 41 | CCTTCGAGTT CCGCGTCCTC |
| | ATCCAATACA AGCTCTGGCA |
| 81 | CGAACCAAAA CGCGACATTA |
| | CCCAAGTCTC CGAGCACCCG |
| 121 | ACTTCAGGAT GGGACCGCCC |
| | TACTATGCGA CGGTGTTGGG |
| 161 | AGTTCCTTGA CCAGACCAGC |
| | CGGAGTTTCT CTGGGGTCAT |
| 201 | CAAGGAAGTG GAGGGTGATT |
| | TAGCAAGAGT GATCTGCTTA |
| 241 | TTCTACCTGG TGCTACGAGG |
| | CCTGGACACG ATCGAAGATG |
| 281 | ACATGACGCT TCCTGACGAG |
| | AAAAAACAAC CCATACTCCG |
| 321 | ACAATTCCAC AAACTCGCCG |
| | TGAAGCCCGG TTGGACATTC |
| 361 | GACGAGTGTG GACCCAAAGA |
| | AAAGGACAGG CAACTCCTCG |
| 401 | TCGAGTGGAC AGTTGTCAGC |
| | GAAGAGCTCA ACCGTCTCGA |
| 441 | CGCATGCTAC CGCGATATTA |
| | TTATCGACAT TGCGGAAAAG |
| 481 | ATGCAGACCG GGATGGCCGA |
| | CTACGCGCAT AAAGCAGCGA |
| 521 | CCACGAATTC GATTTACATC |
| | GGAACCGTCG ACGAGTACAA |
| 561 | CCTCTACTGC CACTACGTCG |
| | CCGGCCTCGT CGGCGAGGGC |
| 601 | CTCACGCGCT TCTGGGCCGC |
| | GTCCGGCAAG GAGGCGGAAT |
| 641 | GGCTGGGGGA CCAGCTCGAG |
| | CTGACGAACG CGATGGGCCT |
| 681 | CATGCTGCAG AAGACGAACA |
| | TTATCCGTGA CTTCCGCGAG |
| 721 | GACGCCGAGG AGCGCCGCTT |
| | CTTCTGGCCG CGCGAGATCT |
| 761 | GGGGGCGCGA CGCATACGGC |
| | AAGGCCGTCG GCCGCGCGAA |
| 801 | CGGGTTCCGC GAGATGCACG |
| | AGCTGTACGA GCGGGGCAAC |
| 341 | GAGAAGCAGG CGCTGTGGGT |
| | GCAGAGCGGG ATGGTCGTTG |
| 881 | ACGTGCTCGG GCACGCTACA |
| | GACTCGCTCG ACTATCTCCG |
| 921 | CCTACTCACG AAGCAGAGCA |
| | TCTTCTGCTT CTGTGCGATC |
| 961 | CCACAAACGA TGGCCATGGC |
| | CACCCTCAGC TTGTGCTTCA |
| 1001 | TGAACTACGA CATGTTCCAC |
| | AACCATATCA AGATCCGCAG |
| 1041 | GGCTGAGGCT GCCTCGCTTA |
| | TTATGCGGTC AACGAACCCC |
| 1081 | CGCGACGTCG CATACATTTT |
| | CCGCGACTAC GCGCGCAAGA |
| 1121 | TGCACGCCCG CGCGCTGCCC |
| | GAGGACCCCT CCTTCCTCCG |
| 1161 | CCTCTCCGTC GCGTGCGGCA |
| | AGATCGAGCA GTGGTGCGAG |
| 1201 | CGCCACTACC CCTCCTTTGT |
| | CCGCCTCCAG CAGGTCTCGG |
| 1241 | GTGGGGGCAT CGTGTTCGAC |
| | CCGAGCGACG CGCGCACCAA |
| 1281 | GGTCGTCGAG GCCGCGCAGG |
| | CCCGCGACAA CGAGCTCGCG |
| 1321 | CGCGAGAAGC GCCTGGCCGA |
| | GCTCCGTGAC AAGACTGGAA |
| 1361 | AGCTTGAGCG CAAGCTGCGG |
| | TGGACTCAAG CCCCATCGAG |
| 1401 | CTGA |

In some cases, the Ganoderma lucidum squalene synthase can have a C-terminal truncation, for example, of about 20-80 amino acids. Such a Ganoderma lucidum squalene synthase can, for example, have 61 amino acids truncated from the C-terminus, to have the following sequence (SEQ ID NO:63) (also called GlSQS CΔ61).

| | |
|---|---|
| 1 | MGATSMLTLL LTHPFEFRVL |
| | IQYKLWHEPK RDITQVSEHP |
| 41 | TSGWDRPTMR RCWEFLDQTS |
| | RSFSGVIKEV EGDLARVICL |
| 81 | FYLVLRGLDT IEDDMTLPDE |
| | KKQPILRQFH KLAVKPGWTF |
| 121 | DECGPKEKDR QLLVEWTVVS |
| | EELNRLDACY RDIIIDIAEK |
| 161 | MQTGMADYAH KAATTNSIYI |
| | GTVDEYNLYC HYVAGLVGEG |
| 201 | LTRFWAASGK EAEWLGDQLE |
| | LTNAMGLMLQ KTNIIRDFRE |
| 241 | DAEERRFFWP REIWGRDAYG |
| | KAVGRANGFR EMHELYERGN |
| 281 | EKQALWVQSG MVVDVLGHAT |
| | DSLDYLRLLT KQSIFCFCAI |
| 321 | PQTMAMATLS LCFMNYDKFH |
| | NHIKIRRAEA ASLIMRSTNP |
| 361 | RDVAYIFRDY ARKMHARALP |
| | EDPSFLRLSV ACGKIEQWCE |
| 401 | RHYPSF |

In another example, a Ganoderma lucidum squalene synthase can, for example, have 30 amino acids truncated from the C-terminus, and the following sequence (SEQ ID NO:64) (also called GISQS CΔ30).

| | |
|---|---|
| 1 | MGATSMLTLL LTHPFEFRVL |
| | IQYKLWHEPK RDITQVSEHP |
| 41 | TSGWDRPTMR RCWEFLDQTS |
| | RSFSGVIKEV EGDLARVICL |
| 81 | FYLVLRGLDT IEDDMTLPDE |
| | KKQPILRQFH KLAVKPGWTF |
| 121 | DECGPKEKDR QLLVEWTVVS |
| | EELNRLDACY RDIIIDIAEK |
| 161 | MQTGMADYAH KAATTNSIYI |
| | GTVDEYNLYC HYVAGLVGEG |
| 201 | LTRFWAASGK EAEWLGDQLE |
| | LTNAMGLMLQ KTNIIRDFRE |
| 241 | DAEERRFFWP REIWGRDAYG |
| | KAVGRANGFR EKHELYERGN |
| 281 | EKQALWVQSG MVVDVLGHAT |
| | DSLDYLRLLT KQSIFCFCAI |
| 321 | PQTMAMATLS LCFMNYDMFH |
| | NHIKIRRAEA ASLIMRSTNP |
| 361 | RDVAYIFRDY ARKMHARALP |
| | EDPSFLRLSV ACGKIEQWCE |
| 401 | RHYPSFVRLQ QVSGGGIVFD |
| | PSDARTKVVE AAQARDN |

In another example, a Mortierella alpina squalene synthase can be used, for example, with the following sequence (SEQ ID NO:65; NCBI accession no. ALA40031.1).

| | |
|---|---|
| 1 | MASAILASLL HPSEVLALVQ YKLSPKTQHD YSNDKTRQRL |
| 41 | YHHLNMTSRS FSAVIQDLDE ELKDAICLFY LVLRGLDTIE |
| 81 | DDMTIDLDTK LPYLRTFHEI IYQKGWLFTK NGPNEKDRQL |
| 121 | LVEFDAIIEG FLQLKPAYQT IIADITKRMG NGMAHYATAG |
| 161 | IHVETNADYD EYCHYVAGLV GIGISEMFSA CGFESPLVAE |
| 201 | RKDLSNSMGL FLQKTNIARD YLEDLRDNRR FWPKEIWGQY |
| 241 | AETMEDLVKP ENKEKALQCL SHMIVNAMEH IRDVLEYLSM |
| 281 | IKNPSCFKFC AIPQVMAMAT LNLLHSNYKV FTHENIKIRK |
| 321 | GETVWLMKES DSMDKVAAIF RLYARQINNK SNSLDPHFVD |
| 361 | IGVICCEIEQ ICVGRFPGST IEMKRMQAGV LGGKTGTVLA |
| 401 | AAAAVAGAVV INNALA |

A nucleotide sequence encoding the Mortierella alpina squalene synthase with SEQ ID NO:65 is shown below as SEQ ID NO:66 (NCBI accession no. KT318395.1).

| | |
|---|---|
| 1 | ATGGCTTCTG CTATCCTCGC CTCGCTCCTC CACCCTTCCG |
| 41 | AGGTGTTGGC CTTGGTCCAG TACAAACTCT CGCCAAAGAC |
| 81 | CCAACACGAC TACAGCAACG ATAAAACCAG GCAGCGCCTC |
| 121 | TACCACCACT TGAACATGAC CTCGCGTAGT TTCTCAGCGG |
| 161 | TCATCCAGGA TCTGGACGAG GAACTGAAGG ATGCGATTTG |
| 201 | CTTGTTCTAC CTCGTCCTTC GTGGACTCGA TACCATTGAG |
| 241 | GACGATATGA CGATTGATTT GGACACCAAG TTGCCATATC |
| 281 | TGAGGACGTT CCACGAAATC ATCTACCAGA AGGGATGGAC |
| 321 | CTTTACGAAG AATGGTCCTA ACGAAAAAGA CCGCCAGTTG |
| 361 | CTGGTTGAGT TTGACGCCAT CATCGAGGGA TTCTTGCAAC |
| 401 | TAAAGCCAGC GTATCAAACC ATCATTGCCG ACATCACTAA |
| 441 | ACGCATGGGC AATGGAATGG CTCACTACGC CACTGCAGGA |
| 481 | ATTCACGTTG AGACTAATGC TGATTATGAC GAATACTGCC |
| 521 | ATTACGTCGC GGGCCTTGTT GGTCTGGGAT TGAGCGAGAT |
| 561 | GTTCAGCGCC TGTGGATTTG AATCGCCTTT GGTAGCCGAG |
| 601 | AGAAAAGACC TCTCAAACTC GATGGGTCTG TTTCTCCAAA |
| 641 | AGACCAACAT CGCACGCGAT TATCTCGAGG ATCTGCGCGA |
| 681 | CAATCGCCGT TTCTGGCCAA AGGAGATCTG GGGCCAGTAT |
| 721 | GCGGAAACGA TGGAGGACCT AGTCAAGCCC GAGAACAAGG |
| 761 | AGAAGGCTCT GCAGTGTCTG AGCCACATGA TCGTCAACGC |
| 801 | CATGGAGCAC ATCCGAGATG TCCTCGAGTA CCTTAGTATG |
| 841 | ATCAAGAACC CGTCCTGCTT TAAGTTCTGT GCGATTCCCC |
| 381 | AGGTTATGGC CATGGCGACT TTGAACCTCC TCCACTCCAA |
| 921 | CTACAAGGTT TTTACGCACG AGAATATCAA AATCCGCAAG |
| 961 | GGCGAGACAG TGTGGCTGAT GAAGGAGTCA GACAGCATGG |
| 1001 | ACAAGGTGGC AGCCATCTTC CGACTTTATG CGCGCCAGAT |
| 1041 | CAACAACAAG TCAAACTCTC TGGACCCCCA CTTTGTTGAC |
| 1081 | ATCGGTGTCA TTTGCGGCGA GATTGAGCAG ATCTGTGTTG |
| 1121 | GAAGGTTCCC AGGATCCACG ATTGAGATGA AGCGCATGCA |
| 1161 | AGCTGGAGTG CTGGGCGGCA AAACCGGAAC CGTGCTTGCT |
| 1201 | GCAGCTGCGG CTGTTGCAGG AGCTGTTGTT ATCAACAATG |
| 1241 | CGCTCGCATA A |

In some cases, the Mortierella alpina squalene synthase can have a C-terminal truncation, for example, of about 10-40 amino acids. Such a Mortierella alpina squalene synthase can, for example, have 37 amino acids truncated from the C-terminus, to have the following sequence (SEQ ID NO:67) (also called MaSQS CΔ37).

| | |
|---|---|
| 1 | MASAILASLL HPSEVLALVQ YKLSPKTQHD YSNDKTRQRL |
| 41 | YHHLNMTSRS FSAVIQDLDE ELKDAICLFY LVLRGLDTIE |
| 81 | DDMTIDLDTK LPYLRTFHEI IYQKGWTFTK NGPNEKDRQL |
| 121 | LVEFDAIIEG FLQLKPAYQT IIADITKRKG NGMAHYATAG |
| 161 | IHVETNADYD EYCHYVAGLV GLGLSEMFSA CGFESPLVAE |
| 201 | RKDLSNSMGL FLQKTNIARD YLEDLRDNRR FWPKEIWGQY |
| 241 | AETMEDLVKP ENKEKALQCL SHMIVNAMEH IRDVLEYLSM |
| 281 | IKNPSCFKFC AIPQVKAKAT LNLLHSNYKV FTHENIKIRK |
| 321 | GETVWLMKES DSMDKVAAIF RLYARQINNK SNSLDPHFVD |
| 361 | IGVICGEIEQ ICVGRFPGS |

In another example, a Mortierella alpina squalene synthase can, for example, have 17 amino acids truncated from the C-terminus, and the following sequence (SEQ ID NO:68) (also called MaSQS CΔ17).

| | |
|---|---|
| 1 | MASAILASLL HPSEVLALVQ YKLSPKTQHD YSNDKTRQRL |
| 41 | YHHLNMTSRS FSAVIQDLDE ELKDAICLFY LVLRGLDTIE |
| 81 | DDMTIDLDTK LPYLRTFHEI IYQKGWTFTK NGPNEKDRQL |
| 121 | LVEFDAIIEG FLQLKPAYQT IIADITKRMG NGMAHYATAG |
| 161 | IHVETNADYD EYCHYVAGLV GLGLSEMFSA CGFESPLVAE |
| 201 | RKDLSNSMGL FLQKTNIARD YLEDLRDNRR FWPKEIWGQY |
| 241 | AETMEDLVKP ENKEKALQCL SHMIVNAMEH IRDVLEYLSM |
| 281 | IKNPSCFKFC AIPQVMAMAT LNLLHSNYKV FTHENIKIRK |
| 321 | GETVWLMKES DSMDKVAAIF RLYARQINNK SNSLDPHFVD |
| 361 | IGVICGEIEQ ICVGRFPGST IEMKRMQAGV LGGKTGTVL |

Hence, a variety of native and modified squalene synthases can be used in the expression systems, cells, and methods described herein.

### WRINKLED (WRI1)

WRINKLED1 (WRI1) is a member of the AP2/EREBP family of transcription factors and master regulator of fatty acid biosynthesis in seeds. Because WRI1 is a transcription factor, it is generally expressed in the cytosol and not expressed as a fusion partner with a lipid droplet surface protein. However, ectopic production of WRI1 in vegetative tissues promotes fatty acid synthesis in plastids and, indirectly, triacylglycerol accumulation in lipid droplets.

As illustrated herein, increased WRI1 expression can increase the synthesis of proteins involved in oil synthesis. The data provided herein also shows that co-expression of WRI1 with ectopic lipid biosynthesis enzymes and a lipid droplet associated protein can improve terpene and terpenoid production.

Plants can be generated as described herein to include WRINKLED1 nucleic acids that encode WRINKLED transcription factors. Plants are especially desirable when the WRINKLED1 nucleic acids are operably linked to control sequences capable of WRINKLED1 expression in a multitude of plant tissues, or in selected tissues and during selected parts of the plant life cycle to optimize the synthesis of oil and terpenoids. Such control sequences are typically heterologous to the coding region of the WRINKLED1 nucleic acids.

One example of an amino acid sequence for a WRINKLED1 (WRI1) sequence from Arabidopsis thaliana is available as accession number AAP80382.1 (GI:32364685) and is reproduced below as SEQ ID NO:69.

| | |
|---|---|
| 1 | MKKRLTTSTC SSSPSSSVSS STTTSSPIQS EAPRPKRAKR |
| 41 | AKKSSPSGDK SHNPTSPAST RRSSIYRGVT RHRWTGRFEA |
| 81 | HLWDKSSWNS IQNKKGKQVY LGAYDSEEAA AHTYDLAALK |
| 121 | YWGPDTILNF PAETYTKELE EMQRVTKEEY LASLRRQSSG |
| 161 | FSRGVSKYRG VARHHHNGRW EARIGRVFGN KYLYLGTYNT |
| 201 | QEEAAAAYDM AAIEYRGANA VTNFDISNYI DRLKKKGVFP |
| 241 | FPVNQANHQE GILVEAKQEV ETREAKEEPR EEVKQQYVEE |
| 281 | PPQEEEEKEE EKAEQQEAEI VGYSEEAAVV NCCIDSSTIM |
| 301 | EMDRCGDNNE LAWNFCMMDT GFSPFLTDQN LANENPIEYP |
| 361 | ELFNELAFED NIDFMFDDGK HECLNLENLD CCVVGRESPP |
| 401 | SSSSPLSCLS TDSASSTTTT TTSVSCNYLV |

A nucleic acid sequence for the above Arabidopsis thaliana WRI1 protein is available as accession number AY254038.2 (GI:51859605), and is reproduced below as SEQ ID NO:70.

| | |
|---|---|
| 1 | AAACCACTCT GGTTCCTCTT CCTCTGAGAA ATCAAATCAC |
| 41 | TCACACTCCA AAAAAAAATC TAAAETTTCT CAGAGTTTAA |
| 81 | TGAAGAAGCG CTTAACCACT TCCACTTGTT CTTCTTCTCC |
| 121 | ATCTTCCTCT GTTTCTTCTT CTACTACTAC TTCCTCTCCT |
| 161 | ATTCACTCGC AGGCTCCAAG CCCTAAACGA GCCAAAACCC |
| 201 | CTAAGAAATC TTCTCCTTCT GGTGATAAAT CTCATAACCC |
| 241 | CACAACCCCT GCTTCTACCC CACGCACCTC TATCTACACA |
| 281 | GCACTCACTA CACATAGATC CACTGCGAGA TTCGAGGCTC |
| 301 | ATCTTTGCGA CAAAAGGTCT TCGAATTCGA TTCAGAACAA |
| 361 | GAAAGGCAAA CAAGTTTATC TGGGAGCATA TGACAGTGAA |
| 401 | GAAGCAGCAG CACATACGTA CGATCTGGCT GCTCTCAAGT |
| 421 | ACTGGGGACC CGACACCATC TTGAATTTTC CGGCAGAGAC |
| 481 | GTACACAAAG GAATTGGAAG AAATGCAGAG AGTGACAAAG |
| 521 | GAAGAATATT TGGCTTCTCT CCGCCGCCAG AGCAGTGGTT |
| 581 | TCTCCAGAGG CGTCTCTAAA TATCGCGGCG TCGCTAGGCA |
| 601 | TCACCAGAAC GGAAGATGGG AGGCTCGGAT CGGAAGAGTG |
| 641 | TTTGGGAACA AGTACTTGTA CCTCGGCACC TATAATAGGC |
| 681 | AGGAGGAAGC TGCTGCAGCA TATGACATGG CTGCGATTGA |
| 721 | GTATCGAGGC GCAAACGCGG TTACTAATTT CGACATTAGT |
| 761 | AATTACATTG ACCGGTTAAA GAAGAAAGGT GTTTTCCCGT |
| 801 | TCCCTGTGAA CCAACCTAAC CATCAAGAGG GTATTCTTCT |
| 841 | TGAAGCCAAA CAAGAAGTTG AAACGAGAGA AGCGAAGGAA |
| 381 | GAGCCTAGAG AAGAAGTGAA ACAACAGTAC GTGGAAGAAC |
| 921 | CACCGGAAGA AGAACAAGAG AAGGAAGAAG AGAAACCACA |
| 961 | GCAACAAGAA GCAGAGATTG TAGGATATTC AGAAGAAGCA |
| 1001 | CCAGTGGTCA ATTGCTGCAT AGACTCTTCA ACCATAATGG |
| 1041 | AAATGGATCG TTGTGGGGAG AACAATGAGC TGGCTTGGAA |
| 1081 | CTTCTGTATG ATGGATACAG GGTTTTCTCC GTTTTTGACT |
| 1121 | GATCAGAATC TCGCGAATGA GAATCCCATA GAGTATCCGG |
| 1141 | AGCTATTCAA TGAGTTAGCA TTTGAGGACA ACATCGACTT |
| 1201 | CATGTTCGAT GATGGGAAGC ACGAGTGCTT GAACTTGGAA |
| 1241 | AATCTGGATT GTTGCGTGGT GGGAAGAGAG AGCCCACCCT |
| 1281 | CTTCTTCTTC ACCATTGTCT TGCTTATCTA CTGACTCTGC |
| 1321 | TTCATCAACA ACAACAACAA CAACCTCGGT TTCTTCTAAC |
| 1361 | TATTTGGTCT GAGAGAGAGA GCTTTGCCTT CTAGTTTGAA |
| 1401 | TTTCTATTTC TTCCGCTTCT TCTTCTTTTT TTTCTTTTGT |
| 1441 | TGGGTTCTGC TTAGGCTTTG TATTTCAGTT TCAGGGCTTC |
| 1481 | TTCGTTGGTT CTGAATAATC AATGTCTTTG CCCCTTTTCT |
| 1501 | AATGGGTACC TGAAGGGCGA |

Yields of triacylglycerol and terpenoids can further increased by removal of an intrinsically disordered C-terminal region of Arabidopsis thaliana WRI1. For example, use of a truncated WRI1 protein with amino acids 1-397 (AtWRI1(1-397)) can increase the WRI1 protein stability and increase the amounts of oils and terpenoids produced by plants and plant cells.

The A. thaliana WRINKLED1 (AtWRI11-397; SEQ ID NO:29) amino acid sequence is shown below.

| | |
|---|---|
| 1 | MKKRLTTSTC SSSPSSSVSS STTTSSPIQS EAPRPKRAKR |
| 41 | AKKSSPSGDK SHNPTSPAST RRSSIYRGVT RHRWTGRFEA |
| 81 | HLWDKSSWNS IQNKKGKQVY LGAYDSEEAA AHTYDLAALK |
| 121 | YWGRDTILNF PAETYTKELE EMQRVTKEEY LASLRRQSSG |
| 161 | FSRGVSKYRG VARHHHNGRW EARIGRVFGN KYLYLGTYNT |
| 201 | QEEAAAAIDM AAIEYRGANA VTNFDISNYI DRLKKKGVFP |
| 241 | FPVNQANHQE GILVEAKQEV ETREAKEEPR EEVKQQYVEE |
| 281 | PPQEEEEKEE EKAEQQEAEI VGYSEEAAVV NCCIDSSTIM |
| 321 | EMDRCGDNNE LAWNFCMMDT GESPFLTDQN LANENPIEYP |
| 361 | ELFNELAFED NIDFMFDDGK HECLNLENLD CCVVGRESPP |
| 401 | SSSSPLSCLS TDSASSTTTT TTSVSCNYLV |

The A. thaliana WRINKLED1 (AtWRI11-397; SEQ ID NO: 30) nucleotide sequence is shown below.

| | |
|---|---|
| 1 | AAACCACTCT GCTTCCTCTT CCTCTGAGAA ATCAAATCAC |
| 41 | TCACACTCCA AAAAAAAATC TAAACTTTCT CAGACTTTAA |
| 81 | TGAAGAAGCG CTTAACCACT TCCACTTGTT CTTCTTCTCC |
| 121 | ATCTTCCTCT GTTTCTTCTT CTACTACTAC TTCCTCTCCT |
| 161 | ATTCAGTCGG AGGCTCCAAG GCCTAAACGA GCCAAAAGGG |
| 201 | CTAAGAAATC TTCTCCTTCT GGTGATAAAT CTCATAACCC |
| 241 | GACAAGCCCT GCTTCTACCC GACGCAGCTC TATCTACAGA |
| 281 | GGAGTCACTA GACATAGATG GACTGGGAGA TTCGAGGCTC |
| 321 | ATCTTTGGGA CAAAAGCTCT TGGAATTCGA TTCAGAACAA |
| 361 | GAAACGCAAA CAAGTTTATC TGGGAGCATA TGACACTGAA |
| 401 | GAAGCAGCAG CACATACGTA CGATCTGGCT CCTCTCAAGT |
| 441 | ACTGCGGACC CGACACCATC TTGAATTTTC CGGCAGAGAC |
| 481 | GTACACAAAG CAATTCCAAG AAATGCAGAG AGTCACAAAG |
| 521 | GAAGAATATT TGGCTTCTCT CCGCCGCCAG AGCACTGGTT |
| 561 | TCTCCAGAGG CGTCTCTAAA TATCGCGGCG TCGCTAGGCA |
| 601 | TCACCACAAC GGAAGATGGG AGGCTCGGAT CGGAAGAGTG |
| 641 | TTTGGGAACA AGTACTTGTA CCTCGGCACC TATAATACGC |
| 681 | AGGAGGAAGC TGCTGCAGCA TATGACATGG CTGCGATTGA |
| 721 | GTATCGAGCC CCAAACCCGC TTACTAATTT CCACATTAGT |
| 761 | AATTACATTG ACCGGTTAAA GAAGAAAGGT GTTTTCCCGT |
| 801 | TCCCTGTGAA CCAAGCTAAC CATCAAGAGG GTATTCTTGT |
| 341 | TGAACCCAAA CAACAAGTTG AAACCACAGA AGCGAACCAA |
| 881 | GAGCCTAGAG AAGAAGTGAA ACAACAGTAC GTGGAAGAAC |
| 921 | CACCGCAAGA AGAAGAAGAG AAGGAAGAAG AGAAAGCAGA |
| 961 | GCAACAAGAA GCAGAGATTG TAGGATATTC AGAAGAAGCA |
| 1001 | GCAGTGGTCA ATTGCTGCAT AGACTCTTCA ACCATAATGG |
| 1041 | AAATGGATCG TTGTGGGGAC AACAATGAGC TGGCTTGGAA |
| 1081 | CTTCTGTATG ATGGATACAG GGTTTTCTCC GTTTTTGACT |
| 1121 | GATCAGAATC TCGCGAATGA GAATCCCATA GAGTATCCGG |
| 1161 | AGCTATTCAA TGAGTTAGCA TTTGAGGACA ACATCGACTT |
| 1201 | CATGTTCGAT GATGGGAAGC ACGAGTGCTT GAACTTGGAA |
| 1241 | AATCTGGATT GTTGCGTGGT GGGAAGAGAG AGCCCACCCT |
| 1281 | CTTCTTCTTC ACCATTGTCT TGCTTATCTA CTGACTCTGC |
| 1321 | TTCATCAACA ACAACAACAA CAACCTCGGT TTCTTCTAAC |
| 1361 | TATTTGGTCT GAGAGAGAGA GCTTTGCCTT CTAGTTTCAA |
| 1401 | TTTCTATTTC TTCCGCTTCT TCTTCTTTTT TTTCTTTTGT |
| 1441 | TGGGTTCTGC TTACGCTTTG TATTTCAGTT TCAGGGCTTG |
| 1481 | TTCGTTGGTT CTGAATAATC AATGTCTTTG CCCCTTTTCT |
| 1521 | AATGGGTACC TGAAGGGCGA |

Other types of WRI1 proteins (e.g., with different sequences) can also be used, such as any of the WRI1 proteins and sequences therefor that are described hereinbelow and in published US Patent Application US 2017/0002371 (which is incorporated by reference herein in its entirety).

For example, the WRI1 protein has a PEST domain that has an amino acid sequence enriched in proline (P), glutamic acid (E), serine (S), and threonine (T)), which is associated with intrinsically disordered regions (IDRs). Removal of the C-terminal PEST domain from WRI1 or use of mutations in such C-terminal PEST domains results in a more stable WRI1 transcription factors and increased oil biosynthesis by plants expressing such deleted or mutated WRINKLED transcription factors.

The Arabidopsis thaliana protein with SEQ ID NO:69 can have C-terminal deletions or mutations, for example in the following PEST sequence (SEQ ID NO:71).

| | |
|---|---|
| 396 | RESPP SSSSPLSCLS TDSASSTTTT TTSVSCNYLV. |

For example, expression of a C-terminally truncated Arabidopsis thaliana WRI1 protein or an Arabidopsis thaliana WRI1 protein with at least four mutations at any of positions 398, 401, 402, 407, 415, 416, 420, 421, 422, and/or 423 increases the content of triacylglycerol in plant tissues such as leaves and seeds. Hence, a mutant WRI1 protein can be used in the systems and methods described herein that includes a substitution, insertion, or deletion in any of the X residues of the following sequence (SEQ ID NO:72):

| | |
|---|---|
| 396 | REXPP XXSSPLXCLS TDSAXXTTTX XXXVSCNYLV. |

For example, at least four of the X residues in the SEQ ID NO:72 sequence can be a substitution, insertion, or deletion compared to the wild type sequence (SEQ ID NO: 71). The X residues are not acidic amino acids, for example, the X residues are not aspartic acid or glutamic acid. However, the X residue can be a small amino acid or a hydrophobic amino acid. For example, the X residues can each separately be alanine, glycine, valine, leucine, isoleucine, methionine, or any mixture thereof. As illustrated herein, WRI1 proteins with an alanine instead of a serine or a threonine at each of positions 398, 401, 402, and 407 have increased stability and, when expressed in plant cells, the cells produce more triacylglycerols than do wild type plants that do not express such a mutant WRI1 protein.

Another aspect of the invention is a mutant WRI1 protein with a truncation at the C terminus of at least 5, or at least 7, or at least 10, or at least 13, or at least 15, or at least 17, or at least 20, or at least 25, or at least 30, or at least 35, or at least 40, or at least 45 amino acids. For example, such deletions can be within the SEQ ID NO:50 portion of the WRI1 protein. Such mutant WRI1 proteins can be expressed in plant tissues to increase the oil/fatty acid/TAG content of those tissues.

Other types of WRI1 proteins also have utility for increasing the oil/fatty acid/TAG content of lipid droplets within plant tissues.

For example, an amino acid sequence for a WRI1 sequence from Brassica napus is available as accession number ADO16346.1 (GI:308193634). This Brassica napus WRINKLED1 sequence is reproduced below as SEQ ID NO:73.

| | |
|---|---|
| 1 | MRRPLTTSPS TSSSTSSSAC ILPTQPETPR PKRAKRAKKS |
| 41 | SIPTDVKPQN PTSPASTRRS STYRGVTRHR WTGRYEAHLW |
| 81 | DKSSWNSIQN KKGKQVYLGA YDSEEAAAHT YDLAALKYWG |
| 121 | PDTILNFPAE TYTKELEEMQ RCTKEEYLAS LRRQSSGFSR |
| 161 | GVSKYRGVAR HHHNGRWEAR IGPVEGNKYL YLGTYNTQEE |
| 201 | AAAAYDMAAI EYRGANAVTN FDISNYIDRL KKKGVFPFPV |
| 241 | SQANHOEAVL AEAKQEVEAK EEPTEEVKQC VEKEEPQEAK |
| 281 | EEKTEKKQQQ DEVEEAVVTC CIDSSESNEL AWDFCMMDSG |
| 301 | FAPFLTDSNL SSENPIEYPE LFNEMGFEDN IDFMFEEGKQ |
| 361 | DCLSLENLDC CDGVVVVGRE SPTSLSSSPL SCLSTDSASS |
| 401 | TTTTTITSVS CNYSV |

A nucleic acid sequence for the above Brassica napus WRI1 protein is available as accession number HM370542.1 (GI:308193633), and is reproduced below as SEQ ID NO:74.

| | |
|---|---|
| 1 | ATGAAGAGAC CCTTAACCAC TTCTCCTTCT ACCTCCTCTT |
| 41 | CTACTTCTTC TTCGGCTTGT ATACTTCCGA CTCAACCAGA |
| 61 | GACTCCAAGG CCCAAACGAG CCAAAAGGGC TAAGAAATCT |
| 121 | TCTATTCCTA CTCATGTTAA ACCACAGAAT CCCACCAGTC |
| 161 | CTGGCTCCAC CAGACGCACC TCTATCTACA CACCACTCAC |
| 201 | TAGACATAGA TGGACAGGGA GATACGAGGC TCATCTATGG |
| 241 | GACAAAAGCT CGTGGAATTC GATTCAGAAG AAGAAAGGCA |
| 281 | AACAAGTTTA TCTGGGAGCA TATGACAGCG AGGAAGCAGC |
| 321 | AGCGCATACG TACGATCTAG CTGCTCTCAA GTACTGGGGT |
| 361 | GCCGACACCA TCTTGAACTT TCCGGCTGAG ACGTACACAA |
| 401 | ACCACTTGGA CGAGATGCAG AGATGTACAA AGGAAGAGTA |
| 441 | TTTGGCTTCT CTCCGCCGCC AGAGCAGTGG TTTCTCTACA |
| 481 | GGCGTCTCTA AATATCGCGG CGTCGCCAGG CATCACCATA |
| 521 | ACGGAAGATG GGAAGCTAGG ATTGGAAGGG TGTTTGGAAA |
| 541 | CAAGTACTTG TACCTCGGCA CTTATAATAC GCAGGAGGAA |
| 601 | GCTGCAGCTG CATATGACAT GGCGGCTATA GAGTACAGAG |
| 641 | GCGCAAACGC AGTGACCAAC TTCGACATTA GTAACTACAT |
| 681 | CCACCGGTTA AAGAAAAAAG GTGTCTTCCC ATTCCCTGTG |
| 721 | AGCCAAGCCA ATCATCAAGA AGCTGTTCTT GCTGAAGCCA |
| 761 | AACAAGAAGT GGAAGCTAAA GAAGAGCCTA CAGAAGAAGT |
| 801 | GAAGCAGTGT GTCGAAAAAG AAGAACCGCA AGAAGCTAAA |
| 841 | GAAGAGAAGA CTGAGAAAAA ACAACAACAA CAAGAAGTGG |
| 881 | AGGAGGCGGT GGTCACTTGC TGCATTGATT CTTCGGAGAG |
| 921 | CAATGAGCTG GCTTGGGACT TCTGTATCAT CGATTCAGGC |
| 961 | TTTGCTCCGT TTTTGACGGA TTCAAATCTC TCGAGTGAGA |
| 1001 | ATCCCATTGA GTATCCTGAG CTTTTCAATG AGATGGGGTT |
| 1041 | TGAGGATAAC ATTGACTTCA TGTTCGAGGA AGGGAAGCAA |
| 1081 | GACTGCTTGA GCTTGGAGAA TCTGGATTGT TGCGATGGTG |
| 1121 | TTGTTGTGGT GGGAAGAGAG AGCCCAACTT CATTGTCGTC |
| 1161 | TTCACCGTTG TCTTGCTTGT CTACTGACTC TGCTTCATCA |
| 1201 | ACAACAACAA CAACAATAAC CTCTGTTTCT TGTAACTATT |
| 1241 | CTGTCTGA |

Expression of a C-terminally truncated Brassica napus WRI1 protein or an Brassica napus WRI1 protein with a mutation (e.g., substitution, insertion, or deletion) at four or more of positions 381, 383, 384, 386, 387, 388, 391, 399, 400, 401, 402, 403, 404, 405, 407, or 408 can increase the content of triacylglycerol in plant tissues such as leaves and seeds. Hence, a mutant WRI1 protein can be used in the systems and methods described herein that includes a mutation (substitution, insertion, or deletion) in the following sequence (SEQ ID NO: 75):

| | |
|---|---|
| 379 | RE SPTSLSSSPL SCLSTDSASS TTTTTITSVS CNYSV |

For example, expression of a C-terminally truncated Brassica napus WRI1 protein or a Brassica napus WRI1 protein with at least four mutations (substitution, insertion, or deletion) at any of positions 381, 383, 384, 386, 387, 388, 391, 399, 400, 401, 402, 403, 404, 405, 407, and/or 408 can increase the content of triacylglycerol in plant tissues such as leaves and seeds. Hence, a mutant WRI1 protein can be used that includes the following sequence (SEQ ID NO: 76):

RE XPXXLXXXPL XCLSTDSAXX XXXXXIXXVS CNYSV

where at least four of the X residues in the SEQ ID NO:76 sequence is a substitution, insertion, or deletion compared to the wild type sequence (SEQ ID NO:75). The X residues are not acidic amino acids such as aspartic acid or glutamic acid. However, the X residue can be a small amino acid or a hydrophobic amino acid. For example, the X residues can each separately be alanine, glycine, valine, leucine, isoleucine, methionine, or any mixture thereof.

Another aspect of the invention is a mutant WRI1 protein with a truncation at the C terminus of the SEQ ID NO:69 (or the SEQ ID NO:73) sequence of at least 4, or at least 5, or at least 7, or at least 10, or at least 13, or at least 15, or at least 17, or at least 20, or at least 25, or at least 30, or at least 35, or at least 40, or at least 45 amino acids. Such mutant WRI1 proteins can be expressed in plant tissues to increase the oil/fatty acid/TAG content of those tissues.

Another example of an amino acid sequence for a WRINKLED1 (WRI1) sequence from Brassica napus is available as accession number ABD16282.1 (GI:87042570), and is reproduced below as SEQ ID NO:77.

| | |
|---|---|
| 1 | MKRPLTTSPS SSSSTSSSAC ILPTQSETPR PKRAKRAKKS |
| 41 | SLRSDVKPQN PTSPASTRRS SIYRGVIRHR WTCRYEAHLW |
| 81 | DKSSWNSIQN KKGYQVYLGA YDSEEAAAHT YDLAALKYWG |
| 121 | PNTILNFPVE TYTKELEEMQ RCTKEEYTAS LRRQSSGFSR |
| 161 | GVSKYRGVAR HHHNGRWEAR IGRVFGNKYL YLGTYNTQEE |
| 201 | AAAAYDMAAI EYRGANAVTN FDIGNYIDRL KKKGVFPFPV |
| 241 | SQANHQEAVL AETKQEVEAK EEPTEEVKQC VEKEEAKEEK |
| 281 | TEKKQQQEVE EAVITCCIDS SESNELAWDF CMMDSGFAPF |
| 321 | LTDSNLSSEN PIEYPELFNE MGFEDNIDFM FEEGKQDCLS |
| 361 | LENLDCCDGV VVVGRESPTS LSSSPLSCLS TDSASSTTTT |
| 401 | ATTVTSVSWN YSV |

A nucleic acid sequence for the above Brassica napus WRI1 protein is available as accession number DQ370141.1 (GI:87042569), and is reproduced below as SEQ ID NO:78.

| | |
|---|---|
| 1 | ATGAAGAGAC CCTTAACCAC TTCTCCTTCT TCCTCCTCTT |
| 41 | CTACTTCTTC TTCGGCCTGT ATACTTCCGA CTCAATCAGA |
| 61 | GACTCCAAGG CCCAAACGAG CCAAAAGGGC TAAGAAATCT |
| 121 | TCTCTGCGTT CTGATGTTAA ACCACAGAAT CCCACCAGTC |
| 161 | CTGCCTCCAC CAGACGCAGC TCTATCTACA GAGGAGTCAC |
| 181 | TAGACATAGA TGGACAGGGA GATACGAAGC TCATCTATGG |
| 241 | GACAAAAGCT CGTGGAATTC GATTCAGAAC AAGAAAGGCA |
| 281 | AACAAGTTTA TCTGGGAGCA TATGACAGCG AGGAAGCAGC |
| 321 | AGCACATACG TACGATCTAG CTGCTCTCAA GTACTGGGGT |
| 361 | CCCAACACCA TCTTGAACTT TCCGGTTGAG ACGTACACAA |
| 401 | AGGAGCTGGA GGAGATGCAG AGATGTACAA AGGAAGAGTA |
| 441 | TTTGGCTTCT CTCCGCCGCC AGAGCAGTGG TTTCTCTAGA |
| 481 | GGCGTCTCTA AATATCGCGG CGTCGCCAGG CATCACCATA |
| 521 | ATGGAAGATG GGAAGCTCGG ATTGGAAGGG TGTTTGGAAA |
| 541 | CAAGTACTTG TACCTCGGCA CCTATAATAC GCAGGAGGAA |
| 601 | GCTGCAGCTG CATATGACAT GGCGGCTATA GAGTACAGAG |
| 641 | GTGCAAACGC AGTGACCAAC TTCGACATTG GTAACTACAT |
| 681 | CGACCGGTTA AAGAAAAAAG GTGTCTTCCC GTTCCCCGTG |
| 721 | AGCCAAGCTA ATCATCAAGA AGCTGTTCTT GCTGAAACCA |
| 761 | AACAAGAAGT GGAAGCTAAA GAAGAGCCTA CAGAAGAAGT |
| 801 | GAAGCAGTGT GTCGAAAAAG AAGAAGCTAA AGAAGAGAAG |
| 841 | ACTGAGAAAA AACAACAACA AGAAGTGGAG GAGGCGGTGA |
| 881 | TCACTTGCTG CATTGATTCT TCAGAGAGCA ATGAGCTGGC |
| 921 | TTGGGACTTC TGTATGATGG ATTCAGGGTT TGCTCCGTTT |
| 961 | TTGACTGATT CAAATCTCTC GAGTGAGAAT CCCATTGAGT |
| 1001 | ATCCTGAGCT TTTCAATGAG ATGGGTTTTG AGGATAACAT |
| 1041 | TGACTTCATG TTCGAGGAAG GGAAGCAAGA CTGCTTGAGC |
| 1081 | TTGGAGAATC TTGATTGTTG CGATGGTGTT CTTGTGGTGG |
| 1121 | GAAGAGAGAG CCCAACTTCA TTGTCGTCTT CTCCGTTGTC |
| 1141 | CTGCTTGTCT ACTGACTCTG CTTCATCAAC AACAACAACA |
| 1201 | GCAACAACAG TAACCTCTGT TTCTTGGAAC TATTCTGTCT |
| 1241 | GA |

Expression of a C-terminally truncated Brassica napus WRI1 protein or a Brassica napus WRI1 protein with a mutation at four or more of positions 381, 383, 384, 385, 387, 388, 391, 394, 399, 400, 401, 402, 403, 404, 406, 407, 409, and/or 410 can increase the content of triacylglycerol in plant tissues such as leaves and seeds. Hence, a mutant WRI1 protein can be used that includes a mutation (substitution, insertion, or deletion) in the following sequence (SEQ ID NO:79):

| | |
|---|---|
| 379 | RE SPTSLSSSPL SCLSTDSASS TTTTATTVTS VSWN |

For example, expression of a C-terminally truncated Brassica napus WRI1 protein or a Brassica napus WRI1 protein with at least four mutations at any of positions 381, 383, 384, 385, 387, 388, 391, 394, 399, 400, 401, 402, 403, 404, 406, 407, 409, and/or 410 can increase the content of triacylglycerol in plant tissues such as leaves and seeds. Hence, a mutant WRI1 protein can be used that includes the following sequence (SEQ ID NO: 80):

| | |
|---|---|
| 379 | RE XPXXLXSSPL XCLXTDSAXX XXXXAXXVXX VSWN |

where at least four of the X residues in the SEQ ID NO:80 sequence is a substitution, insertion, or deletion compared to the wild type sequence (SEQ ID NO:79). The X residues are not acidic amino acids such as aspartic acid or glutamic acid. However, the X residue can be a small amino acid or a hydrophobic amino acid. For example, the X residues can each separately be alanine, glycine, valine, leucine, isoleucine, methionine, or any mixture thereof.

In some cased, a mutant WRI1 protein can be used in the systems and methods that has a truncation at the C terminus of the SEQ ID NO:73 (or from the SEQ ID NO:77) sequence of at least 4, or at least 5, or at least 7, or at least 10, or at least 13, or at least 15, or at least 17, or at least 20, or at least 25, or at least 30, or at least 35, or at least 40, or at least 45 amino acids. Such mutant WRI1 proteins can be expressed in plant tissues to increase the oil/fatty acid/TAG content of those tissues.

Other Brassica napus amino acid and cDNA WRINKLED1 (WRI1) sequences are available as accession numbers ABD72476.1 (GI:89357185) and DQ402050.1 (GI:89357184), respectively.

An example of an amino acid sequence for a WRINKLED1 (WRI1) sequence from Zea mays is available as accession number ACG32367.1 (GI:195621074) and reproduced below as SEQ ID NO:81.

| | |
|---|---|
| 1 | MERSQRQSPP PPSPSSSSSS VSADTVLVPP GKRRRAATAK |
| 41 | AGAEPNKRIR KDPAAAAAGK RSSVYRGVTR HRWTGRFEAH |
| 81 | LWDKHCLAAL HNKKKGRQVY LGAYDSEEAA ARAYDLAALK |
| 121 | YWGPETLLNF PVEDYSSEMP EMEAVSREEY LASLRRRSSG |
| 161 | FSRGVSKYRG VARHHHNGRW EARIGRVFGN KYLYLGTFDT |
| 201 | QEEAAKAYDL AAIEYRGVNA VTNFDISCYL DHPLFLAQLQ |
| 241 | QEPQVVPALN QEPQPDQSET GTTEQEPESS EAKTPDGSAE |
| 281 | PDENAVPDDT AEPLSTVDDS IEEGLWSPCM DYELDTMSRP |
| 321 | NEGSSINLSE WFADADFDCN IGCLFDGCSA ADEGSKDGVG |
| 361 | LADFSLFEAG DVQLKDVLSD MEEGIQPPAM ISVCN |

A nucleic acid sequence for the above Zea mays WRI1 protein sequence is available as accession number EU960249.1 (GI:195621073), and is reproduced below as SEQ ID NO:82.

| | |
|---|---|
| 1 | CTCCCCCGCC TCGCCGCCAG TCAGATTCAC CACCGGCTCC |
| 41 | CCTGCACAAC CGCGTCCGCG CTGCACCACC ACCGTTCATC |
| 81 | GAGGAGGAGG GGGGACGGAG ACCACGGACA TGGAGAGATC |
| 121 | TCAACGGCAG TCTCCTCCGC CACCGTCGCC GTCCTCCTCC |
| 161 | TCGTCCTCCG TCTCCGCGGA CACCGTCCTC GTCCCTCCCG |
| 201 | GAAAGAGGCG GAGGGCGGCG ACGGCCAAGG CCGGCGCCGA |
| 241 | GCCTAATAAG AGGATCCGCA AGGACCCCGC CGCCGCCGCC |
| 281 | GCGGGGAAGA GGAGCTCCGT CTACAGGGGA GTCACCAGGC |
| 321 | ACAGGTGGAC GGGCAGGTTC GAGGCGCATC TCTGGGACAA |
| 361 | GCACTGCCTC GCCGCGCTCC ACAAGAAGAA GAAAGGCAGG |
| 401 | CAAGTCTACC TGGGGGCGTA TGACAGCGAG GAGGCAGCTG |
| 441 | CTCGTGCCTA TGACCTCGCA GCTCTCAAGT ACTGGGGTCC |
| 481 | TGAGACTCTG CTCAACTTCC CTGTGGAGGA TTACTCCAGC |
| 521 | GAGATGCCGG AGATGGAGGC CGTTTCCCGG GAGGAGTACC |
| 561 | TGGCCTCCCT CCGCCGCAGG AGCAGCGGCT TCTCCAGGGG |
| 601 | CGTCTCCAAG TACAGAGGCG TCGCCAGGCA TCACCACAAC |
| 641 | GGGAGGTGGG AGGCACGGAT TGGGCGAGTC TTTGGGAACA |
| 681 | AGTACCTCTA CTTGGGAACA TTTGACACTC AAGAAGAGGC |
| 721 | AGCCAAGGCC TATGACCTTG CGGCCATTGA ATACCGTGGC |
| 761 | GTCAATGCTG TAACCAACTT CGACATCAGC TGCTACCTGG |
| 801 | ACCACCCGCT GTTCCTGGCA CAGCTCCAAC AGGAGCCACA |
| 841 | GGTGGTGCCG GCACTCAACC AAGAACCTCA ACCTGATCAG |
| 881 | AGCGAAACCG GAACTACAGA GCAAGAGCCG GAGTCAAGCG |
| 921 | AAGCCAAGAC ACCGGATGGC AGTGCAGAAC CCGATGAGAA |
| 961 | CGCGGTGCCT GACGACACCG CGGAGCCCCT CAGCACAGTC |
| 1001 | GACGACAGCA TCGAAGAGGG CTTGTGGAGC CCTTGCATGG |
| 1041 | ATTACGAGCT AGACACCATG TCGAGACCAA ACTTTGGCAG |
| 1081 | CTCAATCAAT CTGAGCGAGT GGTTCGCTGA CGCAGACTTC |
| 1121 | GACTGCAACA TCGGGTGCCT GTTCGATGGG TGTTCTGCGG |
| 1161 | CTGACGAAGG AAGCAAGGAT GGTGTAGGTC TGGCAGATTT |
| 1201 | CAGTCTGTTT GAGGCAGGTG ATGTCCAGCT GAAGGATGTT |
| 1241 | CTTTCGGATA TGGAAGAGGG GATACAACCT CCAGCGATGA |
| 1281 | TCAGTGTGTG CAACTAATTC TGGAACCCGA GGAGGTTTTC |
| 1321 | GCTTTCCAGG TGTCCTGTCT TGGGTAATCC TTGATCTGTC |
| 1361 | TAATGCCACA GTGCCACTGC ACCAGAGCAG CTGAGAACTT |
| 1401 | TCTTGTAGAA AGCCCATGGC AGTTTGGCGT TAGACAAGTG |
| 1441 | TGTCGATGTT CTTTAATTCT TTGAATTTGC CCCTAGGCTG |
| 1481 | CTTGGCTAAC GTTAAGGGTT TGTCATTGTC TCACTTAGCC |
| 1521 | TAGATTCAAC TAATCACATC CTGAATCTGA AAAAAAAAAA |
| 1561 | CAAAAAAAAA AAAAAA |

Expression of an internally deleted Zea mays WRI1 protein or a Zea mays WRI1 protein with a mutation at four or more of amino acid positions 358, 360, 362, 363, 369, 370, 374, 378, 395, 395, 400, 407, 416, 418, and/or 419 can increase the content of triacylglycerol in plant tissues such as leaves and seeds. Hence, one aspect of the invention is a mutant WRI1 protein that includes a mutation (substitution, insertion, or deletion) in the following sequence (SEQ ID NO:83):

| | |
|---|---|
| 232 | HPLFLAQLQ |
| 241 | QEPQVVPALN QEPQPDQSET GTTEQEPESS EAKTPDGSAE |
| 281 | PDENAVPDDT AEPLSTVDDS IEEGLWSPCM DYELDTMSR |

For example, expression of an internally deleted Zea mays WRI1 protein or a Zea mays WRI1 protein with a mutation at four or more of the following positions 358, 360, 362, 363, 369, 370, 374, 378, 395, 395, 400, 407, 416, 418, and/or 419 can increase the content of triacylglycerol in plant tissues. Hence, another aspect of the invention is a mutant WRI1 protein that includes a mutation (substitution, insertion, or deletion) in the following sequence (SEQ ID NO: 84):

| | |
|---|---|
| 232 | HPLFLAQLQ |
| 241 | QEPQVVPALN QEPQPDQXEX GXXEQEPEXX EAKXPDGXAE |
| 281 | PDENAVPDDX AEPLXXVDDX IEEGLWXPCM DYELDXMXR |

where at least four of the X residues in the SEQ ID NO:84 sequence is a substitution, insertion, or deletion compared to the wild type sequence (SEQ ID NO:83). The X residues are not acidic amino acids such as aspartic acid or glutamic acid. However, the X residue can be a small amino acid or a hydrophobic amino acid. For example, the X residues can each separately be alanine, glycine, valine, leucine, isoleucine, methionine, or any mixture thereof.

A mutant WRI1 protein with a deletion within the SEQ ID NO:83 portion of the WRI1 protein of at least 3, or at least 4, or at least 5, or at least 6, or at least 7, or at least 8, or at least 10, or at least 13, or at least 15, or at least 17, or at least 20, or at least 25, or at least 30, or at least 35, or at least 40, or at least 45 amino acids. Such mutant WRI1 proteins can be expressed in plant tissues to increase the oil/fatty acid/TAG content of those tissues.

Another example of an amino acid sequence for a WRINKLED1 (WRI1) sequence from Zea mays is available as accession number NP_001131733.1 (GI:212721372) and reproduced below as SEQ ID NO:85.

| | |
|---|---|
| 1 | MTMERSQPQH QQSPPSPSSS SSCVSADTVL VPPGKRRRRA |
| 41 | ATAKANKRAR KDPSDPPPAA GKRSSVYRGV TRHRWTGRFE |
| 81 | AHLWDKHCLA ALHNKKKGRQ VYLGAYDGEE AAARAYDLAA |
| 121 | LKYWGPEALL NFPVEDYSSE MPEMEAASRE EYLASLRRRS |
| 161 | SGFSRGVSKY RGVARHHHNG RWEARIGRVL GNKYLYLGTF |
| 201 | DTQEEAAKAY DLAAIEYRGA NAVTNFDISC YLDHPLFLAQ |
| 241 | LQQEQPQVVP ALDQEPQADQ REPETTAQEP VSSQAKTPAD |
| 281 | DNAEPDDIAE PLITVDNSVE ESLWSPCMDY ELDTMSRSNF |
| 321 | GSSINLSEWF TDADFDSDLG CLFDGRSAVD GGSKGGVGVA |
| 361 | DFSLFEAGDG QLKDVLSDME EGIQPPTIIS VCN |

A nucleic acid sequence for the above Zea mays WRI1 protein sequence is available as accession number NM_001138261.1 (GI:212721371), and is reproduced below as SEQ ID NO:86.

| | |
|---|---|
| 1 | CGTTCATGCA TGACCATGGA GAGATCTCAA CCGCAGCACC |
| 41 | AGCAGTCTCC TCCGTCGCCG TCGTCCTCCT CGTCCTGCGT |
| 81 | CTCCGCGGAG ACCGTCCTCG TCCCTCCGGG AAAGAGGCGG |
| 121 | CGGAGGGCGG CGACAGCCAA GGCCAATAAG AGGGCCCGCA |
| 161 | AGGACCCCTC TGATCCTCCT CCCGCCGCCG GGAAGAGGAG |
| 201 | CTCCGTATAC AGAGGAGTCA CCAGGCACAG CTGGACGGGC |
| 241 | AGGTTCGAGG CGCATCTCTG GGACAAGCAC TGCCTCGCCG |
| 281 | CGCTCCACAA CAAGAAGAAA GGCAGGCAAG TCTATCTGGG |
| 321 | GGCGTACGAC GGCGAGGAGG CAGCGGCTCG TGCCTATGAC |
| 361 | CTTGCAGCTC TCAAGTACTG GGGTCCTGAG GCTCTGCTCA |
| 401 | ACTTCCCTGT GGAGGATTAC TCCAGCGAGA TGCCGGAGAT |
| 441 | GGAGGCAGCG TCCCGGGAGG AGTACCTGGC CTCCCTCCGC |
| 481 | CGCAGGAGCA GCGGCTTCTC CAGGGGGGTC TCCAAGTACA |
| 521 | GAGGCGTCGC CAGGCATCAC CACAACGGGA GATGGGAGGC |
| 561 | ACGGATCGGG CGAGTTTTAG GGAACAAGTA CCTCTACTTG |
| 601 | GGAACATTCG ACACTCAAGA AGAGGCAGCC AAGGCCTATG |
| 641 | ATCTTGCGGC CATCGAATAC CGAGGTGCCA ATGCTGTAAC |
| 681 | CAACTTCGAC ATCAGCTGCT ACCTGGACCA CCCACTGTTC |
| 721 | CTGGCGCAGC TCCAGCAGGA GCAGCCACAG GTGGTGCCAG |
| 761 | CGCTCGACCA AGAACCTCAG GCTGATCAGA GAGAACCTGA |
| 801 | AACCACAGCC CAAGAGCCTG TGTCAAGCCA AGCCAAGACA |
| 841 | CCGGCGGATG ACAATGCAGA GCCTGATGAC ATCGCGGAGC |
| 881 | CCCTCATCAC GGTCGACAAC AGCGTCGAGG AGAGCTTATG |
| 921 | GAGTCCTTGC ATGGATTATG AGCTAGACAC CATGTCGAGA |
| 961 | TCTAACTTTG GCAGCTCGAT CAACCTGAGC GAGTGGTTCA |
| 1001 | CTGACGCAGA CTTCGACAGC GACTTGGGAT GCCTGTTCGA |
| 1041 | CGGGCGCTCT GCAGTTGATG GAGGAAGCAA GGGTGGCGTA |
| 1081 | GGTGTGGCGG ATTTCAGTTT GTTTGAAGCA GGTGATGGTC |
| 1121 | AGCTGAAGGA TGTTCTTTCG GATATGGAAG AGGGGATACA |
| 1161 | ACCTCCAACG ATAATCAGTG TGTGCAATTG ATTCTGAGAC |
| 1201 | CTATGCGTGG CGTGCGACAA GTGTCCTGTC TTTGGGTATA |
| 1241 | CTTGGTTTGT CCAATGCCAC GGTGCCACTG CTGCGAGTCA |
| 1281 | GCTGAACTTC TTGTAGAAAG CACATGGCAG CTTGGCATTA |
| 1321 | GACAAGTGTG TTGGTGTTCC TTAATTCTTT GGATATGCTT |
| 1361 | TAGGCATTGA CTAACCTTAA GGGTTCGTCA CTGTCTCGCT |
| 1401 | TAGCTTAGAT TAGACTAATC ACATCCTTGA ATCTGAAGTA |
| 1441 | GTTGTGCAGT ATCACAGTTT CACATGGCAA TTCTGCCAAT |
| 1481 | GCAGCATAGA TTTGTTCGTT TGAACAGCTG TAACTGTAAC |
| 1521 | CCTATAGCTC CAGATTAAGG AACAGTTTGT TTTTCATCCA |
| 1561 | T |

Expression of an internally deleted Zea mays WRI1 protein or a Zea mays WRI1 protein with a imitation at four or more of positions 265, 266, 272, 273, 277, 294, 298, 302, 305, 314, and/or 316 can increase the content of triacylglycerol in plant tissues such as leaves and seeds. Hence, a mutant WRI1 protein can be used in the systems and methods described herein that includes a mutation (substitution, insertion, or deletion) in the following sequence (SEQ ID NO:87):

| | |
|---|---|
| 261 | REPETTAQEP VSSQAKTPAD |
| 281 | DNAEPDDIAE PLITVDNSVE ESLWSPCMDY ELDTMSR |

For example, expression of an internally deleted Zea mays WRI1 protein or a Zea mays WRI1 protein with a mutation at four or more of positions 265, 266, 272, 273, 277, 294, 298, 302, 305, 314, and/or 316 can increase the content of triacylglycerol in plant tissues. Hence, a mutant WRI1 protein can be used that includes the following sequence (SEQ ID NO:88):

| | |
|---|---|
| 261 | REPEXXAQEP VXXQAKXPAD |
| 281 | DNAEPDDIAE PLIXVDNXVE EXLWXPCMDY ELDXMXR |

where at least four of the X residues in the SEQ ID NO:88 sequence is a substitution, insertion, or deletion compared to the wild type sequence (SEQ ID NO:87). The X residues are not acidic amino acids such as aspartic acid or glutamic acid. However, the X residue can be a small amino acid or a hydrophobic amino acid. For example, the X residues can each separately be alanine, dycine, valine, leucine, isoleucine, methionine, or any mixture thereof.

Another aspect of the invention is a mutant WRI1 protein with a deletion within the SEQ ID NO:85 or SEQ ID NO:88 portion of the WRI1 protein of at least 3, or at least 4, or at least 5, or at least 6, or at least 7, or at least 8, or at least 10, or at least 13, or at least 15, or at least 17, or at least 20, or at least 25, or at least 30, or at least 35, or at least 40, or at least 45 amino acids.

An example of an amino acid sequence for a WRINKLED1 (WRI1) sequence from Elaeis guineensis (palm oil) is available as accession number XP_010922928.1 (GI:743789536) and reproduced below as SEQ ID NO:89.

| | |
|---|---|
| 1 | MTLMKNSPPS TPLPPISPSS SASPSSYAPL SSPNMIPLNK |
| 41 | CKKSKPKHKK AKNSDESSRR RSSIYRGVTR HRGTGRYEAH |
| 81 | LWDKHWQHPV QNKKGRQVYL GAFTDELDAA RAHDLAALKL |
| 121 | WGPETILNFP VEMYREEYKE MQTMSKEEVL ASVRRRSNGF |
| 161 | ARGTSKYRGV ARHHKNGRWE ARLSQDVGCK YIYLGTYATQ |
| 201 | EEAAQAYDLA ALVHKGPNIV TNFASSVYKH RLQPFMQLLV |
| 241 | KPETEPAQED LGVLQMEATE TIDQTMPNYD LPEISWTFDI |
| 281 | DHDLGAYPLL DVPIEDDQHD ILNDLNFEGN IEHLFEEFET |
| 321 | FGGNESGSDG FSASKGA |

A nucleic acid sequence for the above Elaeis guineensis WRI1 protein sequence is available as accession number XM_010924626.1 (GI:743789535), and is reproduced below as SEQ ID NO:90.

| | |
|---|---|
| 1 | AGAGAGAGAG AGATTCCAAC ACAGGGCAGC TGAGATTGAG |
| 41 | CACAAGGCGC CGTGGAAACC ACGAGTTCCA TTGGCAACAT |
| 81 | GGGAAACCTG GTGGCCAAGT GTAGAGCTCT CTCACACAAA |
| 121 | CCCATGCGGC CAACTTGCAG ACCCTCGAGT CATTTGGACT |
| 161 | CTTCCAAGCT CACCAGCCGT AGGGTTTTTT GACAAGAGGG |
| 201 | ACCTCCAGTA AACGTTAAAC AAACTCGCAG CTCCCACCTT |
| 241 | TGGATCCATT CCATCGCTTC AACGGTGGGT TAGAAGCCTC |
| 281 | CGCGCCAAAT GCACGAGTGC TCAACAGCAC GCTCCCCTAA |
| 321 | TTTTTCTCTC TCCACCTCCT CACTTCTCTA TATATAATCC |
| 361 | TCTCTTTGGT GAACCACCAT CAACCAAACC AACGGTATAG |
| 401 | TATACGTAGG AAATAATCCC TTTCTAGAAC ATGACTCTCA |
| 441 | TGAAGAAATC TCCTCCCTCT ACTCCTCTCC CACCAATATC |
| 481 | GCCTTCCTCT TCCGCTTCAC CATCCAGCTA TGCACCCCTT |
| 521 | TCTTCTCCTA ATATGATCCC TCTTAACAAG TGCAAGAAGT |
| 561 | CGAAGCCAAA ACATAAGAAA GCTAAGAACT CAGATGAAAG |
| 601 | CAGTAGGAGA AGAAGCTCTA TCTACAGAGG AGTCACGAGG |
| 641 | CACCGAGGGA CTGGGAGATA TGAAGCTCAC CTGTGGGACA |
| 681 | AGCACTGGCA GCATCCGGTC CAGAACAAGA AAGGCAGGCA |
| 721 | AGTTTACTTG GGAGCCTTTA CTGATGAGTT GGACGCAGCA |
| 761 | CGAGCTCATG ACTTGGCTGC CCTTAAGCTC TGGGGTCCAG |
| 801 | AGACAATTTT AAACTTCCCT GTGGAAATGT ATAGAGAAGA |
| 841 | GTACAAGGAG ATGCAAACCA TGTCAAAGGA AGAGGTGCTG |
| 881 | GCTTCGGTTA GGCGCAGGAG CAACGGCTTT GCCAGGGGTA |
| 921 | CCTCTAAGTA CCGTGGGGTG GCCAGGCATC ACAAAAACGC |
| 961 | CCGGTGGGAG GCCAGGCTTA GCCAGGACGT TGGCTGCAAG |
| 1001 | TACATCTACT TGGGAACATA CGCAACTCAA GAGGAGGCTG |
| 1041 | CCCAAGCTTA TGATTTAGCT GCTCTAGTAC ACAAAGGGCC |
| 1081 | AAATATAGTG ACCAACTTTG CTAGCAGTGT CTATAAGCAT |
| 1121 | CGCCTACAGC CATTCATGCA GCTATTAGTG AAGCCTGAGA |
| 1161 | CGGAGCCAGC ACAAGAAGAC CTGGGGGTTA TGCAAATGGA |
| 1201 | AGCAACCGAG ACAATCGATC AGACCATGCC AAATTACGAC |
| 1241 | CTGCCGGAGA TCTCATGGAC CTTCGACATA GACCATGACT |
| 1281 | TAGGTGCATA TCCTCTCCTT GATGTCCCAA TTGAGGATGA |
| 1321 | TCAACATGAC ATCTTGAATG ATCTCAATTT CGAGGGGAAC |
| 1361 | ATTGAGCACC TCTTTGAAGA GTTTGAGACC TTCGGAGGCA |
| 1401 | ATGAGAGTGG AAGTGATGGT TTCAGTGCAA GCAAAGGTGC |
| 1441 | CTAGCAGAGG AAAGTGGTTT GAAGATGGAG GACATGGCAT |
| 1481 | CTAAAGCGAA CTGAGCCTCC TGGCCTCTTC AAAGTAGTGT |
| 1521 | CTGCTTTTTA GAAATCTTGG TGGGTCGATT TGAGTTAGGA |
| 1561 | GCCCGATACT TCTATCAGGG GATATGTTTA GCTACAATTC |
| 1601 | TAGTTTTTTT TTCTTTTTTT TTTTTCAGCC GGAAGTCTGG |
| 1641 | TACTTCTGTT GAATATTATG ATGTGCTTCT TGCTTAGTTG |
| 1681 | TTCCTGTTCT TCTCCCTTTT AGAGTTCAGC ATATTTATGT |
| 1721 | TTTGATGTAA TGGGGAATGT TGGCAGACAG CTTGATATAT |
| 1761 | GGTTATTTCA TTCTCCATTA AA |

Expression of an internally deleted Elaeis guineensis WRI1 protein or an Elaeis guineensis WRI1 protein with a mutation at four or more of the following positions 244, 259, 261, 265, 275, and/or 277 can increase the content of triacyiglycerol in plant tissues such as leaves and seeds, Hence, in some cases a mutant WRI1 protein is used that includes a mutation (e.g., a substitution, insertion, or deletion) in the following sequence (SEQ ID NO:91):

| | |
|---|---|
| 241 | KPETEPAQED LGVLQMEATE TIDQTMPNYD LPEISWTFDI DH |

For example, expression of an internally deleted Elaeis guineensis WRI1 protein or an Elaeis guineensis WRI1 protein with a mutation at four or more of positions 244, 259, 261, 265, 275, and/or 277 can increase the content of triacylglycerol in plant tissues. Hence, in some cases a mutant WRI1 protein can be used that includes the following sequence (SEQ ID NO: 92):

| | |
|---|---|
| 241 | KPEXEPAQED LGVQMEAXE XIDQXMPNYD LPEIXWXFDI DH |

where at least four of the X residues in the SEQ ID NO:92 sequence is a substitution, insertion, or deletion compared to the wild type sequence (SEQ ID NO:91). The X residues are not acidic amino acids such as aspartic acid or glutamic acid. However, the X residue can be a small amino acid or a hydrophobic amino acid. For example, the X residues can each separately be alanine, glycine, leucine, isoleucine, methionine, and any mixture thereof.

Another aspect of the invention is a mutant WRI1 protein with a deletion within the SEQ ID NO:89 or SEQ ID NO:91 portion of the WRI1 protein of at least 3, or at least 4, or at least 5, or at least 6, or at least 7 or at least 8, or at least 10, or at least 13, or at least 15, or at least 17, or at least 20, or at least 25, or at least 30, or at least 35, or at least 40, or at least 45 amino acids. Such mutant WRI1 proteins can be expressed in plant tissues to increase the oil/fatty acid/TAG content of those tissues.

An example of an amino acid sequence for a WRINKLED1 (WRI1) sequence from Glycine max (soybean) is available as accession number XP_006596987.1 (GI:571513961) and reproduced below as SEQ ID NO:93).

| | |
|---|---|
| 1 | MKRSPASSCS SSTSSVGFEA PIEKRRPKHP RRNNLKSQKC |
| 41 | KQNQTTTGGR RSSIYRGVTR HRWTGRFEAH LWDKSSWNNI |
| 81 | QSKKGRQGAY DTEESAARTY DLAALKYWGK DATLNFPIET |
| 121 | YTKELEEMDK VSREEYLASL RRQSSGFSRG LSKYRGVARH |
| 161 | HHNGRWEARI GRVCGNKYLY LGTYKTQEEA AVAYDMAAIE |
| 201 | YRGVNAVTNF DISNYMDKIK KKNDQTQQQQ TEAQTETVPN |
| 241 | SSDSEEVEVE QQTTTITTPP PSENLHMPPQ QHQVQYTPHV |
| 281 | SPREEESSSL ITIMDHVLEQ DLPWSFMYTG LSQFODPNLA |
| 321 | FCKGDDDLVG MFDSAGFEED IDFLFSTQPG DETESDVNNM |
| 361 | SAVLDSVECG DTNGAGGSMM HVDNEQKIVS FASSPSSTTT |
| 401 | VSCDYALDL |

A nucleic acid sequence for the above Glycine max WRI1 protein sequence is available as accession number XM_006596924.1 (GI:571513960), and is reproduced below as SEQ ID NO:94.

| | |
|---|---|
| 1 | AGTGTTGCTC AAATTCAAGC CACTTAATTA GCCATGGTTG |
| 41 | ATTGATCAAG TTAAATTCCA ACCCAAGGTT AAATCATTAC |
| 81 | TCCCTTCTCA TCCTTCCCAA CCCCAACCCC CAGAAATATT |
| 121 | ACAGATTCAA TTGCTTAATT AAATACTATT TTCCCCTCCT |
| 161 | TCTATAATAC CCTCCAAAAT CTTTTTCCTT CTTCATTCTC |
| 201 | CCTTTCTCTA TGTTTTGGCA AACCACTTTA GGTAACCAGA |
| 241 | TTACTACTAC TATTGCTTCA TATACAAAGA TGCTATCGTA |
| 281 | AAAAAGAGAG AAACTTGGGA AGTGGGAACA CATTCAAAAT |
| 321 | CCTTGTTTTT CTTTTTGGTC TAATTTTTCA TCTCAAAACA |
| 361 | CACACCCATT GAGTATTTTT CATTTTTTTG TTCTTTTGGG |
| 401 | ACAAAAAAGG TGGGTGTTGT TGGCATTATT GAAGATAGAG |
| 441 | GCCCCCAAAA TGAAGAGGTC TCCAGCATCT TCTTGTTCAT |
| 481 | CATCTACTTC CTCTGTTGGG TTTGAAGCTC CCATTGAAAA |
| 521 | AAGAAGGCCT AAGCATCCAA GGAGGAATAA TTTGAAGTCA |
| 561 | CAAAAATGCA AGCAGAACCA AACCACCACT GGTGGCAGAA |
| 601 | GAAGCTCTAT CTATAGAGGA GTTACAAGGC ATAGGTGGAC |
| 641 | AGGGAGGTTT GAAGCTCACC TATGGGATAA GAGCTCTTGG |
| 681 | AACAACATTC AGAGCAAGAA GGGTCGACAA GGGGCATATG |
| 721 | ATACTGAAGA ATCTGCAGCC CGTACCTATG ACCTTGCAGC |
| 761 | CCTTAAATAC TGGGGAAAAG ATGCAACCCT GAATTTCCCG |
| 801 | ATAGAAACTT ATACCAAGGA GCTCGAGGAA ATGGACAAGG |
| 841 | TTTCAAGAGA AGAATATTTG GCTTCTTTGC GGCGCCAAAG |
| 881 | CAGTGGCTTT TCTAGAGGCC TGTCTAAGTA CCGTGGGGTT |
| 921 | GCTAGGCATC ATCATAATGG TCGCTGGGAA GCACGAATTG |
| 961 | GAAGAGTATG CGGAAACAAG TACCTCTACT TGGGGACATA |
| 1001 | TAAAACTCAA GAGGAGGCAG CAGTGGCATA TGACATGGCA |
| 1041 | GCAATACAGT ACCGTCGAGT CAATGCACTG ACCAATTTTG |
| 1081 | ACATAAGCAA CTACATGGAC AAAATAAAGA AGAAAAATGA |
| 1121 | CCAAACCCAA CAACAACAAA CAGAAGCACA AACGGAAACA |
| 1161 | GTTCCTAACT CCTCTGACTC TGAAGAAGTA GAAGTAGAAC |
| 1201 | AACAGACAAC AACAATAACC ACACCACCCC CATCTGAAAA |
| 1241 | TCTCCACATG CCACCACAGC AGCACCAAGT TCAATACACC |
| 1281 | CCCCATGTCT CTCCAAGGGA ACAACAATCA TCATCACTGA |
| 1321 | TCACAATTAT GGACCATGTG CTTGAGCAGG ATCTGCCATG |
| 1361 | GAGCTTCATG TACACTGGCT TGTCTCAGTT TCAAGATCCA |
| 1401 | AACTTGGCTT TCTGCAAAGG TGATGATGAC TTGGTGGGCA |
| 1441 | TGTTTGATAG TGCAGGGTTT GAGGAAGACA TTGATTTTCT |
| 1481 | GTTCAGCACT CAACCTGGTG ATGAGACTGA GAGTGATGTC |
| 1521 | AACAATATGA GCGCAGTTTT GGATAGTGTT GAGTGTGGAG |
| 1561 | ACACAAATGG GGCTGGTGGA AGCATGATGC ATGTGGATAA |
| 1601 | CAAGCAGAAG ATAGTATCAT TTGGTTCTTC ACCATCATCT |
| 1641 | ACAACTACAG TTTCTTGTGA CTATGCTCTA GATCTATGAT |
| 1681 | CTCTTCAGAA GGGTGATGGA TGAGCTACAT GGAATGGAAC |
| 1721 | CTTGTGTAGA TTATTATTGG GTTTGTTATG CATGTTGTTG |
| 1761 | GGGTTTGTTG TGATAGGTTG GTGGATGGGT GTGACTTGTG |
| 1801 | AAAATGTTCA TTGGTTTTAG GATTTTCCTT TCATCCATAC |
| 1841 | TCCGTTGTCG AAAGAAGAAA ATGTTCATTT TAGACTTGGA |
| 1381 | TTTTAGTATA AAAAAAAAGG AGAAAAAACC AAAAATCTGA |
| 1921 | TTTGGGTGCA AACAATGTTT TGTTTTTCTT TTTACTTTTG |
| 1961 | GGGTAAGGAG ATGAAGAGAG GGCAAATTTA AACCATTCCT |
| 2001 | ATTCTTGGGG GATAATGCAG TATAAATTAA GATCAGACTG |
| 2041 | TTTTTAGCAT ATGGAGTGCA AACTGCAAAG GCCAAGTTTC |
| 2081 | CTTTCTTTAA ACAATTTAGG CTTTCTTTTC CTTTGCCTAT |
| 2121 | TTTTTTTTTA TTTTTTTTTT TGTATTGGGG CATAGCAGTT |
| 2161 | AGTGTTGTGT TGAGATCTGA AATCTGATCT CTGGTTTGGT |
| 2201 | TTGTTC |

Expression of an internally deleted Glycine max WRI1 protein or an Glycine max WRI1 protein with a mutation at four or more of the following positions 353, 355, 361, 366, 372, 378, 390, 393, 394, 396, 397, 398, 399, 400 and/or 402 can increase the content of triacylglycerol in plant tissues such as leaves and seeds. Hence, one aspect of the invention is a mutant WRI1 protein that includes a mutation (e.g., a substitution, insertion, or deletion) in the following sequence (SEQ ID NO:95):

| | |
|---|---|
| 351 | DETESDVNNM |
| 361 | SAVLDSVECG DTNGAGGSMM HVDNKQKIVS FASSPSSTTT |
| 401 | VSCDYALDL |

For example, expression of an internally deleted Glycine max WRI1 protein or a Glycine max WRI1 protein with a mutation at four or more of positions 353, 355, 361, 366, 372, 378, 390, 393, 394, 396, 397, 398, 399, 400 and/or 402 can increase the content of triacylglycerol in plant tissues. Hence, a mutant WRI1 protein can be used that includes the following sequence (SEQ ID NO: 96):

| | |
|---|---|
| 351 | DEXEXDVNNM |
| 361 | XAVLDXVECG DXNGAGGXMM HVDNKQKIVX FAXXPXXXXX |
| 401 | VXCDYALDL |

where at least four of the X residues in the SEQ ID NO:96 sequence is a substitution, insertion, or deletion compared to the wild type sequence (SEQ ID NO:95). The X residues are not acidic amino acids such as aspartic acid or glutamic acid. However, the X residue can be a small amino acid or a hydrophobic amino acid. For example, the X residues can each separately be alanine, glycine, valine, leucine, isoleucine, methionine, and any mixture thereof.

In some cases, a mutant WRI1 protein with a deletion within the SEQ ID NO:93 portion of the WRI1 protein of at least 3, or at least 4, or at least 5, or at least 6, or at least 7, or at least 8, or at least 10, or at least 13, or at least 15, or at least 17, or at least 20, or at least 25, or at least 30, or at least 35, or at least 40, or at least 45 amino acids. Such mutant WRI1 proteins can be expressed in plant tissues.

### Expression of Proteins

Also described herein are expression systems that include at least one expression cassette (e.g., expression vectors or transgenes) that encode one or more of the enzymes described herein, transcription factor(s) described herein, LDSP-protein fusion(s) described herein, or combinations thereof. For example, the expression systems can also include one or more expression cassettes encoding LDSP, monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), HMG-CoA synthase, HMG-CoA reductase (HMGR), mevalonic acid kinase (WVK), phosphomevalonate kinase (PMK), mevalonate-5-diphosphate decarboxylase (MPD), isopentenyl diphosphate isomerase, abietadiene synthase (ABS), farnesylpyrophosphate synthase (FPPS), or squalene synthase (SQS), LDSP-protein fusions, or enzymes that facilitate production of terpene precursors or building blocks.

Nucleic acids encoding the proteins can have sequence modifications. For example, nucleic acid sequences described herein can be modified to express enzymes and transcription factors that have modifications. For example, most amino acids can be encoded by more than one codon. When an amino acid is encoded by more than one codon, the codons are referred to as degenerate codons. A listing of degenerate codons is provided in Table 1A below.

TABLE 1A
Degenerate Amino Acid Codons

| Amino Acid | Three Nucleotide Codon |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |
| START | ATG |
| STOP | TAG, TGA, TAA |

Different organisms may translate different codons more or less efficiently (e.g., because they have different ratios of tRNAs) than other organisms. Hence, when some amino acids can be encoded by several codons, a nucleic acid segment can be designed to optimize the efficiency of expression of an enzyme by using codons that are preferred by an organism of interest. For example, the nucleotide coding regions of the enzymes described herein can be codon optimized for expression in various plant species. Such enzymes can be expressed in a variety of host cells, including for example, as Nicotiana benthamiana, Nicotiana tabacum, Nicotiana rustica, Nicotiana excelsior, and Nicotiana excelsiana.

An optimized nucleic acid can have less than 98% less than 97%, less than 95%, or less than 94%, or less than 93%, or less than 92%, or less than 91%, or less than 90%, or less than 89%, or less than 88%, or less than 85%, or less than 83%, or less than 80%, or less than 75% nucleic acid sequence identity to a corresponding non-optimized (e.g., a non-optimized parental or wild type enzyme nucleic acid) sequence.

In some cases, LDSP or enzymes can have conservative changes such as one or more deletions, insertions, replacements, or substitutions that have no significant effect on the activities of the enzymes. Examples of conservative substitutions are provided below in Table 1B.

TABLE 1B
Conservative Substitutions

| Type of Amino Acid | Substitutable Amino Adds |
|---|---|
| Hydrophilic | Ala, Pro, Gly, Glu, Asp, Gln, Asn, Ser, Thr |
| Sulfhydryl | Cys |
| Aliphatic | Val, Ile, Leu, Met |
| Basic | Lys, Arg, His |
| Aromatic | Phe, Tyr, Trp |

The nucleic acids described herein can also be modified to improve or alter the functional properties of the encoded enzymes. Deletions, insertions, or substitutions can be generated by a variety of methods such as, but not limited to, random mutagenesis and/or site-specific recombination-mediated methods. The mutations can range in size from one or two nucleotides to hundreds of nucleotides (or any value there between). Deletions, insertions, and/or substitutions are created at a desired location in a nucleic acid encoding the enzyme(s).

Nucleic acids encoding one or more enzyme(s) can have one or more nucleotide deletions, insertions, replacements, or substitutions. For example, the nucleic acids encoding one or more enzyme(s) can, for example, have less than 95%, or less than 94.8%, or less than 94.5%, or less than 94%, or less than 93.8%, or less than 94.50% nucleic acid sequence identity to a corresponding parental or wild-type sequence. In some cases, the nucleic acids encoding one or more enzyme(s) can have, for example, at least 50%, or at least 55%, or at least 60%, or at least 65%, or at least 70%, or at least 75%, or at least 80%, or at least 85%, or at 90% sequence identity to a corresponding parental or wild-type sequence. Examples of amino acid sequences for parental LDSP and unmodified proteins include amino acid sequences with SEQ ID NOs:1, 3, 5, 7, 9, 11, 13, 15, 17, 19, 21, 23, 25, 27, 29, 31, 33, 35, 37, 39, 41, 43, 45, 47, 49, 51, 52, 53, 54, 55, 56, 59, 61, 63, 64, 65, 67, 68, 69, 71, 72, 73, 75, 76, 77, 79, 80, 81, 83, 84, 85, 87, 89, 91, 92, 93, 95, 96, 97, 99, 101, 104, 105, 107, 108, 110, or 111 include nucleic acid sequence SEQ ID NO:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 66, 70, 74, 78, 82, 87, 88, 90, 94, 98, 100, 102, 103, 106, or 109. Any of these amino acid or nucleic acid sequences can, for example, have or encode enzyme sequences with less than 99%, less than 98%, less than 97%, less than 96%, less than 95%, less than 94.8%, less than 94.5%, less than 94%, less than 93.8%, less than 93.5%, less than 93%, less than 92%, less than 91%, or less than 90% sequence identity to a corresponding parental or wild-type sequence.

Also provided are nucleic acid molecules (polynucleotide molecules) that can include a nucleic acid segment encoding an enzyme with a sequence that is optimized for expression in at least one selected host organism or host cell. Optimized sequences include sequences which are codon optimized, i..e., codons which are employed more frequently in one organism relative to another organism. In some cases, the balance of codon usage is such that the most frequently used codon is not used to exhaustion. Other modifications can include addition or modification of Kozak sequences and/or introns, and/or to remove undesirable sequences, for instance, potential transcription factor binding sites.

The LDSP, enzymes and LDSP-protein fusions described herein can be expressed from an expression cassette and/or an expression vector. Such an expression cassette can include a nucleic acid segment that encodes at least one LDSP, enzyme, or LDSP-protein fusion operably linked to a promoter to drive expression of one or more LDSP, enzyme, or LDSP-protein fusion. Convenient vectors, or expression systems can be used to express such LDSP, enzymes and LDSP-protein fusions. In some instances, the nucleic acid segment encoding one or more LDSP, enzyme, or LDSP-protein fusion is operably linked to a promoter and/or a transcription termination sequence. The promoter and/or the termination sequence can be heterologous to the nucleic acid segment that encodes the LDSP, enzyme, or LDSP-protein fusion. Expression cassettes can have a promoter operably linked to a heterologous open reading frame encoding a LDSP, enzyme, or LDSP-protein fusion. The invention therefore provides expression cassettes or vectors useful for expressing one or more one or more LDSP, enzyme, or LDSP-protein fusion.

Constructs, e.g., expression cassettes, and vectors comprising the isolated nucleic acid molecule, e.g., with optimized nucleic acid sequence, as well as kits comprising the isolated nucleic acid molecule, construct or vector are also provided.

Techniques of molecular biology, microbiology, and recombinant DNA technology which are within the skill of the art can be employed to make and use the enzymes, expression systems, and terpene products described herein. Such techniques available in the literature, See, e.g., Sambrook, Fritsch & Maniatis, Molecular Cloning: A Laboratory Manual, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series Methods In Enzymology (S. Colowick and N. Kaplan eds., Academic Press, Inc.); Current Protocols In Molecular Biology (John Wiley & Sons, Inc), Current Protocols In Protein Science (John Wiley & Sons, Inc), Current Protocols In Microbiology (John Wiley & Sons, Inc), Current Protocols In Nucleic Acid Chemistry (John Wiley & Sons, Inc), and Handbook of Experimental Immunology, Vols. I-IV (D. M. Weir and C. C. Blackwell eds., 1986, Blackwell Scientific Publications).

The expression systems can be introduced into a variety of host cells, host tissues, seeds (e.g., “host seeds”), and host plants.

Examples of host cells, host tissues, host seeds and plants that may be improved by these methods (e.g., by incorporation of nucleic acids and expression systems) include but are not limited to those useful for production of oils such as oilseeds, camelina, canola, castor bean, corn, flax, lupins, peanut, potatoes, safflower, soybean, sunflower, cottonseed, oil firewood trees, rapeseed, rutabaga, sorghum, walnut, and various nut species. Other types host cells, host tissues, host seeds and plants that can be used include fiber-containing plants, trees, flax, grains (maize, wheat, barley, oats, rice, sorghum, millet and rye), grasses (switchgrass, prairie grass, wheat grass, sudangrass, sorghum, straw-producing plants), softwood, hardwood and other woody plants (e.g., poplar, pine, and eucalyptus), oil (oilseeds, camelina, canola, castor bean, lupins, potatoes, soybean, sunflower, cottonseed, oil firewood trees, rapeseed, rutabaga, sorghum), starch plants (wheat, potatoes, lupins, sunflower and cottonseed), and forage plants (alfalfa, clover and fescue). In some embodiments the plant is a gymnosperm. Examples of plants useful for pulp and paper production include most pine species such as loblolly pine, Jack pine, Southern pine, Radiata pine, spruce, Douglas fir and others. Hardwoods that can be modified as described herein include aspen, poplar, eucalyptus, and others. Plants useful for making biofuels and ethanol include corn, grasses (e.g., miscanthus, switchgrass, and the like), as well as trees such as poplar, aspen, pine, oak, maple, walnut, rubber tree, willow, and the like. Plants useful for generating forage include legumes such as alfalfa, as well as forage grasses such as bromegrass, and bluestem. In some cases, the plant is a Brassicaceae or other Solanaceae species. In some embodiments, the plant is not a species of Arabidopsis, for example, in some embodiments, the plant is not Arabidopsis thaliana.

Modified plants that contain nucleic acids encoding one or more LDSP, enzyme, and/or LDSP-protein fusion within their somatic and/or germ cells are described herein. Such genetic modification can be accomplished by available procedures. For example, one of skill in the art can prepare an expression cassette or expression vector that can express one or more encoded LDSP, enzyme, and/or LDSP-protein fusion. Plant cells can be transformed by the expression cassette or expression vector, and whole plants (and their seeds) can be generated from the plant cells that were successfully transformed with one or more LDSP, enzyme, and/or LDSP-protein fusion nucleic acids. Some procedures for making such genetically modified plants and their seeds are described below.

Promoters: The nucleic acids encoding one or more LDSP, enzyme, and/or LDSP-protein fusion can be operably linked to a promoter, which provides for expression of mRNA from the nucleic acids. The promoter is typically a promoter functional in plants and can be a promoter functional during plant growth and development. A nucleic acid segment encoding one or more LDSP, enzyme, and/or LDSP-protein fusion is operably linked to the promoter when it is located downstream from the promoter. The combination of a coding region for an enzyme operably linked to a promoter forms an expression cassette, which can optionally include other elements as well.

Promoter regions are typically found in the flanking DNA upstream from the coding sequence in both the prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, or inducible. A strong promoter provides for a high level of gene expression, whereas a weak promoter provides for a very low level of gene expression. An inducible promoter is a promoter that provides for turning on and off gene expression in response to an exogenously added agent, or to an environmental or developmental stimulus. For example, a bacterial promoter such as the P_{tac} promoter can be induced to varying levels of gene expression depending on the level of isopropyl-beta-D-thiogalactoside added to the transformed cells. Promoters can also provide for tissue specific or developmental regulation. An isolated promoter sequence that is a strong promoter for heterologous DNAs is advantageous because it provides for a sufficient level of gene expression for easy detection and selection of transformed cells and provides for a high level of gene expression when desired.

Expression cassettes generally include, but are not limited to, examples of plant promoters such as the CaMV 35S promoter (Odell et al., Nature. 313:810-812 (1985)), or others such as CaMV 19S (Lawton et al., Plant Molecular Biology. 9:315-324 (1987)), nos (Ebert et al., Proc. Natl. Acad. Sci. USA, 84:5745-5749 (1987)), Adh1 (Walker et al., Proc. Natl. Acad. Sci. USA. 84:6624-6628 (1987)), sucrose synthase (Yang et al., Proc. Natl. Acad. Sci, USA. 87:4144-4148 (1990)), α-tubulin, ubiquitin, actin (Wang et al., Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)) or those associated with the R gene complex (Chandler et al., The Plant Cell. 1:1175-1183 (1989)). Further suitable promoters include a CYP71D16 trichome-specific, promoter and the CBTS (cembratrienol synthase) promotor, cauliflower mosaic virus promoter, the Z10 promoter from a gene encoding a 10 kD zein protein, a Z27 promoter from a gene encoding a 27 kD zein protein, the plastid rRNA-operon (rrn) promoter, inducible promoters, such as the light inducible promoter derived from the pea rbcS gene (Coruzzi et al., EMBO J. 3:1671 (1971)), RUBISCO-SSU light inducible promoter (SSU) from tobacco and the actin promoter from rice (McElroy et al., The Plant Cell. 2:163-171 (1990)). Other promoters that are useful can also be employed.

Examples of leaf-specific promoters include the promoter from the Populus ribulose-1,5-bisphosphate carboxylase small subunit gene (Wang et al. Plant Molec Biol Reporter 31 (1): 120-127 (2013)), the promoter from the Brachypodium distachyon sedoheptulose-1,7-bisphosphatase (SBPase-p) gene (Alotaibi et al. Plants 7(2): 27 (2018)), the fructose-1,6-bisphosphate aldolase (FBPA-p) gene from Brachypodium distachyon (Alotaibi et al. Plants 7(2): 27 (2018)), and the photosystem-II promoter (CAB2-p) of the rice (Oryza sativa L.) light-harvest chlorophyll a/b binding protein (CAB) (Song et al. J Am Soc Hort Sci 132(4): 551-556 (2007)). Additional promoters that can be used include those available in expression databases, see for example, website bar.utoronto.ca/eplant/ which includes poplar or heterologous promoters from Arabidopsis (for example from AT2G26020/PDF1.2b or AT5G44420/LCR77).

Alternatively, novel tissue specific promoter sequences may be employed. cDNA clones from a particular tissue can be isolated and those clones which are expressed specifically in that tissue can be identified, for example, using Northern blotting. Preferably, the gene isolated is not present in a high copy number but is relatively abundant in specific tissues. The promoter and control elements of corresponding genomic clones can then be localized using techniques well known to those of skill in the art.

Plant plastid originated promoters can also be used, for example, to improve expression in plastids, for example, a rice clp promoter, or tobacco rrn promoter. Chloroplast-specific promoters can also be utilized for targeting the foreign protein expression into chloroplasts. Far example, the 16S ribosomal RNA promoter (Prrn) like psbA and atpA gene promoters can be used for chloroplast transformation.

A nucleic acid encoding one or more LDSP, enzyme, and/or LDSP-protein fusion can be combined with the promoter by standard methods to yield an expression cassette, for example, as described in Sambrook et al. (MOLECULAR CLONING: A LABORATORY MANUAL. Second Edition (Cold Spring Harbor, N.Y.: Cold Spring Harbor Press (1989); MOLECULAR CLONING: A LABORATORY MANUAL. Third Edition (Cold Spring Harbor, N.Y.: Cold Spring Harbor Press (2000)). Briefly, a plasmid containing a promoter such as the 35S CAW promoter or the CYP71D16 trichome-specific promoter can be constructed as described in Jefferson (Plant Molecular Biology Reporter 5:387-405 (1987)) or obtained from Clontech Lab in Palo Alto, Calif. (e.g., pBI121 or pBI221). Typically, these plasmids are constructed to have multiple cloning sites having specificity for different restriction enzymes downstream from the promoter.

The nucleic acid sequence encoding one or more LDSP, enzyme, and/or LDSP-protein fusion can be subcloned downstream from the promoter using restriction enzymes and positioned to ensure that the DNA is inserted in proper orientation with respect to the promoter so that the DNA can be expressed as sense RNA. Once the nucleic acid segment encoding the one or more LDSP, enzyme, and/or LDSP-protein fusion is operably linked to a promoter, the expression cassette so formed can be subcloned into a plasmid or other vector (e.g., an expression vector).

In some embodiments, a cDNA clone encoding a LDSP, enzyme, and/or LDSP-protein fusion is isolated from selected plant tissues, or a nucleic acid encoding a wild type, mutant or modified enzyme is prepared by available methods or as described herein. For example, the nucleic acid encoding the enzyme can be any nucleic acid with a coding region that hybridizes to SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 66, 70, 74, 78, 82, 87, 88, 90, 94, 98, 100, 102, 103, 106, or 109, and that encodes a protein with LDSP-anchoring activity and/or enzyme activity. Using restriction endonucleases, the entire coding sequence for the LDSP, enzyme, and/or LDSP-protein fusion is subcloned downstream of the promoter in a 5′ to 3′ sense orientation.

Targeting Sequences: Additionally, expression cassettes can be constructed and employed to target the nucleic acids encoding one or more LDSP, enzyme, and/or LDSP-protein fusion to an intracellular compartment within plant cells or to direct an encoded protein to the extracellular environment. This can generally be achieved by joining a DNA sequence encoding a LDSP, transit or signal peptide sequence to the coding sequence of the nucleic acid encoding the enzyme. The resultant transit, or signal, peptide can transport the protein to a particular intracellular, or extracellular destination, and can then be co-translationally or post-translationally removed.

Transit peptides act by facilitating the transport of proteins through intracellular membranes, e.g., vacuole, vesicle, plastid and mitochondrial membranes, whereas signal peptides direct proteins through the extracellular membrane. By facilitating transport of the protein into compartments inside or outside the cell, these sequences can increase the accumulation of a particular gene product in a particular location. For example, see U.S. Pat. No. 5,258,300. For example, in some cases it may be desirable to localize the enzymes to lipid droplets.

The best compliment of LDSP/transit peptides/secretion peptide/signal peptides can be empirically ascertained. The choices can range from using the native secretion signals akin to the enzyme candidates to be transgenically expressed, to transit peptides from proteins known to be localized into plant organelles such as trichome plastids in general.

For example, transit peptides can be selected from proteins that have a relative high titer in the trichomes. Examples include, but not limited to, transit peptides form a terpenoid cyclase cembratrieneol cyclase), the LTP1 protein, the Chlorophyll a-b binding protein 40, Phylloplanin, Glycine-rich Protein (GRP), Cytochrome P450 (CYP71D16); all from Nicotiana sp. alongside RUBISCO (Ribulose bisphosphate carboxylase) small unit protein from both Arabidopsis and Nicotiana sp.

3′ Sequences: When the expression cassette is to be introduced into a plant cell, the expression cassette can also optionally include 3′ untranslated plant regulatory DNA sequences that act as a signal to terminate transcription and allow for the polyadenylation of the resultant mRNA. The 3′ untranslated regulatory DNA sequence can include from about 300 to 1,000 nucleotide base pairs and can contain plant transcriptional and translational termination sequences. For example, 3′ elements that can be used include those derived from the nopaline synthase gene of Agrobacterium tumefaciens (Bevan et al., Nucleic Acid Research. 11:369-385 (1983)), or the terminator sequences for the T7 transcript from the octopine synthase gene of Agrobacterium tumefaciens, and/or the 3′ end of the protease inhibitor I or II genes from potato or tomato. Other 3′ elements known to those of skill in the art can also be employed. These 3′ untranslated regulatory sequences can be obtained as described in An (Methods in Enzymology. 153:292 (1987)). Many such 3′ untranslated regulatory sequences are already present in plasmids available from commercial sources such as Clontech, Palo Alto, Calif. The 3′ untranslated regulatory sequences can be operably linked to the 3′ terminus of the nucleic acids encoding the LDSP or enzyme.

Selectable and Screenable Marker Sequences: To improve identification of transformants, a selectable or screenable marker gene can be employed with the expressible nucleic acids encoding the LDSP and/or enzyme(s). “Marker genes” are genes that impart a distinct phenotype to cells expressing the marker gene and thus allow such transformed cells to be distinguished from cells that do not have the marker. Such genes may encode either a selectable or a screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the R-locus trait). Of course, many examples of suitable marker genes are available can be employed in the practice of the invention.

Included within the terms ‘selectable or screenable marker genes’ are also genes which encode a “secretable marker” whose secretion can be detected as a means of identifying or selecting for transformed cells. Examples include markers which encode a secretable antigen that can be identified by antibody interaction, or secretable enzymes that can be detected by their catalytic activity. Secretable proteins fall into a number of classes, including small, diffusible proteins detectable, e.g., by ELISA; and proteins that are inserted or trapped in the cell wall (e.g., proteins that include a leader sequence such as that found in the expression unit of extensin or tobacco PR-S).

With regard to selectable secretable markers, the use of an expression system that encodes a polypeptide that becomes sequestered in the cell wall, where the polypeptide includes a unique epitope may be advantageous. Such a cell wall antigen can employ an epitope sequence that would provide low background in plant tissue, a promoter-leader sequence that imparts efficient expression and targeting across the plasma membrane, and that can produce protein that is bound in the cell wall and yet is accessible to antibodies. A normally secreted cell wall protein modified to include a unique epitope would satisfy such requirements.

Examples of protein markers suitable for modification in this manner include extensin or hydroxyproline rich glycoprotein (HPRG). For example, the maize HPRG (Stiefel at al., The Plant Cell, 2:785-793 (1990)) is well characterized in terms of molecular biology, expression, and protein structure and therefore can readily be employed. However, any one of a variety of extensins and/or glycine-rich cell wall proteins (Keller et al., EMBO J. 8:1309-1314 (1989)) could be modified by the addition of an antigenic site to create a screenable marker.

Selectable markers for use in connection with the present invention can include, but are not limited to, a neo gene (Potrykus et al., Mol. Gen. Genet. 199:183-188 (1985)) which codes for kanamycin resistance and can be selected for using kanamycin, G418; a bar gene which codes for bialaphos resistance; a gene which encodes an altered EPSP synthase protein (Hinchee et al., Bio/Technology. 6:915-922 (1988)) thus conferring glyphosate resistance; a nitrilase gene such as bxn from Klebsiella ozaenae which confers resistance to bromoxynil (Stalker et al., Science. 242:419-423 (1988)); a mutant acetolactate synthase gene (ALS) which confers resistance to imidazolinone, sulfonylurea or other ALS-inhibiting chemicals (European Patent Application 154,204 (1985)); a methotrexate-resistant DHFR gene (Thillet et al., J. Biol. Chem, 263:12500-12508 (1988)); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide, CTP (European Patent Application 0 218 571 (1987)).

An illustrative embodiment of a selectable marker gene capable of being used in systems to select transformants is the gene that encode the enzyme phosphinothricin acetyltransferase, such as the bar gene from Streptomyces hygroscopicus or the pat gene from Streptomyces viridochromogenes (U.S. Pat. No. 5,550,318). The enzyme phosphinothricin acetyl transferase (PAT) inactivates the active ingredient in the herbicide bialaphos, phosphinothricin (PPT). PPT inhibits glutamine synthetase, (Murakami et al., Mol. Gen. Genet. 205:42-50 (1986); Twell et al., Plant Physiol. 91:1270-1274 (1989)) causing rapid accumulation of ammonia and cell death.

Screenable markers that may be employed include, but are not limited to, a β-glucuronidase or uidA gene (GUS) that encodes an enzyme for which various chromogenic substrates are known; an R-locus gene, which encodes a product that regulates the production of anthocyanin pigments (red color) in plant tissues (Dellaporta et al., In: Chromosome Structure and Function: Impact of New Concepts, 18^{th} Stadler Genetics Symposium, J. P. Gustafson and R. Appels, eds. (New York: Plenum Press) pp. 263-282 (1988)); a β-lactamase gene (Sutcliffe, Proc. Natl. Acad. Sci, USA. 75:3737-3741 (1978)), which encodes an enzyme for which various chromogenic substrates are known (e.g., PADAC, a chromogenic cephalosporin); a xylE gene (Zukowsky et al., Proc. Natl. Acad. Sci. USA. 80:1101 (1983)) which encodes a catechol dioxygenase that can convert chromogenic catechols; an α-amylase gene (Ikuta et al., Bio/technology 8:241-242 (1990)); a tyrosinase gene (Katz et al., J Gen. Microbial. 129:2703-2714 (1983)) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science. 234:856-859.1986), which allows for bioluminescence detection; or an aequorin gene (Prasher et al., Biochem. Biophys. Res. Comm. 126:1259-1268 (1985)), which may be employed in calcium-sensitive bioluminescence detection, or a green or yellow fluorescent protein gene (Niedz et al., Plant Cell Reports. 14:403 (1995)).

Another screenable marker contemplated for use is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for population screening for bioluminescence, such as on tissue culture plates, or even for whole plant screening.

Other Optional Sequences: An expression cassette of the invention can also include plasmid DNA. Plasmid vectors include additional DNA sequences that provide for easy selection, amplification, and transformation of the expression cassette in prokaryotic and eukaryotic cells, e.g., pUC-derived vectors such as pUC8, pUC9, pUC18, pUC19, pUC23, pUC119, and pUC120, pSK-derived vectors, pGEM-derived vectors, pSP-derived vectors, or pBS-derived vectors. The additional DNA sequences can include origins of replication to provide for autonomous replication of the vector, additional selectable marker genes, for example, encoding antibiotic or herbicide resistance, unique multiple cloning sites providing for multiple sites to insert DNA sequences or genes encoded in the expression cassette and sequences that enhance transformation of prokaryotic and eukaryotic cells.

Another vector that is useful for expression in both plant and prokaryotic cells is the binary Ti plasmid (as disclosed in Schilperoort et al., U.S. Pat. No. 4,940,838) as exemplified by vector pGA582. This binary Ti plasmid vector has been previously characterized by An (Methods in Enzymology. 153:292 (1987)) and is available from Dr. An. This binary Ti vector can be replicated in prokaryotic bacteria such as E. coli and Agrobacterium. The Agrobacterium plasmid vectors can be used to transfer the expression cassette to dicot plant cells, and under certain conditions to monocot cells, such as rice cells. The binary Ti vectors can include the nopaline T DNA right and left borders to provide for efficient plant cell transformation, a selectable marker gene, unique multiple cloning sites in the T border regions, the colE1 replication of origin and a wide host range replicon. The binary Ti vectors carrying an expression cassette of the invention can be used to transform both prokaryotic and eukaryotic cells but is usually used to transform dicot plant cells.

DNA Delivery of the DNA Molecules into Host Cells: Methods described herein can include introducing nucleic acids encoding LDSP and/or enzymes, such as a preselected cDNA encoding the selected LDSP and/or enzyme, into a recipient cell to create a transformed cell. In some instances, the frequency of occurrence of cells taking up exogenous (foreign) DNA may be low. Moreover, it is most likely that not all recipient cells receiving DNA segments or sequences will result in a transformed cell wherein the DNA is stably integrated into the plant genome and/or expressed. Some recipient cells may provide only initial and transient gene expression. However, certain cells from virtually any dicot or monocot species may be stably transformed, and these cells regenerated into transgenic plants, through the application of the techniques disclosed herein.

Another aspect of the invention is a plant or plant cell that can produce terpenes, diterpenes and terpenoids, wherein the plant has introduced nucleic acid sequence(s) encoding one or more enzymes. The plant or plant cell can be a monocotyledon or a dicotyledon.

Another aspect of the invention includes plant cells (e.g., embryonic cells or other cell lines) that can regenerate fertile transgenic plants and/or seeds. The cells can be derived from either monocotyledons or dicotyledons. In some embodiments, the plant or cell is a monocotyledon plant or cell. In some embodiments, the plant or cell is a dicotyledon plant or cell. For example, the plant or cell can be a tobacco plant or cell. The cell(s) may be in a suspension cell culture or may be in an intact plant part, such as an immature embryo, or in a specialized plant tissue, such as callus, such as Type I or Type II callus.

Transformation of plant cells can be conducted by any one of a number of methods available in the art. Examples are: Transformation by direct DNA transfer into plant cells by electroporation (U.S. Pat. Nos. 5,384,253 and 5,472,869, Dekeyser et al., The Plant Cell. 2:591-602 (1990)); direct DNA transfer to plant cells by PEG precipitation (Hayashimoto et al., Plant Physiol. 93:857-863 (1990)); direct DNA transfer to plant cells by microprojectile bombardment (McCabe et al., Bio/Technology. 6:923-926 (1988); Gordon-Kamm et al., The Plant Cell. 2:603-618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack the functions for disease induction.

One method for dicot transformation, for example, involves infection of plant cells with Agrobacterium tumefaciens using the leaf-disk protocol (Horsch et al., Science 227:1229-1231 (1985). Methods for transformation of monocotyledonous plants utilizing Agrobacterium tumefaciens have been described by Hiei et al. (European Patent 0 604 662, 1994) and Saito et al. (European Patent 0 672 752, 1995).

Monocot cells such as various grasses or dicot cells such as tobacco can be transformed via microprojectile bombardment of embryogenic callus tissue or immature embryos, or by electroporation following partial enzymatic degradation of the cell wall with a pectinase-containing enzyme (U.S. Pat. Nos. 5,384,253; and 5,472,869). For example, embryogenic cell lines derived from immature embryos can be transformed by accelerated particle treatment as described by Gordon-Kamm et al. (The Plant Cell. 2:603-618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. application Ser. No. 08/112,245 and PCT publication WO 95/06128.

The choice of plant tissue source for transformation may depend on the nature of the host plant and the transformation protocol. As illustrated herein, leaves were used in some transient expression experiments. Useful tissue sources include callus, suspensions culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells.

The transformation is carried out under conditions directed to the plant tissue of choice. The plant cells or tissue are exposed to the DNA or RNA encoding enzymes for an effective period of time. This may range from a less than one second pulse of electricity for electroporation to a 2-day to 3-day co-cultivation in the presence of plasmid-bearing Agrobacterium cells. Buffers and media used will also vary with the plant tissue source and transformation protocol. Many transformation protocols employ a feeder layer of suspended culture cells (tobacco, for example) on the surface of solid media plates, separated by a sterile filter paper disk from the plant cells or tissues being transformed.

In some cases, plastid expression is desired. Transformation of plastids can be achieved by use of expression cassettes or expression vectors that include one or more of the following: delivery of expression cassettes or expression vectors across cell membranes and intracellular plastid membranes, one or more regions of homology with plastid DNA, enzyme nucleotide sequences optimized for plastid expression, one or more selectable markers for plastid transformation, segregation of genomic copies of the expression cassette within a plastid, or a combination thereof. Particle bombardment can be used for plastid transformation, but other methods can also be used. For example, polyethylene glycol (PEG) treatment of protoplasts has been used to transform plastids.

Electroporation: Where one wishes to introduce DNA by means of electroporation, it is contemplated that the method of Krzyzek et al. (U.S. Pat. No. 5,384,253) may be advantageous. In this method, certain cell wall-degrading enzymes, such as pectin-degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin-degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

Microprojectile Bombardment: A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

In some cases, expression cassette/expression vector nucleic acids can be precipitated onto metal particles for DNA delivery using microprojectile bombardment. However, in some instances DNA precipitation onto metal particles would not be necessary for DNA delivery to a recipient cell using microprojectile bombardment. In an illustrative embodiment, non-embryogenic cells were bombarded with intact cells of the bacteria E. coil or Agrobacterium tumefaciens containing plasmids with either the β-glucoronidase or bar gene engineered for expression in selected plant cells. Bacteria were inactivated by ethanol dehydration prior to bombardment. A low level of transient expression of the β-glucoronidase gene was observed 24-48 hours following DNA delivery. In addition, stable transformants containing the bar gene were recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, microprojectile bombardment does not require the isolation of protoplasts (Christou et al., PNAS. 84:3962-3966 (1987)), the formation of partially degraded cells, and no susceptibility to Agrobacterium infection is required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon-Kamm et al., The Plant Cell. 2:603-618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing the damage inflicted on recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein, one may obtain up to 1000 or more foci of cells transiently expressing a marker gene. The number of cells in a focus which express the exogenous gene product 48 hours post-bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macro- or microprojectiles. Biological factors include all steps involved in manipulation of cells before and immediately after bombardment, the osmotic adjustment of target cells to help alleviate the trauma associated with the bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore, influence transformation and integration efficiencies. For example, the osmotic state, tissue hydration and the subculture stage or cell cycle of the recipient cells may be adjusted for optimum transformation. Execution of such routine adjustments will be known to those of skill in the art.

Selection: An exemplary embodiment of methods for identifying transformed cells involves exposing the bombarded cultures to a selective agent, such as a metabolic inhibitor, an antibiotic, or the like. Cells which have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured. for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations that provide 100% inhibition followed by screening of growing tissue for expression of a screenable marker gene such as luciferase would allow one to recover transformants from cell or tissue types that are not amenable to selection alone.

Regeneration and Seed Production: Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to express the trait. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants to introgress the nucleic acids encoding an enzyme into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced nucleic acids, the plant is self-pollinated at least once in order to produce a homozygous backcross converted inbred containing the nucleic acids encoding the enzyme(s). Progeny of these plants are true breeding.

Alternatively, seed from transformed plants regenerated from transformed tissue cultures is grown in the field and self-pollinated to generate true breeding plants.

Seed from the fertile transgenic plants can then be evaluated for the presence and/or expression of the enzyme(s). Transgenic plant and/or seed tissue can be analyzed for enzyme expression using methods such as SDS polyacrylamide gel electrophoresis, Western blot, liquid chromatography (e.g., HPLC) or other means of detecting an enzyme product (e.g., a terpene, diterpene, terpenoid, or a combination thereof).

Once a transgenic seed expressing the enzyme(s) and producing one or more terpenes, diterpenes, and/or terpenoids in the plant is identified, the seed can be used to develop true breeding plants. The true breeding plants are used to develop a line of plants expressing terpenes, diterpenes, and/or terpenoids in various plant tissues (e.g., in leaves, bracts, and/or trichomes) while still maintaining other desirable functional agronomic traits. Adding the trait of terpene, diterpene, and/or terpenoid production can be accomplished by back-crossing with selected desirable functional agronomic trains) and with plants that do not exhibit such traits and studying the pattern of inheritance in segregating generations. Those plants expressing the target trait(s) in a dominant fashion are preferably selected. Back-crossing is carried out by crossing the original fertile transgenic plants with a plant from an inbred line exhibiting desirable functional agronomic characteristics while not necessarily expressing the trait of terpene, diterpene, and/or terpenoid production in the plant. The resulting progeny can then be crossed back to the parent that expresses the terpenes, diterpenes, and/or terpenoids. The progeny from this cross will also segregate so that some of the progeny carry the trait and some do not. This back-crossing is repeated until the goal of acquiring an inbred line with the desirable functional agronomic traits, and with production of terpenes, diterpenes, and/or terpenoids within various tissues of the plant is achieved. The enzymes can be expressed in a dominant fashion.

Subsequent to back-crossing, the new transgenic plants can be evaluated for synthesis of terpenes, diterpenes, and/or terpenoids in selected plant lines. This can be done, for example, by gas chromatography, mass spectroscopy, or NMR analysis of whole plant cell walls (Kim, H., and Ralph, J. Solution-state 2D NMR of ball-milled plant cell wall gels in DMSO-d₆/pyridine-d₅. (2010) Org. Biomol. Chem. 8(3), 576-591; Yelle, D. J., Ralph, J., and Frihart, C. R. Characterization of non-derivatized plant cell walls using high-resolution solution-state NMR spectroscopy. (2008) Magn. Resort. Chem. 46(6), 508-517; Kim, R, Ralph, J., and Akiyama, T. Solution-state 2D NMR of Ball-milled Plant Cell Wall Gels in DMSO-d₆. (2008) BioEnergy Research 1(1), 56-66; Lu, F., and Ralph, J. Non-degradative dissolution and acetylation of ball-milled plant cell walls; high-resolution solution-state NMR. (2003) Plant J. 35(4), 535-544). The new transgenic plants can also be evaluated for a battery of functional agronomic characteristics such as lodging, yield, resistance to disease, resistance to insect pests, drought resistance, and/or herbicide resistance.

Determination of Stably Transformed Plant Tissues: To confirm the presence of the nucleic acids encoding terpene synthesizing enzymes in the regenerating plants, or seeds or progeny derived from the regenerated plant, a variety of assays may be performed. Such assays include, for example, molecular biological assays, such as Southern and Northern blotting and PCR; biochemical assays, such as detecting the presence of enzyme products, for example, by enzyme assays, by immunological assays (ELISAs and Western blots). Various plant parts can be assayed, such as trichomes, leaves, bracts, seeds or roots. In some cases, the phenotype of the whole regenerated plant can be analyzed.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced nucleic acids. PCR can also be used to reverse transcribe RNA into DNA, using enzymes such as reverse transcriptase, and then this DNA can be amplified through the use of conventional PCR techniques. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and also demonstrate the presence or absence of an RNA species.

While Southern blotting may be used to detect the nucleic acid encoding the enzyme(s) in question, it may not provide information as to whether the preselected DNA segment is being expressed. Expression may be evaluated by specifically identifying the protein products of the introduced nucleic acids or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as, native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the enzyme such as evaluation by amino acid sequencing following purification. Other procedures may be additionally used.

The expression of a gene product can also be determined by evaluating the phenotypic results of its expression. These assays also may take many forms including but not limited to analyzing changes in the chemical composition, morphology, or physiological properties of the plant. Chemical composition may be altered by expression of preselected DNA segments encoding storage proteins which change amino acid composition and may be detected by amino acid analysis.

### Hosts

Terpenes, including diterpenes and terpenoids, can be made in a variety of host organisms. As used herein, a “host” means a cell, tissue or organism capable of replication. The host can have an expression cassette or expression vector that can include a nucleic acid segment encoding an enzyme that is involved in the biosynthesis of terpenes.

The term “host cell”, as used herein, refers to any prokaryotic or eukaryotic cell that can be transformed with an expression cassettes or vector carrying the nucleic acid segment encoding one or more LDSP, enzyme, LDSP-protein fusion, or a combination thereof that is involved in the biosynthesis of one or more terpenes. The host cells can, for example, be a plant, bacterial, insect, or yeast cell. Expression cassettes encoding biosynthetic enzymes can be incorporated or transferred into a host cell to facilitate manufacture of the enzymes described herein or the terpene, diterpene, or terpenoid products of those enzymes.

For example, the enzymes, terpenes, diterpenes, and terpenoids can be made in plants or plant cells. The terpenes, diterpenes, and terpenoids can, for example, be made and extracted from whole plants, plant parts, plant cells, or a combination thereof. Enzymes can also be made, for example, in insect, plant, or fungal (e.g., yeast) cells.

Examples of host cells include, without limitation, tobacco cells such as Nicotiana benthamiana, Nicotiana tabacum, Nicotiana rustica, Nicotiana excelsior, and Nicotiana excelsiana cells; cells of the genus Escherichia such as the species Escherichia coli; cells of the genus Clostridium such as the species Clostridium ljungdahlii, Clostridium autoethanogenum or Clostridium kluyveri; cells of the genus Corynebacterium such as the species Corynebacterium glutamicum; cells of the genus Cupriavidus such as the species Cupriavidus necator or Cupriavidus metallidurans; cells of the genus Pseudomonas such as the species Pseudomonas fluorescens, Pseudomonas putida or Pseudomonas oleavorans; cells of the genus Delftia such as the species Delftia acidovorans; cells of the genus Bacillus such as the species Bacillus subtilis; cells of the genus Lactobacillus such as the species Lactobacillus delbrueckii; or cells of the genus Lactococcus such as the species Lactococcus lactis.

“Host cells” can further include, without limitation, those from yeast and other fungi, as well as, for example, insect cells. Examples of suitable eukaryotic host cells include yeasts and fungi from the genus Aspergillus such as Aspergillus niger; from the genus Saccharomyces such as Saccharomyces cerevisiae; from the genus Candida such as C. tropicalis, C. albicans, C. cloacae, C. guillermondii, C. intermedia, C. maltosa, C. parapsilosis, and C. zeylenoides; from the genus Pichia (or Komagataella) such as Pichia pastoris; from the genus Yarrowia such as Yarrowia lipolytica; from the genus Issatchenkia such as Issathenkia orientalis; from the genus Debaryomyces such as Debaryomyces hansenii; from the genus Arxula such as Arxula adenoinivorans; or from the genus Kluyveromyces such as Kluyveromyces lactis or from the genera Exophiala, Mucor, Trichoderma, Cladosporium, Phanerochaete, Cladophialophora, Paecilomyces, Scedosporium, and Ophiostoma.

The host cells can have organelles that facilitate manufacture or storage of the terpenes, diterpenes, and terpenoids. Such organelles can include lipid droplets. During and after production of the terpenes, diterpenes, and terpenoids these organelles can be isolated as a semi-pure source of the of the terpenes, diterpenes, and terpenoids.

As illustrated herein, terpenoid yields obtained using the methods described herein demonstrate the versatility of the transient N. benthamiana system as a platform to produce terpenaids at industrial scales in economically relevant biomass crops.

### Methods

Methods are described herein that are useful for synthesizing terpenes. The methods can involve incubating cells or tissues having a heterologous at least one expression cassette or expression vector that can express any of the enzymes and/or proteins described herein.

For example, one method can involve (a) incubating a population of host cells or host tissue comprising any of the expression systems, enzymes, lipid droplet, and/or fusion proteins described herein; and (b) isolating lipids from the population of host cells or the host tissue. In some cases, the host cells or the host tissue can be in a plant, in which case the incubating step is a cultivating step where the plant is cultivated in an environment suitable for plant growth.

Another example of a method can involve (a) incubating a population of host cells or a host tissue, or cultivating a host seed or a host plant, where the population of host cells, the host tissue, host seed, or cells of the host plant has an expression system having at least one expression cassette having a heterologous promoter operably linked to a nucleic acid segment encoding a fusion protein comprising a lipid droplet surface protein linked in-frame to one or more a fusion partners such as a monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-Co A reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), patchoulol synthase, or WRI1 protein; and (b) isolating lipids from the population of host cells, the host plant's cells, or the host tissue. In some cases, a combination of enzymes, transcription factors, and lipid droplet proteins can be expressed in host cells, host plant, or host tissues.

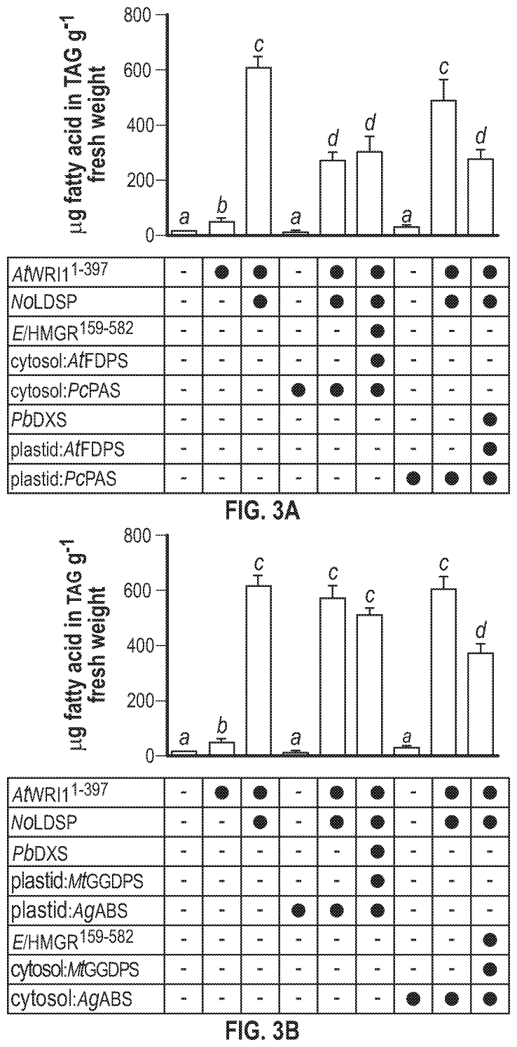

For example, high diterpenoid yields were obtained when cells or tissues were engineered to co-express DXS, GGDPS (MtGGDSP, TsGGDPS, or EpGGDPS2), and AgABS and these enzymes were targeted to plastids by fusion to a plastid-targeting peptide (see FIGS. 2A-2B, and 3B). Added expression of AtWRI(1-397) did not significantly affect diterpenoid production. Hence, it can be useful to use cells or tissues in such methods when the cells or tissues produce enzymes DXS, GGDPS, and ABS in plastids with or without expression of the WRI1 transcription factor.

In another example, high diterpenoid yields were obtained when each of the following was expressed in the cytosol: HMGR159-582, MtGGDPS, and AgABS85-868 (FIG. 2C and FIG. 3B). Added expression of AtWRI1-397 and NoLDSP did not significantly affect diterpenoid production.

In another example, high diterpenoid yields were obtained when cells or tissues were engineered to co-produce cytosolic HMGR (e.g., cytosol:HMGR(159-582)), cytosolic GGDPS (e.g., cytosol:MtGGDPS), LDSP-fused ABS (e.g., LD:AgABS(85-868)), and WRI1 (FIG. 5).

To produce other types terpenes and teipenoids, different types of enzymes can be used. For example, for production of functionalized diterpenoids in lipid droplets the following combinations of enzymes can be used: WRI1, LDSP, DXS (plastid), GGDSP (plastid), ABS (plastid), and either CYP (ER) or [CYP (LD) and CPR(LD)] (see, e.g., FIG. 5). Note that ER means that the enzyme or protein is localized in the endoplasmic reticulum, while LD means that the enzyme or protein is targeted to lipid droplets (e.g. because the enzyme or protein is fused to LDSP).

In another example, the following combinations of enzymes can be used to produce functionalized diterpenoids that are sequestered within or on lipid droplets: WRI1, LDSP, HMGR (cytosol), GGDPS (cytosol), ABS (cytosol), and CYP (ER) (see, e.g., FIG. 5).

In another example, the following combinations of enzymes can be used to produce functionalized diterpenoids in lipid droplets: WRI1, HMGR (cytosol), GGDPS (cytosol), ABS (LD), CYP (LD) and CPR (LD).

### Definitions

As used herein, “isolated” means a nucleic acid, polypeptide, or product has been removed from its natural or native cell. Thus, the nucleic acid, polypeptide, or product can be physically isolated from the cell, or the nucleic acid or polypeptide can be present or maintained in another cell where it is not naturally present or synthesized. The isolated nucleic acid, the isolated polypeptide, or the isolated product can also be a nucleic acid, protein, or product that is modified but has been introduced into a cell where it is or was naturally present. Thus, a modified isolated nucleic acid or an isolated polypeptide expressed from a modified isolated nucleic acid can be present in a cell along with a wild copy of the (unmodified) natural nucleic acid and along with wild type copies of the (natural) polypeptide.

As used herein, a “native” nucleic acid or polypeptide means a DNA, RNA, amino acid sequence or segment thereof that has not been manipulated in vivo or in vitro, i.e., has not been isolated, purified, amplified, mutated, and/or modified.

The term “transgenic” when used in reference to a plant or leaf or vegetative tissue or seed for example a “transgenic plant,” transgenic leaf,” “transgenic vegetative tissue,” “transgenic seed,” or a “transgenic host cell” refers to a plant or leaf or tissue or seed that contains at least one heterologous or foreign gene in one or more of its cells. The term “transgenic plant material” refers broadly to a plant, a plant structure, a plant tissue, a plant seed or a plant cell that contains at least one heterologous gene in one or more of its cells.

The term “transgene” refers to a foreign gene that is placed into an organism or host cell by the process of transfection. The term “foreign nucleic acid” or refers to any nucleic acid (e.g., encoding a promoter or coding region) that is introduced into the genome of an organism or tissue of an organism or a host cell by experimental manipulations, such as those described herein, and may include nucleic acid sequences found in that organism so long as the introduced gene does not reside in the same location, as does the naturally occurring gene.

The term “host cell” refers to any cell capable of replicating and/or transcribing and/or translating a heterologous nucleic acid. Thus, a “host cell” refers to any eukaryotic or prokaryotic cell (e.g., plant cells, algal cells, bacterial cells, yeast cells, E. coli, insect cells, etc.), whether located in vitro or in vivo. For example, a host cell may be located in a transgenic plant or located in a plant part or part of a plant tissue or in cell culture.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene.

As used herein, the term “plant” is used in its broadest sense. It includes, but is not limited to, any species of grass (fodder, ornamental or decorative), crop or cereal, fodder or forage, fruit or vegetable, fruit plant or vegetable plant, herb plant, woody plant, flower plant or tree. It is not meant to limit a plant to any particular structure. It also refers to a unicellular plant (e.g. microalga) and a plurality of plant cells that are largely differentiated into a colony (e.g. volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture.

As used herein, the term “plant part” as used herein refers to a plant structure or a plant tissue, for example, pollen, an ovule, a tissue, a pod, a seed, a leaf and a cell. Plant parts may comprise one or more of a tiller, plug, rhizome, sprig, stolen, meristem, crown, and the like. In some instances, the plant part can include vegetative tissues of the plant.

Vegetative tissues or vegetative plant parts do not include plant seeds, and instead include non-seed tissues or parts of a plant. The vegetative tissues can include reproductive tissues of a plant, but not the mature seeds.

The term “seed” refers to a ripened ovule, consisting of the embryo and a casing.

The term “propagation” refers to the process of producing new plants, either by vegetative means involving the rooting or grafting of pieces of a plant, or by sowing seeds. The terms “vegetative propagation” and “asexual reproduction” refer to the ability of plants to reproduce without sexual reproduction, by producing new plants from existing vegetative structures that are clones, plants that are identical in all attributes to the mother plant and to one another. For example, the division of a clump, rooting of proliferations, or cutting of mature crowns can produce a new plant.

The term “heterologous” when used in reference to a nucleic acid refers to a nucleic acid that has been manipulated in some way. For example, a heterologous nucleic acid includes a nucleic acid from one species introduced into another species. A heterologous nucleic acid also includes a nucleic acid native to an organism that has been altered in some way (e.g., mutated, added in multiple copies, linked to a non-native promoter or enhancer sequence, etc.), Heterologous nucleic acids can include cDNA forms of a nucleic acid; the cDNA may be expressed in either a sense (to produce mRNA) or anti-sense orientation (to produce an anti-sense RNA transcript that is complementary to the mRNA transcript). For example, heterologous nucleic acids can be distinguished from endogenous plant nucleic acids in that the heterologous nucleic acids are typically joined to nucleic acids comprising regulatory elements such as promoters that are not found naturally associated with the natural gene for the protein encoded by the heterologous gene. Heterologous nucleic acids can also be distinguished from endogenous plant nucleic acids in that the heterologous nucleic acids are in an unnatural chromosomal location or are associated with portions of the chromosome not found in nature (e.g., the heterologous nucleic acids are expressed in tissues where the gene is not normally expressed).

The term “expression” when used in reference to a nucleic acid sequence, such as a gene, refers to the process of converting genetic information encoded in a gene into RNA (e.g., mRNA, rRNA, tRNA, or snRNA) through “transcription” of the gene (i.e., via the enzymatic action of an RNA polymerase), and into protein where applicable (as when a gene encodes a protein), through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The terms “in operable combination,” “in operable order,” and “operably linked” refer to the linkage of nucleic acid sequences in such a manner that a nucleic acid molecule capable of directing the transcription of a coding region (e.g., gene) and/or the synthesis of a desired protein molecule is produced. The term also refers to the linkage of amino acid sequences in such a manner so that a functional protein is produced.

Transcriptional control signals in eukaryotes comprise “promoter” and “enhancer” elements. Promoters and enhancers consist of short arrays of DNA sequences that interact specifically with cellular proteins involved in transcription (see, for e.g., Maniatis, et al. (1987) Science 236:1237; herein incorporated by reference). Promoter and enhancer elements have been isolated from a variety of eukaryotic sources including genes in yeast, insect, mammalian and plant cells. Promoter and enhancer elements have also been isolated from viruses and analogous control elements, such as promoters, are also found in prokaryotes. The selection of a particular promoter and enhancer depends on the cell type used to express the protein of interest. Some eukaryotic promoters and enhancers have a broad host range while others are functional in a limited subset of cell types (for review, see Maniatis, et al. (1987), supra; herein incorporated by reference).

The terms “promoter element,” “promoter,” or “promoter sequence” refer to a DNA sequence that is located at the 5′ end of the coding region of a DNA polymer. The location of most promoters known in nature is 5′ to the transcribed region. The promoter functions as a switch, activating the expression of a gene. If the gene is activated, it is said to be transcribed, or is participating in transcription. Transcription involves the synthesis of mRNA from the gene. The promoter, therefore, serves as a transcriptional regulatory element and also provides a site for initiation of transcription of the gene into mRNA.

The term “regulatory region” refers to a gene's 5′ transcribed but untranslated regions, located immediately downstream from the promoter and ending just prior to the translational start of the gene.

The term “promoter region” refers to the region immediately upstream of the coding region of a DNA polymer and is typically between about 500 bp and 4 kb in length and is preferably about 1 to 1.5 kb in length. Promoters may be tissue specific or cell specific.

The term “tissue specific” as it applies to a promoter refers to a promoter that is capable of directing selective expression of a nucleic acid of interest to a specific type of tissue (e.g., vegetative tissues) in the relative absence of expression of the same nucleic acid of interest in a different type of tissue (e.g., seeds). Tissue specificity of a promoter may be evaluated by, for example, operably linking a reporter gene and/or a reporter gene expressing a reporter molecule, to the promoter sequence to generate a reporter construct, introducing the reporter construct into the genome of a plant such that the reporter construct is integrated into every tissue of the resulting transgenic plant, and detecting the expression of the reporter gene detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected.

The term “cell type specific” as applied to a promoter refers to a promoter that is capable of directing selective expression of a nucleic acid of interest in a specific type of cell in the relative absence of expression of the same nucleic acid of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., immunohistochemical staining. Briefly, tissue sections are embedded in paraffin, and paraffin sections are reacted with a primary antibody that is specific for the polypeptide product encoded by the nucleic acid of interest whose expression is controlled by the promoter. A labeled (e.g., peroxidase conjugated) secondary antibody that is specific for the primary antibody is allowed to bind to the sectioned tissue and specific binding detected with avidin/biotin) by microscopy.

Promoters may be “constitutive” or “inducible.” The term “constitutive” when made in reference to a promoter means that the promoter is capable of directing transcription of an operably linked nucleic acid in the absence of a stimulus (e.g., heat shock, chemicals, light, etc.). Typically, constitutive promoters are capable of directing expression of a transgene in substantially any cell and any tissue. Exemplary constitutive plant promoters include, but are not limited to Cauliflower Mosaic Virus (CaMV SD; see e.g., U.S. Pat. No. 5,352,605, incorporated herein by reference), mannopine synthase, octopine synthase (ocs), superpromoter (see e.g., WO 95/14098; herein incorporated by reference), and ubi3 promoters (see e.g., Garbarino and Belknap, Plant Mol. Biol. 24:119-127 (1994); herein incorporated by reference). Such promoters have been used successfully to direct the expression of heterologous nucleic acid sequences in transformed plant tissue.

In contrast, an “inducible” promoter is one that is capable of directing a level of transcription of an operably linked nucleic acid in the presence of a stimulus (e.g., heat shock, chemicals, light, etc.) that is different from the level of transcription of the operably linked nucleic acid in the absence of the stimulus.

The term “vector” refers to nucleic acid molecules that transfer DNA segment(s). Transfer can be into a cell, cell to cell, et cetera. The term “vehicle” is sometimes used interchangeably with “vector.” The vector can, for example, be a plasmid. But the vector need not be plasmid.

As used herein, the singular forms “a,” “an,” and “the” are intended to include the plural forms as well, unless the context clearly indicates otherwise. Also, as used herein, “and/or” refers to, and encompasses, any and all possible combinations of one or more of the associated listed items. Unless otherwise defined, all terms, including technical and scientific terms used in the description, have the same meaning as commonly understood by one of ordinary skill in the art to which this invention pertains.

The term “about”, as used herein, can allow for a degree of variability in a value or range, for example, within 10%, within 5%, or within 1% of a stated value or of a stated limit of a range.

The term “enzyme” or “enzymes”, as used herein, refers to a protein catalyst capable of catalyzing a reaction. Herein, the term does not mean only an isolated enzyme, but also includes a host cell expressing that enzyme. Accordingly, the conversion of A to B by enzyme C should also be construed to encompass the conversion of A to B by a host cell expressing enzyme C.

The terms “identical” or percent “identity”, as used herein, in the context of two or more nucleic acids, or two or more polypeptide sequences, refer to two or more sequences or subsequences that are the same or have a specified percentage of nucleotides or amino acid residues that are the same (e.g., 75% identity, 80% identity, 85% identity, 90% identity, 95% identity, 97% identity, 98% identity, 99% identity, or 100% identity in pairwise comparison). Sequence identity can be determined by comparison and/or alignment of sequences for maximum correspondence over a comparison window, or over a designated region as measured using a sequence comparison algorithm, or by manual alignment and visual inspection. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. A “reference sequence” is a defined sequence used as a basis for a sequence comparison; a reference sequence may be a subset of a larger sequence.

As used herein the term “terpene” includes any type of terpene or terpenoid, including for example any monoterpene, diterpene, sesquiterpene, sesterterpene, triterpene, tetraterpene, polyterpene, and any mixture thereof.

The following non-limiting Examples describe some procedures that can be performed to facilitate making and using the invention.

### EXAMPLE 1

### Materials and Methods

This Example describes some of the materials and methods used in the development of the invention.

Generation of Constructs for Transient Expression Studies in N. benthamiana

The open reading frames encoding truncated A. thaliana WRINKLED1 (AtWRI11-397, AY254038.2) and full-length N. oceanica lipid droplet surface protein (NoLDSP, JQ268559.1) were amplified from existing cDNAs.

The coding sequences for truncated cytosolic E. lathyris HMGR (ElHMGR159-582, JQ694150.1), cytosolic A. thaliana FDPS (cytosol:AtFDPS, NM_117823.4), cytosolic P. cablin patchoulol synthase (cytosol:PcPAS, AY508730), plastidic A. grandis abietadiene synthase (plastid:AgABS, U50768.1), and plastidic P. barbatus (PbDXS) were amplified from cDNAs derived from total RNA of the host organisms.

An amino acid sequence for a cytosolic P. cablin patchoulol synthase (cytosol:PcPAS, AY508730; SEQ ID NO:43) is shown below.

| | |
|---|---|
| 1 | MELYAQSVGV GAASRPLANF HPCVWGDKFI VYNPQSCQAG |
| 41 | EREEAEELKV ELKRELKEAS DNYMRQLKMV DAIQRLGIDY |
| 81 | LFVEDVDEAL KNLFEMFDAF CKNNHDMHAT ALSFRLLRQH |
| 121 | GYRVSCEVFE KFKDGKDGFK VPNEDGAVAV LEFFEATHLR |
| 161 | VHGEDVLDNA FDFTRNYLES VYATLNDPTA KQVHNALNEF |
| 201 | SFRRGLPRVE ARKYISIYEQ YASHHKGLLK LAKLDFNLVQ |
| 241 | ALHRRELSED SRWWKTLQVP TKLSFVRDRL VESYFWASGS |
| 281 | YFEPNYSVAR MILAKGLAVL SLMDDVYDAY GTFEELQMFT |
| 321 | DAIERWDASC LDKLPDYMKI VYKALLDVFE EVDEELIKLG |
| 361 | APYRAYYGKE AMKYAARAYM EEAQWREQKH KPTTKEYMKL |
| 401 | ATKTCGYITL IILSCLGVEE GIVTKEAFDW VFSRPPFIEA |
| 441 | TLIIARLVND ITGHEFEKKR EHVRTAVECY MEEHKVGKQE |
| 481 | VVSEFYNQME SAWKDINEGF LRPVEFPIPL LYLILNSVRT |
| 521 | LEVIYKEGDS YTHVGPAMQN IIKQLYLHPV PY |

A nucleic acid sequence for a cytosolic P. cablin patchoulol synthase (cytosol:PcPAS, AY508730; SEQ ID NO:44) is shown below.

| | |
|---|---|
| 1 | ATGGAGTTGT ATGCCCAAAG TGTTGGAGTG GGTGCTGCTT |
| 41 | CTCGTCCTCT TGCGAATTTT CATCCATGTG TGTGGGGAGA |
| 81 | CAAATTCATT GTCTACAACC CACAATCATG CCAGGCTGGA |
| 121 | GAGAGAGAAG AGGCTGAGGA GCTGAAAGTG GAGCTGAAAA |
| 161 | GAGAGCTGAA GGAAGCATCA GACAACTACA TGCGGCAACT |
| 201 | GAAAATGGTG GATGCAATAC AACGATTAGG CATTGACTAT |
| 241 | CTTTTTGTGG AAGATGTTGA TGAAGCTTTG AAGAATCTGT |
| 281 | TTGAAATGTT TGATGCTTTC TGCAAGAATA ATCATGACAT |
| 321 | GCACGCCACT GCTCTCAGCT TTCGCCTTCT CAGACAACAT |
| 361 | GGATACAGAG TTTCATGTGA AGTTTTTGAA AAGTTTAAGG |
| 401 | ATGGCAAAGA TGGATTTAAG GTTCCAAATG AGGATGGAGC |
| 441 | GGTTGCAGTC CTTGAATTCT TCGAAGCCAC GCATCTCAGA |
| 481 | GTCCATGGAG AAGACGTCCT TGATAATGCT TTTGACTTCA |
| 521 | CTAGGAACTA CTTGGAATCA GTCTATGCAA CTTTGAACGA |
| 561 | TCCAACCGCG AAACAAGTCC ACAACGCATT GAATGAGTTC |
| 601 | TCTTTTCGAA GAGGATTGCC ACGCGTGGAA GCAAGGAAGT |
| 641 | ACATATCAAT CTACGAGCAA TACGCATCTC ATCACAAAGG |
| 681 | CTTGCTCAAA CTTGCTAAGC TGGATTTCAA CTTGGTACAA |
| 721 | GCTTTGCACA GAAGGGAGCT GAGTGAAGAT TCTAGGTGGT |
| 761 | GGAAGACTTT ACAAGTGCCC ACAAAGCTAT CATTCGTTAG |
| 801 | AGATCGATTG GTGGAGTCCT ACTTCTGGGC TTCGGGATCT |
| 841 | TATTTCGAAC CGAATTATTC GGTAGCTAGG ATGATTTTAG |
| 881 | CAAAAGGGCT GGCTGTATTA TCTCTTATGG ATGATGTGTA |
| 921 | TGATGCATAT GGTACTTTTG AGGAATTACA AATGTTCACA |
| 961 | GATGCAATCG AAAGGTGGGA TGCTTCATGT TTAGATAAAC |
| 1001 | TTCCAGATTA CATGAAAATA GTATACAAGG CCCTTTTGGA |
| 1041 | TGTGTTTGAG GAAGTTGACG AGGAGTTGAT CAAGCTAGGC |
| 1081 | GCACCATATC GAGCCTACTA TGGAAAAGAA GCCATGAAAT |
| 1121 | ACGCCGCGAG AGCTTACATG GAAGAGGCCC AATGGAGGGA |
| 1161 | GCAAAAGCAC AAACCCACAA CCAAGGAGTA TATGAAGCTG |
| 1201 | GCAACCAAGA CATGTGGCTA CATAACTCTA ATAATATTAT |
| 1241 | CATGTCTTGG AGTGGAAGAG GGCATTGTGA CCAAAGAAGC |
| 1281 | CTTCGATTGG GTGTTCTCCC GACCTCCTTT CATCGAGGCT |
| 1321 | ACATTAATCA TTGCCAGGCT CGTCAATGAT ATTACAGGAC |
| 1361 | ACGAGTTTGA GAAAAAACGA GAGCACGTTC GCACTGCAGT |
| 1401 | AGAATGCTAC ATGGAAGAGC ACAAAGTGGG GAAGCAAGAG |
| 1441 | GTGGTGTCTG AATTCTACAA CCAAATGGAG TCAGCATGGA |
| 1481 | AGGACATTAA TGAGGGGTTC CTCAGACCAG TTGAATTTCC |
| 1521 | AATCCCTCTA CTTTATCTTA TTCTCAATTC AGTCCGAACA |
| 1561 | CTTGAGGTTA TTTACAAAGA GGGCGATTCG TATACACACG |
| 1601 | TGGGTCCTGC AATGCAAAAC ATCATCAAGC AGTTGTACCT |
| 1641 | TCACCCTGTT CCATATTAA |

The open reading frame encoding a truncated C. acuminata CPR (CaCPR70-708, KP162177) lacking the N-terminal membrane anchor domain was synthesized. Codon optimized open reading frames were synthesized for the type I GGDPSs from S. acidocaldarius (SaGGDPS, D28748.1) and M. thermautotrophicus (MtGGDPS, AE000666.1).

A putative M. elongata AG77 MeGGDPS (type III) was identified through mining of transcriptome data43 and a codon optimized open reading frame was synthesized (Supplemental Data). Two putative type II GGDPSs, EpGGDPS1 and EpGGDPS2, were identified through mining of E. peplus transcriptome data and amplified from leaf cDNA. A putative type II GGDPS was identified in the genome of Tolypothrix sp. PCC 7601 (TsGGDPS) and the coding sequence was amplified from genomic DNA. To target SaGGDPS, MtGGDPS, TsGGDPS, MeGGDPS, AtFDPS and PcPAS to the plastid, the sequences were fused at their N-terminus to the plastid targeting sequence of the Arabidopsis thaliana ribulose bisphosphate carboxylase small chain 1A (NM_105379.4). This Arabidopsis thaliana ribulose bisphosphate carboxylase small chain 1A protein is shown below as SEQ ID NO:49.

| | |
|---|---|
| 1 | MASSMLSSAT MVASPAQATM VAPFNGLKSS AAFPATRKAN |
| 41 | NDITSITSNG GRVNCMQVWP PIGKKKFETL SYLPDLTDSE |
| 81 | LAKEVDYLIR NKWIPCVEFE LEHGFVYREH GNSPGYYDGR |
| 121 | YWTMWKLPLF GCTDSAQVLK EVEECKKEYP NAFIRIIGFD |
| 161 | NTRQVQCISF IAYKPPSFTG |

A nucleotide sequence for the Arabidopsis thaliana ribulose bisphosphate, carboxylase small chain 1A (NM_105379.4) is shown below as SEQ ID NO:50.

| | |
|---|---|
| 1 | CCAAGGTAAA AAAAAGGTAT GAAAGCTCTA TAGTAAGTAA |
| 41 | AATATAAATT CCCCATAAGG AAAGGGCCAA GTCCACCAGG |
| 81 | CAAGTAAAAT GAGCAAGCAC CACTCCACCA TCACACAATT |
| 121 | TCACTCATAG ATAACGATAA GATTCATGGA ATTATCTTCC |
| 161 | ACGTGGCATT ATTCCAGCGG TTCAAGCCGA TAAGGGTCTC |
| 201 | AACACCTCTC CTTAGGCCTT TGTGGCCGTT ACCAAGTAAA |
| 241 | ATTAACCTCA CACATATCCA CACTCAAAAT CCAACGGTGT |
| 281 | AGATCCTAGT CCACTTGAAT CTCATGTATC CTAGACCCTC |
| 321 | CGATCACTCC AAAGCTTGTT CTCATTGTTG TTATCATTAT |
| 361 | ATATAGATGA CCAAAGCACT AGACCAAACC TCAGTCACAC |
| 401 | AAAGAGTAAA GAAGAACAAT GGCTTCCTCT ATGCTCTCTT |
| 441 | CCGCTACTAT GGTTGCCTCT CCGGCTCAGG CCACTATGGT |
| 481 | CGCTCCTTTC AACGGACTTA AGTCCTCCGC TGCCTTCCCA |
| 521 | GCCACCCGCA AGGCTAACAA CGACATTACT TCCATCACAA |
| 561 | GCAACGGCGG AAGAGTTAAC TGCATGCAGG TGTGGCCTCC |
| 601 | GATTGGAAAG AAGAAGTTTG AGACTCTCTC TTACCTTCCT |
| 641 | GACCTTACCG ATTCCGAATT GGCTAAGGAA GTTGACTACC |
| 681 | TTATCCGCAA CAAGTGGATT CCTTGTGTTG AATTCGAGTT |
| 721 | GGAGCACGGA TTTGTGTACC GTGAGCACGG TAACTCACCC |
| 761 | GGATACTATG ATGGACGGTA CTGGACAATG TGGAAGCTTC |
| 801 | CCTTGTTCGG TTGCACCGAC TCCGCTCAAG TGTTGAAGGA |
| 841 | AGTGGAAGAG TGCAAGAAGG AGTACCCCAA TGCCTTCATT |
| 881 | AGGATCATCG GATTCGACAA CACCCGTCAA GTCCAGTGCA |
| 921 | TCAGTTTCAT TGCCTACAAG CCACCAAGCT TCACCGGTTA |
| 961 | ATTTCCCTTT GCTTTTCTGT AAACCTCAAA ACTTTATCCC |
| 1001 | CCATCTTTGA TTTTATCCCT TGTTTTTCTG CTTTTTTCTT |
| 1041 | CTTTCTTGGG TTTTAATTTC CGGACTTAAC GTTTGTTTTC |
| 1081 | CGCTTTGCGA CACATATTCT ATCCGATTCT CAACTCTCTG |
| 1121 | ATGAAATAAA TATGTAATGT TCTATAAGTC TTTCAATTTG |
| 1161 | ATATGCATAT CAACAAAAAG AAAATAGGAC AATGCGGCTA |
| 1201 | CAAATATGAA ATTTACAAGT TTAAGAACCA TGAGTCGCTA |
| 1241 | AAGAAATCAT TAAGAAAATT AGTTTCAC |

In some cases, a portion of the Arabidopsis thaliana ribulose bisphosphate carboxylase small chain 1A protein was used as a chloroplast transit peptide to re-localize cytosolic proteins to the chloroplast. Such an Arabidopsis thaliana ribulose bisphosphate carboxylase small chain 1A peptide can have SEQ ID NO:101 (shown below).

| | |
|---|---|
| 1 | MASSMLSSAT MVASPAQATM VAPFNGLKSS AAFPATRKAN |
| 41 | NDITSITSNG GRVN |

A nucleic acid segment that encodes the Arabidopsis thaliana ribulose bisphosphate carboxylase small chain 1A peptide with SEQ ID NO:101 is shown below as SEQ ID NO:102.

| | |
|---|---|
| 1 | ATGGCTTCCT CTATGCTCTC TTCCGCTACT ATGGTTGCCT |
| 41 | CTCCGGCTCA GGCCACTATG GTCGCTCCTT TCAACGGACT |
| 81 | TAAGTCCTCC GCTGCCTTCC CAGCCACCCG CAAGGCTAAC |
| 121 | AACGACATTA CTTCCATCAC AAGCAACGGC GGAAGAGTTA |
| 161 | AC |

Examples of plastid-targeted proteins are referred to as plastid:SaGGDPS, plastid:MtGGDPS, plastid:TsGGDPS plastid:MeGGDPS, plastid:AtFDPS and plastid:PcPAS.

The coding sequences of A. grandis abietadiene synthase (SEQ ID NO:31) and P. sitchensis CYP720B4 (ER:PcCYP720B4; SEQ ID NO:35) were truncated to target the enzymes to the cytosol, in this study referred to as cytosol:AgABS(85-868) (SEQ ID NO:33) and cytosol:PsCYP720B4(30-483) (SEQ ID NO:37), respectively.

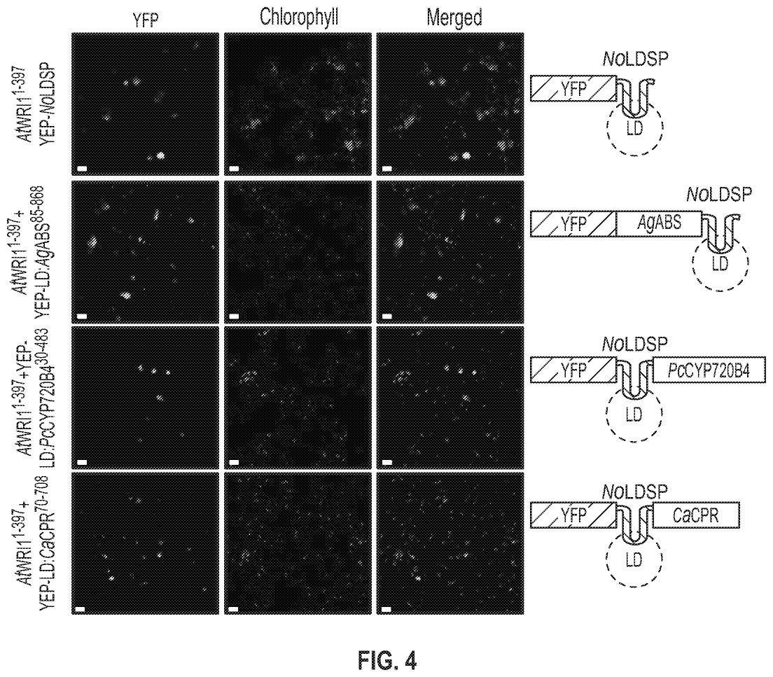

For lipid droplet targeting, truncated A. grandis abietadiene synthase, P. sitchensis CYP720B4 and C. acuminata CPR were either fused to the N-terminus or C-terminus of N. oceanica lipid droplet surface protein resulting in LD:AgABS85-868, LD:PsCYP720B4(30-483) and LD:CaCPR(70-708), respectively (FIG. 4). The full-length and modified coding sequences were verified by sequencing, inserted into pENTR4 (Invitrogen), and subsequently transferred into the Gateway vectors pEarleygate 100 and pEarleygate 104 (N-terminal YFP-tag), each under control of a 35S promoter for strong constitutive expression (Earley et al. Plant J. 45, 616-629 (2006)). These constructs were introduced into A. tumefaciens LBA4404 for transient expression studies in Nicotiana benthamiana.

Agrobacterium-Mediated Transient Expression in N. benthamiana Leaves

Transformants of A. tumefaciens LBA4404 carrying selected binary vectors were grown overnight at 28° C. in Luria-Bertani medium containing 50 μg/mL rifampicin and 50 μg/mL kanamycin. Prior to infiltration into N. benthamiana leaves, the A. tumefaciens cells were sedimented by centrifugation at 3800×g for 10 min, washed, resuspended in infiltration buffer (10 mM MES-KOH pH 5.7, 10 mM MgCl₂, 200 μM acetosyringone) to an optical density at 600 nm (OD₆₀₀) 0.8 and incubated for approximately 30 min at 30° C. To test various gene combinations, equal volumes of the selected bacterial suspensions were mixed and infiltrated into N. benthamiana leaves using a syringe without a needle. A. tumefaciens LBA4404 carrying the tomato bushy stunt virus gene P19 (Voinnet et al. Proc. Natl. Acad. Sci. 96, 14147-14152 (1999)); Voinnet et al. Proc. Natl. Acad. Sci. 112, E4812 (2015)) was included in all infiltrations to suppress RNA silencing in N. benthamiana. The N. benthamiana plants were grown for 3.5 to 4 weeks in soil at 25° C. under a 12-hour photoperiod at 150 μmol m⁻² s⁻¹. After infiltration, the plants were grown for 4 additional days in the growth chamber. Samples from the infiltrated leaves were subsequently analyzed for terpenoid or triacylglycerol content.

### Lipid Analysis

Triacylglycerol analyses were performed essentially as described by Yang et al. (Plant Physiol. 169, 1836-1847 (2015)) with minor modifications. For each sample, one N. benthamiana leaf was freshly harvested and total lipids were extracted with 4 mL chloroform/methanol/formic acid (10:20:1, by volume). Ten micrograms tri-17:0 TAG (Sigma) was added as internal standard to each sample.

### Statistical Analyses

Statistical analyses were conducted using two-tailed unpaired Student's t-tests. A P-value of <0.05 was considered statistically significant.

Terpenoid Analyses in N. benthamiana Leaves

For each sample, one leaf disc (˜100 mg fresh weight) was incubated with 1 mL hexane containing 2 mg/mL1-eicosene (internal standard, TCI America) on a shaker for 15 min at room temperature prior to incubation in the dark for 16 hours at room temperature. The reaction products were separated and analyzed by GC-MS using an Agilent 7890A GC system coupled to an Agilent 5975C MS detector. Chromatography was performed with an Agilent VF-5 ms column (40 m×0.25 mm×0.25 μm) at 1.2 mL/min helium flow. The injection volume was 1 μL in splitless mode at an injector temperature of 250° C. The following oven program was used (run time 18.74 min): 1 min isothermal at 40° C., 40° C. per minute to 180° C., 2 min isothermal at 180° C., 15° C. per minute to 300° C., 1 min isothermal at 300° C., 100° C. per minute to 325° C. and 3 minutes isothermal at 325° C. The mass spectrometer was operated at 70 eV electron ionization mode, a solvent delay of 3 minutes, ion source temperature at 230° C., and quadrupole temperature at 150° C. Mass spectra were recorded from m/z 30 to 600. Terpenoid products were identified based on retention times, mass spectra published in relevant literature and through comparison with the NIST Mass Spectral Library v17 (National Institute of Standards and Technology, USA). Quantitation of diterpenoid products as well as patchoulol was based on 1-eicosene standard curves. The extracted ion chromatograms for each target compound were integrated, and compounds were quantified using QuanLynx tool (Waters) with a mass window allowance of 0.2 and a signal-to-noise ratio greater than or equal to 10. All calculated peak areas were normalized to the peak area for the internal standard 1-eicosene and tissue fresh weight.

Diterpenoid resin acids and glycosylated derivatives were analyzed by UHPLC/MS/MS to confirm accurate masses and fragments. For each sample, one leaf disc (˜100 mg fresh weight) was incubated with 1 mL methanol containing 1.25 μM telmisartan (internal standard, Toronto Research Chemicals) in the dark for 16 h at room temperature. A 10-μL volume of each extract was subsequently analyzed using a 31-min gradient elution method on an Acquity BEH C18 UHPLC column (2.1×100 mm, 1.7 μm, Waters) with mobile phases consisting of 0.15% formic acid in water (solvent A) and acetonitrile (solvent B). The method involved a 31-minute gradient employing 1% B at 0.00 to 1 min, linear gradient to 99% B at 28.00 min, with a hold until 30 min, followed by a return to 1% B and a hold from 30.10 to 31 minutes. The flow rate was 0.3 mL/min and the column temperature was 40° C. The mass spectrometer (Xevo G2-XS QTOF, Waters) was equipped with an electrospray ionization source and operated in negative-ion mode. Source parameters were as follows: capillary voltage 2500 V, cone voltage 40 V, desolvation temperature 300° C., source temperature 100° C., cone gas flow 50 L/h, and desolvation gas flow 600 L/h. Mass spectrum acquisition was performed in negative ion mode over m/z 50 to 1500 with scan time of 0.2 seconds using a collision energy ramp 20 to 80 V.

### Isolation of Lipid Droplets

Lipid droplets were isolated as previously described with minor adjustments (Ding, Y. et al. Nat. Protoc. 8: 43 (2012)). For each sample, 1 g infiltrated N. benthamiana leaf tissue was ground with mortar and pestle in 20 mL ice-cold buffer A (20 mM tricine, 250 mM sucrose, 0.2 mM phenylmethylsulfonyl fluoride pH 7.8). The homogenate was filtered through Miracloth (Calbiochem) and centrifuged in a 50-mL tube at 3,400 g for 10 min at 4° C. to remove cell debris. From each tube, 10 mL supernatant was collected and transferred to a 15-mL tube. The supernatant fraction was then overlaid with 3 mL buffer B (20 mM HEPES, 100 mM KCl, 2 mM MgCl₂, pH 7.4) and centrifuged for 1 hour at 5,000 g. After centrifugation, 2 mL from the top of each gradient containing floating lipid droplets were collected. For terpenoid analysis, each lipid droplet fraction was extracted with 1 mL hexane containing 2 μg/mL 1-eicosene (internal standard, TCI America) prior to GC-MS analysis.

### Confocal Imaging

For lipid droplet visualization, freshly harvested leaf samples were stained with Nile red as described by Sanjaya et al. (Plant Biotechnol. J. 9, 874-883 (2011)). Imaging of Nile red, chlorophyll and enhanced yellow fluorescent protein (EYFP) fluorescence was conducted with a confocal laser scanning microscope FluoView VF1000 (Olympus) at excitation 559 nm/emission 570-630 nm, excitation 559 nm/emission 655-755 nm and excitation 515 nm/emission 527 nm, respectively. Images were processed using the FV10-ASW 3.0 microscopy software (Olympus).

### EXAMPLE 2

### Expression of a Microalgal Lipid Droplet Surface Protein Increases WRINKLED1-Initiated Triacylglycerol Accumulation

To assess the impact of NoLDSP on AtWRI1(1-397)-initiated triacylglycerol accumulation, leaves of N. benthamiana were infiltrated with Agrobacterium tumefaciens suspensions for transient production of AtWRI1(1-397) alone or in combination with a lipid droplet surface protein (NoLDSP) encoding cDNA from the microalga Nannochloropsis oceanica (AtWRI1(1-397)+NoLDSP). NoLDSP possesses a hydrophobic central region that likely mediates the anchoring on lipid droplets.

In leaves producing AtWRI1(1-397) or AtWRI1(1-397) with NoLDSP, the triacylglycerol level was at least 3-fold higher and about 12-fold higher, respectively, than in control leaves without AtWRI11-397 (FIG. 1A).

These results clearly demonstrated the beneficial impact of the microalgal NoLDSP on lipid droplet accumulation. NoLDSP had no negative impact on triacylglycerol production and enhanced the accumulation of lipid droplets in infiltrated N. benthamiana leaves.

### EXAMPLE 3

### Engineered Sesquiterpenoid Production in the Cytosol and Plastids

Different engineering strategies were then tested for the production of sesquiterpenoids using patchoulol as a model compound. Like many other sesquiterpenoids, patchoulol is volatile. Previous work has shown that engineered production of patchoulol in transgenic lines of N. tabacum resulted in significant losses from volatile emission (Wu et al. Nat. Biotechnol. 24: 1441-1447 (2006)). In the experiments described here, losses of atmospheric terpenoid emission were not recorded because the engineering strategies were designed to sequester target terpenoids in lipid droplets in the plant biomass.

Transient production of cytosolic Pogostemon cablin patchoulol synthase (cytosol:PcPAS) led to formation of a single low-level product, patchoulol, which was not detected in wild-type control plants (FIG. 1B).

To enhance the precursor availability for sesquiterpenoid synthesis, feedback-insensitive forms of Euphorbia lathyris HMGR (ElHMGR(159-582)) and A. thaliana FDPS (cytosol:AtFDPS) were included in the transient assays. Some reports indicate that E. lathyris accumulates high levels of triterpenoids and their esters (Skrukrud et al. in The Metabolism, Structure, and Function of Plant Lipids (eds. Paul K. Stumpf, J. Brian Mudd, & W. David Nes) 115-118 (Springer New York, 1987)), suggesting that its HMGR could be a robust enzyme for sesquiterpenoid production in N. benthamiana. The selection of the A. thaliana FDPS was based on its relatively high thermal stability (Keim et al. PloS One 7, e49109 (2012)).

The patchoulol content in N. benthamiana leaves producing ElHMGR(159-582) with cytosol:AtFDPS and cytosol:PcPAS was at least 5-fold higher than in leaves with cytosol:PcPAS alone, which is consistent with enhanced precursor flux. However, co-engineering of patchoulol and triacylglycerol synthesis impaired cytosolic terpenoid accumulation, independent of whether precursor availability was increased or not (FIG. 1B).

A previous study demonstrated that re-direction of PcPAS and avian FDPS to the plastid increased the retained patchoulol levels in leaves of stable transgenic N. tabacum lines up to approximately 30 μg patchoulol per gram fresh weight (Wu et al. Nat. Biotechnol. 24, 1441-1447 (2006)). This approach was modified to further examine engineering strategies for the co-production of patchoulol and lipid droplets in N. benthamiana leaves.

Targeting of patchoulol synthase to plastids (plastid:PcPAS) led to accumulation of approximately 0.5 μg patchoulol per gram fresh weight (FIG. 1C). To increase the precursor flux in the plastids, P. barbatus DXS (PbDXS) and plastid-targeted AtFDPS (plastid:AtFDPS) were combined with plastid:PcPAS in the assays. This strategy resulted in a 60-fold increase in the level of patchoulol (FIG. 1C), Synthetic lipid droplet accumulation impaired patchoulol production in leaves in the absence of PbDXS and plastid:AtFDPS, when precursor synthesis was not co-engineered (FIG. 1C). The negative impact on patchoulol synthesis was rescued when plastid:AtFDPS or PbDXS with plastid:AtFDPS were included in the assay.

Leaves transiently producing PbDXS with plastid:AtFDPS, plastid:PcPAS, AtWRI1(1-397), and NoLDSP yielded the highest patchoulol level retained in leaves, up to about 45 ug patchoulol per gram fresh weight, an average 90-fold and 1.5-fold higher compared to leaves producing plastid:PcPAS and PbDXS with plastid:AtFDPS, and plastid:PcPAS, respectively.

### EXAMPLE 4

### Diterpenoid Scaffold Production in Plastids and Cytosol

Strategies for diterpenoid production in the N. benthamiana system were examined using the Abies grandis abietadiene synthase (AgABS) as diterpene synthase. This bifunctional enzyme has class II and class I terpene synthase activity and catalyzes both the bicyclization of GGDP to a (+)-copalyl diphosphate intermediate and the subsequent secondary cyclization and further rearrangement.

Transient production of the native plastidial A. grandis abietadiene synthase (plastid:AgABS) resulted in the accumulation of abietadiene (abieta-7,13-diene), levopimaradiene (abieta-8(14),12-diene), neoabietadiene (abieta-8(14),13(15)-diene) and, as minor product, palustradiene (abieta-8,13-diene). These diterpenoids were not detected in wild-type control leaves of N. benthamiana.

Sole production of plastid:AgABS yielded about 40 μg diterpenoids per gram fresh weight (FIG. 2A). To enhance the production of diterpenoids, plastid:AgABS was co-produced in different combinations with PbDXS and a plastid GGDPS.

GGDPSs are differentiated into three types (type I-III) according to their amino acid sequences around the first aspartate-rich motif. These three types differ in their mechanism of determining product chain-length (Noike et al. J. Biosci. Bioeng. 107, 235-239 (2009); Chang et al. J. Biol. Chem. 281, 14991-15000 (2006)). Plant GGDPSs are type II enzymes that are regulated on gene expression, transcript and protein level (Xu et al. BMC Genomics 11, 246-246 (2010); Thou et al. Proc. Natl. Acad. Sci. 114, 6866-6871 (2017); Ruiz-Sola et al. New Phytol. 209, 252-264 (2016)).

The inventors hypothesized that inclusion of distantly related type I and type III GGDPSs or a cyanobacterial type II GGDPS may bypass potential regulatory steps that can limit diterpenoid production in N. benthamiana. Six GGDPSs were selected based on GenBank and BLAST searches as well as analysis of transcriptome data, a GGDPS from the archaea Sulfolobus acidocaldarius (SaGGDPS, type I) and five predicted GGDPSs from the archaea Methanothermobacter thermautotrophicus (MtGGDPS, type I), the cyanobacterium Tolypothrix sp. PCC 7601 (TsGGDPS, type II), the plant Euphorbia peplus (EpGGDPS1 and EpGGDPS2, type II), and the fungus Mortierella elongata AG77 (MeGGDPS, type III). The sequences of SaGGDPS, MtGGDPS, and MeGGDPS enzymes share only 24%, 25% and 17% amino acid identities with EpGGDPS1, respectively, whereas TsGGDPS and EpGGDPS2 share 48% and 58% identities with EpGGDPS1, respectively.

For transient assays in N. benthamiana, the coding sequences for the bacterial and fungal GGDPSs were codon-optimized (except for TsGGDPS) and modified to target the enzymes to the plastids, referred to as plastid:SaGGDPS, plastid:MtGGDPS, plastid:TsGGDPS, and plastid:MeGGDPS. Co-production of PbDXS with plastid:AgABS or plastid:GGDPS with plastid:AgABS was insufficient to increase the diterpenoid content in N. benthamiana leaves more than 2-fold compared to the diterpenoid level in plastid:AgABS-producing leaves (FIG. 2A).

In contrast, co-production of PbDXS with GGDPS and plastid:AgABS enhanced diterpenoid production to up to 6.5-fold compared to leaves producing plastid:AgABS). Significant differences in diterpenoid yields were obtained depending on which GGDPS was included, apparently unrelated to a specific type of GGDPS (FIG. 2A). The highest diterpenoid levels were in N. benthamiana leaves co-producing PbDXS with plastid:AgABS, plastid:MtGGDPS (type I), plastid:TsGGDPS (type II), or EpGGDPS2 (type II), with similar yield between these combinations (FIG. 2A).

Diterpenoid accumulation was further evaluated in the presence of lipid droplets. Co-production of plastid:AgABS with AtWRI1 (1-397) had no significant impact on the diterpenoid level compared to control leaves producing plastid:AgABS alone. However, in leaves producing plastid:AgABS with AtWRI1-397 and NoLDSP, the diterpenoid content was increased 2-fold (FIG. 2B). Similarly, co-production of plastid:MtGGDPS with plastid:AgABS, AtWRI1(1-397) and NoLDSP increased the diterpenoid level 2.5-fold compared to plastid:MtGGDPS with plastid:AgABS-producing leaves.

These results indicated that the increased abundance of lipid droplets was beneficial for, and contributed to, the accumulation of diterpenoid products. Sequestration of the lipophilic diterpenoids into lipid droplets may have helped to circumvent negative feedback regulatory mechanisms and served as “pull force” in diterpenoid production.

In fact, isolated lipid droplet fractions from leaves producing plastid:AgABS with AtWRI1(1-397) and plastid:AgABS with AtWRI1(1-397) and NoLDSP contained at least 35-fold and 420-fold more diterpenoids, respectively, than control fractions from leaves with plastid:AgABS, consistent with the sequestration of diterpenoids in lipid droplets (FIG. 2D-2E). NoLDSP promotes clustering of small lipid droplets (FIG. 2F). The localization of yellow fluorescent fusion protein-tagged NoLDSP (YFP-NoLDSP) in clustered lipid droplets was observed by confocal laser scanning microscopy on a collected lipid droplet fraction.

Co-production of PbDXS and plastid:MtGGDPS together with plastid:AgABS yielded the highest diterpenoid level (FIG. 2B), independent of whether AtWRI1(1-397) was included for lipid droplet synthesis. in the transient assays yielded the highest diterpenoid level independent of whether lipid droplets were co-engineered (FIG. 2B). In contrast, co-production of PbDXS with plastid:MtGGDPS and plastid:AgABS together with AtWRI1(1-397) and NoLDSP resulted in a significant reduction of the diterpenoid level (compared to leaves producing PbDXS with plastid:MtGGDPS and plastid:AgABS).

When A. grandis abietadiene synthase was targeted to the cytosol (cytosol:AgABS(85-868)), leaves accumulated approximately 0.2 μg diterpenoids per gram fresh weight and addition of precursor pathway genes enhanced diterpenoid synthesis (FIG. 2C). Co-production of cytosol:AgABS(85-868) together with ElHMGR(159-582) and cytosolic M. thermautotrophicus GGDPS (cytosol:MtGGDPS) increased the diterpenoid yield more than 400-fold (relative to cytosol:AgABS(85-868) containing leaves) and, thus, close to the highest diterpenoid yield achieved with plastid engineering approaches (FIGS. 2B-2C).

Moreover, these data indicated that lipid droplets exhibited an enhancing effect of accumulation on terpenoid production when cytosol:AgABS(85-868) was co-produced with AtWRI1(1-397) or AtWRI1(1-397) with NoLDSP (FIG. 2C). Under these conditions, terpenoid production was increased up to approximately 3-fold which is consistent with diterpenoids being sequestered in lipid droplets.

When ElHMGR(159-582) with cytosol:MtGGDPS, cytosol:AgABS(85-868), AtWRI1(1-397) and NoLDSP were co-produced, no additive effects of lipid droplet engineering on terpenoid yield were detected (relative to ElHMGR(159-582) with cytosol:MtGGDPS and cytosol:AgABS85-868) (FIG. 2C).

### EXAMPLE 5

### Triacylglycerol Analysis of N. benthamiana Leaves Engineered for Terpenoid and Lipid Droplet Production

To examine a potential impact of terpenoid engineering on triacylglycerol yield, the established approaches for low-yield or high-yield terpenoid synthesis combined with lipid droplet production were further tested.

Four days after A. tumefaciens infiltration into N. benthamiana to engineer the N. benthamiana to express various enzyme expression systems, N. benthamiana leaves were subjected to triacylglycerol analysis. Leaves co-engineered for lipid droplet and high-yield patchoulol production in the cytosol contained approximately 50% less triacylglycerol than leaves producing just AtWRI1(1-397) with NoLDSP (FIG. 3A). A significant decrease in the triacylglycerol level was also detected when leaves were engineered for cytosol-targeted high-yield production of diterpenoids (compared to leaves producing AtWRI11-397 with NoLDSP) (FIG. 3B). When lipid droplet production was combined with a plastid-targeted approach for high-yield terpenoid synthesis, no negative impact on triacylglycerol accumulation was observed compared to control plants (FIG. 3A-3B).

In the cytosol, low-yield terpenoid production of diterpenoid had no impact on TAG yield; low-yield of sesquiterpenoid also had little or no significant impact on triacylglycerol yield. High-yield production of sesquiterpenoids and diterpenoids in the cytosol led to approximately 50% less triacylglycerol.

Under certain conditions, terpenoid production may compete with triacylglycerol biosynthesis for carbon from the plastid. The different triacylglycerol yields in cytosolic approaches (low yield vs. high yield) suggest regulatory mechanisms may exist to control the partitioning of carbon between plastid and cytosol. As both FDP and GGDP serve as prenyl donors for protein prenylation in the cytosol, protein prenylation may be involved in these regulatory networks. Alterations in the cytosolic levels of FDP and GGDP may have indirectly contributed to the decrease in triacylglycerol yields.

### EXAMPLE 6

### Targeting Diterpenoid and Diterpenoid Acid Production to Lipid Droplets

This Example describes experiments designed to determine whether lipid droplets in the cytosol can be used as platform to anchor biosynthetic pathways for the production of functionalized diterpenoids. The proof-of-concept experiments included use of Picea sitchensis cytochrome P450 PsCYP720B4 (ER:PsCYP720B4) that can convert abietadiene and several isomers to the corresponding diterpene resin acids as well as a modified A. grandis abietadiene synthase.

To target terpenoid synthesis to lipid droplets, A. grandis abietadiene synthase lacking the N-terminal plastid targeting sequence (cytosol:AgABS(85-868)) and truncated PsCYP720B4 lacking the N-terminal membrane-binding domain (cytosol:PsCYP720B4(30-483)) were produced as C-terminal and N-terminal NoLDSP-fusion proteins, respectively. The NoLDSP-fusion proteins are herein referred to as LD:AgABS(85-868) and LD:PsCYP720B4(30-483).

Inclusion of cytochrome P450 reductases (CPRs) can help drive metabolic fluxes in cytochrome P450 (CYP)-mediated production of high-value target compounds in non-native hosts and synthetic compartments. Camptotheca acuminata CPR (cytosol:CaCPR(70-708)) was included the experiments as NoLDSP-fusion protein to co-localize the CaCPR and PsCYP720B4 activities on lipid droplets and facilitate the CYP-catalyzed production of functionalized terpenoids. As the C-terminus of CPRs is pivotal for catalytic activity and not suitable for modifications, the predicted N-terminal hydrophobic domain of native CaCPR was replaced by NoLDSP to produce the fusion protein LD:CaCPR(70-708).

To determine the localization in planta, the NoLDSP-fusion proteins were each produced as yellow fluorescent protein (YFP)-tagged proteins together with AtWRI1(1-397) for lipid droplet production. The YFP-signals in infiltrated leaves were subsequently compared to the signals obtained for YFP-tagged NoLDSP, which indicated that all three YFP-tagged NoLDSP-fusion proteins were targeted to the surface of the lipid droplets (FIG. 4). It is noteworthy that production of the YFP-tagged NoLDSP and NoLDSP-fusion proteins promoted clustering of small lipid droplets in planta and in isolated lipid droplet fractions (FIG. 4, FIG. 2D-2F). As confirmed for NoLDSP, the clustering of small lipid droplets was independent of the presence or absence of the YFP-tag (FIG. 2F).

To compare different engineering approaches, the A. grandis abietadiene synthase was produced as plastid:AgABS (native), cytosol:AgABS(85-868), or LD:AgABS85-868, each alone or combined with ER:PsCYP720B4 (native), cytosol:PsCYP720B4(30-483), or LD:PsCYP720B4(30-483), with LD:CaCPR(70-708) (FIG. 5). Note that these assays also included either PbDXS with plastid:MtGGDPS, or ElHMGR(159-582) with cytosol:MtGGDPS to increase the precursor flux, and AtWRI1(1-397) to initiate lipid droplet accumulation. NoLDSP was included in those assays that lacked any NoLDSP-fusion proteins. NoLDSP was included in those assays that lacked any NoLDSP-fusion proteins.

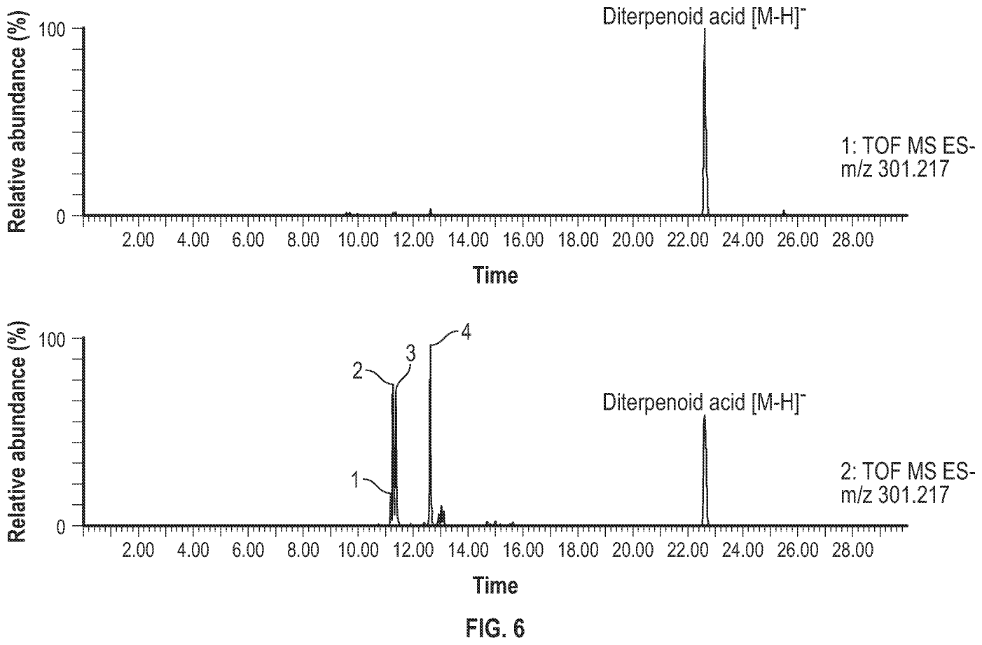

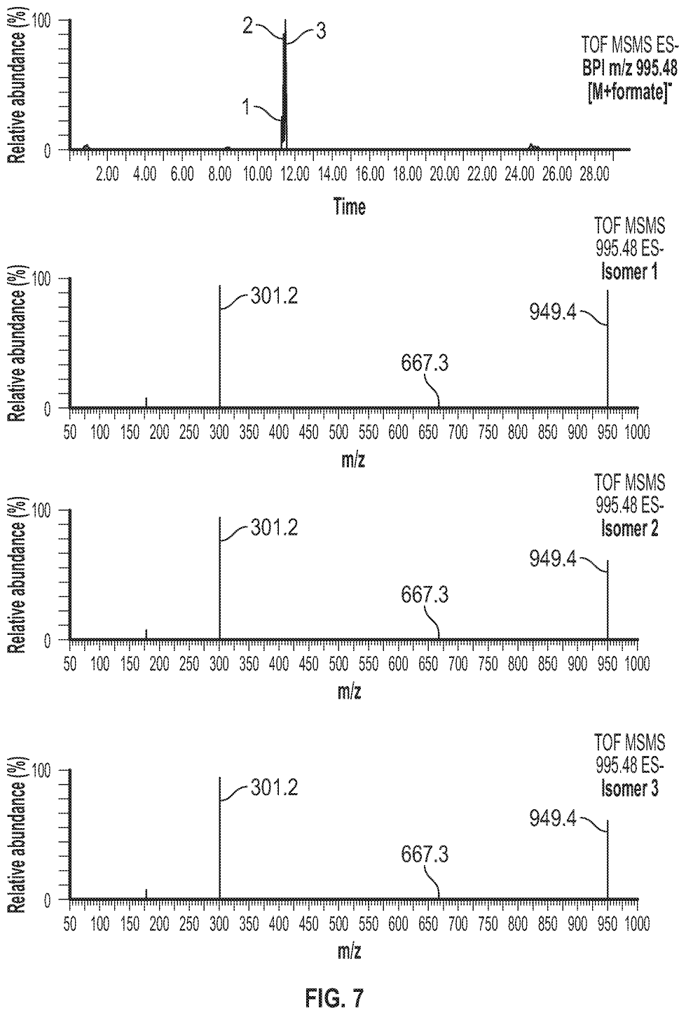

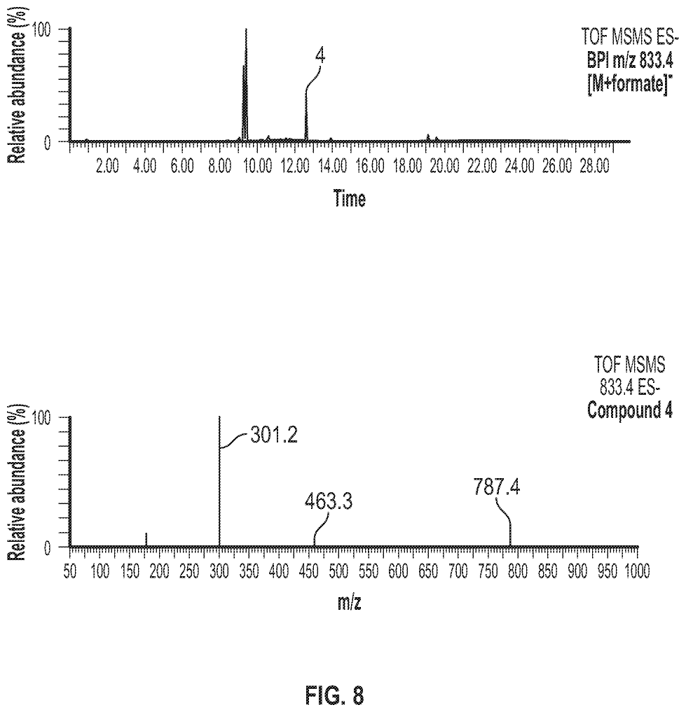

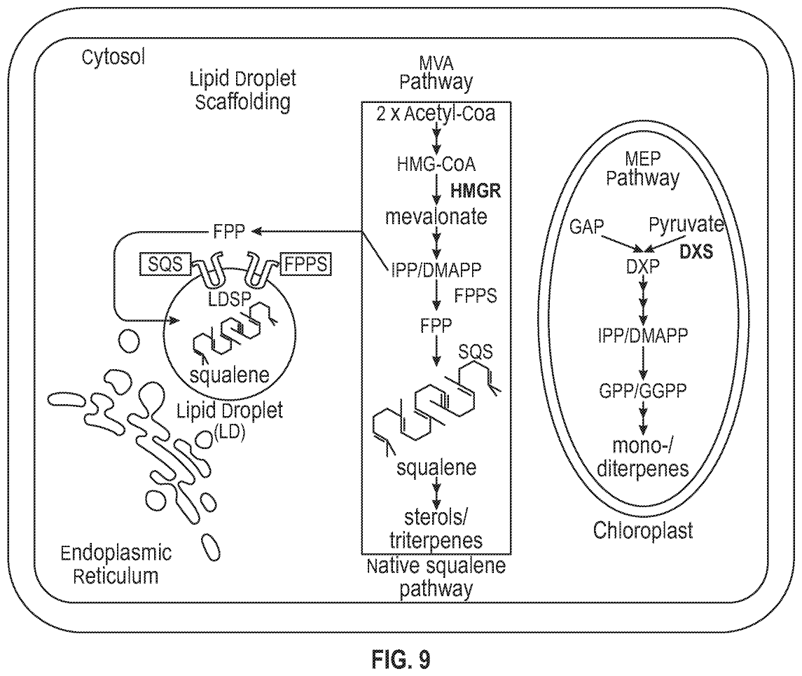

Compared to the assays with plastid:AgABS, use of cytosol:AgABS(85-868) and LD:AgABS(85-868) resulted in similar diterpenoid yield. When native or modified A. grandis abietadiene synthase was co-produced with native or modified P. sitchensis PsCYP720B4, the leaves accumulated diterpene resin acids in free and glycosylated forms (FIGS. 6-8).

The glycosyl modifications of the diterpenoid acids are likely the result of intrinsic defense/detoxification mechanisms in N. benthamiana. Incubation of leaf extracts with Viscozyme® L resulted in the hydrolysis of the glycosylated diterpenoid acids to free diterpenoid resin acids which allowed determination of the level of total diterpenoid acids produced in infiltrated leaves.

To facilitate the comparison between the different engineering strategies, the level of diterpenoids and total diterpenoid acids were quantified for each infiltrated leaf (FIG. 5). Co-production of plastid:AgABS with ER:PsCYP720B4, cytosol:PsCYP720B4(30-483) or LD:PsCYP720B4(30-483) decreased the diterpenoid level (compared to controls with plastid:AgABS) and resulted in the accumulation of diterpenoid acids, consistent with diterpenoids being converted to diterpenoid acids. The level of diterpenoid acids was about 4-fold and 3-fold higher in transient assays with plastid:AgABS including ER:PsCYP720B4 and plastid:AgABS, LD:PsCYP720B4(30-483), LD:CaCPR(70-708) compared to assays including cytosol:PsCYP720B4(30-483). The highest diterpenoid acid yield in transient assays with cytosolAgABS(85-868) was achieved in combination with ER:PsCYP720B4 which was at least 2-fold or at least 3-fold higher than with cytosol:AgABS(85-868) and LD:PsCYP720B4(30-483) with LD:CaCPR(70-708), respectively (FIG. 5). In transient assays with LD:AgABS(85-868), the diterpenoid acid level was 2-fold higher in assays with ER:PsCYP720B4 than in assays with either cytosol:PsCYP720B4(30-483) or LD:PsCYP720B4(30-483) with LD:CaCPR(70-708) (FIG. 5).

### EXAMPLE 7

### Screening DXS Variants

1-Deoxy-D-xylulose 5-phosphate synthase (DXS) is the entry step to the plastidial 2-C-methyl-D-erythritol 4-phosphate (MEP) pathway. DXS variants were screened to increase availability of IPP/DMAPP for terpene biosynthesis.

Candidate DXS and DXS alternatives were agrobacterium-transformed into Nicotiana benthamiana for transient expression of a Coleus forskohlii GGPPS (CfGGPPS) and a casbene synthase (CasS) recently discovered by the inventors (unpublished). Casbene was used as a proxy of DXS activities to evaluate DXS candidates for improving flux through the MEP pathway.

Three DXS enzymes were screened; Coleus forskohlii DXS (CfDXS), Populus trichocarpa DXS (PtDXS), and PtDXS with two-point mutations (PtDXS A147G:A352G) to reduce feedback inhibition by IPP/DMAPP. Additionally, two genes from E. coli (ribB and yajO) were also screened, as they provide a route to DXP, the first compound in the MEP pathway, via different substrates. These enzymes were also screened as fusions to DXP reductase (DXR), the next step in the MEP pathway.

Ratios of the product, casbene, were measured by GC-FID, compared to the internal standard ledol (IS), to determine the relative yields of casbene.

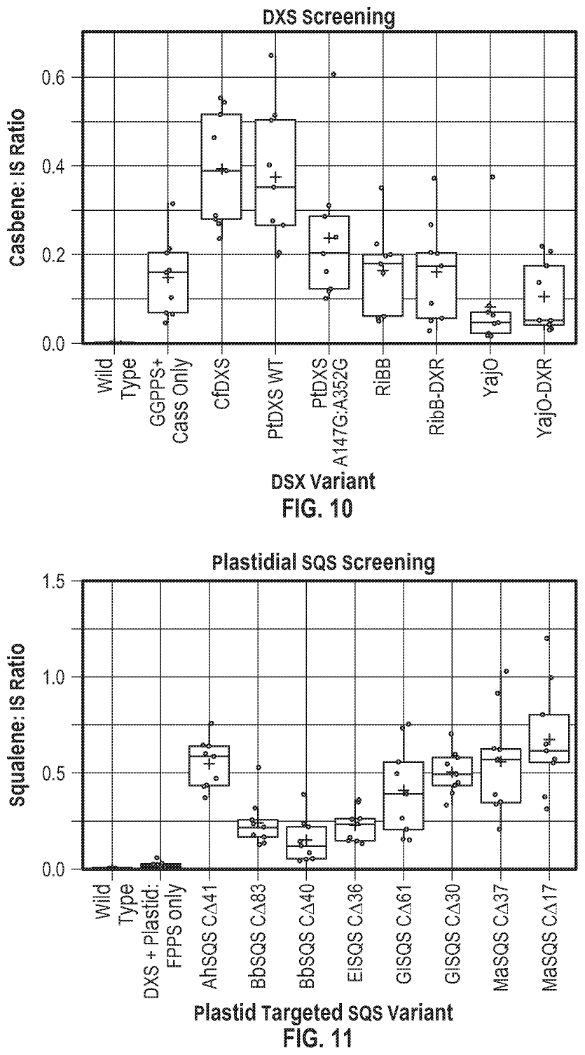

As shown in FIG. 10, the most casbene was produced by the Coleus forskohli DXS and the Populus trichocarpa DXS (PtDXS).

### EXAMPLE 8

### Screening Squalene Synthase (SQS) Candidates

Squalene synthase (SQS) candidates were screened to identify highly enzymes. Candidates that can increase squalene yields can be integrated into the lipid droplet scaffolding platform.

The squalene synthases evaluated included squalene synthases from Amaranthus hybridus, Botryococcus braunii, Euphorbia lathyrism, Ganoderma lucidum, and Mortierella alpine. All SQS candidates were natively ER bound but were modified to target them to plastids to reduce interference from the native, cytosolic N. benthamiana SQS. The following SQS candidates with truncations to remove endoplasmic reticulum (ER) targeting peptide were evaluated: Amaranthus hybridus SQS with a 41-amino acid, C-terminal truncation (AhSQS CΔ41), Botryococcus braunii SQS with an 83-amino acid, C-terminal truncation (BbSQS CΔ83), Botryococcus braunii SQS with an 40-amino acid, C-terminal truncation (BbSQS CΔ40), Euphorbia lathyris SQS with an 36-amino acid, C-terminal truncation (EISQS CΔ36), Ganoderma lucidum SQS with an 61-amino acid, C-terminal truncation (GlSQS CΔ61), Ganodenna lucidum SQS with a 30-amino acid, C-terminal truncation (GlSQS CΔ30), and Mortierella alpina SQS with a 37-amino acid, C-terminal truncation (MaSQS CΔ37), and Mortierella alpina SQS with a 17-amino acid, C-terminal truncation (MaSQS CΔ17).

Candidates were co-expressed with CfDXS and plastidial targeted Arabidopsis thaliana farnesyl diphosphate synthase (AtFPPS) to provide the squalene precursor, farnesyl diphosphate (FPP).

FIG. 11 shows the squalene yields as determined by GC-FID, where the relative yields are reported as the ratio of squalene to the internal standard, n-hexacosane. As shown, a Mortierella alpina squalene synthase with 17 amino acids truncated from the C-terminus had the highest squalene synthase activity. Such a truncated Mortierella alpina squalene synthase can have the following sequence (SEQ ID NO:68) (also called MaSQS CΔ17).

| | |
|---|---|
| 1 | MASAILASLL HPSEVLALVQ YKLSPKTQHD YSNDKTRQRL |
| 41 | YHHLNMTSRS FSAVIQDLDE ELKDAICLFY LVLRGLDTIE |
| 81 | DDMTIDLDTK LPYLRTFHEI IYQKGWTFTK NGPNEKDRQL |
| 121 | LVEFDAIIEG FLQLKPAYQT IIADITKRMG NGMAHYATAG |
| 161 | IHVETNADYD EYCHYVAGLV GLGISEMFSA CGFESPLVAE |
| 201 | RKDLSNSMGL FLQKTNIARD YLEDLRDNRR FWPKEIWGQY |
| 241 | AETMEDLVKP ENKEKALQCL SHMIVNAMEH IRDVLEYLSM |
| 281 | IKNPSCFKFC AIPQVMAMAT LNLLHSNYKV FTHENIKIRK |
| 321 | GETVWLMKES DSMDKVAAIF RLYARQINNK SNSLDPHFVD |
| 361 | IGVICGEIEQ ICVGRFPGST IEMKRMQAGV LGGKTGTVL |

Hence squalene synthases from various species can be evaluated or modified and then evaluated to optimize production of squalene.

### EXAMPLE 9

### Screening of Farnesyl Diphosphate Synthase (FPPS) Candidates

This Example describes screening of farnesyl diphosphate synthase (FPPS) candidates to increase yields of squalene prior to integration into the lipid droplet scaffolding platform.

Three FPPS candidates were evaluated: Arabidopsis thaliana FPPS (AtFPPS), Picea abies FPPS (PaFPPS), and Gallus gallus FPPS (GgPPS). An example of a Picea abies FPPS (PaFPPS) sequence is shown below as SEQ ID NO:97 (NCBI accession no. ACΔ21460.1).

| | |
|---|---|
| 1 | MASNGIVDVK TKFEEIYLEL KAQILNDPAF DYTEDARQWV |
| 41 | EKMLDYTVPG GKLNRGLSVI DSYRLLKAGK EISEDEVFLG |
| 81 | CVLGWCIEWL QAYFLILDDI MDSSHTRRGQ PCWFRLPKVG |
| 121 | LIAVNDGILL RNHICRILKK HFRTKPYYVD LLDLFNEVEF |
| 161 | QTASGQLLDL ITTHEGATDL SKYKMPTYVR IVQYKTAYYS |
| 201 | FYLPVACALV MAGENLDNHV DVKNILVEMG TYFQVQDDYL |
| 241 | DCFGDPEVIG KIGTDIEDFK CSWLVVQALE RANESQLQRL |
| 281 | YANYGKKDPS CVAEVKAVYR DLGLQDVFLE YERTSHKELI |
| 321 | SSIEAQENES LQLVLKSFLG KIYKRQK |

A cDNA encoding the Picea abies FPPS (PaFPPS) with SEQ ID NO:90 is shown below as SEQ ID NO:98.

| | |
|---|---|
| 1 | ATGGCTTCAA ACGGCATCGT CGACGTGAAA ACCAAGTTTG |
| 41 | AGGAAATCTA TCTTGAGCTT AAGGCTCAGA TTCTGAACGA |
| 81 | TCCTGCCTTC GATTACACCG AAGACGCCCG TCAATGGGTC |
| 121 | GAGAAGATGC TGGACTACAC GGTGCCCGGA GGAAAGCTGA |
| 161 | ACCGCGGTCT GTCTCTAATA CACAGCTACA GGCTATTGAA |
| 201 | AGCAGGAAAG GAAATATCAG AAGATGAAGT CTTTCTTGGA |
| 241 | TCTCTGCTTC GCTGGTGTAT TCAATGGCTT CAAGCATATT |
| 281 | TCCTCATATT AGATCACATC ATCGACACCT CTCACACTAC |
| 321 | GCGTGGACAA CCTTGTTGGT TCAGATTACC TAAGGTTGGC |
| 361 | TTAATTGCTG TTAATGATGG AATATTGCTT CGTAACCACA |
| 401 | TATGCAGAAT TCTGAAAAAG CATTTTCGCA CTAAGCCTTA |
| 441 | CTATGTGGAT CTCCTTGATT TATTCAATGA GGTTGAGTTT |
| 481 | CAAACAGCTA GTGGACAGTT GCTGGACCTT ATCACTACTC |
| 521 | ATGAAGGAGC AACTGACCTT TCAAAGTACA AAATGCCAAC |
| 561 | TTATGTTCGT ATAGTTCAAT ACAAGACTGC CTACTATTCA |
| 601 | TTCTATCTGC CGGTTGCCTG TGCACTGGTA ATGGCAGGGG |
| 641 | AAAATTTAGA TAATCACGTA GATGTCAAGA ATATTTTAGT |
| 681 | CGAAATGGGA ACCTATTTTC AAGTACAGGA TGATTATCTT |
| 721 | GATTGCTTTG GTGATCCAGA AGTGATTGGG AAGATTGGAA |
| 761 | CTGATATCGA AGACTTCAAG TGCTCTTGGT TGGTGGTGCA |
| 801 | ACCCCTTCAA CGGGCAAATG AGAGCCAACT TCAACCATTA |
| 841 | TATGCCAATT ATGGAAAGAA AGATCCTTCT TGTGTTGCAG |
| 881 | AAGTCAAGGC TGTATATAGG GATCTTCGAC TTCAGGATGT |
| 921 | TTTTCTGCAA TACGACCGTA CTAGTCACAA GGAGCTCATT |
| 961 | TCTTCCATCG AGGGTCAGGA GAATGAATCT TTGCAGCTTG |
| 1001 | TTCTGAAGTC CTTCCTAGGG AAGATATACA AGCGACAGAA |
| 1041 | GTAA |

An example of a Gallus gallus FPPS (GgFPPS) polypeptide sequence is shown below as SEQ ID NO:99 (NCBI accession no. XP_015154133.1).

| | |
|---|---|
| 1 | MSADGAKRTA AEREREEFVG FFPQIVRDLT EDGIGHPEVG |
| 41 | DAVARLKEVL QYNAPGGKCN RGLTVVAAYR ELSGPGQKDA |
| 81 | ESLRCALAVG WCIELFQAFF LVADDIMDQS LTRRGQLCWY |
| 121 | KKEGVGLDAI NDSFLLESSV YRVLKKYCGQ RPYYVHLLEL |
| 161 | FLQTAYQTEL GQMLDLITAP VSKVDLSHFS EERYKAIVKY |
| 201 | KTAFYSFYLP VAAAMYMVGI DSKEEHENAK AILLEMGEYF |
| 241 | QIQDDYLDCF GDPALTGKVG TDIQDNKCSW LVVQCLQRVT |
| 281 | PEQRQLLEDN YGRKEPEKVA KVKELYEAVG MRAAFQQYEE |
| 321 | SSYRRLQELI EKHSNRLPKE IFLGLAQKIY KRQK |

A cDNA encoding the Gallus gallus FPPS (GgFPPS) with SEQ ID NO:92 is shown below as SEQ ID NO:100.

| | |
|---|---|
| 1 | AGAATGCCCC GCGCGGCGCC GGGCGGAGCG CACGGAAAGG |
| 41 | TCGCGGGGCA AAAAGCGGCG CTGAGCGGAC GGGGCCGAAC |
| 81 | GCGTCGGGGT CGCCATGAGC GCGGATGGGG CGAAGCGGAC |
| 121 | GGCGGCCGAG AGGGAGAGGG AGGAGTTCGT GGGGTTCTTC |
| 161 | CCGCAGATCG TCCGCGATCT GACCGAGGAC GGCATCGGAC |
| 201 | ACCCGGAGGT GGGCGACGCT GTGGCGCGGC TGAAGGAGGT |
| 241 | GCTGCAATAC AACGCTCCCG GTGGGAAATG CAACCGTGGG |
| 281 | CTGACGGTGG TGGCTGCGTA CCGGGAGCTG TCGGGGCCGG |
| 321 | GGCAGAAGGA TGCTGAGAGC CTGCGGTGCG CGCTGGCCGT |
| 361 | GGGTTGGTGC ATCGAGTTGT TCCAGGCCTT CTTCCTGGTG |
| 401 | GCTGATGATA TCATGGATCA GTCCCTCACG CGCCGGGGGC |
| 441 | AGCTGTGTTG GTATAAGAAG GAGGGGGTCG GTTTGGATGC |
| 481 | CATCAACGAC TCCTTCCTCC TCGAGTCCTC TGTGTACAGA |
| 521 | GTGCTGAAGA AGTACTGCGG GCAGCGGCCG TATTACGTGC |
| 561 | ATCTGTTGGA GCTCTTCCTG CAGACCGCCT ACCAGACTGA |
| 601 | GCTCGGGCAG ATGCTGGACC TCATCACAGC TCCCGTCTCC |
| 641 | AAAGTGGATT TGAGTCACTT CAGCGAGGAG AGGTACAAAG |
| 681 | CCATCGTTAA GTACAAGACT GCCTTCTACT CCTTCTACCT |
| 721 | ACCCGTGGCT GCTGCCATGT ATATGGTTGG GATCGACAGT |
| 761 | AAGGAAGAAC ACGAGAATGC CAAAGCCATC CTGCTGGAGA |
| 801 | TGGGGGAATA CTTCCAGATC CAGGATGATT ACCTGGACTG |
| 341 | CTTTGGGGAC CCGGCGCTCA CGGGGAAGGT GGGCACCGAC |
| 881 | ATCCAGGACA ATAAATGCAG CTGGCTCGTG GTGCAGTGCC |
| 921 | TGCAGCGCGT CACGCCGGAG CAGCGGCAGC TCCTGGAGGA |
| 961 | CAACTACGGC CGTAAGGAGC CCGAGAAGGT GGCGAAGGTG |
| 1001 | AAGGAGCTGT ATGAGGCCGT GGGGATGAGG GCTGCGTTCC |
| 1041 | AGCAGTACGA GGAGAGCAGC TACCGGCGCC TGCAGGAACT |
| 1081 | GATAGAGAAG CACTCGAACC GCCTCCCGAA GGAGATCTTC |
| 1121 | CTCGGCCTGG CACAGAAGAT CTACAAACGC CAGAAATGAG |
| 1161 | GGGTGGGGGC GGCAGCGGCT CTGTGCTTCG CGCTGTGTTG |
| 1201 | GGTGGCTTCG CAGCCCCGGA CCCGGTGCTC CCCCCACCCG |
| 1241 | TTATCCCCGG AGATGCGGGG GGGGGGCGGT GCGGGGCGCG |
| 1281 | CATCCATCGG TGCCGTCAGA CTGTGTGTCA ATAAACGTTA |
| 1321 | ATTTATTGCC |

These farnesyl diphosphate synthases are natively cytosolic. However, these farnesyl diphosphate synthases were modified to be targeted to plastids.

The plastid-targeted farnesyl diphosphate synthases were co-expressed with CfDXS and MaSQS CΔ17 and squalene yields were measured by GC-FID.

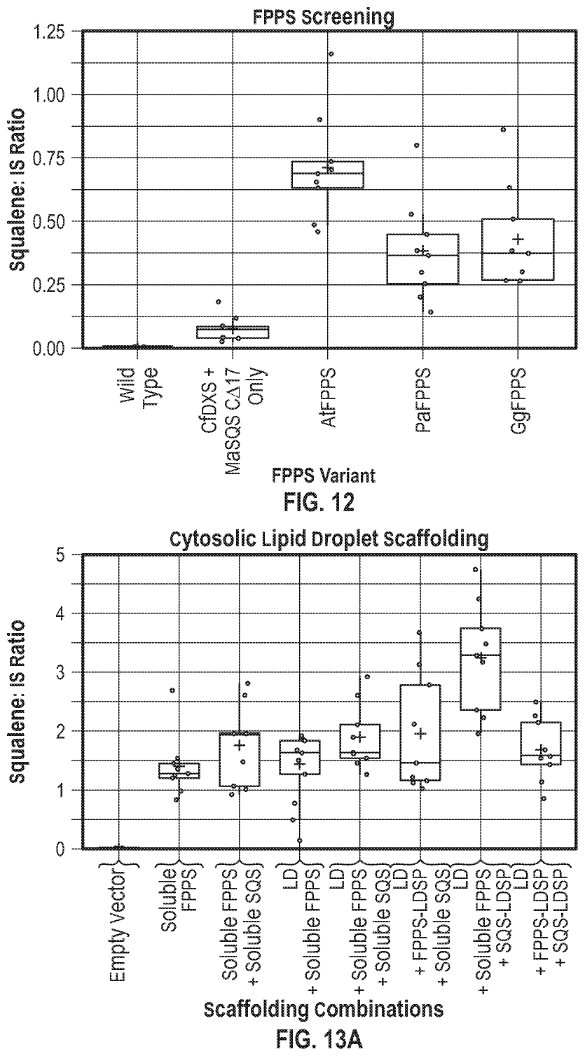

The squalene yields are reported in FIG. 12 as a ratio to the internal standard, n-hexacosane. As shown in FIG. 12, in this experiment, an Arabidopsis thaliana FPPS provided the highest squalene production.

### EXAMPLE 10

### Linking SQS and/or FFPS to Lipid Droplet Surface Proteins Improves Squalene Yields

This Example illustrates that linkage of lipid droplet surface protein to enzymes can optimize production of lipophilic products.

In a first experiment, AtFPPS and MaSQS CΔ17 were transiently expressed in Nicotiana benthamiana in cytosolic or soluble form, or in fusion with lipid droplet surface protein. LDSP fusions were to the C-terminal ends of AtFPPS and MaSQS CΔ17. Constructs excluding the empty vector were co-expressed with an N-terminally truncated Euphorbia lathyris HMG-CoA reductase (ElHMGR¹⁵⁹⁻⁵⁸²) to increase flux through the cytosolic MVA pathway, thereby increasing IPP/DMAPP availability. AtWRI1¹⁻³⁹⁷, lipid droplet surface protein (not fused to an enzyme), or a combination thereof was also expressed in some assays.

Table 2 summarizes the amounts of squalene that accumulated in cells expressing various constructs and combinations of proteins.

TABLE 2
Ratios of Squalene:Standard

| | Median | Mean |
|---|---|---|
| | Squalene:Standard | Squalene:Standard |
| Proteins Expressed | Ratio | Ratio |
| Empty Vector | 0 | 0 |
| ElHMGR + AtFPPS | 1.277 | 1.400 |
| ElHMGR + AtFPPS + | 1.950 | 1.749 |
| MaSQS CΔ17 | | |
| AtWRI1 + NoLDSP + | 1.632 | 1.438 |
| ElHMGR + AtFPPS | | |
| AtWRI1 + NoLDSP + | 1.634 | 1.891 |
| ElHMGR + AtFPPS + | | |
| MaSQS CΔ17 | | |
| AtWRI1 + ElHMGR + | 1.458 | 1.962 |
| AtFPPS-NoLDSP + | | |
| MaSQS CΔ17 | | |
| AtWRI1 + ElHMGR + | 3.268 | 3.232 |
| AtFPPS + MaSQS CΔ17- | | |
| NoLDSP | | |
| AtWRI1 + ElHMGR + | 1.576 | 1.678 |
| AtFPPS-NoLDSP + | | |
| MaSQS CΔ17-NoLDSP | | |

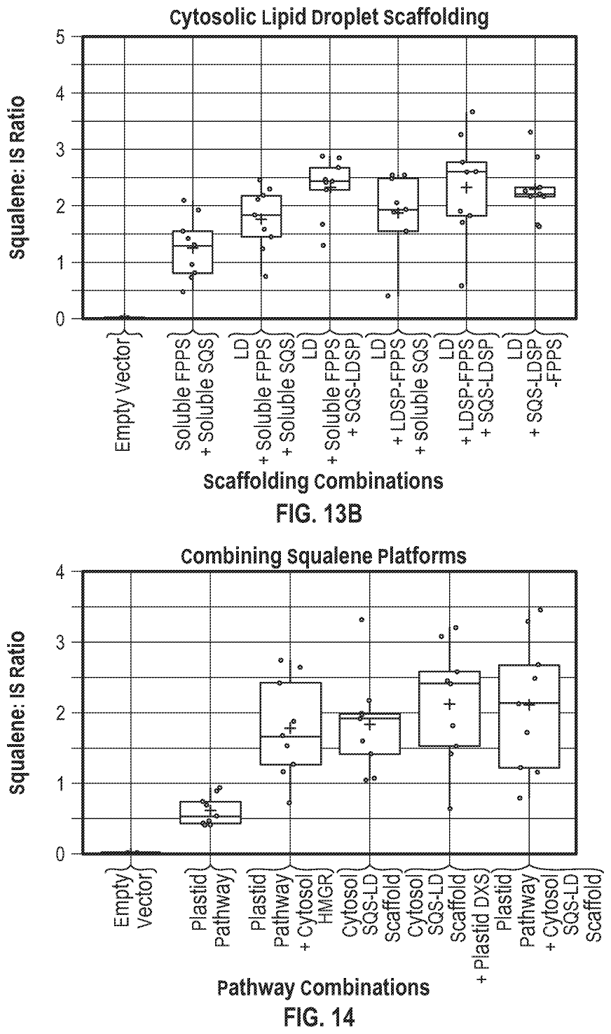

These data are graphically illustrated in FIG. 13A, demonstrating that in this experiment, the combination which yields the highest levels of squalene included expression of AtWRI1¹⁻³⁹⁷, MaSQS CΔ17-NoLDSP, ElHMGR¹⁵⁹⁻⁵⁸², and AtFPPS.

In a second experiment, NoLDSP was fused to either the C-terminus of MaSQS CΔ17, the N-terminus of AtFPPS, or NoLDSP was linked to both MaSQS and AtFPPS to form a single fusion of all three proteins with NoLDSP in between AtWRI1¹⁻³⁹⁷ was expressed in samples indicated with “LD” alongside either NoLDSP alone, or NoLDSP fused to AtFPPS and MaSQS CΔ17 as indicated. All samples co-expressed with ElHMGR¹⁵⁹⁻⁵⁸² except for the empty vector.

Table 3 summarizes the amounts of squalene that accumulated in cells expressing various constructs and combinations of proteins.

TABLE 3
Ratios of Squalene:Standard

| | Median | Mean |
|---|---|---|
| | Squalene:Standard | Squalene:Standard |
| Genes | Ratio | Ratio |
| Empty Vector | 0 | 0.002 |
| ElHMGR + AtFPPS + | 1.299 | 1.249 |
| MaSQS CΔ17 | | |
| AtWRI1 + NoLDSP + | 1.837 | 1.764 |
| ElHMGR + AtFPPS + | | |
| MaSQS CΔ17 | | |
| AtWRI1 + ElHMGR + | 2.430 | 2.327 |
| AtFPPS + | | |
| MaSQS CΔ17-NoLDSP | | |
| AtWRI1 + ElHMGR + | 1.928 | 1.866 |
| NoLDSP-AtFPPS + | | |
| MaSQS CΔ17 | | |
| AtWRI1 + ElHMGR + | 2.599 | 2.323 |
| NoLDSP-AtFPPS + | | |
| MaSQS CΔ17-NoLDSP | | |
| AtWRI1 + ElHMGR + | 2.206 | 2.284 |
| MaSQS CΔ17-NoLDSP- | | |
| AtFPPS | | |

These data are graphically illustrated in FIG. 13B, showing that cellular accumulation of squalene was improved by linkage of either of the two final enzymes in the squalene pathway to lipid droplet surface protein. But squalene accumulation was comparable in cells with either of the two final enzymes in the squalene pathway fused with lipid droplet surface protein. The methods and expression systems described herein can readily be adapted to optimize squalene and triterpene biosynthesis. Linkage of enzymes in the squalene biosynthesis pathway to lipid droplet surface protein increased squalene accumulation compared to the amounts of squalene that accumulated in Nicotiana benthamiana cells when such enzymes are expressed in soluble, non-fused form.

### EXAMPLE 11

### Improved Capacity of the Lipid Droplet Scaffolding Platform

This Example illustrates that contributions from the MEP pathway with plastidial expression and use of enzyme fusions to lipid droplet surface protein can further boost squalene biosynthesis.

The contributions of plastidial IPP/DMAPP or the MEP pathway were evaluated while using the following expression systems.

A “Cytosol SQS-LD Scaffold” system included a lipid droplet surface protein fused to a MaSQS CΔ17squalene synthase (MaSQS CΔ17-NoLDSP). The AtWRI1¹⁻³⁹⁷, ElHMGR¹⁵⁹⁻⁵⁸², and AtFPPS were expressed with the Cytosol SQS-LD Scaffold.

A “Plastid Pathway” system involved use of components of a plastidial targeted squalene pathway consisting of CfDXS, plastidial AtFPPS, and plastidial MaSQS CΔ17. Additionally, CfDXS alone was co-expressed with the SQS-LD scaffold.

Table 4 summarizes the amounts of squalene that accumulated in cells expressing various constructs and combinations of proteins.

TABLE 4
Ratios of Squalene:Standard

| | Median | Mean |
|---|---|---|
| | Squalene:Standard | Squalene:Standard |
| Genes | Ratio | Ratio |
| Empty Vector | 0 | 0 |
| Plastid Pathway | 0.534 | 0.615 |
| HMGR + Plastid Pathway | 1.669 | 1.778 |
| Cytosolic:SQS-LD scaffold | 1.912 | 1.828 |
| Cytosolic:SQS-LD | 2.403 | 2.120 |
| scaffold + DXS | | |
| Plastid Pathway + | 2.123 | 2.099 |
| Cytosolic:SQS-LD scaffold | | |

These data are graphically illustrated, in FIG. 14, illustrating that increased plastidial IPP/DMAPP availability when using the cytosolic LD scaffolding platform can influence and increase accumulation of terpenes.

### EXAMPLE 12

### LDSP-Fusions Increase Lipid Accumulation in Poplar Leaves

This Example illustrates that expression of lipid droplet surface protein fusions provides accumulation of lipid droplets within poplar leaves.

AtWRI1¹⁻³⁹⁷ was linked to eYFP-NoLDSP by the “self-cleaving” LP4/2A hybrid linker. This AtWRI1¹⁻³⁹⁷-eYFP-NoLDSP fusion or an eYFP-NoLDSP fusion was expressed in poplar NM6 leaves by Agrobacterium-mediated transient expression.

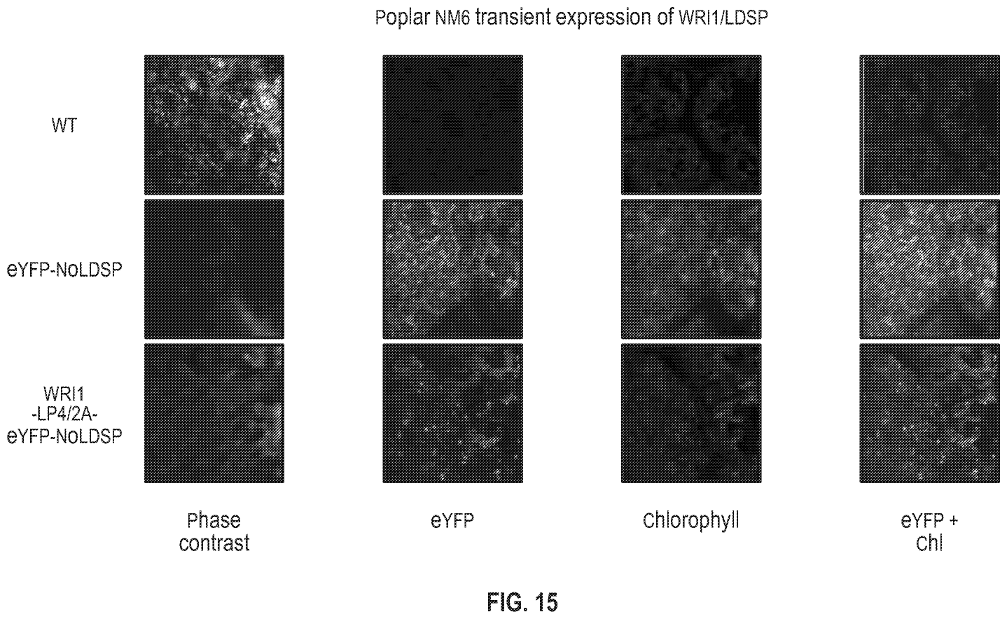

FIG. 15 shows images of wild type, non-infiltrated poplar leaves (top row). The middle row in FIG. 15 shows images of leaves transiently expressing eYFP-NoLDSP fusion gene from pEAQ vector, while the bottom row images show leaves transiently expressing AtWRI1¹⁻³⁹⁷ linked to eYFP-NoLDSP by the “self-cleaving” LP4/2A hybrid linker, which is cleaved during translation to form the two separate protein products.

Punctae are present in the bottom row images of FIG. 15 indicating formation of lipid droplets in leaves of poplar NM6.

### EXAMPLE 13

### Constructs and Vectors

This Example describes some of the constructs and vectors that have been made and used in the development of the systems and methods described herein. The pEAQ vectors (see, e.g., Sainsbury et al. (Plant Biotechnology Journal 7: 682-693 (2009)) were used as a basis for these constructs and expression vectors.

Table 5 describes the proteins and/or fusion proteins encoded within several pEAQ-ht or pEAQ vectors.

TABLE 5
Constructs and Vectors

| Construct name | Description |
|---|---|
| peaq-ht_atwri1- | pEAQ: AtWRI1 (1-397) linked to eYFP-NoLDSP |
| 397_lp42a_noldsp-yfp | by LP4/2A v1 linker |
| peaq-ht_masqs-noldsp | pEAQ: MaSQS CΔ17 with C-terminal NoLDSP |
| | fusion |
| peaq-ht_atfpps-noldsp | pEAQ: AtFPPS with C-terminal NoLDSP fusion |
| *peaq-ht_noldsp-atfpps | pEAQ: AtFPPS with N-terminal NoLDSP fusion |
| *peaq-ht_masqs-noldsp- | pEAQ: N-terminal MaSQS CΔ17 - NoLDSP - |
| atfpps | AtFPPS C-terminal |
| pld1hfs2-peaq-ld-sq | Modified pEAQ: AtWRI1(1-397)-LP4/2Av1-eYFP- |
| | NoLDSP in site 1, |
| | Soluble ElHMGR(159-582)-LP4/2Av1-AtFPPS- |
| | LP4/2Av2-MaSQS CΔ17 in site 2 |
| plds1hf2- | Modified pEAQ: AtWRI1(1-397)-LP4/2Av1-MaSQS |
| peaq_wri1lv1sqs- | CΔ17-NoLDSP in site 1, |
| ldspmcs1_hmgrlv1fppsmcs2 | ElHMGR(159-582)-LP4/2Av1-AtFPPS in site 2 |
| pwh1slf2- | Modified pEAQ: AtWRI1(1-397)-LP4/2Av1- |
| peaq_wri1lv1hmgrmcs1_sqs- | ElHMGR(159-582) in site 1, |
| ldsp-fppsmcs2 | MaSQS CΔ17-NoLDSP-AtFPPS in site 2 |

As indicated, an additional cloning site was inserted into a pEAQ vector to facilitate expression of more than one protein or fusion protein. The LP4/2A v1 linker, which undergoes cleavage during translation was used in some cases. For example, a soluble ElHMGR(159-582) was linked to an AtFPPS via the LP4/2Av1 linker and the AtFPPS was linked to MaSQS CΔ17 via a LP4/2Av2 linker, allowing these three proteins to be expressed together and then to be separated as they were translated.

An example of a sequence for the pld1hfs2-peaq-ld-sq plasmid is shown below as SEQ ID NO:103.

cctgtggttggcatgcacatacaaatggacgaacggataaaccttttcacgcccttt
taaatatccgattattctaataaacgctcttttctcttaggtttacccgccaatata
tcctgtcaaacactgatagtttgtgaaccatcacccaaatcaagttttttggggtcg
aggtgccgtaaagcactaaatcggaaccctaaagggagcccccgatttagagcttga
cggggaaagccggcgaacgtggcgagaaaggaagggaagaaagcgaaaggagcgggc
gccattcaggctgcgcaactgttgggaagggcgatcggtgcgggcctcttcgctatt
acgccagctggcgaaagggggatgtgctgcaaggcgattaagttgggtaacgccagg
gttttcccagtcacgacgttgtaaaacgacggccagtgaattgttaattaagaattc
gagctccaccgcggaaacctcctcggattccattgcccagctatctgtcactttatt
gagaagatagtggaaaaggaaggtggctcctacaaatgccatcattgcgataaagga
aaggccatcgttgaagatgcctctgccgacagtggtcccaaagatggacccccaccc
acgaggagcatcgtggaaaaagaagacgttccaaccacgtcttcaaagcaagtggat
tgatgtgatatctccactgacgtaagggatgacgcacaatcccactatccttcgcaa
gacccttcctctatataaggaagttcatttcatttggagaggtattaaaatcttaat
aggttttgataaaagcgaacgtggggaaacccgaaccaaaccttcttctaaactctc
tctcatctctcttaaagcaaacttctctcttgtctttcttgcgtgagcgatcttcaa
cgttgtcagatcgtgcttcggcaccagtacaacgttttctttcactgaagcgaaatc
aaagatctctttgtggacacgtagtgcggcgccattaaataacgtgtacttgtccta
ttcttgtcggtgtggtcttgggaaaagaaagcttgctggaggctgctgttcagcccc
atacattacttgttacgattctgctgactttcggcgggtgcaatatctctacttctg
cttgacgaggtattgttgcctgtacttctttcttcttcttcttgctgattggttcta
taagaaatctagtattttctttgaaacagagttttcccgtggttttcgaacttggag
aaagattgttaagcttctgtatattctgcccaaattcgcgATGAAGAAGCGCTTAAC
CACTTCCACTTGTTCTTCTTCTCCATCTTCCTCTGTTTCTTCTTCTACTACTACTTC
CTCTCCTATTCAGTCGGAGGCTCCAAGGCCTAAACGAGCCAAAAGGGCTAAGAAATC
TTCTCCTTCTGGTGATAAATCTCATAACCCGACAAGCCCTGCTTCTACCCGACGCAG
CTCTATCTACAGAGGAGTCACTAGACATAGATGGACTGGGAGATTCGAGGCTCATCT
TTGGGACAAAAGCTCTTGGAATTCGATTCAGAACAAGAAAGGCAAACAAGTTTATCT
GGGAGCATATGACAGTGAAGAAGCAGCAGCACATACGTACGATCTGGCTGCTCTCAA
GTACTGGGGACCCGACACCATCTTCAATTTTCCGGCAGAGACGTACACAAAGGAATT
GGAAGAAATGCAGAGAGTGACAAAGGAAGAATATTTGGCTTCTCTCCGCCGCCAGAG
CAGTGGTTTCTCCAGAGGCGTCTCTAAATATCGCGGCGTCGCTAGGCATCACCACAA
CGGAAGATGGGAGGCTCGGATCGGAAGAGTGTTTGGGAACAAGTACTTGTACCTCGG
CACCTATAATACGCAGGAGGAAGCTGCTGCAGCATATGACATGGCTGCGATTGAGTA
TCGAGGCGCAAACGCGGTTACTAATTTCGACATTAGTAATTACATTGACCGGTTAAA
GAAGAAAGGTGTTTTCCCGTTCCCTGTGAACCAAGCTAACCATCAAGAGGGTATTCT
TGTTGAAGCCAAACAAGAAGTTGAAACGAGAGAAGCGAAGGAAGAGCCTAGAGAAGA
AGTGAAACAACAGTACGTGGAAGAACCACCGCAAGAAGAAGAAGAGAAGGAAGAAGA
GAAAGCAGAGCAACAAGAAGCAGAGATTGTAGGATATTCAGAAGAAGCAGCAGTGGT
CAATTGCTGCATAGACTCTTCAACCATAATGGAAATGGATCGTTGTGGGGACAACAA
TGAGCTGGCTTGGAACTTCTGTATGATGGATACAGGGTTTTCTCCGTTTTTGACTGA
TCAGAATCTCGCGAATGAGAATCCCATAGAGTATCCGGAGCTATTCAATGAGTTAGC
ATTTGAGGACAACATCGACTTCATGTTCGATGATGGGAAGCACGAGTGCTTGAACTT
GGAAAATCTGGATTGTTGCGTGGTGGGAAGAGAGTCAAATGCAGCAGACGAAGTTGC
TACTCAACTTTTGAATTTTGACTTGCTGAAGTTGGCTGGTGATGTTGAGTCAAACCC
TGGACCTATGGGCAAGGGCGAGGAGCTGTTCACCGGGGTGGTGCCCATCCTGGTCGA
GCTGGACGGCGACGTAAACGGCCACAAGTTCAGCGTGTCCGGCGAGGGCGAGGGCGA
TGCCACCTACGGCAAGCTGACCCTGAAGTTCATCTGCACCACCGGCAAGCTGCCCGT
GCCCTGGCCCACCCTCGTGACCACCTTCGGCTACGGCCTGCAGTGCTTCGCCCGCTA
CCCCGACCACATGAAGCAGCACGACTTCTTCAAGTCCGCCATGCCCGAAGGCTACGT
CCAGGAGCGCACCATCTTCTTCAAGGACGACGGCAACTACAAGACCCGCGCCGAGGT
GAAGTTCGAGGGCGACACCCTGGTGAACCGCATCGAGCTGAAGGGCATCGACTTCAA
GGAGGACGGCAACATCCTGGGGCACAAGCTGGAGTACAACTACAACAGCCACAACGT
CTATATCATGGCCGACAAGCAGAAGAACGGCATCAAGGTGAACTTCAAGATCCGCCA
CAACAXCGAGGACGGCAGCGTGCAGCTCGCCGACCACTACCAGCAGAACACCCCCAT
CGGCGACGGCCCCGTGCTGCTGCCCGACAACCACTACCTGAGCTACCAGTCCGCCCT
GAGCAAAGACCCCAACGAGAAGCGCGATCACATGGTCCTGCTGGAGTTCGTGACCGC
CGCCGGGATCACTCTCGGCATGGACGAGCTGTACAAGTCCGGACTCAGATCTCGAGC
TCAAGCTTCGAATTCTGCAGTCGACGGTACCGCGGGCCCGGGATCATCAACAAGTTT
GTACAAAAAAGCAGGCTCCACCATGGCCGGCCCCATCATGACCTCTGCGCCCTCCGC
GACCACGCCCACGGGCAAGACAATGCCGTTCAAGCAGCCTTTCAAGACTGTGGCCAC
GCTGTCCGCCAAGACTGGCAACATTACCAAGCCCATCGACCCTGCCATCTCCAAGAC
CATTGACTTCGTCTACAATGGTTACTCGACGGTCAAGACCAAGGTTGACAAGGCCCC
TAAGGTAAACCCCTACCTGCTCATTGCCGGCGGCCTCGTCCTCTCGTGCATCATCTC
CATGTGCCTGCTCGTCCCGGCCGTGATCTTCTTCCCCGTCACCATCTTCCTGGGTGT
CGCTACGTCGTTTGCGCTCATTGCATTGGCCCCCGTGGCTTTTGTGTTCGGGTGGAT
CCTGATCTCCTCTGCTCCGATCCAGGATAAGGTGGTGGTGCCCGCCTTGGACAAGGT
GCTGGCCAATAAGAAGGTGGCGAAGTTCCTCCTCAAGGAGTAAtcgaggcctttaac
tctggtttcattaaattttctttagtttgaatttactgttattcggtgtgcatttct
atgtttggtgagcggttttctgtgctcagagtgtgtttattttatgtaatttaattt
ctttgtgagctcctgtttagcaggtcgtcccttcagcaaggacacaaaaagatttta
attttattaaaaaaaaaaaaaaaaaagaccgggaattcgatatcaagcttatcgacc
tgcagatcgttcaaacatttggcaataaagtttcttaagattgaatcctgttgccgg
tcttgcgatgattatcatataatttctgttgaattacgttaagcatgtaataattaa
catgtaatgcatgacgttatttatgagatgggtttttatgattagagtcccgcaatt
atacatttaatacgcgatagaaaacaaaatatagcgcgcaaactaggataaattatc
gcgcgcggtgtcatctatgttactagatctctagagtctcaagcttggcgcgccagc
ttggcgtaatcatggtcatagctgttgcgattaagaattcgagctcggtacccccct
actccaaaaatgtcaaagatacagtctcagaagaccaaagggctattgagacttttc
aacaaagggtaatttcgggaaacctcctcggattccattgcccagctatctgtcact
tcatcgaaaggacagtagaaaaggaaggtggctcctacaaatgccatcattgcgata
aaggaaaggctatcattcaagatgcctctgccgacagtggtcccaaagatggacccc
cacccacgaggagcatcgtggaaaaagaagacgttccaaccacgtcttcaaagcaag
tggattgatgtgacatctccactgacgtaagggatgacgcacaatcccactatcctt
cgcaagacccttcctctatataaggaagttcatttcatttggagaggacagcccaag
cttcgactctagaggatccccttaaatcgatATTTATGATTTCGCCTCTGGCATCCG
AGGAGGATGAGGAAATTGTTAAATCTGTTGTTAATGGAACGATTCCTTCGTATTCGT
TGGAATCGAAGCTTGGGGATTGTAAAAGAGCGGCTGAGATTCGACGGGAGGCTTTGC
AGAGAATGATGGGGAGGTCGTTGGAGGGTTTACCTGTTGAAGGATTCGATTATGAGT
CGATTTTAGGTCAGTGCTGTGAAATGCCTGTTGGTTATGTGCAGATTCCGGTTGGAA
TTGCTGGGCCGTTGCTGCTAGACGGGCAAGAGTACTCTGTTCCGATGGCGACCACCG
AGGGTTGTTTGGTTGCTAGCACTAATAGAGGGTGTAAAGCGATCCATTTGTCAGGTG
GTGCTAGTAGTGTCTTGTTGAAGGATGGCATGACTAGAGCTCCCGTTGTTCGATTCG
CCTCGGCCATGAGGGCCGCGGATTTGAAGTTTTTCTTAGAGAATCCTGAGAATTTCG
ATAGCTTGTCCATCGCTTTCAATAGGTCCAGTAGATTTGCAAAGCTCCAAAGCATAC
AATGTTCTATTGCTGGAAAGAATCTATATATGAGATTCACCTGCAGCACTGGTGATG
CAATGGGGATGAACATGGTTTCCAAAGGGGTTCAAAACGTTCTTGACTTCCTTCAAA
GTGATTTCCCTGACATGGATGTTATTGGCATCTCAGGAAATTTTTGTTCGGACAAGA
AGCCAGCTGCTGTGAACTGGATTCAAGGGCGAGGCAAATCGGTTGTTTGCGAGGCAA
TTATCAAGGAAGAGGTGGTGAAGAAGGTATTGAAATCAAGTGTTGCTTCACTAGTAG
AGCTGAACATGCTCAAGAATCTTACTGGTTCAGCTATTGCTGGAGCTCTTGGTGGAT
TCAATGCACATGCTGGCAACATAGTCTCTGCAATTTTCATTGCCACTGGCCAGGATC
CAGCCCAGAATGTTGAGAGTTCTCATTGCATCACCATGATGGAAGCTGTCAATGATG
GAAAAGATCTCCACATCTCTGTAACCATGCCTTCAATCGAGGTAGGAACAGTTGGAG
GAGGGACACAACTAGCATCCCAATCAGCATGTCTGAACCTACTCGGTGTAAAAGGAG
CAAGTAAAGAATCACCAGGAGCAAACTCAACCCTCCTAGCCACAATAGTAGCTGGTT
CAGTCCTAGCTGGTGAACTCTCCCTAATGTCAGCCATAGCAGCAGGACAACTAGTCC
GGAGCCACATGAAGTACAACAGATCCAGCAAAGATGTAACCAAATTTGCATCATCTT
CAAATGCAGCAGACGAAGTTGCTACTCAACTTTTGAATTTTGACTTGCTGAAGTTGG
CTGGTGATGTTGAGTCAAACCCTGGACCTATGGCGGATCTGAAATCAACCTTCCTCG
ACGTTTACTCTGTTCTCAAGTCTGATCTGCTTCAAGATCCTTCCTTTGAATTCACCC
ACGAATCTCGTCAATGGCTTGAACGGATGCTTGACTACAATGTACGCGGAGGGAAGC
TAAATCGTGGTCTCTCTGTGGTTGATAGCTACAAGCTGTTGAAGCAAGGTCAAGACT
TGACGGAGAAAGAGACTTTCCTCTCATGTGCTCTTGGTTGGTGCATTGAATGGCTTC
AAGCTTATTTCCTTGTGCTTGATGACATCATGGACAACTCTGTCACACGCCGTGGCC
AGCCTTGTTGGTTTAGAAAGCCAAAGGTTGGTATGATTGCCATTAACGATGGGATTC
TACTTCGCAATCATATCCACAGGATTCTCAAAAAGCACTTCAGGGAAATGCCTTACT
ATGTTGACCTCGTTGATTTGTTTAACGAGGTAGAGTTTCAAACAGCTTGCGGCCAGA
TGATTGATTTGATCACCACCTTTGATGGAGAAAAAGATTTGTCTAAGTACTCCTTGC
AAATCCATCGGCGTATTGTTGAGTACAAAACAGCTTATTACTCATTTTATCTTCCTG
TTGCTTCCGCATTGCTCATGCCCGCAGAAAATTTGGAAAACCATACTGATGTCAAGA
CTGTTCTTGTTGACATGGGAATTTACTTTCAAGTACAGGATGATTATCTGGACTGTT
TTGCTGATCCTGAGACACTTGGCAAGATAGGGACAGACATAGAAGATTTCAAATGCT
CCTGGTTGGTAGTTAAGGCATTGGAACGCTGCAGTGAAGAACAAACTAAGATACTAT
ACGAGAACTATGGTAAAGCCGAACCATCAAACGTTGCTAAGGTGAAAGCTCTCTACA
AAGAGCTTGATCTCGAGGGAGCGTTCATGGAATATGAGAAGGAAAGCTATGAGAAGC
TGACAAAGTTGATCGAAGCTCACCAGAGTAAAGCAATTCAAGCAGTGCTAAAATCTT
TCTTGGCTAAGATCTACAAGAGGCAGAAGAAATCCTCATCTAACGCTGCTGATGAGG
TGGCAACACAGTTGCTGAACTTCGATCTTTTGAAACTTGCAGGAGACGTGGAATCTA
ATCCAGGCCCAATGGCCAGTGCTATTCTTGCTTCATTACTCCACCCATCAGAAGTGT
TGGCACTTGTGCAGTACAAGCTTTCACCCAAAACCCAGCATGATTACTCTAACGACA
AAACTAGGCAAAGACTTTATCATCATCTTAATATGACTTCCCGATCCTTCTCTGCCG
TCATACAGGACCTTGATGAAGAGTTAAAGGATGCTATATGCTTATTCTATCTGGTGC
TGAGAGGCTTAGATACTATAGAAGACGACATGACCATCGACCTTGACACTAAATTGC
CTTACCTTCGTACGTTCCACGAAATCATATACCAGAAAGGCTGGACTTTCACTAAGA
ACGGCCCAAATGAAAAAGATAGGCAATTACTGGTAGAATTTGACGCCATCATAGAGG
GCTTCCTTCAATTGAAGCCAGCCTATCAGACTATCATTGCCGATATAACCAAACGTA
TGGGGAACGGAATGGCACACTACGCTACGGCAGGGATACATGTTGAGACCAACGCAG
ACTACGACGAGTACTGCCACTATGTCGCTGGTTTGGTGGGGCTGGGTCTCTCTGAAA
TGTTTTCCGCATGTGGGTTCGAAAGTCCTCTTGTGGCAGAAAGAAAAGACCTTAGCA
ACAGCATGGGACTTTTCCTTCAGAAGACGAACATTGCACGTGATTATCTTGAAGACC
TCAGAGACAATCGTCGATTTTGGCCCAAGGAAATATGGGGGCAGTATGCTGAGACTA
TGGAGGACTTGGTAAAGCCCGAAAATAAAGAAAAGGCCCTCCAATGCCTCTCCCATA
TGATCGTCAATGCAATGGAGCATATCAGAGACGTTTTGGAGTATCTCTCTATCATAA
AGAATCCGAGCTGCTTCAAATTTTGTGCTATTCCACAAGTCATGGCTATGGCCACAT
TAAACCTGCTTCATTCCAACTACAAAGTGTTCACGCATGAGAATATCAAGATCCGTA
AAGGTGAGACAGTGTGGCTTATGAAAGAAAGTGACAGTATGGACAAGGTAGCTGCTA
TCTTTAGGTTGTACGCCCGACAAATTAACAACAAGTCCAACTCTCTTGATCCCCATT
TTGTGGATATAGGGGTGATTTGCGGTGAGATCGAGCAAATTTGCGTAGGAAGGTTCC
CTGGCTCCACAATAGAAATGAAGCGAATGCAGGCTGGAGTCTTAGGGGGGAAAACTG
GAACGGTCCTGTAATCAGCAATTGggggagctcgaattcgctgaaatcaccagtctc
tctctacaaatctatctctctctattttctccataaataatgtatgagtagtttccc
gataagggaaattagggttcttatagggtttcgctcatgtgttgagcatataagaaa
cccttagtatgtatttgtatttgtaaaatacttctatcaataaaatttctaattcct
aaaaccaaaatccagtactaaaatccagatctcctaaagtccctatagatctttgtc
gtgaatataaaccagacacgagacgactaaacctggagcccagacgccgttcgaagc
tagaagtaccgcttaggcaggaggccgttagggaaaagatgctaaggcagggttggt
tacgttgactcccccgtaggtttggtttaaatatgatgaagtggacggaaggaagga
ggaagacaaggaaggataaggttgcaggccctgtgcaaggtaagaagatggaaattt
gatagaggtacgctactatacttatactatacgctaagggaatgcttgtatttatac
cctataccccctaataaccccttatcaatttaagaaataatccgcataagcccccgc
ttaaaaattggtatcagagccatgaataggtctatgaccaaaactcaagaggataaa
acctcaccaaaatacgaaagagttcttaactctaaagataaaagatggcgcgtggcc
ggcctacagtatgagcggagaattaagggagtcacgttatgacccccgccgatgacg
cgggacaagccgttttacgtttggaactgacagaaccgcaacgttgaaggagccact
cagccgcgggtttctggagtttaatgagctaagcacatacgtcagaaaccattattg
cgcattcaaaagtcgcctaaggtcactatcagctagcaaatatttcttgtcaaaaat
gctccactgacgttccataaattcccctcggtatccaattagagtctcatattcact
ctcaatccaaataatctgcaccggatctggatcgtttcgcatgattgaacaagatgg
attgcacgcaggttctccggccgcttgggtggagaggctattcggctatgactgggc
acaacagacaatcggctgctctgatgccgccgtgttccggctgtcagcgcaggggcg
cccggttctttttgtcaagaccgacctgtccggtgccctgaatgaactgcaggacga
ggcagcgcggctatcgtggctggccacgacgggcgttccttgcgcagctgtgctcga
cgttgtcactgaagcgggaagggactggctgctattgggcgaagtgccggggcagga
tctcctgtcatctcaccttgctcctgccgagaaagtatccatcatggctgatgcaat
gcggcggctgcatacgcttgatccggctacctgcccattcgaccaccaagcgaaaca
tcgcatcgagcgagcacgtactcggatggaagccggtcttgtcgatcaggatgatct
ggacgaagagcatcaggggctcgcgccagccgaactgttcgccaggctcaaggcgcg
catgcccgacggcgatgatctcgtcgtgacccatggcgatgcctgcttgccgaatat
catggtggaaaatggccgcttttctggattcatcgactgtggccggctgggtgtggc
ggaccgctatcaggacatagcgttggctacccgtgatattgctgaagagcttggcgg
cgaatgggctgaccgcttcctcgtgctttacggtatcgccgctcccgattcgcagcg
catcgccttctatcgccttcttgacgagttcttctgagcgggactctggggttcgaa
atgaccgaccaagcgacgcccaacctgccatcacgagatttcgattccaccgccgcc
ttctatgaaaggttgggcttcggaatcgttttccgggacgccggctggatgatcctc
cagcgcggggatctcatgctggagttcttcgcccacaggatctctgcggaacaggcg
gtcgaaggtgccgatatcattacgacagcaacggccgacaagcacaacgccacgatc
ctgagcgacaatatgatcgcggcgtccacatcaacggcgtcggcggcgactgcccag
gcaagaccgagatgcaccgcgatatcttgctgcgttcggatattttcgtggagttcc
cgccacagacccggatgatccccgatcgttcaaacatttggcaataaagtttcttaa
gattgaatcctgttgccggtcttgcgatgattatcatataatttctgttgaattacg
ttaagcatgtaataattaacatgtaatgcatgacgttatttatgagatgggttttta
tgattagagtcccgcaattatacatttaatacgcgatagaaaacaaaatatagcgcg
caaactaggataaattatcgcgcgcggtgtcatctatgttactagatcgggactgta
ggccggccctcactggtgaaaagaaaaaccaccccagtacattaaaaacgtccgcaa
tgtgttattaagttgtctaagcgtcaatttgtttacaccacaatatatcctgccacc
agccagccaacagctccccgaccggcagctcggcacaaaatcaccactcgatacagg
cagcccatcagtccgggacggcgtcagcgggagagccgttgtaaggcggcagacttt
gctcatgttaccgatgctattcggaagaacggcaactaagctgccgggtttgaaaca
cggatgatctcgcggagggtagcatgttgattgtaacgatgacagagcgttgctgcc
tgtgatcaaatatcatctccctcgcagagatccgaattatcagccttcttattcatt
tctcgcttaaccgtgacagagtagacaggctgtctcgcggccgaggggcgcagcccc
tgggggggatgggaggcccgcgttagcgggccgggagggttcgagaagggggggcac
cccccttcggcgtgcgcggtcacgcgcacagggcgcagccctggttaaaaacaaggt
ttataaatattggtttaaaagcaggttaaaagacaggttagcggtggccgaaaaacg
ggcggaaacccttgcaaatgctggattttctgcctgtggacagcccctcaaatgtca
ataggtgcgcccctcatctgtcagcactctgcccctcaagtgtcaaggatcgcgccc
ctcatctgtcagtagtcgcgcccctcaagtgtcaataccgcagggcacttatcccca
ggcttgtccacatcatctgtgggaaactcgcgtaaaatcaggcgttttcgccgattt
gcgaggctggccagctccacgtcgccggccgaaatcgagcctgcccctcatctgtca
acgccgcgccgggtgagtcggcccctcaagtgtcaacgtccgcccctcatctgtcag
tgagggccaagttttccgcgaggtatccacaacgccggcggccgcggtgtctcgcac
acggcttcgacggcgtttctggcgcgtttgcagggccatagacggccgccagcccag
cggcgagggcaaccagcccggtgagcgtcggaaaggcgctcggtcttgccttgctcg
tcggtgatgtacactagtcgctggctgctgaacccccagccggaactgaccccacaa
ggccctagcgtttgcaatgcaccaggtcatcattgacccaggcgtgttccaccaggc
cgctgcctcgcaactcttcgcaggcttcgccgacctgctcgcgccacttcttcacgc
gggtggaatccgatccgcacatgaggcggaaggtttccagcttgagcgggtacggct
cccggtgcgagctgaaatagtcgaacatccgtcgggccgtcggcgacagcttgcggt
acttctcccatatgaatttcgtgtagtggtcgccagcaaacagcacgacgatttcct
cgtcgatcaggacctggcaacgggacgttttcttgccacggtccaggacgcggaagc
ggtgcagcagcgacaccgattccaggtgcccaacgcggtcggacgtgaagcccatcg
ccgtcgcctgtaggcgcgacaggcattcctcggccttcgtgtaataccggccattga
tcgaccagcccaggtcctggcaaagctcgtagaacgtgaaggtgatcggctcgccga
taggggtgcgcttcgcgtactccaacacctgctgccacaccagttcgtcatcgtcgg
cccgcagctcgacgccggtgtaggtgatcttcacgtccttgttgacgtggaaaatga
ccttgttttgcagcgcctcgcgcgggattttcttgttgcgcgtggtgaacagggcag
agcgggccgtgtcgtttggcatcgctcgcatcgtgtccggccacggcgcaatatcga
acaaggaaagctgcatttccttgatctgctgcttcgtgtgtttcagcaacgcggcct
gcttggcctcgctgacctgttttgccaggtcctcgccggcggtttttcgcttcttgg
tcgtcatagttcctcgcgtgtcgatggtcatcgacttcgccaaacctgccgcctcct
gttcgagacgacgcgaacgctccacggcggccgatggcgcgggcagggcagggggag
ccagttgcacgctgtcgcgctcgatcttggccgtagcttgctggaccatcgagccga
cggactggaaggtttcgcggggcgcacgcatgacggtgcggcttgcgatggtttcgg
catcctcggcggaaaaccccgcgtcgatcagttcttgcctgtatgccttccggtcaa
acgtccgattcattcaccctccttgcgggattgccccgactcacgccggggcaatgt
gcccttattcctgatttgacccgcctggtgccttggtgtccagataatccaccttat
cggcaatgaagtcggtcccgtagaccgtctggccgtccttctcgtacttggtattcc
gaatcttgccctgcacgaataccagcgaccccttgcccaaatacttgccgtgggcct
cggcctgagagccaaaacacttgatgcggaagaagtcggtgcgctcctgcttgtcgc
cggcatcgttgcgccacatctaggtactaaaacaattcatccagtaaaatataatat
tttattttctcccaatcaggcttgatccccagtaagtcaaaaaatagctcgacatac
tgttcttccccgatatcctccctgatcgaccggacgcagaaggcaatgtcataccac
ttgtccgccctgccgcttctcccaagatcaataaagccacttactttgccatctttc
acaaagatgttgctgtctcccaggtcgccgtgggaaaagacaagttcctcttcgggc
ttttccgtctttaaaaaatcatacagctcgcgcggatctttaaatggagtgtcttct
tcccagttttcgcaatccacatcggccagatcgttattcagtaagtaatccaattcg
gctaagcggctgtctaagctattcgtatagggacaatccgatatgtcgatggagtga
aagagcctgatgcactccgcatacagctcgataatcttttcagggctttgttcatct
tcatactcttccgagcaaaggacgccatcggcctcactcatgagcagattgctccag
ccatcatgccgttcaaagtgcaggacctttggaacaggcagctttccttccagccat
agcatcatgtccttttcccgttccacatcataggtggtccctttataccggctgtcc
gtcatttttaaatataggttttcattttctcccaccagcttatataccttagcagga
gacattccttccgtatcttttacgcagcggtatttttcgatcagttttttcaattcc
ggtgatattctcattttagccatttattatttccttcctcttttctacagtatttaa
agataccccaagaagctaattataacaagacgaactccaattcactgttccttgcat
tctaaaaccttaaataccagaaaacagctttttcaaagttgttttcaaagttggcgt
ataacatagtatcgacggagccgattttgaaaccacaattatgggtgatgctgccaa
cttactgatttagtgtatgatggtgtttttgaggtgctccagtggcttctgtttcta
tcagctgtccctcctgttcagctactgacggggtggtgcgtaacggcaaaagcaccg
ccggacatcagcgctatctctgctctcactgccgtaaaacatggcaactgcagttca
cttacaccgcttctcaacccggtacgcaccagaaaatcattgatatggccatgaatg
gcgttggatgccgggcaaaagcccgcattatgggcgttggcctcaacacgattttac
gtcacttaaaaaactcaggccgcagtcggtaactatgcggtgtgaaataccgcacag
atgcgtaaggagaaaataccgcatcaggcgctcttccgcttcctcgctcactgactc
gctgcgctcggtcgttcggctgcggcgagcggtatcagctcactcaaaggcggtaat
acggttatccacagaatcaggggataacgcaggaaagaacatgtgagcaaaaggcca
gcaaaaggccaggaaccgtaaaaaggccgcgttgctggcgtttttccataggctccg
cccccctgacgagcatcacaaaaatcgacgctcaagtcagaggtggcgaaacccgac
aggactataaagataccaggcgtttccccctggaagctccctcgtgcgctctcctgt
tccgaccctgccgcttaccggatacctgtccgcctttctcccttcgggaagcgtggc
gctttctcatagctcacgctgtaggtatctcagttcggtgtaggtcgttcgctccaa
gctgggctgtgtgcacgaaccccccgttcagcccgaccgctgcgccttatccggtaa
ctatcgtcttgagtccaacccggtaagacacgacttatcgccactggcagcaggtaa
cctcgcgcatacagccgggcagtgacgtcatcgtctgcgcggaaatggacgggcccc
cggcgccagatctggggaac

The pld1hfs2-peaq-ld-sq plasmid encodes the following in multi-cloning site within site 1 (SEQ ID NO:104).

MKKRLTTSTC SSSPSSSVSS STTTSSPIQS EAPRPKRAKR
AKKSSPSGDK SHNPTSPAST RRSSIYRGVT RHRWTGRFEA
HLWDKSSWNS IQNKKGKQVY LGAYDSEEAA AHTYDLAALK
YWGPDTILNF PAETYTKELE EMQRVTKEEY LASLRRQSSG
FSRGVSKYRG VARHHHNGRW EARIGRVFGN KYLYLGTYNT
QEEAAAAYDM AAIEYRGANA VTNFDISNYI DRLKKKGVFP
FPVNQANHQE GILVEAKQEV ETREAKEEPR EEVKQQYVEE
PPQEEEFKEE EKAEQQEAEI VGYSEFAAVV NCCIDSSTIM
EMDRCGDNNE LAWNFCMMDT GFSPFLTDQN LANENPIEYP
ELFNELAFED NIDFMFDDGK HECLNLENLD CCVVGRESNA
ADEVATQLLN FDLLKLAGDV ESNPGPMGKG EELFTGVVPI
LVELDGDVNG HKFSVSGEGE GDATYGKLTL KFICTTGKLP
VPWPTLVTTF GYGLQCFARY PDHMKQHDFF KSAMPEGYVQ
ERTIFFKDDG NYKTRAEVKF EGDTLVNRIE LKGIDFKEDG
NILGHKLEYN YNSHNVYIMA DKQKNGIKVN FKIRHNIEDG
SVQLADHYQQ NTPIGDGPVL LPDNHYLSYQ SALSKDPNEK
RDHMVLLEFV TAAGITLGMD ELYKSCLRSR AQASNSAVDG
TAGPGSSTSL YKKAGSTMAG PIMTSAPSAT TPTGKTMPFK
QPFKTVATLS AKTGNITKPI DPAISKTIDF VYNGYSTVKT
KVDKAPKVNP YLLIAGGLVL SCIISMCLLV PAVIFFPVTI
FLGVATSFAL IALAPVAFVF GWILISSAPI QDKVVVPALD
KVLANEKVAK FLLKE

The pld1hfs2-peaq-ld-sq plasmid encodes the following in site 2 (SEQ ID NO:105).

MISPLASEED EEIVKSVVNG TIPSYSLESK LGDCKRAAEI
RREALQRMMG RSLEGLPVEG FDYESILGQC CEMPVGYVQI
PVGIAGPLLL DGQEYSVPMA TTEGCLVAST NRGCKAIHLS
GGASSVLLKD GMTRAPVVRF ASAMRAADLK FFLENPENFD
SLSIAFNRSS RFAKLQSIQC SIAGKNLYMR FTCSTGDAMG
MNMVSKGVQN VLDFLOSDFP DMDVIGISGN FCSDKKPAAV
NWIQGRGKSV VCEAIIKEEV VKKVLKSSVA SLVELNMLKN
LTGSAIAGAL GGFNAHAGNI VSAIFIATGQ DPAQNVESSH
CITMMEAVND GKDLHISVTM PSIEVGTVGG GTQLASQSAC
LNLLGVKGAS KESPGANSRL LATIVAGSVL AGELSLMSAI
AAGQLVRSHM KYNRSSKDVT KFASSSNAAD EVATQLLNFD
LLKLAGDVES NPGPMADLKS TFLDVYSVLK SDLLQDPSFE
FTHESRQWLE RMLDYNVRGG KLNRGLSVVD SYKLLKQGQD
LTEKETFLSC ALGWCIEWLQ AYFLVLDDIM DNSVTRRGQP
CWFRKPKVGM IAINDGILLR NHIHRILKKH FREMPYYVDL
VDLFNEVEFQ TACGQMIDLI TTFDGEKDLS KYSLQIHRRI
VEYKTAYYSF YLPVACALLM AGENLENHTD VKTVLVDMGI
YFQVQDDYLD CFADPETLGK IGTDIEDFKC SWLVVKALER
CSEEQTKILY ENYGKAEPSN VAKVKALYKE LDLEGAFMEY
EKESYEKLTK LIEAHQSKAI QAVLKSFLAK IYKRQKKSSS
NAADEVATQL LNFDLLKLAG DVESNPGPMA SAILASLLHP
SEVLALVQYK LSPKTQHDYS NDKTRQRLYH HLNMTSRSFS
AVIQDLDEEL KDAICLFYLV LRGLDTIEDD MTIDLDTKLP
YLRTFHEIIY QKGWTFTKNG PNEKDRQLLV EFDAIIEGFL
QLKPAYQTII ADITKRMGNG MAHYATAGIH VETNADYDEY
CHYVAGLVGL GLSEMFSACG FESPLVAERK DLSNSMGLFL
QKTNIARDYL EDLRDNRRFW PKEIWGQYAE TMEDLVKPEN
KEKALQCLSH MIVNAMEHIR DVLEYLSMIK NPSCFKFCAI
PQVMAMATLN LLHSNYKVFT HENIKIRKGE TVWLMKESDS
MDKVAAIFRL YARQINNKSN SLDPHFVDIG VICGEIEQIC
VGRFPGSTIE MKRMQAGVLG GKTGTVL

The plds1hf2-peaq_wr1lv1sqs-ldspmcs1_hmgrlv1fppsmcs2 plasmid has the following sequence (SEQ ID NO:106)

cctgtggttggcatgcacatacaaatggacgaacggataaaccttttcacgcccttt
taaatatccgattattctaataaacgctcttttctcttaggtttacccgccaatata
tcctgtcaaacactgatagtttgtgaaccatcacccaaatcaagttttttggggtcg
aggtgccgtaaagcactaaatcggaaccctaaagggagcccccgatttagagcttga
cggggaaagccggcgaacgtggcgagaaaggaagggaagaaagcgaaaggagcgggc
gccattcaggctgcgcaactgttgggaagggcgatcggtgcgggcctcttcgctatt
acgccagctggcgaaagggggatgtgctgcaaggcgattaagttgggtaacgccagg
gttttcccagtcacgacgttgtaaaacgacggccagtgaattgttaattaagaattc
gagctccaccgcggaaacctcctcggattccattgcccagctatctgtcactttatt
gagaagatagtggaaaaggaaggtggctcctacaaatgccatcattgcgataaagga
aaggccatcgttgaagatgcctctgccgacagtggtcccaaagatggacccccaccc
acgaggagcatcgtggaaaaagaagacgttccaaccacgtcttcaaagcaagtggat
tgatgtgatatctccactgacgtaagggatgacgcacaatcccactatccttcgcaa
gacccttcctctatataaggaagttcatttcatttggagaggtattaaaatcttaat
aggttttgataaaagcgaacgtggggaaacccgaaccaaaccttcttctaaactctc
tctcatctctcttaaagcaaacttctctcttgtctttcttgcgtgagcgatcttcaa
cgttgtcagatcgtgcttcggcaccagtacaacgttttctttcactgaagcgaaatc
aaagatctctttgtggacacgtagtgcggcgccattaaataacgtgtacttgtccta
ttcttgtcggtgtggtcttgggaaaagaaagcttgctggaggctgctgttcagcccc
atacattacttgttacgattctgctgactttcggcgggtgcaatatctctacttctg
cttgacgaggtattgttgcctgtacttctttcttcttcttcttgctgattggttcta
taagaaatctagtattttctttgaaacagagttttcccgtggttttcgaacttggag
aaagattgttaagcttctgtatattctgcccaaattcgcgATGAAGAAGCGCTTAAC
CACTTCCACTTGTTCTTCTTCTCCATCTTCCTCTGTTTCTTCTTCTACTACTACTTC
CTCTCCTATTCAGTCGGAGGCTCCAAGGCCTAAACGAGCCAAAAGGGCTAAGAAATC
TTCTCCTTCTGGTGATAAATCTCATAACCCGACAAGCCCTGCTTCTACCCGACGCAG
CTCTATCTACAGAGGAGTCACTAGACATAGATGGACTGGGAGATTCGAGGCTCATCT
TTGGGACAAAAGCTCTTGGAATTCGATTCAGAACAAGAAAGGCAAACAAGTTTATCT
GGGAGCATATGACAGTGAAGAAGCAGCAGCACATACGTACGATCTGGCTGCTCTCAA
GTACTGGGGACCCGACACCATCTTGAATTTTCCGGCAGAGACGTACACAAAGGAATT
GGAAGAAATGCAGAGAGTGACAAAGGAAGAATATTTGGCTTCTCTCCGCCGCCAGAG
CAGTGGTTTCTCCAGAGGCGTCTCTAAATATCGCGGCGTCGCTAGGCATCACCACAA
CGGAAGATGGGAGGCTCGGATCGGAAGAGTGTTTGGGAACAAGTACTTGTACCTCGG
CACCTATAATACGCAGGAGGAAGCTGCTGCAGCATATGACATGGCTGCGATTGAGTA
TCGAGGCGCAAACGCGGTTACTAATTTCGACATTAGTAATTACATTGACCGGTTAAA
GAAGAAAGGTGTTTTCCCGTTCCCTGTGAACCAAGCTAACCATCAAGAGGGTATTCT
TGTTGAAGCCAAACAAGAAGTTGAAACGAGAGAAGCGAAGGAAGAGCCTAGAGAAGA
AGTGAAACAACAGTACGTGGAAGAACCACCGCAAGAAGAAGAAGAGAAGGAAGAAGA
GAAAGCAGAGCAACAAGAAGCAGAGATTGTAGGATATTCAGAAGAAGCAGCAGTGGT
CAATTGCTGCATAGACTCTTCAACCATAATGGAAATGGATCGTTGTGGGGACAACAA
TGAGCTGGCTTGGAACTTCTGTATGATGGATACAGGGTTTTCTCCGTTTTTGACTGA
TCAGAATCTCGCGAATGAGAATCCCATAGAGTATCCGGAGCTATTCAATGAGTTAGC
ATTTGAGGACAACATCGACTTCATGTTCGATGATGGGAAGCACGAGTGCTTGAACTT
GGAAAATCTGGATTGTTGCGTGGTGGGAAGAGAGTCAAATGCAGCAGACGAAGTTGC
TACTCAACTTTTGAATTTTGACTTGCTGAAGTTGGCTGGTGATGTTGAGTCAAACCG
TGGACCTATGGCCAGTGCTATTCTTGCTTCATTACTCCACCCATCAGAAGTGTTGGC
ACTTGTGCAGTACAAGCTTTCACCCAAAACCCAGCATGATTACTCTAACGACAAAAC
TAGGCAAAGAGTTTATGATGATGTTAATATGACTTCCCGATCCTTCTCTGCCGTCAT
ACAGGACCTTGATGAAGAGTTAAAGGATGCTATATGCTTATTCTATCTGGTGCTGAG
AGGCTTAGATACTATAGAAGACGACATGACCATCGACCTTGACACTAAATTGCCTTA
CCTTCGTACGTTCCACGAAATCATATACCAGAAAGGCTGGACTTTCACTAAGAACGG
CCCAAATGAAAAAGATAGGCAATTACTGGTAGAATTTGACGCCATCATAGAGGGCTT
CCTTCAATTGAAGCCAGCCTATCAGACTATCATTGCCGATATAACCAAACGTATGGG
GAACGGAATGGCACACTACGCTACGGCAGGGATACATGTTGAGACCAACGCAGACTA
CGACGAGTACTGCCACTATGTCGCTGGTTTGGTGGGGCTGGGTCTCTCTGAAATGTT
TTCCGCATGTGGGTTCGAAAGTCCTCTTGTGGCAGAAAGAAAAGACCTTAGCAACAG
CATGGGACTTTTCCTTCAGAAGACGAACATTGCACGTGATTATCTTGAAGACCTCAG
AGACAATCGTCGATTTTGGCCCAAGGAAATATGGGGGCAGTATGCTGAGACTATGGA
GGACTTGGTAAAGCCCGAAAATAAAGAAAAGGCCCTCCAATGCCTCTCCCATATGAT
CGTCAATGCAATGGAGCATATCAGAGACGTTTTGGAGTATCTCTCTATGATAAAGAA
TCCGAGCTGCTTCAAATTTTGTGCTATTCCACAAGTCATGGCTATGGCCACATTAAA
CCTGCTTCATTCCAACTACAAAGTGTTCACGCATGAGAATATCAAGATCCGTAAAGG
TGAGACAGTGTGGCTTATGAAAGAAAGTGACAGTATGGACAAGGTAGCTGCTATCTT
TAGGTTGTACGCCCGACAAATTAACAACAAGTCCAACTCTCTTGATCCCCATTTTGT
GGATATAGGGGTGATTTGCGGTGAGATCGAGCAAATTTGCGTAGGAAGGTTCCCTGG
CTCCACAATAGAAATGAAGCGAATGCAGGCTGGAGTCTTAGGGGGGAAAACTGGAAC
GGTCCTGATGGCCGGCCCCATCATGACCTCTGCGCCCTCCGCGACCACGCCCACGGG
CAAGACAATGCCGTTCAAGCAGCCTTTCAAGACTGTGGCCACGCTGTCCGCCAAGAC
TGGCAACATTACCAAGCCCATCGACCCTGCCATCTCCAAGACCATTGACTTCGTCTA
CAATGGTTACTCGACGGTCAAGACCAAGGTTGACAAGGCCCCTAAGGTAAACCCCTA
CCTGCTCATTGCCGGCGGCCTCGTCCTCTCGTGCATCATCTCCATGTGCCTGCTCGT
CCCGGCCGTGATCTTCTTCCCCGTCACCATCTTCCTGGGTGTCGCTACGTCGTTTGC
GCTCATTGCATTGGCCCCCGTGGCTTTTGTGTTCGGGTGGATCCTGATCTCCTCTGC
TCCGATCCAGGATAAGGTGGTGGTGCCCGCCTTGGACAAGGTGCTGGCCAATAAGAA
GGTGGCGAAGTTCCTCCTCAAGGAGTAAtcgaggcctttaactctggtttcattaaa
ttttctttagtttgaatttactgttattcggtgtgcatttctatgtttggtgagcgg
ttttctgtgctcagagtgtgtttattttatgtaatttaatttctttgtgagctcctg
tttagcaggtcgtcccttcagcaaggacacaaaaagattttaattttattaaaaaaa
aaaaaaaaaaagaccgggaattcgatatcaagcttatcgacctgcagatcgttcaaa
catttggcaataaagtttcttaagattgaatcctgttgccggtcttgcgatgattat
catataatttctgttgaattacgttaagcatgtaataattaacatgtaatgcatgac
gttatttatgagatgggtttttatgattagagtcccgcaattatacatttaatacgc
gatagaaaacaaaatatagcgcgcaaactaggataaattatcgcgcgcggtgtcatc
tatgttactagatctctagagtctcaagcttggcgcgccagcttggcgtaatcatgg
tcatagctgttgcgattaagaattcgagctcggtacccccctactccaaaaatgtca
aagatacagtctcagaagaccaaagggctattgagacttttcaacaaagggtaattt
cgggaaacctcctcggattccattgcccagctatctgtcacttcatcgaaaggacag
tagaaaaggaaggtggctcctacaaatgccatcattgcgataaaggaaaggctatca
ttcaagatgcctctgccgacagtggtcccaaagatggacccccacccacgaggagca
tcgtggaaaaagaagacgttccaaccacgtcttcaaagcaagtggattgatgtgaca
tctccactgacgtaagggatgacgcacaatcccactatccttcgcaagacccttcct
ctatataaggaagttcatttcatttggagaggacagcccaagcttcgactctagagg
atccccttaaatcgatATTTATGATTTCGCCTCTGGCATCCGAGGAGGATGAGGAAA
TTGTTAAATCTGTTGTTAATGGAACGATTCCTTCGTATTCGTTGGAATCGAAGCTTG
GGGATTGTAAAAGAGCGGCTGAGATTCGACGGGAGGCTTTGCAGAGAATGATGGGGA
GGTCGTTGGAGGGTTTACCTGTTGAAGGATTCGATTATGAGTCGATTTTAGGTCAGT
GCTGTGAAATGCCTGTTGGTTATGTGCAGATTCCGGTTGGAATTGCTGGGCCGTTGC
TGCTAGACGGGCAAGAGTACTCTGTTCCGATGGCGACCACCGAGGGTTGTTTGGTTG
CTAGCACTAATAGAGGGTGTAAAGCGATCCATTTGTCAGGTGGTGCTAGTAGTGTCT
TGTTGAAGGATGGCATGACTAGAGCTCCCGTTGTTCGATTCGCCTCGGCCATGAGGG
CCGCGGATTTGAAGTTTTTCTTAGAGAATCCTGAGAATTTCGATAGCTTGTCCATCG
CTTTCAATAGGTCCAGTAGATTTGCAAAGCTCCAAAGCATACAATGTTCTATTGCTG
GAAAGAATCTATATATGAGATTCACCTGCAGCACTGGTGATGCAATGGGGATGAACA
TGGTTTCCAAAGGGGTTCAAAACGTTCTTGACTTCCTTCAAAGTGATTTCCCTGACA
TGGATGTTATTGGCATCTCAGGAAATTTTTGTTCGGACAAGAAGCCAGCTGCTGTGA
ACTGGATTCAAGGGCGAGGCAAATCGGTTGTTTGCGAGGCAATTATCAAGGAAGAGG
TGGTGAAGAAGGTATTGAAATCAAGTGTTGCTTCACTAGTAGAGCTGAACATGCTCA
AGAATCTTACTGGTTCAGCTATTGCTGGAGCTCTTGGTGGATTCAATGCACATGCTG
GCAACATAGTCTCTGCAATTTTCATTGCCACTGGCCAGGATCCAGCCCAGAATGTTG
AGAGTTCTCATTGCATCACCATGATGGAAGCTGTCAATGATGGAAAAGATCTCCACA
TCTCTGTAACCATGCCTTCAATCGAGGTAGGAACAGTTGGAGGAGGGACACAACTAG
CATCCCAATCAGCATGTCTGAACCTACTCGGTGTAAAAGGAGCAAGTAAAGAATCAC
CAGGAGCAAACTCAAGGCTCCTAGCCACAATAGTAGCTGGTTCAGTCCTAGCTGGTG
AACTCTCCCTAATGTCAGCCATAGCAGCAGGACAACTAGTCCGGAGCCACATGAAGT
ACAACAGATCCAGCAAAGATGTAACCAAATTTGCATCATCTTCAAATGCAGCAGACG
AAGTTGCTACTCAACTTTTGAATTTTGACTTGCTGAAGTTGGCTGGTGATGTTGAGT
CAAACCCTGGACCTATGGCGGATCTGAAATCAACCTTCCTCGACGTTTACTCTGTTC
TCAAGTCTGATCTGCTTCAAGATCCTTCCTTTGAATTCACCCACGAATCTCGTCAAT
GGCTTGAACGGATGCTTGACTAGAATGTACGCGGAGGGAAGCTAAATCGTGGTCTCT
CTGTGGTTGATAGCTACAAGCTGTTGAAGCAAGGTCAAGACTTGACGGAGAAAGAGA
CTTTCCTCTCATGTGCTCTTGGTTGGTGCATTGAATGGCTTCAAGCTTATTTCCTTG
TGCTTGATGACATCATGGACAACTCTGTCACACGCCGTGGCCAGCCTTGTTGGTTTA
GAAAGCCAAAGGTTGGTATGATTGCCATTAACGATGGGATTCTACTTCGCAATCATA
TCCACAGGATTCTCAAAAAGCACTTCAGGGAAATGCCTTACTATGTTGACCTCGTTG
ATTTGTTTAACGAGGTAGAGTTTCAAACAGCTTGCGGCCAGATGATTGATTTGATCA
CCACCTTTGATGGAGAAAAAGATTTGTCTAAGTACTCCTTGCAAATCCATCGGCGTA
TTGTTGAGTACAAAACAGCTTATTACTCATTTTATCTTCCTGTTGCTTGCGCATTGC
TCATGGCGGGAGAAAATTTGGAAAACCATAGTGATGTGAAGACTGTTCTTGTTGACA
TGGGAATTTACTTTCAAGTACAGGATGATTATCTGGACTGTTTTGCTGATCCTGAGA
CACTTGGCAAGATAGGGACAGACATAGAAGATTTCAAATGCTCCTGGTTGGTAGTTA
AGGCATTGGAACGCTGCAGTGAAGAACAAACTAAGATACTATACGAGAACTATGGTA
AAGCCGAACCATCAAACGTTGCTAAGGTGAAAGCTCTCTACAAAGAGCTTGATCTCG
AGGGAGCGTTCATGGAATATGAGAAGGAAAGCTATGAGAAGCTGACAAAGTTGATCG
AAGCTCACCAGAGTAAAGCAATTCAAGCAGTGCTAAAATCTTTCTTGGCTAAGATCT
ACAAGAGGCAGAAGTAAAAATCCTCAGCAATTGggggagctcgaattcgctgaaatc
accagtctctctctacaaatctatctctctctattttctccataaataatgtgtgag
tagtttcccgataagggaaattagggttcttatagggtttcgctcatgtgttgagca
tataagaaacccttagtatgtatttgtatttgtaaaatacttctatcaataaaattt
ctaattcctaaaaccaaaatccagtactaaaatccagatctcctaaagtccctatag
atctttgtcgtgaatataaaccagacacgagacgactaaacctggagcccagacgcc
gttcgaagctagaagtaccgcttaggcaggaggccgttagggaaaagatgctaaggc
agggttggttacgttgactcccccgtaggtttggtttaaatatgatgaagtggacgg
aaggaaggaggaagacaaggaaggataaggttgcaggccctgtgcaaggtaagaaga
tggaaatttgatagaggtacgctactatacttatactatacgctaagggaatgcttg
tatttataccctataccccctaataaccccttatcaatttaagaaataatccgcata
agcccccgcttaaaaattggtatcagagccatgaataggtctatgaccaaaactcaa
gaggataaaacctcaccaaaatacgaaagagttcttaactctaaagataaaagatgg
cgcgtggccggcctacagtatgagcggagaattaagggagtcacgttatgacccccg
ccgatgacgcgggacaagccgttttacgtttggaactgacagaaccgcaacgttgaa
ggagccactcagccgcgggtttctggagtttaatgagctaagcacatacgtcagaaa
ccattattgcgcgttcaaaagtcgcctaaggtcactatcagctagcaaatatttctt
gtcaaaaatgctccactgacgttccataaattcccctcggtatccaattagagtctc
atattcactctcaatccaaataatctgcaccggatctggatcgtttcgcatgattga
acaagatggattgcacgcaggttctccggccgcttgggtggagaggctattcggcta
tgactgggcacaacagacaatcggctgctctgatgccgccgtgttccggctgtcagc
gcaggggcgcccggttctttttgtcaagaccgacctgtccggtgccctgaatgaact
gcaggacgaggcagcgcggctatcgtggctggccacgacgggcgttccttgcgcagc
tgtgctcgacgttgtcactgaagcgggaagggactggctgctattgggcgaagtgcc
ggggcaggatctcctgtcatctcaccttgctcctgccgagaaagtatccatcatggc
tgatgcaatgcggcggctgcatacgcttgatccggctacctgcccattcgaccacca
agcgaaacatcgcatcgagcgagcacgtactcggatggaagccggtcttgtcgatca
ggatgatctggacgaagagcatcaggggctcgcgccagccgaactgttcgccaggct
caaggcgcgcatgcccgacggcgatgatctcgtcgtgacccatggcgatgcctgctt
gccgaatatcatggtggaaaatggccgcttttctggattcatcgactgtggccggct
gggtgtggcggaccgctatcaggacatagcgttggctacccgtgatattgctgaaga
gcttggcggcgaatgggctgaccgcttcctcgtgctttacggtatcgccgctcccga
ttcgcagcgcatcgccttctatcgccttcttgacgagttcttctgagcgggactctg
gggttcgaaatgaccgaccaagcgacgcccaacctgccatcacgagatttcgattcc
accgccgccttctatgaaaggttgggcttcggaatcgttttccgggacgccggctgg
atgatcctccagcgcggggatctcatgctggagttcttcgcccacgggatctctgcg
gaacaggcggtcgaaggtgccgatatcattacgacagcaacggccgacaagcacaac
gccacgatcctgagcgacaatatgatcgcggcgtccacatcaacggcgtcggcggcg
actgcccaggcaagaccgagatgcaccgcgatatcttgctgcgttcggatattttcg
tggagttcccgccacagacccggatgatccccgatcgttcaaacatttggcaataaa
gtttcttaagattgaatcctgttgccggtcttgcgatgattatcatataatttctgt
tgaattacgttaagcatgtaataattaacatgtaatgcatgacgttatttatgagat
gggtttttatgattagagtcccgcaattatacatttaatacgcgatagaaaacaaaa
tatagcgcgcaaactaggataaattatcgcgcgcggtgtcatctatgttactagatc
gggactgtaggccggccctcactggtgaaaagaaaaaccaccccagtacattaaaaa
cgtccgcaatgtgttattaagttgtctaagcgtcaatttgtttacaccacaatatat
cctgccaccagccagccaacagctccccgaccggcagctcggcacaaaatcaccact
cgatacaggcagcccatcagtccgggacggcgtcagcgggagagccgttgtaaggcg
gcagactttgctcatgttaccgatgctattcggaagaacggcaactaagctgccggg
tttgaaacacggatgatctcgcggagggtagcatgttgattgtaacgatgacagagc
gttgctgcctgtgatcaaatatcatctccctcgcagagatccgaattatcagccttc
ttattcatttctcgcttaaccgtgacagagtagacaggctgtctcgcggccgagggg
cgcagcccctgggggggatgggaggcccgcgttagcgggccgggagggttcgagaag
ggggggcaccccccttcggcgtgcgcggtcacgcgcacagggcgcagccctggttaa
aaacaaggtttataaatattggtttaaaagcaggttaaaagacaggttagcggtggc
cgaaaaacgggcggaaacccttgcaaatgctggattttctgcctgtggacagcccct
caaatgtcaataggtgcgcccctcatctgtcagcactctgcccctcaagtgtcaagg
atcgcgcccctcatctgtcagtagtcgcgcccctcaagtgtcaataccgcagggcac
ttatccccaggcttgtccacatcatctgtgggaaactcgcgtaaaatcaggcgtttt
cgccgatttgcgaggctggccagctccacgtcgccggccgaaatcgagcctgcccct
catctgtcaacgccgcgccgggtgagtcggcccctcaagtgtcaacgtccgcccctc
atctgtcagtgagggccaagttttccgcgaggtatccacaacgccggcggccgcggt
gtctcgcacacggcttcgacggcgtttctggcgcgtttgcagggccatagacggccg
ccagcccagcggcgagggcaaccagcccggtgagcgtcggaaaggcgctcggtcttg
ccttgctcgtcggtgatgtacactagtcgctggctgctgaacccccagccggaactg
accccacaaggccctagcgtttgcaatgcaccaggtcatcattgacccaggcgtgtt
ccaccaggccgctgcctcgcaactcttcgcaggcttcgccgacctgctcgcgccact
tcttcacgcgggtggaatccgatccgcacatgaggcggaaggtttccagcttgagcg
ggtacggctcccggtgcgagctgaaatagtcgaacatccgtcgggccgtcggcgaca
gcttgcggtacttctcccatatgaatttcgtgtagtggtcgccagcaaacagcacga
cgatttcctcgtcgatcaggacctggcaacgggacgttttcttgccacggtccagga
cgcggaagcggtgcagcagcgacaccgattccaggtgcccaacgcggtcggacgtga
agcccatcgccgtcgcctgtaggcgcgacaggcattcctcggccttcgtgtaatacc
ggccattgatcgaccagcccaggtcctggcaaagctcgtagaacgtgaaggtgatcg
gctcgccgataggggtgcgcttcgcgtactccaacacctgctgccacaccagttcgt
catcgtcggcccgcagctcgacgccggtgtaggtgatcttcacgtccttgttgacgt
ggaaaatgaccttgttttgcagcgcctcgcgcgggattttcttgttgcgcgtggtga
acagggcagagcgggccgtgtcgtttggcatcgctcgcatcgtgtccggccacggcg
caatatcgaacaaggaaagctgcatttccttgatctgctgcttcgtgtgtttcagca
acgcggcctgcttggcctcgctgacctgttttgccaggtcctcgccggcggtttttc
gcttcttggtcgtcatagttcctcgcgtgtcgatggtcatcgacttcgccaaacctg
ccgcctcctgttcgagacgacgcgaacgctccacggcggccgatggcgcgggcaggg
cagggggagccagttgcacgctgtcgcgctcgatcttggccgtagcttgctggacca
tcgagccgacggactggaaggtttcgcggggcgcacgcatgacggtgcggcttgcga
tggtttcggcatcctcggcggaaaaccccgcgtcgatcagttcttgcctgtatgcct
tccggtcaaacgtccgattcattcaccctccttgcgggattgccccgactcacgccg
gggcaatgtgcccttattcctgatttgacccgcctggtgccttggtgtccagataat
ccaccttatcggcaatgaagtcggtcccgtagaccgtctggccgtccttctcgtact
tggtattccgaatcttgccctgcacgaataccagcgaccccttgcccaaatacttgc
cgtgggcctcggcctgagagccaaaacacttgatgcggaagaagtcggtgcgctcct
gcttgtcgccggcatcgttgcgccacatctaggtactaaaacaattcatccagtaaa
atataatattttattttctcccaatcaggcttgatccccagtaagtcaaaaaatagc
tcgacatactgttcttccccgatatcctccctgatcgaccggacgcagaaggcaatg
tcataccacttgtccgccctgccgcttctcccaagatcaataaagccacttactttg
ccatctttcacaaagatgttgctgtctcccaggtcgccgtgggaaaagacaagttcc
tcttcgggcttttccgtctttaaaaaatcatacagctcgcgcggatctttaaatgga
gtgtcttcttcccagttttcgcaatccacatcggccagatcgttattcagtaagtaa
tccaattcggctaagcggctgtctaagctattcgtatagggacaatccgatatgtcg
atggagtgaaagagcctgatgcactccgcatacagctcgataatcttttcagggctt
tgttcatcttcatactcttccgagcaaaggacgccatcggcctcactcatgagcaga
ttgctccagccatcatgccgttcaaagtgcaggacctttggaacaggcagctttcct
tccagccatagcatcatgtccttttcccgttccacatcataggtggtccctttatac
cggctgtccgtcatttttaaatataggttttcattttctcccaccagcttatatacc
ttagcaggagacattccttccgtatcttttacgcagcggtatttttcgatcagtttt
ttcaattccggtgatattctcattttagccatttattatttccttcctcttttctac
agtatttaaagataccccaagaagctaattataacaagacgaactccaattcactgt
tccttgcattctaaaaccttaaataccagaaaacagctttttcaaagttgttttcaa
agttggcgtataacatagtatcgacggagccgattttgaaaccacaattatgggtga
tgctgccaacttactgatttagtgtatgatggtgtttttgaggtgctccagtggctt
ctgtttctatcagctgtccctcctgttcagctactgacggggtggtgcgtaacggca
aaagcaccgccggacatcagcgctatctctgctctcactgccgtaaaacatggcaac
tgcagttcacttacaccgcttctcaacccggtacgcaccagaaaatcattgatatgg
ccatgaatggcgttggatgccgggcaacagcccgcattatgggcgttggcctcaaca
cgattttacgtcacttaaaaaactcaggccgcagtcggtaactatgcggtgtgaaat
accgcacagatgcgtaaggagaaaataccgcatcaggcgctcttccgcttcctcgct
cactgactcgctgcgctcggtcgttcggctgcggcgagcggtatcagctcactcaaa
ggcggtaatacggttatccacagaatcaggggataacgcaggaaagaacatgtgagc
aaaaggccagcaaaaggccaggaaccgtaaaaaggccgcgttgctggcgtttttcca
taggctccgcccccctgacgagcatcacaaaaatcgacgctcaagtcagaggtggcg
aaacccgacaggactataaagataccaggcgtttccccctggaagctccctcgtgcg
ctctcctgttccgaccctgccgcttaccggatacctgtccgcctttctcccttcggg
aagcgtggcgctttctcatagctcacgctgtaggtatctcagttcggtgtaggtcgt
tcgctccaagctgggctgtgtgcacgaaccccccgttcagcccgaccgctgcgcctt
atccggtaactatcgtcttgagtccaacccggtaagacacgacttatcgccactggc
agcaggtaacctcgcgcatacagccgggcagtgacgtcatcgtctgcgcggaaatgg
acgggcccccggcgccagatctggggaac

The plds1hf2-peaq_wri1lv1sqs-ldspmcs1_hmgrlv1fppsmcs2 plasmid encodes the following in multi-cloning site within site 1 (SEQ ID NO:107).

MKKRLTTSTC SSSPSSSVSS STTTSSPIQS EAPRPKRAKR
AKKSSPSGDK SHNPTSPAST RRSSIYRGVT RHRWTGRFEA
HLWDKSSWNS IQNKKGKQVY LGAYDSEEAA AHTYDLAALK
YWGPDTILNF PAETYTKELE EMQRVTKEEY LASLRRQSSG
FSRGVSKYRG VARHHHNGRW EARIGRVFGN KYLYLGTYNT
QEEAAAAYDM AAIEYRGANA VTNFDISNYI DRLKKKGVFP
EPVNOANHQE GILVEAKQEV ETREAKEEPR EEVKQQYVEE
PPQEEEEKEE EKAEQQEAEI VGYSEEAAVV NCCIDSSTIM
EMDRCGDNNE LAWNFCMMDT GFSPFLTDQN LANENPIEYP
ELFNELAFED NIDFMFDDGK HECLNLENLD CCVVGRESNA
ADEVATQLLN FDLLKLAGDV ESNPGPMASA ILASLLHPSE
VLALVQYKLS PKTQHDYSND KTRQRLYHHL NMTSRSFSAV
IQDLDEELKD AICLFYLVLR GLDTIEDDMT IDLDTKLPYL
RTFHEIIYQK GWTFTKNGPN EKDRQLLVEF DAIIEGFLQL
KPAYQTIIAD ITKRMGNGMA HYATAGIHVE TNADYDEYCH
YVAGLVGLGL SEMFSACGFE SPLVAERKDL SNSMGLFLQK
TNIARDYLED LRDNRRFWPK EIWGQYAETM EDLVKPENKE
KALQCLSHMI VNAMEHIRDV LEYLSMIKNP SCFKFCAIPQ
VMAMATLNLL HSNYKVFTHE NIKIRKGETV WLMKESDSMD
KVAAIFRLYA RQINNKSNSL DPHFVDIGVI CGEIEQICVG
REPGSTIEMK RMQAGVLGGK TGTVLMAGPI MTSAPSATTP
TGKTMPFKQP FKTVATLSAK TGNITKPIDP AISKTIDFVY
NGYSTVKTKV DKAPKVNPYL LIAGGLVLSC IISMCLLVPA
VIFFPVTIFL GVATSFAIIA LAPVAFVFGW ILISSAPIQD
KVVVPALDKV LANKKVAKFL LKE-

The plds1hf2-peaq_wri1lv1sqs-ldspmcs1_hmgrlv1fppsmcs2 plasmid encodes the following in site 2 (SEQ ID NO:108).

MISPLASEED EEIVKSVVNG TIPSYSLESK LGDCKRAAEI
RREALQRMMG RSLEGLPVEG FDYESILGQC CEMPVGYVQI
PVGIAGPLLL DGQEYSVPMA TTEGCLVAST NRGCKAIHLS
GGASSVLLKD GMTRAPVVRF ASAMRAADLK FFLENPENFD
SLSIAFNRSS RFAKLQSIQC SIAGKNLYMR FTCSTGDAMG
MNMVSKGVQN VLDFLQSDFP DMDVIGISGN FCSDKKPAAV
NWIQGRGKSV VCEAIIKEEV VKKVLKSSVA SLVELNMLKN
LTGSAIAGAL GGFNAHAGNI VSAIFIATGQ DPAQNVESSH
CITMMEAVND GKDLHISVTM PSIEVGTVGG GTQLASQSAC
LNLLGVKGAS KESPGANSRL LATIVAGSVL AGELSLMSAI
AAGQLVRSHM KYNRSSKDVT KFASSSNAAD EVATQLLNFD
LLKLAGDVES NPGPMADLKS TFLDVYSVLK SDLLQDPSFE
FTHESRQWLE RMLDYNVRGG KLNRGLSVVD SYKLLKQGQD
LTEKETFLSC ALGWCIEWLQ AYFLVLDDIM DNSVTRRGQP
CWFRKPKVGM IAINDGILLR NHIHRILKKH FREMPYYVDL
VDLFNEVEFQ TACGQMIDLI TTFDGEKDLS KYSLQIHRRI
VEYKTAYYSF YLPVACALLM AGENLENHTD VKTVLVDMGI
YFQVQDDYLD CFADPETLGK IGTDIEDFKC SWLVVKALER
CSEEQTKILY ENYGKAEPSN VAKVKALYKE LDLEGAFMEY
EKESYEKLTK LIEAHQSKAI QAVLKSFLAK IYKRQK

The pwh1slf2-peaq_wri1lv1hmgrmcs1_sqs-ldsp-fppsmcs2 plasmid has the following sequence (SEQ ID NO:109)

cctgtggttggcatgcacatacaaatggacgaacggataaaccttttcacgcccttt
taaatatccgattattctaataaacgctcttttctcttaggtttacccgccaatata
tcctgtcaaacactgatagtttgtgaaccatcacccaaatcaagttttttggggtcg
aggtgccgtaaagcactaaatcggaaccctaaagggagcccccgatttagagcttga
cggggaaagccggcgaacgtggcgagaaaggaagggaagaaagcgaaaggagcgggc
gccattcaggctgcgcaactgttgggaagggcgatcggtgcgggcctcttcgctatt
acgccagctggcgaaagggggatgtgctgcaaggcgattaagttgggtaacgccagg
gttttcccagtcacgacgttgtaaaacgacggccagtgaattgttaattaagaattc
gagctccaccgcggaaacctcctcggattccattgcccagctatctgtcactttatt
gagaagatagtggaaaaggaaggtggctcctacaaatgccatcattgcgataaagga
aaggccatcgttgaagatgcctctgccgacagtggtcccaaagatggacccccaccc
acgaggagcatcgtggaaaaagaagacgttccaaccacgtcttcaaagcaagtggat
tgatgtgatatctccactgacgtaagggatgacgcacaatcccactatccttcgcaa
gacccttcctctatataaggaagttcatttcatttggagaggtattaaaatcttaat
aggttttgataaaagcgaacgtggggaaacccgaaccaaaccttcttctaaactctc
tctcatctctcttaaagcaaacttctctcttgtctttcttgcgtgagcgatcttcaa
cgttgtcagatcgtgcttcggcaccagtacaacgttttctttcactgaagcgaaatc
aaagatctctttgtggacacgtagtgcggcgccattaaataacgtgtacttgtccta
ttcttgtcggtgtggtcttgggaaaagaaagcttgctggaggctgctgttcagcccc
atacattacttgttacgattctgctgactttcggcgggtgcaatatctctacttctg
cttgacgaggtattgttgcctgtacttctttcttcttcttcttgctgattggttcta
taagaaatctagtattttctttgaaacagagttttcccgtggttttcgaacttggag
aaagattgttaagcttctgtatattctgcccaaattcgcgATGAAGAAGCGCTTAAC
CACTTCCACTTGTTCTTCTTCTCCATCTTCCTCTGTTTCTTCTTCTACTACTACTTC
CTCTCCTATTCAGTCGGAGGCTCCAAGGCCTAAACGAGCCAAAAGGGCTAAGAAATC
TTCTCCTTCTGGTGATAAATCTCATAACCCGACAAGCCCTGCTTCTACCCGACGCAG
CTCTATCTACAGAGGAGTCACTAGACATAGATGGACTGGGAGATTCGAGGCTCATCT
TTGGGACAAAAGCTCTTGGAATTCGATTCAGAACAAGAAAGGCAAACAAGTTTATCT
GGGAGCATATGACAGTGAAGAAGCAGCAGCACATACGTACGATCTGGCTGCTCTCAA
GTACTGGGGACCCGACACCATCTTGAATTTTCCGGCAGAGACGTACACAAAGGAATT
GGAAGAAATGCAGAGAGTGACAAAGGAAGAATATTTGGCTTCTCTCCGCCGCCAGAG
CAGTGGTTTCTCCAGAGGCGTCTCTAAATATCGCGGCGTCGCTAGGCATCACCACAA
CGGAAGATGGGAGGCTCGGATCGGAAGAGTGTTTGGGAACAAGTACTTGTACCTCGG
CACCTATAATACGCAGGAGGAAGCTGCTGCAGCATATGACATGGCTGCGATTGAGTA
TCGAGGCGCAAACGCGGTTACTAATTTCGACATTAGTAATTACATTGACCGGTTAAA
GAAGAAAGGTGTTTTCCCGTTCCCTGTGAACCAAGCTAACCATCAAGAGGGTATTCT
TGTTGAAGCCAAACAAGAAGTTGAAACGAGAGAAGCGAAGGAAGAGCCTAGAGAAGA
AGTGAAACAACAGTACGTGGAAGAACCACCGCAAGAAGAAGAAGAGAAGGAAGAAGA
GAAAGCAGAGCAACAAGAAGCAGAGATTGTAGGATATTCAGAAGAAGCAGCAGTGGT
CAATTGCTGCATAGACTCTTCAACCATAATGGAAATGGATCGTTGTGGGGACAACAA
TGAGCTGGCTTGGAACTTCTGTATGATGGATACAGGGTTTTCTCCGTTTTTGACTGA
TCAGAATCTCGCGAATGAGAATCCCATAGAGTATCCGGAGCTATTCAATGAGTTAGC
ATTTGAGGACAACATCGACTTCATGTTCGATGATGGGAAGCACGAGTGCTTGAACTT
GGAAAATCTGGATTGTTGCGTGGTGGGAAGAGAGTCAAATGCAGCAGACGAAGTTGC
TACTCAACTTTTGAATTTTGACTTGCTGAAGTTGGCTGGTGATGTTGAGTCAAACCC
TGGACCTATGATTTCGCCTCTGGCATCCGAGGAGGATGAGGAAATTGTTAAATCTGT
TGTTAATGGAACGATTCCTTCGTATTCGTTGGAATCGAAGCTTGGGGATTGTAAAAG
AGCGGCTGAGATTCGACGGGAGGCTTTGCAGAGAATGATGGGGAGGTCGTTGGAGGG
TTTACCTGTTGAAGGATTCGATTATGAGTCGATTTTAGGTCAGTGCTGTGAAATGCC
TGTTGGTTATGTGCAGATTCCGGTTGGAATTGCTGGGCCGTTGCTGCTAGACGGGCA
AGAGTACTCTGTTCCGATGGCGACCACCGAGGGTTGTTTGGTTGCTAGCACTAATAG
AGGGTGTAAAGCGATCCATTTGTCAGGTGGTGCTAGTAGTGTCTTGTTGAAGGATGG
CATGACTAGAGCTCCCGTTGTTCGATTCGCCTCGGCCATGAGGGCCGCGGATTTGAA
GTTTTTCTTAGAGAATCCTGAGAATTTCGATAGCTTGTCCATCGCTTTCAATAGGTC
CAGTAGATTTGCAAAGCTCCAAAGCATACAATGTTCTATTGCTGGAAAGAATCTATA
TATGAGATTCACCTGCAGCACTGGTGATGCAATGGGGATGAACATGGTTTCCAAAGG
GGTTCAAAACGTTCTTGACTTCCTTCAAAGTGATTTCCCTGACATGGATGTTATTGG
CATCTCAGGAAATTTTTGTTCGGACAAGAAGCCAGCTGCTGTGAACTGGATTCAAGG
GCGAGGCAAATCGGTTGTTTGCGAGGCAATTATCAAGGAAGAGGTGGTGAAGAAGGT
ATTGAAATCAAGTGTTGCTTCACTAGTAGAGCTGAACATGCTCAAGAATCTTACTGG
TTCAGCTATTGCTGGAGCTCTTGGTGGATTCAATGCACATGCTGGCAACATAGTCTC
TGCAATTTTCATTGCCACTGGCCAGGATCCAGCCCAGAATGTTGAGAGTTCTCATTG
CATCACCATGATGGAAGCTGTCAATGATGGAAAAGATCTCCACATCTCTGTAACCAT
GCCTTCAATCGAGGTAGGAACAGTTGGAGGAGGGACACAACTAGCATCCCAATCAGC
ATGTCTGAACCTACTCGGTGTAAAAGGAGCAAGTAAAGAATCACCAGGAGCAAACTC
AAGGCTCCTAGCCACAATAGTAGCTGGTTCAGTCCTAGCTGGTGAACTCTCCCTAAT
GTCAGCCATAGCAGCAGGACAACTAGTCCGGAGCCACATGAAGTACAACAGATCCAG
CAAAGATGTAACCAAATTTGCATCATCTTAAtcgaggcctttaactctggtttcatt
aaattttctttagtttgaatttactgttattcggtgtgcatttctatgtttggtgag
cggttttctgtgctcagagtgtgtttattttatgtaatttaatttctttgtgagctc
ctgtttagcaggtcgtcccttcagcaaggacacaaaaagattttaattttattaaaa
aaaaaaaaaaaaaagaccgggaattcgatatcaagcttatcgacctgcagatcgttc
aaacatttggcaataaagtttcttaagattgaatcctgttgccggtcttgcgatgat
tatcatataatttctgttgaattacgttaagcatgtaataattaacatgtaatgcat
gacgttatttatgagatgggtttttatgattagagtcccgcaattatacatttaata
cgcgatagaaaacaaaatatagcgcgcaaactaggataaattatcgcgcgcggtgtc
atctatgttactagatctctagagtctcaagcttggcgcgccagcttggcgtaatca
tggtcatagctgttgcgattaagaattcgagctcggtacccccctactccaaaaatg
tcaaagatacagtctcagaagaccaaagggctattgagacttttcaacaaagggtaa
tttcgggaaacctcctcggattccattgcccagctatctgtcacttcatcgaaagga
cagtagaaaaggaaggtggctcctacaaatgccatcattgcgataaaggaaaggcta
tcattcaagatgcctctgccgacagtggtcccaaagatggacccccacccacgagga
gcatcgtggaaaaagaagacgttccaaccacgtcttcaaagcaagtggattgatgtg
acatctccactgacgtaagggatgacgcacaatcccactatccttcgcaagaccctt
cctctatataaggaagttcatttcatttggagaggacagcccaagcttcgactctag
aggatccccttaaatcgatATTTATGGCCAGTGCTATTCTTGCTTCATTACTCCACC
CATCAGAAGTGTTGGCACTTGTGCAGTACAAGCTTTCACCCAAAACCCAGCATGATT
ACTCTAACGACAAAACTAGGCAAAGACTTTATCATCATCTTAATATGACTTCCCGAT
CCTTCTCTGCCGTCATACAGGACCTTGATGAAGAGTTAAAGGATGCTATATGCTTAT
TCTATCTGGTGCTGAGAGGCTTAGATACTATAGAAGACGACATGAGCATCGACCTTG
ACACTAAATTGCCTTACCTTCGTACGTTCCACGAAATCATATACCAGAAAGGCTGGA
CTTTCACTAAGAACGGCCCAAATGAAAAAGATAGGCAATTACTGGTAGAATTTGACG
CCATCATAGAGGGCTTCCTTCAATTGAAGCCAGCCTATCAGACTATCATTGCCGATA
TAACCAAACGTATGGGGAACGGAATGGCACACTACGCTACGGCAGGGATACATGTTG
AGACCAACGCAGACTACGACGAGTACTGCCACTATGTCGCTGGTTTGGTGGGGCTGG
GTCTCTCTGAAATGTTTTCCGCATGTGGGTTCGAAAGTCCTCTTGTGGCAGAAAGAA
AAGACCTTAGCAACAGCATGGGACTTTTCCTTCAGAAGACGAACATTGCACGTGATT
ATCTTGAAGACCTCAGAGACAATCGTCGATTTTGGCCCAAGGAAATATGGGGGCAGT
ATGCTGAGACTATGGAGGACTTGGTAAAGCCCGAAAATAAAGAAAAGGCCCTCCAAT
GCCTCTCCCATATGATCGTCAATGCAATGGAGCATATCAGAGACGTTTTGGAGTATC
TCTCTATGATAAAGAATCCGAGCTGCTTCAAATTTTGTGCTATTCCACAAGTCATGG
CTATGGCCACATTAAACCTGCTTCATTCCAACTACAAAGTGTTCACGCATGAGAATA
tcaagatccgtaaaggtgagacagtgtggcttatgaaagaaagtgacagtatggaca
AGGTAGCTGCTATCTTTAGGTTGTACGCCCGACAAATTAACAACAAGTCCAACTCTC
ttgatccccattttgtggatataggggtgatttgcggtgagatcgagcaaatttgcg
TAGGAAGGTTCCCTGGCTCCACAATAGAAATGAAGCGAATGCAGGCTGGAGTCTTAG
GGGGGAAAACTGGAACGGTCCTGATGGCCGGCCCCATCATGACCTCTGCGCCCTCCG
CGACCACGCCCACGGGCAAGACAATGCCGTTCAAGCAGCCTTTCAAGACTGTGGCCA
CGCTGTCCGCCAAGACTGGCAACATTACCAAGCCCATCGACCCTGCCATCTCCAAGA
CCATTGACTTCGTCTACAATGGTTACTCGACGGTCAAGACCAAGGTTGACAAGGCCC
CTAAGGTAAACCCCTACCTGCTCATTGCCGGCGGCCTCGTCCTCTCGTGCATCATCT
CCATGTGCCTGCTCGTCCCGGCCGTGATCTTCTTCCCCGTCACCATCTTCCTGGGTG
TCGCTACGTCGTTTGCGCTCATTGCATTGGCCCCCGTGGCTTTTGTGTTCGGGTGGA
TCCTGATCTCCTCTGCTCCGATCCAGGATAAGGTGGTGGTGCCCGCCTTGGACAAGG
TGCTGGCCAATAAGAAGGTGGCGAAGTTCCTCCTCAAGGAGATGGCGGATCTGAAAT
CAACCTTCCTCGACGTTTACTCTGTTCTCAAGTCTGATCTGCTTCAAGATCCTTCCT
TTGAATTCACCCACGAATCTCGTCAATGGCTTGAACGGATGCTTGACTACAATGTAC
GCGGAGGGAAGCTAAATCGTGGTCTCTCTGTGGTTGATAGCTACAAGCTGTTGAAGC
AAGGTCAAGACTTGACGGAGAAAGAGACTTTCCTCTCATGTGCTCTTGGTTGGTGCA
TTGAATGGCTTCAAGCTTATTTCCTTGTGCTTGATGACATCATGGACAACTCTGTCA
CACGCCGTGGCCAGCCTTGTTGGTTTAGAAAGCCAAAGGTTGGTATGATTGCCATTA
ACGATGGGATTCTACTTCGCAATCATATCCACAGGATTCTCAAAAAGCACTTCAGGG
AAATGCCTTACTATGTTGACCTCGTTGATTTGTTTAACGAGGTAGAGTTTCAAACAG
CTTGCGGCCAGATGATTGATTTGATCACCACCTTTGATGGAGAAAAAGATTTGTCTA
AGTACTCCTTGCAAATCCATCGGCGTATTGTTGAGTACAAAACAGCTTATTACTCAT
TTTATCTTCCTGTTGCTTGCGCATTGCTCATGGCGGGAGAAAATTTGGAAAACCATA
CTGATGTGAAGACTGTTCTTGTTGACATGGGAATTTACTTTCAAGTACAGGATGATT
ATCTGGACTGTTTTGCTGATCCTGAGACACTTGGCAAGATAGGGACAGACATAGAAG
ATTTCAAATGCTCCTGGTTGGTAGTTAAGGCATTGGAACGCTGCAGTGAAGAACAAA
CTAAGATACTATACGAGAACTATGGTAAAGCCGAACCATCAAACGTTGCTAAGGTGA
AAGCTCTCTACAAAGAGCTTGATCTCGAGGGAGCGTTCATGGAATATGAGAAGGAAA
GCTATGAGAAGCTGACAAAGTTGATCGAAGCTCACCAGAGTAAAGCAATTCAAGCAG
TGCTAAAATCTTTCTTGGCTAAGATCTACAAGAGGCAGAAGTAAAAATCCTCAGCAA
TTGggggagctcgaattcgctgaaatcaccagtctctctctacaaatctatctctct
ctattttctccataaataatgtgtgagtagtttcccgataagggaaattagggttct
tatagggtttcgctcatgtgttgagcatataagaaacccttagtatgtatttgtatt
tgtaaaatacttctatcaataaaatttctaattcctaaaaccaaaatccagtactaa
aatccagatctcctaaagtccctatagatctttgtcgtgaatataaaccagacacga
gacgactaaacctggagcccagacgccgttcgaagctagaagtaccgcttaggcagg
aggccgttagggaaaagatgctaaggcagggttggttacgttgactcccccgtaggt
ttggtttaaatatgatgaagtggacggaaggaaggaggaagacaaggaaggataagg
ttgcaggccctgtgcaaggtaagaagatggaaatttgatagaggtacgctactatac
ttatactatacgctaagggaatgcttgtatttataccctataccccctaataacccc
ttatcaatttaagaaataatccgcataagcccccgcttaaaaattggtatcagagcc
atgaataggtctatgaccaaaactcaagaggataaaacctcaccaaaatacgaaaga
gttcttaactctaaagataaaagatggcgcgtggccggcctacagtatgagcggaga
attaagggagtcacgttatgacccccgccgatgacgcgggacaagccgttttacgtt
tggaactgacagaaccgcaacgttgaaggagccactcagccgcgggtttctggagtt
taatgagctaagcacatacgtcagaaaccattattgcgcgttcaaaagtcgcctaag
gtcactatcagctagcaaatatttcttgtcaaaaatgctccactgacgttccataaa
ttcccctcggtatccaattagagtctcatattcactctcaatccaaataatctgcac
cggatctggatcgtttcgcatgattgaacaagatggattgcacgcaggttctccggc
cgcttgggtggagaggctattcggctatgactgggcacaacagacaatcggctgctc
tgatgccgccgtgttccggctgtcagcgcaggggcgcccggttctttttgtcaagac
cgacctgtccggtgccctgaatgaactgcaggacgaggcagcgcggctatcgtggct
ggccacgacgggcgttccttgcgcagctgtgctcgacgttgtcactgaagcgggaag
ggactggctgctattgggcgaagtgccggggcaggatctcctgtcatctcaccttgc
tcctgccgagaaagtatccatcatggctgatgcaatgcggcggctgcatacgcttga
tccggctacctgcccattcgaccaccaagcgaaacatcgcatcgagcgagcacgtac
tcggatggaagccggtcttgtcgatcaggatgatctggacgaagagcatcaggggct
cgcgccagccgaactgttcgccaggctcaaggcgcgcatgcccgacggcgatgatct
catcgtgacccatggcgatgcctgcttgccgaatatcatggtggaaaatggccgctt
ttctggattcatcgactgtggccggctgggtgtggcggaccgctatcaggacatagc
gttggctacccgtgatattgctgaagagcttggcggcgaatgggctgaccgcttcct
cgtgctttacggtatcgccgctcccgattcgcagcgcatcgccttctatcgccttct
tgacgagttcttctgagcgggactctggggttcgaaatgaccgaccaagcgacgccc
aacctgccatcacgagatttcgattccaccgccgccttctatgaaaggttgggcttc
ggaatcgttttccgggacgccggctggatgatcctccagcgcggggatctcatgctg
gagttcttcgcccacgggatctctgcggaacaggcggtcgaaggtgccgatatcatt
acgacagcaacggccgacaagcacaacgccacgatcctgagcgacaatatgatcgcg
gcgtccacatcaacggcgtcggcggcgactgcccaggcaagaccgagatgcaccgcg
atatcttgctgcgttcggatattttcgtggagttcccgccacagacccggatgatcc
ccgatcgttcaaacatttggcaataaagtttcttaagattgaatcctgttgccggtc
ttgcgatgattatcatataatttctgttgaattacgttaagcatgtaataattaaca
tgtaatgcatgacgttatttatgagatgggtttttatgattagagtcccgcaattat
acatttaatacgcgatagaaaacaaaatatagcgcgcaaactaggataaattatcgc
gcgcggtgtcatctatgttactagatcgggactgtaggccggccctcactggtgaaa
agaaaaaccaccccagtacattaaaaacgtccgcaatgtgttattaagttgtctaag
cgtcaatttgtttacaccacaatatatcctgccaccagccagccaacagctccccga
ccggcagctcggcacaaaatcaccactcgatacaggcagcccatcagtccgggacgg
cgtcagcgggagagccgttgtaaggcggcagactttgctcatgttaccgatgctatt
cggaagaacggcaactaagctgccgggtttgaaacacggatgatctcgcggagggta
gcatgttgattgtaacgatgacagagcgttgctgcctgtgatcaaatatcatctccc
tcgcagagatccgaattatcagccttcttattcatttctcgcttaaccgtgacagag
tagacaggctgtctcgcggccgaggggcgcagcccctgggggggatgggaggcccgc
gttagcgggccgggagggttcgagaagggggggcaccccccttcggcgtgcgcggtc
acgcgcacagggcgcagccctggttaaaaacaaggtttataaatattggtttaaaag
caggttaaaagacaggttagcggtggccgaaaaacgggcggaaacccttgcaaatgc
tggattttctgcctgtggacagcccctcaaatgtcaataggtgcgcccctcatctgt
cagcactctgcccctcaagtgtcaaggatcgcgcccctcatctgtcagtagtcgcgc
ccctcaagtgtcaataccgcagggcacttatccccaggcttgtccacatcatctgtg
ggaaactcgcgtaaaatcaggcgttttcgccgatttgcgaggctggccagctccacg
tcgccggccgaaatcgagcctgcccctcatctgtcaacgccgcgccgggtgagtcgg
cccctcaagtgtcaacgtccgcccctcatctgtcagtgagggccaagttttccgcga
ggtatccacaacgccggcggccgcggtgtctcgcacacggcttcgacggcgtttctg
gcgcgtttgcagggccatagacggccgccagcccagcggcgagggcaaccagcccgg
tgagcgtcggaaaggcgctcggtcttgccttgctcgtcggtgatgtacactagtcgc
tggctgctgaacccccagccggaactgaccccacaaggccctagcgtttgcaatgca
ccaggtcatcattgacccaggcgtgttccaccaggccgctgcctcgcaactcttcgc
aggcttcgccgacctgctcgcgccacttcttcacgcgggtggaatccgatccgcaca
tgaggcggaaggtttccagcttgagcgggtacggctcccggtgcgagctgaaatagt
cgaacatccgtcgggccgtcggcgacagcttgcggtacttctcccatatgaatttcg
tgtagtggtcgccagcaaacagcacgacgatttcctcgtcgatcaggacctggcaac
gggacgttttcttgccacggtccaggacgcggaagcggtgcagcagcgacaccgatt
ccaggtgcccaacgcggtcggacgtgaagcccatcgccgtcgcctgtaggcgcgaca
ggcattcctcggccttcgtgtaataccggccattgatcgaccagcccaggtcctggc
aaagctcgtagaacgtgaaggtgatcggctcgccgataggggtgcgcttcgcgtact
ccaacacctgctgccacaccagttcgtcatcgtcggcccgcagctcgacgccggtgt
aggtgatcttcacgtccttgttgacgtggaaaatgaccttgttttgcagcgcctcgc
gcgggattttcttgttgcgcgtggtgaacagggcagagcgggccgtgtcgtttggca
tcgctcgcatcgtgtccggccacggcgcaatatcgaacaaggaaagctgcatttcct
tgatctgctgcttcgtgtgtttcagcaacgcggcctgcttggcctcgctgacctgtt
ttgccaggtcctcgccggcggtttttcgcttcttggtcatcatagttcctcgcgtgt
cgatggtcatcgacttcgccaaacctgccgcctcctgttcgagacgacgcgaacgct
ccacggcggccgatggcgcgggcagggcagggggagccagttgcacgctgtcgcgct
cgatcttggccgtagcttgctggaccatcgagccgacggactggaaggtttcgcggg
gcgcacgcatgacggtgcggcttgcgatggtttcggcatcctcggcggaaaaccccg
cgtcgatcagttcttgcctgtatgccttccggtcaaacgtccgattcattcaccctc
cttgcgggattgccccgactcacgccggggcaatgtgcccttattcctgatttgacc
cgcctggtgccttggtgtccagataatccaccttatcggcaatgaagtcggtcccgt
agaccgtctggccgtccttctcgtacttggtattccgaatcttgccctgcacgaata
ccagcgaccccttgcccaaatacttgccgtgggcctcggcctgagagccaaaacact
tgatgcggaagaagtcggtgcgctcctgcttgtcgccggcatcgttgcgccacatct
aggtactaaaacaattcatccagtaaaatataatattttattttctcccaatcaggc
ttgatccccagtaagtcaaaaaatagctcgacatactgttcttccccgatatcctcc
ctgatcgaccggacgcagaaggcaatgtcataccacttgtccgccctgccgcttctc
ccaagatcaataaagccacttactttgccatctttcacaaagatgttgctgtctccc
aggtcgccgtgggaaaagacaagttcctcttcgggcttttccgtctttaaaaaatca
tacagctcgcgcggatctttaaatggagtgtcttcttcccagttttcgcaatccaca
tcggccagatcgttattcagtaagtaatccaattcggctaagcggctgtctaagcta
ttcgtatagggacaatccgatatgtcgatggagtgaaagagcctgatgcactccgca
tacagctcgataatcttttcagggctttgttcatcttcatactcttccgagcaaagg
acgccatcggcctcactcatgagcagattgctccagccatcatgccgttcaaagtgc
aggacctttggaacaggcagctttccttccagccatagcatcatgtccttttcccgt
tccacatcataggtggtccctttataccggctgtccgtcatttttaaatataggttt
tcattttctcccaccagcttatataccttagcaggagacattccttccgtatctttt
acgcagcggtatttttcgatcagttttttcaattccggtgatattctcattttagcc
atttattatttccttcctcttttctacagtatttaaagataccccaagaagctaatt
ataacaagacgaactccaattcactgttccttgcattctaaaaccttaaataccaga
aaacagctttttcaaagttgttttcaaagttggcgtataacatagtatcgacggagc
cgattttgaaaccacaattatgggtgatgctgccaacttactgatttagtgtatgat
ggtgtttttgaggtgctccagtggcttctgtttctatcagctgtccctcctgttcag
ctactgacggggtggtgcgtaacggcaaaagcaccgccggacatcagcgctatctct
gctctcactgccgtaaaacatggcaactgcagttcacttacaccgcttctcaacccg
gtacgcaccagaaaatcattgatatggccatgaatggcgttggatgccgggcaacag
cccgcattatgggcgttggcctcaacacgattttacgtcacttaaaaaactcaggcc
gcagtcggtaactatgcggtgtgaaataccgcacagatgcgtaaggagaaaataccg
catcaggcgctcttccgcttcctcgctcactgactcgctgcgctcggtcgttcggct
gcggcgagcggtatcagctcactcaaaggcggtaatacggttatccacagaatcagg
ggataacgcaggaaagaacatgtgagcaaaaggccagcaaaaggccaggaaccgtaa
aaaggccgcgttgctggcgtttttccataggctccgcccccctgacgagcatcacaa
aaatcgacgctcaagtcagaggtggcgaaacccgacaggactataaagataccaggc
gtttccccctggaagctccctcgtgcgctctcctgttccgaccctgccgcttaccgg
atacctgtccgcctttctcccttcgggaagcgtggcgctttctcatagctcacgctg
taggtatctcagttcggtgtaggtcgttcgctccaagctgggctgtgtgcacgaacc
ccccgttcagcccgaccgctgcgccttatccggtaactatcgtcttgagtccaaccc
ggtaagacacgacttatcgccactggcagcaggtaacctcgcgcatacagccgggca
gtgacgtcatcgtctgcgcggaaatggacgggcccccggcgccagatctggggaac

The pwh1slf2-peaq_wri1lv1hmgrmcs1_sqs-ldsp-fppsmcs2 plasmid encodes the following in multi-cloning site within site 1 (SEQ ID NO:110).

MKKRLTTSTC SSSPSSSVSS STTTSSPIQS EAPRPKRAKR
AKKSSPSGDK SHNPTSPAST RRSSIYRGVT RHRWTGRFEA
HLWDKSSWNS IQNKKGKQVY LGAYDSEEAA AHTYDLAALK
YWGPDTILNF PAETYTKELE EMQRVTKEEY LASLRRQSSG
FSRGVSKYRG VARHHHNGRW EARIGRVFGN KYLYLGTYNT
QEEAAAAYDM AAIEYRGANA VTNFDISNYI DRLKKKGVFP
FPVNQANHQE GILVEAKQEV ETREAKEEPR EEVKQQYVEE
PPQEEEEKEE EKAEQQEAEI VGYSFEAAVV NCCIDSSTIM
EMDRCGDNNE LAWNFCMMDT GFSPFLTDQN LANENPIEYP
ELFNELAFED NIDFMFDDGK HECLNLENLD CCVVGRESNA
ADEVATQLLN FDLLKLAGDV ESNPGPMISP LASEEDEEIV
KSVVNGTIPS YSLESKLGDC KRAAEIRREA LQRMMGRSLE
GLPVEGFDYE SILGQCGEMP VGYVQIPVGI AGPLLLDGQE
YSVPMATTEG CLVASTNRGC KAIHLSGGAS SVLLKDGMTR
APVVRFASAM RAADLKFFLE NPENFDSLSI AFNRSSRFAK
LQSIQCSIAG KNLYMRFTCS TGDAMGMNMV SKGVQNVLDF
LQSDFPDMDV IGISGNFCSD KKPAAVNWIQ GRGKSVVCEA
IIKEEVVKKV LKSSVASLVE LNMLKNLTGS AIAGALGGFN
AHAGNIVSAI FIATCQDPAQ NVESSHCITM MEAVNDGKDL
HISVTMPSIE VGTVGGGTQL ASQSACLNLL GVKGASKESP
GANSRLLATI VAGSVLAGEL SLMSAIAAGQ LVRSHMKYNR
SSKDVTKFAS S

The pwh1slf2-peaq_wr1lv1hmgrmcs1_sqs-ldsp-fppsmcs2 plasmid encodes the following in multi-cloning site within site 2 (SEQ ID NO:111)

MASAILASLL HPSEVLALVQ YKLSPKTQHD YSNDKTRQRL
YHHLNMTSRS FSAVIQDLDE ELKDAICLFY LVLRGLDTIE
DDMTIDLDTK LPYLRTFHEI IYQKGWTFTK NGPNEKDRQL
LVEFDAIIEG FLQLKPAYQT IIADITKRMG NGMAHYATAG
IHVETNADYD EYCHYVAGLV GLGISEMFSA CGFESPLVAE
RKDLSNSMGL FLQKTNIARD YLEDLRDNRR FWPKEIWGQY
AETMEDLVKP ENKEKALQCL SHMIVNAMEH IRDVLEYLSM
IKNPSCFKFC AIPQVMAMAT LNLLHSNYKV FTHENIKIRK
GETVWLMKES DSMDKVAAIF RLYARQINNK SNSLDPHFVD
IGVICGEIEQ ICVGRFPGST IEMKRMQAGV LGGKTGTVLM
AGPIMTSAPS ATTPTGKTMP FKQPFKTVAT LSAKTGNITK
PIDPAISKTI DFVYNGYSTV KTKVDKAPKV NPYLLIAGGL
VLSCIISMCL LVPAVIFFPV TIFLGVATSF ALIALAPVAF
VEGWILISSA PIQDKVVVPA LDKVLANKKV AKFLLKEMAD
LKSTFLDVYS VLKSDLLQDP SFEFTHESRQ WLERMLDYNV
RGGKLNRGLS VVDSYKLLKQ GQDLTEKETF LSCALGWCIE
WLQAYFLVLD DIMDNSVTRR GQPCWFRKPK VGMIAINDGI
LLRNHIHRIL KKHFREMPYY VDLVDLFNEV EFQTACGQMI
DLITTFDGEK DLSKYSLQIH RRIVEYKTAY YSFYLPVACA
LLMAGENLEN HTDVKTVLVD MGIYFQVQDD YLDCFADPET
LGKIGTDIED FKCSWLVVKA LERCSEEQTK ILYENYGKAE
PSNVAKVKAL YKELDLEGAF MEYEKESYEK LTKLIEAHQS
KAIQAVLKSF LAKIYKRQK

### REFERENCES

1. Chapman, K. D. & Ohlrogge, J. B. Compartmentation of triacylglycerol accumulation in plants, J. Biol. Chem. 287, 2288-2294 (2012).
2. Li, M. et al. Purification and structural characterization of the central hydrophobic domain of oleosin. J. Biol. Chem. 277, 37888-37895 (2002).
3. Zale, J. et al. Metabolic engineering of sugarcane to accumulate energy-dense triacylglycerols in vegetative biomass. Plant Biotechnol. J. 14, 661-669 (2016).
4. Yang, Y. et al. Ectopic expression of WRI1 affects fatty acid homeostasis in Brachypodium distachyon vegetative tissues. Plant Physiol. 169, 1836-1847 (2015).
5. Du, Z. Y. & Benning, C. Triacylglycerol accumulation in photosynthetic cells in plants and algae. Subcell. Biochem. 86, 179-205 (2016).
6. Cernac, A. & Benning, C. WRINKLED1 encodes an AP2/EREB domain protein involved in the control of storage compound biosynthesis in Arabidopsis. Plant J. 40, 575-585 (2004).
7. Maeo, K. et al. An AP2-type transcription factor, WRINKLED1, of Arabidopsis thaliana binds to the AW-box sequence conserved among proximal upstream regions of genes involved in fatty acid synthesis. Plant J. 60, 476-487 (2009).
8. Sanjaya, Durrett, T. P., Weise, S. E. & Benning, C. Increasing the energy density of vegetative tissues by diverting carbon from starch to oil biosynthesis in transgenic Arabidopsis. Plant Biotechnol. J. 9, 874-883 (2011).
9. Vanhercke, T. et al. Metabolic engineering of biomass for high energy density: oilseed-like triacylglycerol yields from plant leaves. Plant Biotechnol. J. 12, 231-239 (2014).
10. Grimberg, A., Carlsson, A. S., Marttila, S., Bhalerao, R. & Hofvander, P. Transcriptional transitions in Nicotiana benthamiana leaves upon induction of oil synthesis by WRINKLED1 homologs from diverse species and tissues. BMC Plant Biol. 15, 192 (2015).
11. Ma, W. et al. Deletion of a C-terminal intrinsically disordered region of WRINKLED1 affects its stability and enhances oil accumulation in Arabidopsis. Plant J. 83, 864-874 (2015).
12. Fan, J., Yan, C., Zhang, X. & Xu, C. Dual role for phospholipid:diacylglycerol acyltransferase: enhancing fatty acid synthesis and diverting fatty acids from membrane lipids to triacylglycerol in Arabidopsis leaves. Plant Cell 25, 3506-3518 (2013).
13. Lange, B. M. & Ahkarni, A. Metabolic engineering of plant monoterpenes, sesquiterpenes and diterpenes-current status and future opportunities. Plant Biotechnol. J. 11, 169-196 (2013).
14. Augustin, J. M., Higashi, Y., Feng, X. & Kutchan, T. M. Production of mono- and sesquiterpenes in Camelina sativa oilseed. Planta 242, 693-708 (2015).
15. Reed, J. et al. A translational synthetic biology platform for rapid access to gram-scale quantities of novel drug-like molecules. Metab. Eng. 42, 185-193 (2017).
16. Wu, S. et al. Redirection of cytosolic or plastidic isoprenoid precursors elevates terpene production in plants. Nat. Biotechnol. 24, 1441-1447 (2006).
17. Pateraki, I. et al. Manoyl oxide (13R), the biosynthetic precursor of forskolin, is synthesized in specialized root cork cells in Coleus forskohlii. Plant Physiol. 164, 1222-1236 (2014).
18. Liao, P., Hemmerlin, A., Bach, T. J. & Chye, M. L. The potential of the mevalonate pathway for enhanced isoprenoid production. Biotechnol. Adv. 34, 697-713 (2016).
19. Frank, A. & Groll, M. The Methylerythritol Phosphate Pathway to Isoprenoids. Chem. Rev. 117, 5675-5703 (2017).
20. Banerjee, A. & Sharkey. T. D. Methylerythritol 4-phosphate (MEP) pathway metabolic regulation. Nat. Prod. Rep. 31, 1043-1055 (2014).
21. Chappell., J., Wolf, F., Proulx, J., Cuellar, R. & Saunders, C. Is the reaction catalyzed by 3-hydroxy-3-methylglutaryl coenzyme A reductase a rate-limiting step for isoprenoid biosynthesis in plants? Plant Physiol. 109, 1337-1343 (1995).
22. Estevez, J. M., Cantero, A., Reindl, A., Reichler, S. & Leon, P. 1-Deoxy-D-xylulose-5-phosphate synthase, a limiting enzyme for plastidic isoprenoid biosynthesis in plants. J. Biol. Chem. 276, 22901-22909 (2001).
23. Bruckner, K. & Tissier, A. High-level diterpene production by transient expression in Nicotiana benthamiana. Plant Methods 9, 46 (2013).
24. Vieler, A., Brubaker, S. B., Vick, B. & Benning, C. A lipid droplet protein of Nannochloropsis with functions partially analogous to plant oleosins. Plant Physiol. 158, 1562-1569 (2012).
25. Skrukrud, C. L,, Taylor, S. E., Hawkins, D. R. & Galvin, M. in The Metabolism Structure, and Function of Plant Lipids (eds. Paul K. Stumpf, J. Brian Mudd, & W. David Nes) 115-118 (Springer New York, 1987).
26. Keim, V. et al. Characterization of Arabidopsis FPS isozymes and FPS gene expression analysis provide insight into the biosynthesis of isoprenoid precursors in seeds. PloS One 7, e49109 (2012).
27. Vogel, B. S., Wildung, M. R., Vogel, G. & Croteau, R. Abietadiene synthase from grand fir (Abies grandis): cDNA isolation, characterization, and bacterial expression of a bifunctional diterpene cyclase involved in resin acid biosynthesis. J. Biol. Chem. 271, 23262-23268 (1996).
28. Peters, R. J. et al. Abietadiene synthase from grand fir (Abies grandis): characterization and mechanism of action of the “pseudomature” recombinant enzyme. Biochem. 39, 15592-15602 (2000).
29. Keeling, C. I., Madilao, L. L., Zerbe, P., Dullat, H. K. & Bohlmann, J. The primary diterpene synthase products of Picea abies levopimaradiene/abietadiene synthase (PaLAS) are epimers of a thermally unstable diterpenol. J. Biol. Chem. 286, 21145-21153 (2011).
30. Noike, M., Katagiri, T., Nakayama, T., Nishino, T. & Hemmi, H. Effect of mutagenesis at the region upstream from the G(Q/E) motif of three types of geranylgeranyl diphosphate synthase on product chain-length. J. Biosci. Bioeng. 107, 235-239 (2009).
31. Chang, T. H., Guo, R. I., Ko, T. P., Wang, A. H. & Liang, P. H. Crystal structure of type-III geranylgeranyl pyrophosphate synthase from Saccharomyces cerevisiae and the mechanism of product chain length determination. J. Biol. Chem. 281, 14991-15000 (2006).
32. Xu, Q. et al. Discovery and comparative profiling of microRNAs in a sweet orange red-flesh mutant and its wild type. BMC Genomics 11, 246-246 (2010).
33. Zhou, F. et al. A recruiting protein of geranylgeranyl diphosphate synthase controls metabolic flux toward chlorophyll biosynthesis in rice. Proc. Natl. Acad. Sci. 114, 6866-6871 (2017).
34. Ruiz-Sola, M. A. et al. Arabidopsis GERANYLGERANYL DIPHOSPHATE SYNTHASE 11 is a hub isozyme required for the production of most photosynthesis-related isoprenoids. New Phytol. 209, 252-264 (2016).
35. Hamberger, B., Ohnishi, T., Hamberger, B., Seguin, A. & Bohlmann, J. Evolution of diterpene metabolism: Sitka spruce CYP720B4 catalyzes multiple oxidations in resin acid biosynthesis of conifer defense against insects. Plant Physiol. 157, 1677-1695 (2011).
36. Dong, L., Jongedijk, E., Bouwmeester, H. & Van Der Krol, A. Monoterpene biosynthesis potential of plant subcellular compartments. New Phytol. 209, 679-690 (2016),
37. van Herpen, T. W. et al. Nicotiana benthamiana as a production platform for artemisinin precursors. PloS One 5, e14222 (2010).
38. Gnanasekaran, T. et al. Heterologous expression of the isopimaric acid pathway in Nicotiana benthamiana and the effect of N-terminal modifications of the involved cytochrome P450 enzyme. J. Biol. Eng. 9, 24 (2015).
39. Jagalski, V. et al. Biophysical study of resin acid effects on phospholipid membrane structure and properties. Biochim. Biophys. Acta 1858, 2827-2838 (2016).
40. Delatte, T. L. et al. Engineering storage capacity for volatile sesquiterpenes in Nicotiana benthamiana leaves. Plant Biotechnol. J. (2018) Epub ahead of print.
41. Zhao, C. et al. Co-Compartmentation of terpene biosynthesis and storage via synthetic droplet, ACS Synth. Biol. 7,774-781 (2018).
42. Tissier, A., Morgan, J. A. & Dudareva, N. Plant Volatiles: Going ‘in’ but not ‘out’ of trichome cavities. Trends Plant Sci. 22, 930-938 (2017).
43. Uehling, J. et al. Comparative genomics of Mortierella elongata and its bacterial endosymbiont Mycoavidus cysteinexigens. Environ. Microbiol. 19, 2964-2983 (2017).
44. Xiao, M. et al. Transcriptome analysis based on next-generation sequencing of non-model plants producing specialized metabolites of biotechnological interest. J. Biotechnol. 166, 122-134 (2013).
45. Yerrapragada, S. et al. Extreme sensory complexity encoded in the 10-megabase draft genome sequence of the chromatically acclimating cyanobacterium Tolypothrix sp. PCC 7601. Genome Announc. 3, e00355-15 (2015).
46. Earley, K. W. et al. Gateway-compatible vectors for plant functional genomics and proteomics. Plant J. 45, 616-629 (2006).
47. Voinnet, O., Pinto, Y. M. & Baulcombe, D. C. Suppression of gene silencing: a general strategy used by diverse DNA and RNA viruses of plants. Proc. Natl. Acad. Sci. 96, 14147-14152 (1999).
48. Voinnet, O., Pinto, Y. M. & Baulcombe, D. C. Correction for Yoinnet et al., Suppression of gene silencing: A general strategy used by diverse DNA and RNA viruses of plants. Proc. Natl. Acad. Sci. 112, E4812 (2015).
49. Ding, Y. et al. Isolating lipid droplets from multiple species. Nat. Protoc. 8, 43 (2012).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various features of the invention according to the foregoing description provided in the specification and figures.

### Statements:

1. A fusion protein comprising a lipid droplet surface protein linked in-frame to one or more a fusion partners comprising a monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR) mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), patchoulol synthase, or WRI1 protein.
2. The fusion protein of statement 1, wherein the lipid droplet surface protein has a sequence with at least 90% sequence identity to SEQ ID NO:1, or a truncated sequence with at least 90% sequence identity to a sequence consisting of less than 120 contiguous amino acids, or less than 110 contiguous amino acids, or less than 105 contiguous amino acids, or less than 100 contiguous amino acids, or less than 95 contiguous amino acids, or less than 90 contiguous amino acids, or less than 85 contiguous amino acids, or less than 80 contiguous amino acids, or less than 75 continuous amino acids of SEQ ID NO:1.
3. The fusion protein of statement 1 and 2, wherein the fusion partner is a polypeptide with at least 95% sequence identity to a sequence comprising SEQ ID NO:3, 5, 7, 9, 11, 13, 15, 17, 19, 21, 23, 25, 27, 29, 31, 33, 35, 37, 39, 41, 43, 45, 47, 49, 51, 52, 53, 54, 55, 56, 59, 61, 63, 64, 65, 67, 68, 69, 71, 72, 73, 75, 76, 77, 79, 80, 81, 83, 84, 85, 87, 89, 91, 92, 93, 95, 96, 97, 99, 101, 104, 105, 107, 108, 110, or 111.
4. An expression system comprising at least one expression cassette (or expression vector) having a heterologous promoter operably linked to a nucleic acid segment encoding a lipid droplet surface protein and another expression cassette (or expression vector) comprising a heterologous promoter operably linked to a nucleic acid segment encoding one or more of the following proteins: monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), patchoulol synthase, or WRI1 protein.
5. An expression system comprising at least one expression cassette (or expression vector) having a heterologous promoter operably linked to a nucleic acid segment encoding a fusion protein, the fusion protein comprising a lipid droplet surface protein linked in-frame to one or more a fusion partners comprising a monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphornevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), patchoulol synthase, or WRI1 protein.
6. The expression system of statement 4 or 5, further comprising at least one expression cassette (or expression vector), each having a heterologous promoter operably linked to a nucleic acid segment encoding a protein selected from geranylgeranyl diphosphate synthase (GGDPS), farnesylpyrophosphate synthase (FPPS), 1-deoxy-D-xylulose 5-phosphate synthase (DXS), abietadiene synthase (ABS), cytochrome P450, cytochrome P450 reductase, mevalonic acid kinase (MVK), phosphomevalonate kinase (PMK), mevalonate-5-diphosphate decarboxylase (MPD), cytidine 5′-diphosphate-methylerythritol (CDP-ME) synthetase (IspD), 2-C-methyl-d-erythritol 2,4-cyclodiphosphate synthase (IspF), isopentenyl diphosphate isomerase (IDI), ribulose bisphosphate carboxylase, or WRI1 protein.
7. The expression system of statement 4, 5 or 6, wherein the fusion protein and protein are encoded by separate expression cassettes (or expression vectors).
8. The expression system of statement 4-6 or 7, wherein the fusion protein and each protein are encoded within one expression cassette (or expression vector), wherein expression of the fusion protein and at least one protein is from one promoter that drives expression of the fusion protein and the at least one protein.
9. An expression system comprising a first expression cassette or first expression vector comprising a heterologous promoter operably linked to a nucleic acid segment encoding a WRINKLED (WRI1) transcription factor, and a second expression cassette or second expression vector comprising a heterologous promoter operably linked to a nucleic acid segment encoding a lipid droplet surface protein (LDSP).
10. The expression system of statement 9, further comprising an expression cassette or expression vector comprising a heterologous promoter operably linked to a nucleic acid segment encoding a abietadiene synthase (ABS).
11. An expression system comprising at least one expression cassette or expression vector comprising one or more nucleic acid segment, each nucleic acid segment encoding one or more of the following proteins: encoding one or more of the following proteins: a HMG-CoA reductase (HMGR), farnesylpyrophosphate synthase (FPPS), patchoulol synthase, or a combination thereof, wherein a heterologous promoter is operable linked to each of the nucleic acid segments encoding a protein.
12. An expression system comprising at least one expression cassette or expression vector comprising one or more nucleic acid segment, each nucleic acid segment encoding one or more of the following proteins: 1-deoxy-D-xylulose 5-phosphate synthase (DXS), farnesylpyrophosphate synthase (FPPS), patchoulol synthase, lipid droplet surface protein (LDSP), WRINKLED, or a combination thereof, wherein a heterologous promoter is operable linked to each of the nucleic acid segments encoding a protein.
13. An expression system comprising at least one expression cassette or expression vector comprising one or more nucleic acid segment, each nucleic acid segment encoding one or more of the following proteins: 1-deoxy-D-xylulose 5-phosphate synthase (DXS), geranylgeranyl diphosphate synthase (GGDPS), abietadiene synthase (ABS), or a combination thereof, wherein a heterologous promoter is operable linked to each of the nucleic acid segments encoding a protein.
14. An expression system comprising at least one expression cassette or expression vector comprising one or more nucleic acid segment, each nucleic acid segment encoding one or more of the following proteins: HMG-CoA reductase (HMGR), geranylgeranyl diphosphate synthase (GGDPS), abietadiene synthase (ABS), or a combination thereof, wherein a heterologous promoter is operable linked to each of the nucleic acid segments encoding a protein.
15. The expression system of statement 11-14, further comprising an expression cassette or expression vector comprising one or more nucleic acid segments encoding at least one of the following proteins cytochrome P450, cytochrome P450 reductase, or a combination thereof, wherein optionally one or more nucleic acid segments encoding the cytochrome P450, cytochrome P450 reductase, or both are linked to in-frame to a nucleic acid segment encoding lipid surface droplet protein.
16. The expression system of statement 4-14 or 15, wherein the fusion partner or the at least one protein is linked in-frame to a plastid targeting segment.
17. The expression system of statement 4-14 or 15, wherein the fusion partner or the protein is not linked in-frame to a plastid targeting segment.
18. The expression system of statement 4-16 or 17, wherein a plastid targeting region or a hydrophobic region is removed from the nucleic acid segment encoding the one or more protein.
19. The expression system of statement 4-17 or 18, further comprising an expression cassette comprising a promoter operably linked to a nucleic acid encoding a WRI1 transcription factor.

20. The expression system of statement 4-18 or 19, further comprising an expression cassette (or expression vector) comprising a promoter operably linked to a nucleic acid encoding a lipid droplet surface protein.
21. The expression system of statement 4-19 or 20, wherein the fusion partner or protein has at least 90% sequence identity to a sequence comprising SEQ ID NO: 3, 5, 7, 9, 11, 13, 15, 17, 19, 21, 23, 25, 27, 29, 31, 33, 35, 37, 39, 41, 43, 45, 47, 49, 51, 52, 53, 54, 55, 56, 59, 61, 63, 64, 65, 67, 68, 69, 71, 72, 73, 75, 76, 77, 79, 80, 81, 83, 84, 85, 87, 89, 91, 92, 93, 95, 96, 97, 99, 101, 104, 105, 107, 108, 110, or 111.
22. The expression system of statement 4-20 or 21, wherein the nucleic acid segment is codon-optimized for expression in plastid or in a host cell.
23. The expression system of statement 4-21 or 22, wherein one or more of the heterologous promoters is active in plant plastids.
24. A host cell, host tissue, host seed, or a host plant comprising the expression system of statement 4-22 or 23.
25. The host cell, host tissue, host seed, or a host plant of statement 24, each comprising insect cells, plant cells, fungal cells, insect tissues, plant tissues, or fungal tissues.
26. The host cell, host tissue, host seed, or a host plant of statement 24 or 25, which is an oil-producing plant species.
27. The host cell, host tissue, host seed, or a host plant of statement 24, 25 or 26, which is an oilseed, camelina, canola, castor bean, corn, flax, lupin, peanut, potatoe, safflower, soybean, sunflower, cottonseed, oil firewood tree, rapeseed, rutabaga, sorghum, walnut, or nut species.
28. The host cell, host tissue, host seed, or a host plant of statement 24, 25 or 26, which is a Nicotiana benthamiana, Nicotiana tabacum, Nicotiana rustica, Nicotiana excelsior, or Nicotiana excelsiana species.
29. The host cell, host tissue, host seed, or a host plant of statement 24-26 or 27, which is not a Nicotiana benthamiana species.
30. A method comprising (a) incubating a population of host cells or a host tissue comprising an expression system of statement 4-22 or 23; and (b) isolating lipids from the population of host cells or the host tissue.
31. The method of statement 30 comprising (a) incubating a population of host cells or a host tissue comprising an expression system that includes at least one expression cassette having a heterologous promoter operably linked to a nucleic acid segment encoding a fusion protein comprising a lipid droplet surface protein linked in-frame to one or more a fusion partners comprising a monoterpene synthase, diteipene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase abietadiene synthase (ABS), farnesylpyrophosphate synthase (FPPS), ribulose bisphosphate carboxylase, squalene synthase (SQS), patchoulol synthase, or WRI1 protein; and (h) isolating lipids from the population of host cells or the host tissue.
32. The method of statement or 31, wherein the population of host cells or the host tissue is within a plant.
33. The method of statement 30, 31 or 32, wherein the population of host cells or the host tissue is within a plant and the incubating comprises cultivating the plant or a seed of the plant.
34. A method comprising (a) cultivating a plant or a seed, the plant or the seed comprising an expression system of statement 4-22 or 23 to generate a plant comprising lipid droplets within the plant's cells; and (b) isolating lipids from the plant or the plant's cells.
35. The method of statement 30-33 or 34, wherein the population of host cells, or the host tissue, or the cells of the plant further comprise at least one expression cassette (or expression vector), each having a heterologous promoter operably linked to a nucleic acid segment encoding a protein selected from geranylgeranyl diphosphate synthase (GGDPS), farnesylpyrophosphate synthase (FPPS), 1-deoxy-D-xylulose 5-phosphate synthase (DXS), abietadiene synthase (ABS), cytochrome P450, cytochrome P450 reductase, mevalonic acid kinase (MVK), phosphomevalonate kinase (PMK), mevalonate-5-diphosphate decarboxylase (MPD), cytidine 5′-diphosphate-methylerythritol (CDP-ME) synthetase (IspD), 2-C-methyl-d-erythritol 2,4-cyclodiphosphate synthase (IspF), isopentenyl diphosphate isomerase (IDI), ribulose bisphosphate carboxylase, or WRI1 protein.
36. The method of statement 30-34 or 35, wherein each fusion protein or protein is encoded by a separate expression cassette (or expression vector).
37. The method of statement 30-34 or 35, wherein at least two fusion proteins or proteins are encoded in a single expression vector.
38. The method of statement 30-36 or 37, wherein the population of host cells or the host tissue further comprises a heterologous expression cassette (or expression vector) comprising a promoter operably linked to a nucleic acid encoding a WRI1 transcription factor.
39. The method of statement 30-37 or 38, wherein the population of host cells or the host tissue further comprises a heterologous expression cassette (or expression vector) comprising a promoter operably linked to a nucleic acid encoding a lipid droplet surface protein.
40. The method of statement 30-38 or 39, wherein a segment encoding a plastid targeting region or a hydrophobic region is removed from the nucleic acid segment encoding the one or more fusion partner or protein.
41. The method of statement 30-39 or 40, wherein one or more nucleic acid segment encoding the fusion protein, or the protein is codon-optimized for expression in plant plastids or in a host cell.
42. The method of statement 30-40 or 42, wherein the expression system comprises an expression cassette comprising a promoter operably linked to a nucleic acid segment encoding an enzyme with at least 90% sequence identity to a sequence comprising SEQ ID NO: 3, 5, 7, 9, 11, 13, 15, 17, 19, 21, 23, 25, 27, 29, 31, 33, 35, 37, 39, 41, 43, 45, 47, 49, 51, 52, 53, 54, 55, 56, 59, 61, 63, 64, 65, 67, 68, 69, 71, 72, 73, 75, 76, 77, 79, 80, 81, 83, 84, 85, 87, 89, 91, 92, 93, 95, 96, 97, 99, 101, 104, 105, 107, 108, 110, or 111.

43. The method of statement 30-41 or 42, wherein the lipids isolated from the population of host cells comprise one or more types of terpene.
44. The method of statement 30-42 or 43, further comprising isolating terpenes from the lipids isolated from the population of host cells or tissues.
45. The method of statement 30-43 or 44, wherein the lipids isolated from the population of host cells comprise one or more types of monoterpene, diterpene, sesquiterpene, sesterterpene, triterpene, tetraterpene, polyterpene, or a mixture thereof.
46. The method of statement 30-44 or 45, wherein after incubation, the host cells or tissues have at least 0.05%, at least 0.1%, at least 0.2%, at least 0.25%, or at least 0.3% fresh weight monoterpene, diterpene, sesquiterpene, sesterterpene, triterpene, tetraterpene, polyterpene, or a mixture thereof.

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised. material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A fusion protein comprising a lipid droplet surface protein linked e to one or more of the following fusion partners: a monoterpene synthase, diterpene, synthase, sesquiterpene synthase, sesterterpene synthase, triterpene synthase, tetraterpene synthase, polyterpene synthase, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase abietadiene synthase (ABS), farnesylpyrophosphate synthase (FPPS), ribulose bisphosphate carboxylase, squalene synthase (SQS), or patchoulol synthase.

2. The fusion protein of claim 1, wherein the lipid droplet surface protein has a sequence with at least 95% sequence identity to SEQ ID NO:1, or a truncated sequence with at least 95% sequence identity to a sequence consisting of at least 70 contiguous amino acids of SEQ ID NO:1.

3. The fusion protein of claim 1, wherein the fusion partner comprises a polypeptide with at least 95% sequence identity to SEQ ID NO:3, 5, 7, 9, 11, 13, 15, 17, 19, 21, 23, 25, 27, 29, 31, 31 35, 37, 39, 41, 43, 45, 47, 49, 51, 52, 53, 54, 55, 56, 59, 61, 63, 64, 65, 67, 68, 97, 99, 101, 104, 105, 107, 108, 110, or 111.

4. An expression system comprising at least one expression vector comprising a first nucleic acid segment encoding a lipid droplet surface protein and at least one second nucleic acid segment encoding one or more of the following proteins: monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase abietadiene synthase (ABS), farnesylpyrophosphate synthase (FPPS), ribulose bisphosphate carboxylase, squalene synthase (SQS), or patchoulol synthase, wherein the first nucleic segment, the at least one second nucleic acid segment, or a combination thereof are operably linked to a heterologous promoter.

5. The expression system of claim 4, wherein the lipid droplet surface protein has a sequence with at least 90% sequence identity to SEQ ID NO:1 or a truncated sequence with at least 95% sequence identity to a sequence consisting of at least 70 contiguous amino acids of SEQ ID NO:1.

6. The expression system of claim 4, wherein first nucleic acid segment encoding a lipid droplet surface protein is linked in frame with at least one second nucleic acid segment encoding at least one of the proteins, such that the expression system expresses a fusion protein comprising the lipid droplet surface protein and at least one of the proteins.

7. The expression system of claim 4, comprising two or more expression cassettes or two or more expression vectors, a first expression cassette or expression vector comprising a heterologous promoter operably linked to a nucleic acid segment encoding a fusion protein comprising a lipid droplet surface protein linked in-frame to one or more of the following fusion partners: a monoterpene synthase, diterpene synthase, sesquiterpene synthase, sesterterpene synthase, triterpene synthase, tetraterpene synthase, polyterpene synthase, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase abietadiene synthase (ABS), farnesylpyrophosphate synthase (FPPS), ribulose bisphosphate carboxylase, squalene synthase (SQS), or patchoulol synthase; and a second expression cassette or expression vector comprising a heterologous promoter operably linked to a nucleic acid segment encoding one or more of the following proteins: a monoterpene synthase, diterpene synthase, sesquiterpene synthase, sesterterpene synthase, triterpene synthase, tetraterpene synthase, polyterpene synthase, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), or patchoulol synthase.

8. The expression system of claim 4, further comprising at least one expression cassette or at least one expression vector comprising a heterologous promoter operably linked to a nucleic acid segment encoding an enzyme selected from geranylgeranyl diphosphate synthase (GGDPS), 1-deoxy-D-xylulose 5-phosphate synthase (DXS), abietadiene synthase (ABS), 3-hydroxy-3-methylglutaryl-CoA reductase (HMGR), farnesyl diphosphate synthase (FDPS), cytochrome P450, NADPH-dependent cytochrome P450 reductase (CPR), each nucleic acid segment encoding an enzyme optionally linked in frame to a lipid droplet surface protein.

9. The expression system of claim 4, further comprising an expression cassette comprising a promoter operably linked to a nucleic acid encoding a WRI1 transcription factor.

10. The expression system of claim 4, further comprising an expression cassette comprising a promoter operably linked to a nucleic acid encoding a lipid droplet surface protein.

11. The expression system of claim 4, wherein an encoded plastid targeting region or an encoded hydrophobic region is removed from the nucleic acid segment encoding the one or more of the proteins.

12. The expression system of claim 4, further comprising an encoded plastid targeting region or an encoded hydrophobic region linked in frame with nucleic acid segment encoding the one or more of the proteins.

13. The expression system of claim 4, wherein one or more of the proteins has an amino acid sequence at least 95% sequence identity to SEQ ID NO:3, 5, 7, 9, 11, 13, 15, 17, 19, 21, 23, 25, 27, 29, 31, 33, 35, 37, 39, 41, 43, 45, 47, 49, 51, 52, 53, 54, 55, 56.59, 61, 63, 64, 65, 67, 68, 97, 99, 101, 104, 105, 107, 108, 110, or 111.

14. The expression system of claim 4, wherein the first nucleic acid segment or at least one of the second nucleic acid segments is codon-optimized for expression in plastid or in a host cell.

15. The expression system of claim 4, wherein at least of the heterologous promoters is active in plant plastids.

16. A host cell, host tissue, host seed, or host plant comprising the expression system of claim 4.

17. The host cell, host tissue, host seed, or a host plant of claim 16, which is an oilseed, carnelina, canola, castor bean, corn, flax, lupin, peanut, potatoe, safflower, soybean, sunflower, cottonseed, oil firewood tree, rapeseed, rutabaga, sorghum, walnut, or nut species.

18. The host cell, host tissue, host seed, or a host plant of claim 16, which is a Nicotiana benthamiana, Nicotiana tabacum, Nicotiana rustica, Nicotiana excelsior, or Nicotiana excelsiana species.

19. A method comprising:
(a) incubating or cultivating one or more host cells, host tissues, host seeds, or host plants, each comprising expression system comprising at least one expression vector comprising a a first nucleic acid segment encoding a lipid droplet surface protein and at least one second nucleic acid segment encoding one or more of the following proteins: monoterpene synthase, diterpene synthase, sesquiterpene synthase, sesterterpene synthase, triterpene synthase, tetraterpene synthase, polyterpene synthase, transcription factor, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), or patchoulol synthase, wherein the first nucleic segment, the at least one second nucleic acid segment, or a combination thereof are operably linked to a heterologous promoter; and
(b) isolating lipids from the host cell, host tissue, host seed, or host plant,

20. The method of claim 19, wherein the lipid droplet surface protein has a sequence with at least 90% sequence identity to SEQ ID NO:1 or a truncated sequence with at least 95% sequence identity to a sequence consisting of at least 70 contiguous amino acids of SEQ ID NO:1.

21. The method of claim 19, wherein first nucleic acid segment encoding a lipid droplet surface protein is linked in frame with at least one second nucleic acid segment encoding at least one of the proteins, such that the expression system expresses a fusion protein comprising the lipid droplet surface protein and at least one of the proteins.

22. The method of claim 19, comprising two or more expression cassettes or two or more expression vectors, a first expression cassette or expression vector comprising a heterologous promoter operably linked to a nucleic acid segment encoding a fusion protein comprising a lipid droplet surface protein linked in-frame to one or more of the following fusion partners: a monoterpene synthase, diterpene synthase, sesquiterpene synthase, sesterterpene synthase, triterpene synthase, tetraterpene synthase, polyterpene synthase, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-methyl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase abietadiene synthase (ABS), farnesylpyrophosphate synthase (FPPS), ribulose bisphosphate carboxylase, squalene synthase (SQS), or patchoulol synthase; and a second expression cassette or expression vector comprising a heterologous promoter operably linked to a nucleic acid segment encoding one or more of the following proteins: a monoterpene synthase, diterpene synthase, sesquiterpene synthase, sesterterpene synthase, triterpene synthase, tetraterpene synthase, polyterpene synthase, cytochrome P450, cytochrome P450 reductase, 1-deoxy-D-xylulose 5-phosphate synthase (DXS), 1-deoxy-D-xylulose 5-phosphate-reducto-isomerase, cytidine 5′-diphosphate-methylerythritol (CDP-ME) synthetase (IspD), 2-C-meth yl-d-erythritol 2,4-cyclodiphosphate synthase (IspF), geranylgeranyl diphosphate synthase (GGDPS), HMG-CoA synthase, HMG-CoA reductase (HMGR), mevalonic acid kinase (MVK), phosphomevalonate kinase (PMK), mevalonate-5-diphosphate decarboxylase (MPD), isopentenyl diphosphate isomerase (IDI), abietadiene synthase (ABS), farnesylpyrophosphate synthase (FPPS), ribulose bisphosphate carboxylase, squalene synthase (SQS), or patchoulol synthase.

23. The method of claim 19, further comprising at least one expression cassette or at least one expression vector comprising a heterologous promoter operably linked to a nucleic acid segment encoding an enzyme selected from geranylgeranyl diphosphate synthase (GGDPS), 1-deoxy-D-xylulose 5-phosphate synthase (DXS), abietadiene synthase (ABS), 3-hydroxy-3-methylglutaryl-CoA reductase (HMGR), farnesyl diphosphate synthase (FDPS), cytochrome P450, NADPH-dependent cytochrome P450 reductase (CPR), each nucleic acid segment encoding an enzyme optionally linked in frame to a lipid droplet surface protein.

24. The method of claim 19, further comprising an expression cassette comprising a promoter operably linked to a nucleic acid encoding a WRI1 transcription factor.

25. The method of claim 19, further comprising an expression cassette comprising a promoter operably linked to a nucleic acid encoding a lipid droplet surface protein.

26. The method of claim 19, wherein an encoded plastid targeting region or an encoded hydrophobic region is removed from the nucleic acid segment encoding the one or more of the proteins.

27. The method of claim 19, further comprising an encoded plastid targeting region or an encoded hydrophobic region linked in frame with nucleic acid segment encoding the one or more of the proteins.

28. The method of claim 19, wherein one or more of the proteins has an amino acid sequence at least 95% sequence identity to SEQ ID NO:3, 5, 7, 9, 11, 13, 15, 17, 19, 21, 23, 25, 27, 29, 31, 33, 35, 37, 39, 41, 43, 45, 47, 49, 51, 52, 53, 54, 55, 56, 59, 61, 63, 64, 65, 67, 68, 97, 99, 101, 104, 105, 107, 108, 110, or 111.

29. The method of claim 19, wherein the first nucleic acid segment or at least one of the second nucleic acid segments is codon-optimized for expression in plastid or in a host cell.

30. The method of claim 19, wherein at least of the heterologous promoters is active in plant plastids.

31. The method of claim 19, wherein the lipids isolated from one or more host cells, host tissues, host seeds, or host plants comprise one or more types of monoterpene, diterpene, sesquiterpene, sesterterpene, triterpene, tetraterpene, polyterpene, or a mixture thereof.

32. The method of claim 19, wherein after incubation or cultivation, one or more host cells, host tissues, host seeds, or host plants has at least 300 micrograms terpenoids per gram fresh weight or at least 0.03% fresh weight monoterpene, diterpene, sesquiterpene, sesterterpene, triterpene, tetraterpene, polyterpene, or a mixture thereof.


SEQUENCE LISTING (111 sequences)

SEQ ID NO: 1 - protein, 158 aa, Nannochloropsis oceanica
MAGPIMTSAPSATTPTGKTMPFKQPFKTVATLSAKTGNITKPIDPAISKTIDFVYNGYSTVKTKVDKAPKVNPYLLIAGGLVLSCIISMCLLVPAVIFFPVTIFLGVATSFALIALAPVAFVFGWILISSAPIQDKVVVPALDKVLANKKVAKFLLKE

SEQ ID NO: 2 - DNA, 561 bp, Nannochloropsis oceanica
tttaaaggaaaaacaacagaccaccaccaatctcagcccgcatcaacaatggccggccccatcatgacctctgcgccctccgcgaccacgcccacgggcaagacaatgccgttcaagcagcctttcaagactgtggccacgctgtccgccaagactggcaacattaccaagcccatcgaccctgccatctccaagaccattgacttcgtctacaatggttactcgacggtcaagaccaaggttgacaaggcccctaaggtaaacccctacctgctcattgccggcggcctcgtcctctcgtgcatcatctccatgtgcctgctcgtcccggccgtgatcttcttccccgtcaccatcttcctgggtgtcgctacgtcgtttgcgctcattgcattggcccccgtggcttttgtgttcgggtggatcctgatctcctctgctccgatccaggataaggtggtggtgcccgccttggacaaggtgctggccaataagaaggtggcgaagttcctcctcaaggagtaagaaagatccaagagagacgagtagagattttttttt

SEQ ID NO: 3 - protein, 722 aa, Plectranthus barbatus
MASCGAIGSSFLPLLHSDESSFLSRHTAALHIKKQKFSVGAALYQDNTNDVVPSGEGLTRQKPRTLSFTGEKPSTPILDTINYPIHMKNLSVEELERLADELREEIVYTVSKTGGHLSSSLGVSELTVALHHVFNTPDDKIIWDVGHQAYPHKILTGRRSRMHTIRQTFGLAGFPKRDESPHDAFGAGHSSTSISAGLGMAVGRDLLQKNNHVISVIGDGAMTAGQAYEALNNAGFLDSNLIIVLNDNKQVSLPTATVDGPAPPVGALSKALTKLQASRKFRQLREAAKGMTKQMGNQAHEIASKVDTYVKGMMGKPGASLFEELGIYYIGPVDGHNIEDLVYIFKKVKEMPAPGPVLIHIITEKGKGYPPAEVAADKMHGVVKFDPTTGKQMKVKAKTQSYTQYFAESLVAEAEQDEKVVAIHAAMGGGTGLNIFQKRFPDRCFDVGIAEQHAVTFAAGLATEGLKPFCTIYSSFLQRGYDQVVHDVDLQKLPVRFMMDRAGLVGADGPTHCGAFDTTYMACLPNMVVMAPSDEAELMHMVATAAVIDDRPSCVRYPRGNGIGVPLPPNNKGIPLEVGKGRILKEGNRVAILGFGTIVQNCLAAAQLLQEHGISVSVADARFCKPLDGDLIKNLVKEHEVLITVEEGSIGGFSAHVSHFLSLNGLLDGNLKWRPMVLPDRYIDHGAYPDQIEEAGLSSKHIAGTVLSLIGGGKDSLHLINM

SEQ ID NO: 4 - DNA, 2166 bp, Plectranthus barbatus
atggcgtcttgtggagctatcgggagtagtttcttgccactgctccattccgacgagtcaagcttcttatctcggcacactgctgctcttcacatcaagaagcagaagttttctgtgggagctgctctgtaccaggataacacgaacgatgtcgttccgagtggagagggtctgacgaggcagaaaccaagaactctgagtttcacgggagagaagccttcaactccaattttggataccatcaactatccaatccacatgaagaatctgtccgtggaggaactggagagattggccgatgaactgagggaggagatagtttacacggtgtcgaaaacgggagggcatttgagctcaagcttgggtgtatcagagctcaccgttgcactgcatcatgtattcaacacacccgatgacaaaatcatctgggatgttggacatcaggcgtatccacacaaaatcttgacagggaggaggtccagaatgcacaccatccgacagactttcgggcttgcagggttccccaagagggatgagagcccgcacgacgccttcggagctggtcacagctccactagtatttcagctggtctagggatggcggtggggagggacttgctgcagaagaacaaccacgtgatctcggtgatcggcgacggggccatgacagcggggcaggcatacgaggccttgaacaatgcaggatttcttgattccaatctgatcatcgtgttgaacgacaacaaacaagtgtccctgcctacagccacagtcgacggccctgctcctcccgtcggagccttgagcaaagccctcaccaagctgcaagcaagcaggaagttccggcagctacgagaagcagcaaaaggcatgactaagcagatgggaaaccaagcacacgaaattgcatccaaggtagacacttacgttaaaggaatgatggggaaaccaggcgcctccctcttcgaggagctcgggatttattacatcggccctgtagatggacataacatcgaagatcttgtctatattttcaagaaagttaaggagatgcctgcgcccggccctgttcttattcacatcatcaccgagaagggcaaaggctaccctccagctgaagttgctgctgacaaaatgcatggtgtggtgaagtttgatccaacaacggggaaacagatgaaggtgaaagcgaagactcaatcatacacccaatacttcgcggagtctctggttgcagaagcagagcaggacgagaaagtggtggcgatccacgcggccatgggaggcggaacggggctgaacatcttccagaaacggtttcccgaccgatgtttcgatgtcgggatagccgagcagcatgcagtcaccttcgccgcgggtcttgcaacggaaggcctcaagcccttctgcacaatctactcttccttcctgcagcgaggctatgatcaggtggtgcacgatgtggatcttcagaaactcccggtgagattcatgatggacagagctggactggtgggagctgacggcccaacccattgcggcgccttcgacaccacctacatggcctgcctgcccaacatggtggtcatggctccctcagatgaggctgagctcatgcacatggtcgccaccgccgccgtcattgatgatcgccctagctgcgttaggtaccctagaggaaacggtataggggtgcccctccctccaaacaacaaaggaattccattagaggttgggaagggaaggattttgaaagagggtaaccgagttgccattctaggcttcggaactatcgtgcaaaactgtctagcagcagcccaacttcttcaagaacacggcatatccgtgagcgtagccgatgcgagattctgcaagcctctggatggagatctgatcaagaatcttgtgaaggagcacgaagttctcatcactgtggaagagggatccattggaggattcagtgcacatgtctctcatttcttgtccctcaatggactcctcgacggcaatcttaagtggaggcctatggtgctcccagataggtacattgatcatggagcataccctgatcagattgaggaagcagggctgagctcaaagcatattgcaggaactgttttgtcacttattggtggagggaaagacagtcttcatttgatcaacatg

SEQ ID NO: 5 - protein, 722 aa, Plectranthus barbatus
MASCGAIGSSFLPLLHSDESSLLSRPTAALHIKKQKFSVGAALYQDNTNDVVPSGEGLTRQKPRTLSFTGEKPSTPILDTINYPIHMKNLSVEELEILADELREEIVYTVSKTGGHLSSSLGVSELTVALHHVFNTPDDKIIWDVGHQAYPHKILTGRRSRMHTIRQTFGLAGFPKRDESPHDAFGAGHSSTSISAGLGMAVGRDLLQKNNHVISVIGDGAMTAGQAYEAMNNAGFLDSNLIIVLNDNKQVSLPTATVDGPAPPVGALSKALTKLQASRKFRQLREAAKGMTKQMGNQAHEIASKVDTYVKGMMGKPGASLFEELGIYYIGPVDGHNIEDLVYIFKKVKEMPAPGPVLIHIITEKGKGYPPAEVAADKMHGVVKFDPTTGKQMKVKTKTQSYTQYFAESLVAEAEQDEKVVAIHAAMGGGTGLNIFQKRFPDRCFDVGIAEQHAVTFAAGLATEGLKPFCTIYSSFLQRGYDQVVHDVDLQKLPVRFMMDRAGLVGADGPTHCGAFDTTYMACLPNMVVMAPSDEAELMHMVATAAVIDDRPSCVRYPRGNGIGVPLPPNNKGIPLEVGKGRILKEGNRVAILGFGTIVQNCLAAAQLLQEHGISVSVADARFCKPLDGDLIKNLVKEHEVLITVEEGSIGGFSAHVSHFLSLNGLLDGNLKWRPMVLPDRYIDHGAYPDQIEEAGLSSKHIAGTVLSLIGGGKDSLHLINM

SEQ ID NO: 6 - DNA, 2169 bp, Plectranthus barbatus
atggcgtcttgtggagctatcgggagtagtttcttgccactgctccattccgacgagtcaagcttgttatctcggcccactgctgctcttcacatcaagaagcagaagttttctgtgggagctgctctgtaccaggataacacgaacgatgtcgttccgagtggagagggtctgacgaggcagaaaccaagaactctgagtttcacgggagagaagccttcaactccaattttggataccatcaactatccaatccacatgaagaatctgtccgtggaggaactggagatattggccgatgaactgagggaggagatagtttacacggtgtcgaaaacgggagggcatttgagctcaagcttgggtgtatcagagctcaccgttgcactgcatcatgtattcaacacacccgatgacaaaatcatctgggatgttggacatcaggcgtatccacacaaaatcttgacagggaggaggtccagaatgcacaccatccgacagactttcgggcttgcagggttccccaagagggatgagagcccgcacgacgcgttcggagctggtcacagctccactagtatttcagctggtctagggatggcggtggggagggacttgctacagaagaacaaccacgtgatctcggtgatcggagacggagccatgacagcggggcaggcatacgaggccatgaacaatgcaggatttcttgattccaatctgatcatcgtgttgaacgacaacaaacaagtgtccctgcctacagccaccgtcgacggccctgctcctcccgtcggagccttgagcaaagccctcaccaagctgcaagcaagcaggaagttccggcagctacgagaagcagcaaaaggcatgactaagcagatgggaaaccaagcacacgaaattgcatccaaggtagacacttacgttaaaggaatgatggggaaaccaggcgcctccctcttcgaggagctcgggatttattacatcggccctgtagatggacataacatcgaagatcttgtctatattttcaagaaagttaaggagatgcctgcgcccggccctgttcttattcacatcatcaccgagaagggcaaaggctaccctccagctgaagttgctgctgacaaaatgcatggtgtggtgaagtttgatccaacaacggggaaacagatgaaggtgaaaacgaagactcaatcatacacccaatacttcgcggagtctctggttgcagaagcagagcaggacgagaaagtggtggcgatccacgcggcgatgggaggcggaacggggctgaacatcttccagaaacggtttcccgaccgatgtttcgatgtcgggatagccgagcagcatgcagtcaccttcgccgcgggtcttgcaacggaaggcctcaagcccttctgcacaatctactcttccttcctgcagcgaggttatgatcaggtggtgcacgatgtggatcttcagaaactcccggtgagattcatgatggacagagctggacttgtgggagctgacggcccaacccattgcggcgccttcgacaccacctacatggcctgcctgcccaacatggtcgtcatggctccctccgatgaggctgagctcatgcacatggtcgccactgccgctgtcattgatgatcgccctagctgcgttaggtaccctagaggaaacggtataggggtgcccctccctccaaacaataaaggaattccattagaggttgggaagggaaggattttgaaagagggtaaccgagttgccattctaggcttcggaactatcgtgcaaaactgtctagcagcagcccaacttcttcaagaacacggcatatccgtgagcgtagccgatgcgagattctgcaagcctctggatggagatctgatcaagaatcttgtgaaggagcacgaagttctcatcactgtggaagagggatccattggaggattcagtgcacatgtctctcatttcttgtccctcaatggactcctcgacggcaatcttaagtggaggcctatggtgctcccagataggtacattgatcatggagcataccctgatcagattgaggaagcagggctgagctcaaagcatattgcaggaactgttttgtcacttattggtggagggaaagacagtcttcatttgatcaacatgtaa

SEQ ID NO: 7 - protein, 722 aa, Isodon rubescens
MASCGAIRSSFLPLLHSDDSSLLSRTAAALPIKKQKFSVGAALQQDNSNDVAANGESLTRQKPRALSFTGEKPSTPILDTINYPNHMKNLSVEELERLADELREEIVYSVSKTGGHLSSSLGVSELTVALHHVFNTPDDKIIWDVGHQAYPHKILTGRRSRMNTIRQTFGLAGFPKRDESAHDAFGAGHSSTSISAGLGMAVGRDLLKKNNHVISVIGDGAMTAGQAYEALNNAGFLDSNLIVVLNDNKQVSLPTATVDGPAPPVGALSKALTRLQASRKFRQLREAAKGMTKQMGNQAHEVASKVDTYVKGMMGKPGASLFEELGIYYIGPVDGHSMEDLVYIFQKVKEMPAPGPVLIHIITEKGKGYPPAEVAADKMHGVVKFDPTTGKQMKTKTKTQSYTQYFAESLVAEAEQDEKVVAIHAAMGGGTGLNIFQKRFPERCFDVGIAEQHAVTFAAGLATEGLKPFCTIYSSFLQRGYDQVVHDVDLQKLPVRFMMDRAGLVGADGPTHCGAFDTTYMACLPNMVVMAPSDEAELMHMVATAGVIDDRPSCVRYPRGNGIGVPLPPNNKGNPLEIGKGRILKEGSRVAILGFGTIVQNCLAAAQLLQEHGISVSVADARFCKPLDGDLIKKLVKEHEVLITVEEGSIGGFSAHVSHFLSLNGLLDGNLKWRPMVLPDRYIDHGAYPDQIEEAGLSSKHIAGTVLSLIGGGKDSLHLINM

SEQ ID NO: 8 - DNA, 2169 bp, Isodon rubescens
atggcatcttgtggagctatcaggagcagtttcctgccattgctccattctgacgattctagcttgttatcccgcactgctgctgctcttcccatcaaaaagcaaaagttctctgtgggagcagctcttcaacaggataacagcaacgatgtggcggcgaatggagagagtctcacgaggcagaagccaagagctctcagttttacgggagaaaagccttcaactccaattttggatactattaactatccaaaccacatgaaaaatctttccgtcgaggaactagagagattggctgatgaattgagggaagagatagtttactcggtgtccaaaacgggagggcatttaagttcaagcctaggtgtatcagagctcacagttgcacttcatcatgtattcaacacacctgatgataaaatcatttgggatgtcggacatcaggcgtatccacacaaaatcttgacggggaggaggtcaagaatgaacacgattcgacagactttcgggttagccgggttccccaagagggatgagagcgcgcacgatgcgtttggagctggtcacagttcaactagcatttcagctggtctagggatggcggtggggagggacttgctaaagaagaacaaccacgtcatatcagtgatcggagatggggccatgacagccggacaggcatatgaggctttgaacaatgcaggattcctggactccaatctcatcgtcgtcttgaacgacaacaagcaagtgtccctgcccactgccaccgtcgacggccctgctccccccgttggagccctcagcaaagccctcaccagactgcaagccagcagaaaattccgccagctccgtgaagcagctaaaggcatgactaagcagatgggaaaccaagcccacgaagttgcatcaaaggtggacacttatgtgaagggaatgatggggaaacccggcgcctccctcttcgaggagcttgggatttattacatcggccctgtagatggccacagtatggaagatcttgtctatattttccagaaagttaaggagatgccggcgcctggacctgttctcattcacatcataaccgagaagggcaaaggctatcctcctgctgaagttgctgcggataaaatgcatggtgtggtgaagtttgatccaacgacagggaaacagatgaagactaaaacgaagacacaatcatacactcaatacttcgcggagtccctagttgcagaagcagagcaggacgagaaggtggtggcgatccacgcggcaatgggaggcgggacgggcctcaacatcttccagaagcggtttcctgagcgatgttttgatgttgggattgcagagcagcacgcagtcacctttgccgcgggtcttgcaactgaaggcctcaagcctttctgcacaatctactcttccttcctgcagagaggctacgatcaggtggttcacgatgtagaccttcagaagctccccgtgagattcatgatggacagagctggactggtgggagcagacggccccacccattgcggcgccttcgacaccacctacatggcctgcctccccaacatggtggtcatggctccctccgacgaggccgagctcatgcacatggtcgccaccgctggagtcattgatgaccgccccagttgcgtcagataccctagaggaaacggtataggggtacctcttccaccaaacaacaaaggaaatccattggagattgggaagggaaggatcttaaaagaggggagtagagttgccattttaggcttcgggactatcgttcaaaactgtttggcagcagcccaacttcttcaagaacacggcatatctgtgagcgtggctgatgcaagattctgcaagcccctggatggagatctgatcaagaaactggttaaggagcatgaagttctaatcactgtggaagagggatccattggcggattcagtgcacatgtttctcatttcttgtccctcaatggactgctggatgggaatcttaagtggaggccgatggtgctccctgataggtatattgatcatggagcataccctgatcagattgaagaagcagggctgagttcaaagcatattgcaggcactgttttgtcactgattggtggaggaaaagacagtcttcatttgatcaacatgtaa

SEQ ID NO: 9 - protein, 325 aa, Methanothermobacter thermautotrophicus
MMEVMDILRKYSEMADERIRESISDITPETLLRASEHLITAGGKKIRPSLALLSSEAVGGDPGDAAGVAAAIELIHTFSLIHDDIMDDDEIRRGEPAVHVLWGEPMAILAGDVLFSKAFEAVIRNGDSEMVKEALAVVVDSCVKICEGQALDMGFEERLDVTEEEYMEMIYKKTAALIAAATKAGAIMGGGSPQEIAALEDYGRCIGLAFQIHDDYLDVVSDEESLGKPVGSDIAEGKMTLMVVKALERASEKDRERLISILGSGDEKLVAEAIEIFERYGATEYAHAVALDHVRMAKERLEVLEESDAREALAMIADFVLEREH

SEQ ID NO: 10 - DNA, 978 bp, Methanothermobacter thermautotrophicus
atgatggaggtaatggacatactccgaaagtattcagaaatggcagatgagaggatccgagagtctataagtgatattactcctgaaacgctgcttagagcatcagagcacctgataacagccggaggcaagaaaatcaggccgagccttgctctcttatccagcgaagctgtgggcggggaccccggagacgctgctggagtcgccgccgcaatagagttgatacatacattctccttaatacatgatgatatcatggacgatgacgagatcaggaggggtgagccagccgtccatgtcttgtggggtgagccgatggctattctcgcaggtgacgtcttgtttagtaaggcttttgaggccgtaattagaaatggggattcagagatggtcaaagaagcccttgctgttgtggtggattcatgtgtcaagatatgcgagggtcaagctcttgacatgggtttcgaagagcgactggacgtaaccgaggaagagtatatggagatgatatataaaaaaactgcagcattgattgctgctgctacaaaggcaggagccatcatgggtggcggatcaccccaggaaatcgcagctcttgaagactatgggagatgtattgggttggcatttcaaatccacgacgactatttagatgtagtttctgatgaggaaagtctgggaaagcccgttgggtctgacatagcagaaggcaagatgacactgatggtcgtcaaagccttagagagagcttctgaaaaagatagggagaggttgatctctatactcgggagtggcgacgagaagcttgtggccgaagccatcgaaattttcgaacgatacggagcaactgaatatgctcacgccgtggccctggatcatgtgcgtatggctaaggagcgtttggaagtcctcgaagagtccgatgccagggaagctttagccatgattgcagattttgtgttagagcgtgaacactaa

SEQ ID NO: 11 - protein, 372 aa, Euphorbia peplus
MAFSATFSSCDYSLLLKKSSVNGLKNHPKVPFSGQHFKLMKANFTTRALTVSKSSAVQQPPLTAADSQGSNSNTIPLPPFAFDEYMKTKAKSVNKALDDAIPIQHPIKIHESMRYSLLAGGKRVRPVLCIAACELVGGDEAAAMPSACAMEMIHTMSLIHDDLPCMDNDDLRRGKPTNHIKYGEETAILAGDALLSFSFEHVARATKNVSPDRMIRVIGELGSAVGSEGLVAGQIVDIDSEGKEVSLSDLEYIHIHKTAKLLEAAVVCGAIVGGADDESVERMRKYARCIGLLFQVVDDILDVTKSSEELGKTAGKDLATDKATYPKLLGIDEARKLAAKLVEQANQELAYFDAAKAAPLYHFANYIASRQN

SEQ ID NO: 12 - DNA, 1119 bp, Euphorbia peplus
atggccttctccgcgacattttccagctgcgactactcacttcttttaaaaaaatcatccgtcaatggcctcaaaaaccacccgaaagttccattttctggtcaacacttcaagttaatgaaagccaacttcaccacccgtgccctgaccgtttccaaatcctccgcggtgcagcaaccaccgctcactgcggcggattctcaaggatcaaattccaatactatccctcttcctccattcgcattcgacgaatacatgaaaaccaaggctaaaagcgtcaacaaagcattagacgacgctattccgattcaacatccgatcaaaatccatgaatccatgagatactctctcctcgccggcggcaagcgtgtccggccagttttatgtatagctgcttgtgaactagtcggaggagacgaagcagcagctatgccgtcagcatgtgctatggaaatgatccataccatgtcattaatccacgacgatcttccttgtatggacaacgacgatcttcgtcgcggaaaaccaacaaaccacataaaatacggggaagaaaccgccattcttgccggcgatgcactcctttcattttcctttgaacacgtagctagggcaacaaaaaacgtttccccggaccggatgatccgagtcataggggagctaggttcagctgtgggttcggaaggtttagtcgcgggacaaatcgtggacatcgatagcgaggggaaggaagtgagtttaagtgatttggagtatattcatattcataagacggctaagcttttggaagcagccgtcgtgtgtggtgcgatagtcggtggcgccgacgatgaaagtgtggagagaatgaggaaatatgctagatgtataggcctattgttccaagttgtggatgatatattagatgtgacaaagtcatcggaggagctcgggaagaccgcggggaaagatttagcgacggataaagcgacgtatccgaagttgttggggattgacgaggcgaggaaacttgcagctaaattggtggagcaagctaatcaagaacttgcttattttgatgctgctaaggctgctccgttatatcattttgctaattatattgctagtaggcaaaattga

SEQ ID NO: 13 - protein, 352 aa, Euphorbia peplus
MNSMNLGSWLNTSSIFNQSTRSRSPPLKSFSIRLPRHKPRFISSIMTKEEETLTQKPQFDFKSYMLQKAASIHQALDAAVSIKEPAKIHESMRYSLLAGGKRVRPALCLAACELVGGNDSQAMPAACAVEMVHTMSLIHDDLPCMDNDDLRRGKPTNHIVFGEDVAVLAGDALLSFAFEHIAVATVNVSPERIVRAIGELASAIGAEGLVAGQVVDIACEKACDVGLETLEFIHVHKTAKLLECAVVLGAILGGGKDDEIEKLRKYARGIGLLFQVVDDILDVTKSSEELGKTAGKDLVADKVTYPKLLGIEKSREFAEKLNREAQQQLSEFDVEKAAPLIALANYIAYRQN

SEQ ID NO: 14 - DNA, 1059 bp, Euphorbia peplus
atgaactccatgaatttgggttcatggctcaacacttcttcaatcttcaaccaatctaccagatccagatccccgccattaaaatccttctcaattcgtcttccccgtcacaaacccagattcatttcttcaattatgaccaaagaagaagaaaccctaacccaaaaaccccaatttgatttcaaatcttacatgctccaaaaagctgcttccattcatcaagctctagacgccgccgtttcgatcaaagaacccgctaaaatccatgaatccatgcggtattccctcttagccggcgggaaaagagtccggccagcgttatgtttagccgcgtgtgagctcgtcggcgggaacgattctcaggcgatgccggcggcttgcgcggtggaaatggtccacacgatgtctcttattcacgatgatctcccctgtatggataacgatgatctacgccgcggaaaacccacgaaccatatcgtgttcggggaagacgtggcggttctcgctggggatgcgttgctctcgttcgcattcgagcacattgcggttgctacggtgaatgtgtcaccggagaggattgtccgggccatcggggaattagccagcgcgattggggcagaagggttagttgctggacaagtggttgatatagcttgtgagaaagcttgtgatgtgggattagaaacgttggagttcattcatgttcacaaaacggcgaaattgctggaatgcgctgtcgtattgggggcaatattagggggaggaaaggatgatgagattgagaagttgaggaaatatgcaagaggaatagggttgttgtttcaagtagtggatgatattttagatgtcacaaaatcatcggaagagttggggaaaactgcagggaaagatttggtggcggataaggtaacataccctaaacttttagggattgaaaaatcaagggaatttgctgagaaattgaatagggaagctcaacaacagttgagtgagtttgatgtggaaaaggcagctcctttgattgctttggctaattatattgcttataggcagaattga

SEQ ID NO: 15 - protein, 330 aa, Sulfolobus acidocaldarius
MSYFDNYFNEIVNSVNDIIKSYISGDVPKLYEASYHLFTSGGKRLRPLILTISSDLFGGQRERAYYAGAAIEVLHTFTLVHDDIMDQDNIRRGLPTVHVKYGLPLAILAGDLLHAKAFQLLTQALRGLPSETIIKAFDIFTRSIIIISEGQAVDMEFEDRIDIKEQEYLDMISRKTAALFSASSSIGALIAGANDNDVRLMSDFGTNLGIAFQIVDDILGLTADEKELGKPVFSDIREGKKTILVIKTLELCKEDEKKIVLKALGNKSASKEELMSSADIIKKYSLDYAYNLAEKYYKNAIDSLNQVSSKSDIPGKALKYLAEFTIRRRK

SEQ ID NO: 16 - DNA, 993 bp, Sulfolobus acidocaldarius
atgagttattttgacaactacttcaatgaaatagtcaacagcgtcaatgatataatcaaatcctacatcagtggagacgtgccaaaactctacgaagcatcataccacctgttcacatctggaggaaaacgattgagacccttgatattaaccataagtagcgacctctttgggggccagagagaaagagcatattacgctggagcagctatcgaggtgttacatacattcaccttggtgcatgatgacattatggatcaggacaatataaggcgaggtttaccgactgtgcatgtgaaatacggtctgccgctggctattctggccggcgatttactccatgccaaggccttccagttgctcacccaggcactccgtggactgcccagcgagacaattatcaaagcctttgacattttcacgagatccataataattatttccgagggccaagctgtcgatatggaatttgaagataggatagatattaaagagcaggaatatctcgacatgattagccgaaaaaccgctgctctcttcagtgcctctagctccatcggcgctttaatcgccggcgcaaacgataatgacgtcagacttatgtctgatttcgggactaatctcggcatcgcctttcagatcgtagacgatattcttggtctgactgcagatgaaaaggagcttgggaagccggtgttctccgacatccgtgaaggtaaaaagacgatcttggtcatcaagacgctggaactttgcaaagaagatgagaagaagatcgtgctcaaggccttaggcaacaagagcgccagtaaggaggagctcatgtctagtgctgatatcattaaaaagtacagccttgactacgcctataacctcgcagagaaatactataagaacgctatcgattctttaaaccaagtcagctctaagagcgatatccctggtaaagcactgaagtatctcgctgaatttacaataaggagacgtaagtaa

SEQ ID NO: 17 - protein, 324 aa, Mortierella elongata
MAIPSIYPTDHDEAALLEPYTYICSNPGKEMRTELIEAFNIWIKVPPQELAIITKVVKMLHTSSLLVDDIEDDSILRRGEPVAHKIFGVPATINCANYVYFLALAELSKISNPKMLTIFTEELLCLHRGQGMELLWRDSLTCPTEEEYIAMVNDKTGGLLRLAVKLMQAASDSTVDYVPMVELIGIHFQIRDDYLNLQSSQYSANKGFCEDLTEGKFSYPIIHSIRAAPNSRKLLNILKQKPKDHELKVYAVSLMNATKTFEYCRQQLTLYEERARAEVRRLGGNARLEKIIDRLSIPDPDSADAEKDVVPMFVATSTAGGAAK

SEQ ID NO: 18 - DNA, 975 bp, Mortierella elongata
atggctataccttctatttaccctacggatcacgatgaagctgcccttctggagccgtacacgtatatatgcagtaatccgggaaaggagatgaggaccgagttaatagaagcctttaatatctggatcaaagtgccccctcaggagttggcaatcatcacaaaggtcgttaagatgttacatacaagctcactcttggtagatgacattgaagatgatagtattctccgtcgaggcgagccagttgcacacaaaatattcggtgttccggcaactataaactgtgctaattatgtttacttcctcgccttagctgaattgtctaagatatctaatccaaaaatgcttacgatatttaccgaagagcttctttgccttcataggggacaaggcatggagctcctttggcgtgatagcttaacgtgcccgaccgaggaagagtatatagctatggtgaacgataaaactggaggccttcttagactggccgttaagctcatgcaggcagctagtgactctaccgtagactacgtcccaatggtggaactcattggcattcattttcaaataagggacgattacttaaaccttcagagttctcagtacagtgcaaacaaaggtttttgcgaggacctgactgagggcaagttttcctatccgattattcactccataagggcagcacctaatagtcgaaagttgttgaacatcttgaagcagaaacctaaagatcatgaactcaaggtttatgccgtgtcattaatgaacgctacgaaaacatttgagtattgtaggcagcagctgaccctttacgaggaacgtgcccgagcagaagtgaggcgtttgggagggaatgctaggctcgaaaaaatcatcgacagactctctattccagaccccgacagcgcagatgcagagaaggacgtggttcctatgttcgttgcaacgtcaactgctggtggagctgcaaagtaa

SEQ ID NO: 19 - protein, 309 aa, Tolypothrix sp.
MVATDKFKKMPETATFNLSAYLKERQQLCETALDQALPVSYPEKIYESMRYSLLAGGKRVRPILCLATSEMMGGTIEMAMPTACAVEMIHTMSLIHDDLPAMDNDDYRRGKLTNHKVYGEDIAILAGDGLLAYAFEFVAIATPLTVPRDRVLQVVARLARALGAAGLVGGQVVDLESEGKTDTSLETLNYIHNHKTAALLEACVVCGGILAGASVEDVQRLTRYAQNIGLAFQIVDDILDITATQEQLGKTAGKDLKAQKVTYPSLWGIEESRVKAEQLIEAACAELDVFGEKAQPLKAIAHFIISRNH

SEQ ID NO: 20 - DNA, 930 bp, Tolypothrix sp.
atggtagcaactgataagtttaaaaagatgccagagacagccacgtttaacctatcagcgtatctcaaagagcgtcaacagctttgtgaaactgctttggatcaagcgcttcccgtttcctatccagagaagatttacgagtcgatgcgctattctctcttagctggtggcaaacgtgtgcgtcctatcctgtgccttgctaccagtgaaatgatgggcggcacaatcgaaatggcaatgccaacagcttgtgcggtggaaatgatccacacaatgtcattaattcatgatgatttgccagcgatggataatgacgattaccgtcggggtaagctgacaaaccacaaggtttatggcgaagatatcgcgattttagctggcgatggtttgttggcctatgcttttgaatttgttgcgatcgccacccctttaactgtccctagagatagagtattgcaggtagtagcgcgtcttgctcgggcattaggggctgctggcttggttgggggccaagtagtggatctagaatcagaaggtaaaacagatacttccctagagactctgaattacattcataaccacaaaacagctgcccttttggaagcttgtgttgtttgtggtggtattttagcgggagcatctgttgaagatgtacaaagactaactcggtatgctcagaatattggtctggcattccaaattgttgatgatattttagatatcaccgctactcaagaacaattaggcaaaactgctggcaaggatttgaaagcgcagaaagttacttatcccagcctgtggggaattgaagaatctcgcgttaaagccgaacaactcattgaagcagcatgtgcggaattagacgtatttggagaaaaagcacaacctttaaaagcgatcgctcattttattatcagccgcaatcactaa

SEQ ID NO: 21 - protein, 582 aa, Euphorbia lathyris
MDSTRPESKLRRPIRRISDEVDHHGRCLSPPPKASDALPLPLYLTNAVFFTLFFSVAYYLLHRWRDKIRNSTPLHVVTLSEIAAIVSLIASFIYLLGFFGIDFVQSFIARASHDTWDLDDADRNYLIDGDHRLVTCSPAKISPINSLPPKMSSPPEPIISPLASEEDEEIVKSVVNGTIPSYSLESKLGDCKRAAEIRREALQRMMGRSLEGLPVEGFDYESILGQCCEMPVGYVQIPVGIAGPLLLDGQEYSVPMATTEGCLVASTNRGCKAIHLSGGASSVLLKDGMTRAPVVRFASAMRAADLKFFLENPENFDSLSIAFNRSSRFAKLQSIQCSIAGKNLYMRFTCSTGDAMGMNMVSKGVQNVLDFLQSDFPDMDVIGISGNFCSDKKPAAVNWIQGRGKSVVCEAIIKEEVVKKVLKSSVASLVELNMLKNLTGSAIAGALGGFNAHAGNIVSAIFIATGQDPAQNVESSHCITMMEAVNDGKDLHISVTMPSIEVGTVGGGTQLASQSACLNLLGVKGASKESPGANSRLLATIVAGSVLAGELSLMSAIAAGQLVRSHMKYNRSSKDVTKFASS

SEQ ID NO: 22 - DNA, 2358 bp, Euphorbia lathyris
acgcataaacacattcaaacagctactcttccagctcttccttttttcccccatttccacttccattattttatccccccttttttctctcttcttctcgattcatccatggattccactcggccggaatccaaactccggcgaccgatccgccgcatctcggacgaggttgaccaccacggccgctgtctctctccgcctcctaaagcctccgatgctctccctctcccgttgtatttaaccaatgcggttttctttactctctttttctccgtcgcgtactatcttctccaccggtggagagataagatccgtaattctactcctcttcatgtcgttactctctctgaaattgccgccattgtttctctcattgcgtctttcatctacctgcttggattcttcgggattgatttcgttcagtctttcattgcacgcgcttctcatgacacgtgggaccttgatgatgcggatcgtaactacctcattgatggagatcaccgtctcgttacttgctctcctgcgaagatttctccgattaattctcttcctcctaaaatgtcttccccgccggaaccgattatttcgcctctggcatccgaggaggatgaggaaattgttaaatctgttgttaatggaacgattccttcgtattcgttggaatcgaagcttggggattgtaaaagagcggctgagattcgacgggaggctttgcagagaatgatggggaggtcgttggagggtttacctgttgaaggattcgattatgagtcgattttaggtcagtgctgtgaaatgcctgttggttatgtgcagattccggttggaattgctgggccgttgctgctagacgggcaagagtactctgttccgatggcgaccaccgagggttgtttggttgctagcactaatagagggtgtaaagcgatccatttgtcaggtggtgctagtagtgtcttgttgaaggatggcatgactagagctcccgttgttcgattcgcctcggccatgagggccgcggatttgaagtttttcttagagaatcctgagaatttcgatagcttgtccatcgctttcaataggtccagtagatttgcaaagctccaaagcatacaatgttctattgctggaaagaatctatatatgagattcacctgcagcactggtgatgcaatggggatgaacatggtttccaaaggggttcaaaacgttcttgacttccttcaaagtgatttccctgacatggatgttattggcatctcaggaaatttttgttcggacaagaagccagctgctgtgaactggattcaagggcgaggcaaatcggttgtttgcgaggcaattatcaaggaagaggtggtgaagaaggtattgaaatcaagtgttgcttcactagtagagctgaacatgctcaagaatcttactggttcagctattgctggagctcttggtggattcaatgcacatgctggcaacatagtctctgcaattttcattgccactggccaggatccagcccagaatgttgagagttctcattgcatcaccatgatggaagctgtcaatgatggaaaagatctccacatctctgtaaccatgccttcaatcgaggtaggaacagttggaggagggacacaactagcatcccaatcagcatgtctgaacctactcggtgtaaaaggagcaagtaaagaatcaccaggagcaaactcaaggctcctagccacaatagtagctggttcagtcctagctggtgaactctccctaatgtcagccatagcagcaggacaactagtccggagccacatgaagtacaacagatccagcaaagatgtaaccaaatttgcatcatcttaatcaaaactggttcacaataataaaagcgtccgaaccaaacctcatagacagagagccagatagacagagccagaaagagaaaggggaagaaaatggaagaagaagactgtactgtagggtacctaccccatgtgagtttttttattttttttcaaagcttttaatagctgtaaagttgcttaatcatatggagagaagaaagaagaattaggtacacaaaacttttgaaaatctccattttcttaccccaaatttgagaagtgggtgtactgtattagtatgttggtgagcacatgtgagcaaaaaaggtccccactatctactacctagtgttttttgtgtatgtttgtgtcctaatttatttgttaatgtttagttgctttctttcttctattttttgcatacatatgttgtgtacacttgtttttgtgtttgaacttacctggggctgacatgtgacacgtggcgtgatattgtttgttgttgatttccttttttttt

SEQ ID NO: 23 - protein, 425 aa, Euphorbia lathyris
MISPLASEEDEEIVKSVVNGTIPSYSLESKLGDCKRAAEIRREALQRMMGRSLEGLPVEGFDYESILGQCCEMPVGYVQIPVGIAGPLLLDGQEYSVPMATTEGCLVASTNRGCKAIHLSGGASSVLLKDGMTRAPVVRFASAMRAADLKFFLENPENFDSLSIAFNRSSRFAKLQSIQCSIAGKNLYMRFTCSTGDAMGMNMVSKGVQNVLDFLQSDFPDMDVIGISGNFCSDKKPAAVNWIQGRGKSVVCEAIIKEEVVKKVLKSSVASLVELNMLKNLTGSAIAGALGGFNAHAGNIVSAIFIATGQDPAQNVESSHCITMMEAVNDGKDLHISVTMPSIEVGTVGGGTQLASQSACLNLLGVKGASKESPGANSRLLATIVAGSVLAGELSLMSAIAAGQLVRSHMKYNRSSKDVTKFASS

SEQ ID NO: 24 - DNA, 1278 bp, Euphorbia lathyris
atgatttcgcctctggcatccgaggaggatgaggaaattgttaaatctgttgttaatggaacgattccttcgtattcgttggaatcgaagcttggggattgtaaaagagcggctgagattcgacgggaggctttgcagagaatgatggggaggtcgttggagggtttacctgttgaaggattcgattatgagtcgattttaggtcagtgctgtgaaatgcctgttggttatgtgcagattccggttggaattgctgggccgttgctgctagacgggcaagagtactctgttccgatggcgaccaccgagggttgtttggttgctagcactaatagagggtgtaaagcgatccatttgtcaggtggtgctagtagtgtcttgttgaaggatggcatgactagagctcccgttgttcgattcgcctcggccatgagggccgcggatttgaagtttttcttagagaatcctgagaatttcgatagcttgtccatcgctttcaataggtccagtagatttgcaaagctccaaagcatacaatgttctattgctggaaagaatctatatatgagattcacctgcagcactggtgatgcaatggggatgaacatggtttccaaaggggttcaaaacgttcttgacttccttcaaagtgatttccctgacatggatgttattggcatctcaggaaatttttgttcggacaagaagccagctgctgtgaactggattcaagggcgaggcaaatcggttgtttgcgaggcaattatcaaggaagaggtggtgaagaaggtattgaaatcaagtgttgcttcactagtagagctgaacatgctcaagaatcttactggttcagctattgctggagctcttggtggattcaatgcacatgctggcaacatagtctctgcaattttcattgccactggccaggatccagcccagaatgttgagagttctcattgcatcaccatgatggaagctgtcaatgatggaaaagatctccacatctctgtaaccatgccttcaatcgaggtaggaacagttggaggagggacacaactagcatcccaatcagcatgtctgaacctactcggtgtaaaaggagcaagtaaagaatcaccaggagcaaactcaaggctcctagccacaatagtagctggttcagtcctagctggtgaactctccctaatgtcagccatagcagcaggacaactagtccggagccacatgaagtacaacagatccagcaaagatgtaaccaaatttgcatcatcttaa

SEQ ID NO: 25 - protein, 384 aa, Arabidopsis thaliana
MSVSCCCRNLGKTIKKAIPSHHLHLRSLGGSLYRRRIQSSSMETDLKSTFLNVYSVLKSDLLHDPSFEFTNESRLWVDRMLDYNVRGGKLNRGLSVVDSFKLLKQGNDLTEQEVFLSCALGWCIEWLQAYFLVLDDIMDNSVTRRGQPCWFRVPQVGMVAINDGILLRNHIHRILKKHFRDKPYYVDLVDLFNEVELQTACGQMIDLITTFEGEKDLAKYSLSIHRRIVQYKTAYYSFYLPVACALLMAGENLENHIDVKNVLVDMGIYFQVQDDYLDCFADPETLGKIGTDIEDFKCSWLVVKALERCSEEQTKILYENYGKPDPSNVAKVKDLYKELDLEGVFMEYESKSYEKLTGAIEGHQSKAIQAVLKSFLAKIYKRQK

SEQ ID NO: 26 - DNA, 1396 bp, Arabidopsis thaliana
ggcgttttcgggagaagaaggaggaatatgagtgtgagttgttgttgtaggaatctgggcaagacaataaaaaaggcaataccttcacatcatttgcatctgagaagtcttggtgggagtctctatcgtcgtcgtatccaaagctcttcaatggagaccgatctcaagtcaacctttctcaacgtttattctgttctcaagtctgaccttcttcatgacccttccttcgaattcaccaatgaatctcgtctctgggttgatcggatgctggactacaatgtacgtggagggaaactcaatcggggtctctctgttgttgacagtttcaaacttttgaagcaaggcaatgatttgactgagcaagaggttttcctctcttgtgctctcggttggtgcattgaatggctccaagcttatttccttgtgcttgatgatattatggataactctgtcactcgccgtggtcaaccttgctggttcagagttcctcaggttggtatggttgccatcaatgatgggattctacttcgcaatcacatccacaggattctcaaaaagcatttccgtgataagccttactatgttgaccttgttgatttgtttaatgaggttgagttgcaaacagcttgtggccagatgatagatttgatcaccacctttgaaggagaaaaggatttggccaagtactcattgtcaatccaccgtcgtattgtccagtacaaaacggcttattactcattttatctccctgttgcttgtgcgttgcttatggcgggcgaaaatttggaaaaccatattgacgtgaaaaatgttcttgttgacatgggaatctacttccaagtgcaggatgattatctggattgttttgctgatcccgagacgcttggcaagataggaacagatatagaagatttcaaatgctcgtggttggtggttaaggcattagagcgctgcagcgaagaacaaactaagatattatatgagaactatggtaaacccgacccatcgaacgttgctaaagtgaaggatctctacaaagagctggatcttgagggagttttcatggagtatgagagcaaaagctacgagaagctgactggagcgattgagggacaccaaagtaaagcaatccaagcagtgctaaaatccttcttggctaagatctacaagaggcagaagtagtagagacagacaaacataagtctcagccctcaaaaatttcctgttatgtctttgattcttggttggtgatttgtgtaattctgttaagtgctctgattttcagggggaataataaacctgcctcacttttattcttgtgttacaattgtatttgtttcatgactatgatcttcttctttcatcagttatatgaatttgagattcttgttggttg

SEQ ID NO: 27 - protein, 342 aa, Arabidopsis thaliana
MADLKSTFLDVYSVLKSDLLQDPSFEFTHESRQWLERMLDYNVRGGKLNRGLSVVDSYKLLKQGQDLTEKETFLSCALGWCIEWLQAYFLVLDDIMDNSVTRRGQPCWFRKPKVGMIAINDGILLRNHIHRILKKHFREMPYYVDLVDLFNEVEFQTACGQMIDLITTFDGEKDLSKYSLQIHRRIVEYKTAYYSFYLPVACALLMAGENLENHTDVKTVLVDMGIYFQVQDDYLDCFADPETLGKIGTDIEDFKCSWLVVKALERCSEEQTKILYENYGKAEPSNVAKVKALYKELDLEGAFMEYEKESYEKLTKLIEAHQSKAIQAVLKSFLAKIYKRQK

SEQ ID NO: 28 - DNA, 1352 bp, Arabidopsis thaliana
caatcaggttccacatttggctttgcacaccttccttgatcctatcaatggcggatctgaaatcaaccttcctcgacgtttactctgttctcaagtctgatctgcttcaagatccttcctttgaattcacccacgaatctcgtcaatggcttgaacggatgcttgactacaatgtacgcggagggaagctaaatcgtggtctctctgtggttgatagctacaagctgttgaagcaaggtcaagacttgacggagaaagagactttcctctcatgtgctcttggttggtgcattgaatggcttcaagcttatttccttgtgcttgatgacatcatggacaactctgtcacacgccgtggccagccttgttggtttagaaagccaaaggttggtatgattgccattaacgatgggattctacttcgcaatcatatccacaggattctcaaaaagcacttcagggaaatgccttactatgttgacctcgttgatttgtttaacgaggtagagtttcaaacagcttgcggccagatgattgatttgatcaccacctttgatggagaaaaagatttgtctaagtactccttgcaaatccatcggcgtattgttgagtacaaaacagcttattactcattttatcttcctgttgcttgcgcattgctcatggcgggagaaaatttggaaaaccatactgatgtgaagactgttcttgttgacatgggaatttactttcaagtacaggatgattatctggactgttttgctgatcctgagacacttggcaagatagggacagacatagaagatttcaaatgctcctggttggtagttaaggcattggaacgctgcagtgaagaacaaactaagatactatacgagaactatggtaaagccgaaccatcaaacgttgctaaggtgaaagctctctacaaagagcttgatctcgagggagcgttcatggaatatgagaaggaaagctatgagaagctgacaaagttgatcgaagctcaccagagtaaagcaattcaagcagtgctaaaatctttcttggctaagatctacaagaggcagaagtagagacatactcgggcctctctccgttttattcttctgacatttatgtattggtgcatgacttcttttgccttagatcttatgttcccttccgaaaatagaatttgagattcttgttcatgcttatagtatagagacttagaaaatgtctatgtttcttttaatttctgaataaaaaatgtgcaatcagtgataaattgatacttgttaatgtggcaaaaattttgtgtcacatgagggtgcaacagaaatttggaaggacctgaggctgtttgagct

SEQ ID NO: 29 - protein, 430 aa, Arabidopsis thaliana
MKKRLTTSTCSSSPSSSVSSSTTTSSPIQSEAPRPKRAKRAKKSSPSGDKSHNPTSPASTRRSSIYRGVTRHRWTGRFEAHLWDKSSWNSIQNKKGKQVYLGAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESPPSSSSPLSCLSTDSASSTTTTTTSVSCNYLV

SEQ ID NO: 30 - DNA, 1540 bp, Arabidopsis thaliana
aaaccactctgcttcctcttcctctgagaaatcaaatcactcacactccaaaaaaaaatctaaactttctcagagtttaatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagagcccaccctcttcttcttcaccattgtcttgcttatctactgactctgcttcatcaacaacaacaacaacaacctcggtttcttgtaactatttggtctgagagagagagctttgccttctagtttgaatttctatttcttccgcttcttcttcttttttttcttttgttgggttctgcttagggtttgtatttcagtttcagggcttgttcgttggttctgaataatcaatgtctttgccccttttctaatgggtacctgaagggcga

SEQ ID NO: 31 - protein, 868 aa, Abies grandis
MAMPSSSLSSQIPTAAHHLTANAQSIPHFSTTLNAGSSASKRRSLYLRWGKGSNKIIACVGEGGATSVPYQSAEKNDSLSSSTLVKREFPPGFWKDDLIDSLTSSHKVAASDEKRIETLISEIKNMFRCMGYGETNPSAYDTAWVARIPAVDGSDNPHFPETVEWILQNQLKDGSWGEGFYFLAYDRILATLACIITLTLWRTGETQVQKGIEFFRTQAGKMEDEADSHRPSGFEIVFPAMLKEAKILGLDLPYDLPFLKQIIEKREAKLKRIPTDVLYALPTTLLYSLEGLQEIVDWQKIMKLQSKDGSFLSSPASTAAVFMRTGNKKCLDFLNFVLKKFGNHVPCHYPLDLFERLWAVDTVERLGIDRHFKEEIKEALDYVYSHWDERGIGWARENPVPDIDDTAMGLRILRLHGYNVSSDVLKTFRDENGEFFCFLGQTQRGVTDMLNVNRCSHVSFPGETIMEEAKLCTERYLRNALENVDAFDKWAFKKNIRGEVEYALKYPWHKSMPRLEARSYIENYGPDDVWLGKTVYMMPYISNEKYLELAKLDFNKVQSIHQTELQDLRRWWKSSGFTDLNFTRERVTEIYFSPASFIFEPEFSKCREVYTKTSNFTVILDDLYDAHGSLDDLKLFTESVKRWDLSLVDQMPQQMKICFVGFYNTFNDIAKEGRERQGRDVLGYIQNVWKVQLEAYTKEAEWSEAKYVPSFNEYIENASVSIALGTVVLISALFTGEVLTDEVLSKIDRESRFLQLMGLTGRLVNDTKTYQAERGQGEVASAIQCYMKDHPKISEEEALQHVYSVMENALEELNREFVNNKIPDIYKRLVFETARIMQLFYMQGDGLTLSHDMEIKEHVKNCLFQPVA

SEQ ID NO: 32 - DNA, 2861 bp, Abies grandis
agatggccatgccttcctcttcattgtcatcacagattcccactgctgctcatcatctaactgctaacgcacaatccattccgcatttctccacgacgctgaatgctggaagcagtgctagcaaacggagaagcttgtacctacgatggggtaaaggttcaaacaagatcattgcctgtgttggagaaggtggtgcaacctctgttccttatcagtctgctgaaaagaatgattcgctttcttcttctacattggtgaaacgagaatttcctccaggattttggaaggatgatcttatcgattctctaacgtcatctcacaaggttgcagcatcagacgagaagcgtatcgagacattaatatccgagattaagaatatgtttagatgtatgggctatggcgaaacgaatccctctgcatatgacactgcttgggtagcaaggattccagcagttgatggctctgacaaccctcactttcctgagacggttgaatggattcttcaaaatcagttgaaagatgggtcttggggtgaaggattctacttcttggcatatgacagaatactggctacacttgcatgtattattacccttaccctctggcgtactggggagacacaagtacagaaaggtattgaattcttcaggacacaagctggaaagatggaagatgaagctgatagtcataggccaagtggatttgaaatagtatttcctgcaatgctaaaggaagctaaaatcttaggcttggatctgccttacgatttgccattcctgaaacaaatcatcgaaaagcgggaggctaagcttaaaaggattcccactgatgttctctatgcccttccaacaacgttattgtattctttggaaggtttacaagaaatagtagactggcagaaaataatgaaacttcaatccaaggatggatcatttctcagctctccggcatctacagcggctgtattcatgcgtacagggaacaaaaagtgcttggatttcttgaactttgtcttgaagaaattcggaaaccatgtgccttgtcactatccgcttgatctatttgaacgtttgtgggcggttgatacagttgagcggctaggtatcgatcgtcatttcaaagaggagatcaaggaagcattggattatgtttacagccattgggacgaaagaggcattggatgggcgagagagaatcctgttcctgatattgatgatacagccatgggccttcgaatcttgagattacatggatacaatgtatcctcagatgttttaaaaacatttagagatgagaatggggagttcttttgcttcttgggtcaaacacagagaggagttacagacatgttaaacgtcaatcgttgttcacatgtttcatttccgggagaaacgatcatggaagaagcaaaactctgtaccgaaaggtatctgaggaatgctctggaaaatgtggatgcctttgacaaatgggcttttaaaaagaatattcggggagaggtagagtatgcactcaaatatccctggcataagagtatgccaaggttggaggctagaagctatattgaaaactatgggccagatgatgtgtggcttggaaaaactgtatatatgatgccatacatttcgaatgaaaagtatttagaactagcgaaactggacttcaataaggtgcagtctatacaccaaacagagcttcaagatcttcgaaggtggtggaaatcatccggtttcacggatctgaatttcactcgtgagcgtgtgacggaaatatatttctcaccggcatcctttatctttgagcccgagttttctaagtgcagagaggtttatacaaaaacttccaatttcactgttattttagatgatctttatgacgcccatggatctttagacgatcttaagttgttcacagaatcagtcaaaagatgggatctatcactagtggaccaaatgccacaacaaatgaaaatatgttttgtgggtttctacaatacttttaatgatatagcaaaagaaggacgtgagaggcaagggcgcgatgtgctaggctacattcaaaatgtttggaaagtccaacttgaagcttacacgaaagaagcagaatggtctgaagctaaatatgtgccatccttcaatgaatacatagagaatgcgagtgtgtcaatagcattgggaacagtcgttctcattagtgctcttttcactggggaggttcttacagatgaagtactctccaaaattgatcgcgaatctagatttcttcaactcatgggcttaacagggcgtttggtgaatgacaccaaaacttatcaggcagagagaggtcaaggtgaggtggcttctgccatacaatgttatatgaaggaccatcctaaaatctctgaagaagaagctctacaacatgtctatagtgtcatggaaaatgccctcgaagagttgaatagggagtttgtgaataacaaaataccggatatttacaaaagactggtttttgaaactgcaagaataatgcaactcttttatatgcaaggggatggtttgacactatcacatgatatggaaattaaagagcatgtcaaaaattgcctcttccaaccagttgcctagattaaattattcagttaaaggccctcatggtattgtgttaacattataataacagatgctcaaaagctttgagcggtatttgttaaggctatctttgtttgtttgtttgtttactgccaaccaaaaagcgttcctaaacctttgaagacatttccatccaagagatggagtctacattttatttatgagattgaattatttcaagagaatatactacatatatttaaaagtaaaaaaaaaaaaaaaaaaaaa

SEQ ID NO: 33 - protein, 784 aa, Abies grandis
VKREFPPGFWKDDLIDSLTSSHKVAASDEKRIETLISEIKNMFRCMGYGETNPSAYDTAWVARIPAVDGSDNPHFPETVEWILQNQLKDGSWGEGFYFLAYDRILATLACIITLTLWRTGETQVQKGIEFFRTQAGKMEDEADSHRPSGFEIVFPAMLKEAKILGLDLPYDLPFLKQIIEKREAKLKRIPTDVLYALPTTLLYSLEGLQEIVDWQKIMKLQSKDGSFLSSPASTAAVFMRTGNKKCLDFLNFVLKKFGNHVPCHYPLDLFERLWAVDTVERLGIDRHFKEEIKEALDYVYSHWDERGIGWARENPVPDIDDTAMGLRILRLHGYNVSSDVLKTFRDENGEFFCFLGQTQRGVTDMLNVNRCSHVSFPGETIMEEAKLCTERYLRNALENVDAFDKWAFKKNIRGEVEYALKYPWHKSMPRLEARSYIENYGPDDVWLGKTVYMMPYISNEKYLELAKLDFNKVQSIHQTELQDLRRWWKSSGFTDLNFTRERVTEIYFSPASFIFEPEFSKCREVYTKTSNFTVILDDLYDAHGSLDDLKLFTESVKRWDLSLVDQMPQQMKICFVGFYNTFNDIAKEGRERQGRDVLGYIQNVWKVQLEAYTKEAEWSEAKYVPSFNEYIENASVSIALGTVVLISALFTGEVLTDEVLSKIDRESRFLQLMGLTGRLVNDTKTYQAERGQGEVASAIQCYMKDHPKISEEEALQHVYSVMENALEELNREFVNNKIPDIYKRLVFETARIMQLFYMQGDGLTLSHDMEIKEHVKNCLFQPVA

SEQ ID NO: 34 - DNA, 2352 bp, Abies grandis
gtgaaacgagaatttcctccaggattttggaaggatgatcttatcgattctctaacgtcatctcacaaggttgcagcatcagacgagaagcgtatcgagacattaatatccgagattaagaatatgtttagatgtatgggctatggcgaaacgaatccctctgcatatgacactgcttgggtagcaaggattccagcagttgatggctctgacaaccctcactttcctgagacggttgaatggattcttcaaaatcagttgaaagatgggtcttggggtgaaggattctacttcttggcatatgacagaatactggctacacttgcatgtattattacccttaccctctggcgtactggggagacacaagtacagaaaggtattgaattcttcaggacacaagctggaaagatggaagatgaagctgatagtcataggccaagtggatttgaaatagtatttcctgcaatgctaaaggaagctaaaatcttaggcttggatctgccttacgatttgccattcctgaaacaaatcatcgaaaagcgggaggctaagcttaaaaggattcccactgatgttctctatgcccttccaacaacgttattgtattctttggaaggtttacaagaaatagtagactggcagaaaataatgaaacttcaatccaaggatggatcatttctcagctctccggcatctacagcggctgtattcatgcgtacagggaacaaaaagtgcttggatttcttgaactttgtcttgaagaaattcggaaaccatgtgccttgtcactatccgcttgatctatttgaacgtttgtgggcggttgatacagttgagcggctaggtatcgatcgtcatttcaaagaggagatcaaggaagcattggattatgtttacagccattgggacgaaagaggcattggatgggcgagagagaatcctgttcctgatattgatgatacagccatgggccttcgaatcttgagattacatggatacaatgtatcctcagatgttttaaaaacatttagagatgagaatggggagttcttttgcttcttgggtcaaacacagagaggagttacagacatgttaaacgtcaatcgttgttcacatgtttcatttccgggagaaacgatcatggaagaagcaaaactctgtaccgaaaggtatctgaggaatgctctggaaaatgtggatgcctttgacaaatgggcttttaaaaagaatattcggggagaggtagagtatgcactcaaatatccctggcataagagtatgccaaggttggaggctagaagctatattgaaaactatgggccagatgatgtgtggcttggaaaaactgtatatatgatgccatacatttcgaatgaaaagtatttagaactagcgaaactggacttcaataaggtgcagtctatacaccaaacagagcttcaagatcttcgaaggtggtggaaatcatccggtttcacggatctgaatttcactcgtgagcgtgtgacggaaatatatttctcaccggcatcctttatctttgagcccgagttttctaagtgcagagaggtttatacaaaaacttccaatttcactgttattttagatgatctttatgacgcccatggatctttagacgatcttaagttgttcacagaatcagtcaaaagatgggatctatcactagtggaccaaatgccacaacaaatgaaaatatgttttgtgggtttctacaatacttttaatgatatagcaaaagaaggacgtgagaggcaagggcgcgatgtgctaggctacattcaaaatgtttggaaagtccaacttgaagcttacacgaaagaagcagaatggtctgaagctaaatatgtgccatccttcaatgaatacatagagaatgcgagtgtgtcaatagcattgggaacagtcgttctcattagtgctcttttcactggggaggttcttacagatgaagtactctccaaaattgatcgcgaatctagatttcttcaactcatgggcttaacagggcgtttggtgaatgacaccaaaacttatcaggcagagagaggtcaaggtgaggtggcttctgccatacaatgttatatgaaggaccatcctaaaatctctgaagaagaagctctacaacatgtctatagtgtcatggaaaatgccctcgaagagttgaatagggagtttgtgaataacaaaataccggatatttacaaaagactggtttttgaaactgcaagaataatgcaactcttttatatgcaaggggatggtttgacactatcacatgatatggaaattaaagagcatgtcaaaaattgcctcttccaaccagttgcc

SEQ ID NO: 35 - protein, 483 aa, Picea sitchensis
MAPMADQISLLLVVFTVAVALLHLIHRWWNIQRGPKMSNKEVHLPPGSTGWPLIGETFSYYRSMTSNHPRKFIDDREKRYDSDIFISHLFGGRTVVSADPQFNKFVLQNEGRFFQAQYPKALKALIGNYGLLSVHGDLQRKLHGIAVNLLRFERLKVDFMEEIQNLVHSTLDRWADMKEISLQNECHQMVLNLMAKQLLDLSPSKETSDICELFVDYTNAVIAIPIKIPGSTYAKGLKARELLIKKISEMIKERRNHPEVVHNDLLTKLVEEGLISDEIICDFILFLLFAGHETSSRAMTFAIKFLTYCPKALKQMKEEHDAILKSKGGHKKLNWDDYKSMAFTQCVINETLRLGNFGPGVFREAKEDTKVKDCLIPKGWVVFAFLTATHLHEKFHNEALTFNPWRWQLDKDVPDDSLFSPFGGGARLCPGSHLAKLELSLFLHIFITRFSWEARADDRTSYFPLPYLTKGFPISLHGRVENE

SEQ ID NO: 36 - DNA, 1452 bp, Picea sitchensis
atggcgcccatggcagaccaaatatcattactgttggtggtgttcacggtagcggtggcgctcctccaccttattcacaggtggtggaatatccagagaggcccaaaaatgagtaataaggaggttcatctgcctcctgggtcgactggatggccgcttattggcgaaaccttcagttattatcgctccatgaccagcaatcatcccaggaaattcatcgacgacagagagaaaagatatgattcggacattttcatatctcatctatttggaggccggacggttgtatcagcggatccccagttcaacaagtttgttctacaaaacgaggggagattctttcaagcccaatacccaaaggcactgaaggctttgataggcaactacgggctgctctctgtgcatggagatctccagagaaagctccacggaatagctgtgaatttgctgaggtttgagagactgaaagtcgatttcatggaggagatacagaatctcgtgcactccacgttggatagatgggcagatatgaaggaaatttctctgcagaatgaatgtcaccagatggttctcaacttgatggccaaacaactgctggatttatctccttccaaagagacgagtgatatttgcgagctattcgttgactataccaatgcagtgattgccattcccatcaaaatcccaggttccacctatgcaaaggggcttaaggcaagggagcttctcataaaaaagatttcagaaatgataaaagagagaaggaatcatcctgaagttgttcataatgatttgttaactaaacttgtggaagaggggctcatttcagatgaaattatttgtgattttattttatttttactttttgctggacatgagacttcctctagagccatgacatttgctatcaagtttcttacctattgccccaaggcattgaagcaaatgaaggaagagcatgatgctatattaaaatcaaagggaggtcataagaaacttaattgggatgactacaaatcaatggcattcactcaatgtgttataaatgaaacacttcgattaggtaactttggtccaggggtgtttagagaagctaaagaagacactaaagtaaaagattgtctcattccaaaaggatgggtggtatttgcttttctgactgcaacacatctacatgaaaagtttcataatgaagctcttacttttaacccatggcgatggcaattggataaagatgtaccagatgatagtttgttttcaccttttggaggtggagctaggctttgtccaggatctcatctagctaaacttgaattgtcactttttcttcacatatttatcacaagattcagttgggaagcgcgtgcagatgatcgtacctcatattttccattaccttatttaactaaaggctttcccattagccttcatggtagagtagagaatgaataa

SEQ ID NO: 37 - protein, 454 aa, Picea sitchensis
NIQRGPKMSNKEVHLPPGSTGWPLIGETFSYYRSMTSNHPRKFIDDREKRYDSDIFISHLFGGRTVVSADPQFNKFVLQNEGRFFQAQYPKALKALIGNYGLLSVHGDLQRKLHGIAVNLLRFERLKVDFMEEIQNLVHSTLDRWADMKEISLQNECHQMVLNLMAKQLLDLSPSKETSDICELFVDYTNAVIAIPIKIPGSTYAKGLKARELLIKKISEMIKERRNHPEVVHNDLLTKLVEEGLISDEIICDFILFLLFAGHETSSRAMTFAIKFLTYCPKALKQMKEEHDAILKSKGGHKKLNWDDYKSMAFTQCVINETLRLGNFGPGVFREAKEDTKVKDCLIPKGWVVFAFLTATHLHEKFHNEALTFNPWRWQLDKDVPDDSLFSPFGGGARLCPGSHLAKLELSLFLHIFITRFSWEARADDRTSYFPLPYLTKGFPISLHGRVENE

SEQ ID NO: 38 - DNA, 1365 bp, Picea sitchensis
aatatccagagaggcccaaaaatgagtaataaggaggttcatctgcctcctgggtcgactggatggccgcttattggcgaaaccttcagttattatcgctccatgaccagcaatcatcccaggaaattcatcgacgacagagagaaaagatatgattcggacattttcatatctcatctatttggaggccggacggttgtatcagcggatccccagttcaacaagtttgttctacaaaacgaggggagattctttcaagcccaatacccaaaggcactgaaggctttgataggcaactacgggctgctctctgtgcatggagatctccagagaaagctccacggaatagctgtgaatttgctgaggtttgagagactgaaagtcgatttcatggaggagatacagaatctcgtgcactccacgttggatagatgggcagatatgaaggaaatttctctgcagaatgaatgtcaccagatggttctcaacttgatggccaaacaactgctggatttatctccttccaaagagacgagtgatatttgcgagctattcgttgactataccaatgcagtgattgccattcccatcaaaatcccaggttccacctatgcaaaggggcttaaggcaagggagcttctcataaaaaagatttcagaaatgataaaagagagaaggaatcatcctgaagttgttcataatgatttgttaactaaacttgtggaagaggggctcatttcagatgaaattatttgtgattttattttatttttactttttgctggacatgagacttcctctagagccatgacatttgctatcaagtttcttacctattgccccaaggcattgaagcaaatgaaggaagagcatgatgctatattaaaatcaaagggaggtcataagaaacttaattgggatgactacaaatcaatggcattcactcaatgtgttataaatgaaacacttcgattaggtaactttggtccaggggtgtttagagaagctaaagaagacactaaagtaaaagattgtctcattccaaaaggatgggtggtatttgcttttctgactgcaacacatctacatgaaaagtttcataatgaagctcttacttttaacccatggcgatggcaattggataaagatgtaccagatgatagtttgttttcaccttttggaggtggagctaggctttgtccaggatctcatctagctaaacttgaattgtcactttttcttcacatatttatcacaagattcagttgggaagcgcgtgcagatgatcgtacctcatattttccattaccttatttaactaaaggctttcccattagccttcatggtagagtagagaatgaataa

SEQ ID NO: 39 - protein, 708 aa, Camptotheca acuminata
MQSSSVKVSTFDLMSAILRGRSMDQTNVSFESGESPALAMLIENRELVMILTTSVAVLIGCFVVLLWRRSSGKSGKVTEPPKPLMVKTEPEPEVDDGKKKVSIFYGTQTGTAEGFAKALAEEAKVRYEKASFKVIDLDDYAADDEEYEEKLKKETLTFFFLATYGDGEPTDNAARFYKWFMEGKERGDWLKNLHYGVFGLGNRQYEHFNRIAKVVDDTIAEQGGKRLIPVGLGDDDQCIEDDFAAWRELLWPELDQLLQDEDGTTVATPYTAAVLEYRVVFHDSPDASLLDKSFSKSNGHAVHDAQHPCRANVAVRRELHTPASDRSCTHLEFDISGTGLVYETGDHVGVYCENLIEVVEEAEMLLGLSPDTFFSIHTDKEDGTPLSGSSLPPPFPPCTLRRALTQYADLLSSPKKSSLLALAAHCSDPSEADRLRHLASPSGKDEYAQWVVASQRSLLEVMAEFPSAKPPIGAFFAGVAPRLQPRYYSISSSPRMAPSRIHVTCALVFEKTPVGRIHKGVCSTWMKNAVPLDESRDCSWAPIFVRQSNFKLPADTKVPVLMIGPGTGLAPFRGFLQERLALKEAGAELGPAILFFGCRNRQMDYIYEDELNNFVETGALSELIVAFSREGPKKEYVQHKMMEKASDIWNMISQEGYIYVCGDAKGMARDVHRTLHTIVQEQGSLDSSKTESMVKNLQMNGRYLRDVW

SEQ ID NO: 40 - DNA, 2606 bp, Camptotheca acuminata
agtctctgcaaccataaccataaccagaaccagaaccaggaagccagaggctctcttttctttctctctctctcattaccaattctccggtaattttctagccggccacaggacctttatttttttcccggtaagatgcaatcgagttcggttaaggtgtcgacgtttgatttgatgtcagcgattttgagggggaggagtatggatcagaccaacgtgtcgttcgaatccggcgagtctccggcgttggcgatgttgatcgagaatcgggagctggtgatgatcctgacgacgtctgtggcggtgttgatagggtgttttgtagtgttgttgtggcggagatcgtcaggaaagtcggggaaagtgacagaacctccgaagccgctgatggtgaagactgagccggagccggaagttgatgacggcaagaagaaggtttctatcttctatggcacgcagaccggtaccgccgaaggtttcgcaaaggcactcgccgaggaagcaaaagtgagatacgaaaaggcgtcatttaaagtgatagatttggatgattatgccgccgacgatgaagaatacgaagagaaattgaagaaagaaactttaacatttttcttcttagctacatacggagatggagaaccaactgacaatgccgccagattctacaaatggtttatggagggaaaagagagaggggactggcttaagaatctccattacggagtatttggtctcggcaacaggcagtatgagcatttcaacaggattgcaaaggtggtggatgataccattgccgagcagggtgggaagcgcctcattcctgtgggccttggagatgatgatcaatgcattgaagatgattttgctgcatggcgggagttattgtggcccgagttggatcagttgcttcaagatgaagatggcacaactgttgctactccttacactgccgctgtattggaatatcgtgttgtattccatgacagcccagatgcatcattactggacaagagcttcagtaagtcaaatggtcatgctgttcatgatgctcaacatccatgcagagctaacgtggctgtgagaagggagcttcacactcccgcatctgatcgttcttgcactcatctggaatttgatatttctggcactggacttgtatatgaaactggggaccatgttggtgtgtattgtgagaatttaattgaagttgtggaggaggcagaaatgttattaggtttatcaccagatacctttttctccattcacactgataaggaggatggcacaccacttagtggaagctccttgccacctcctttccccccctgtactttaagaagagcgctgactcaatatgcagatcttttgagttctcccaaaaagtcctctttgcttgctctagcagctcattgttctgatccaagtgaagctgatcgattaagacaccttgcatctccttctggaaaggatgaatatgcacagtgggtagttgcaagtcagagaagtctccttgaggtcatggcagaatttccatcagcaaagcccccgattggagctttctttgccggagttgccccacgtctgcaacccagatactattcaatttcatcctccccaaggatggcaccatctagaatccacgttacttgtgcattagtttttgagaaaacacctgtaggacggattcacaagggtgtgtgttcaacttggatgaagaatgctgtgccactagatgagagccgtgattgcagctgggcacctatttttgttaggcaatctaacttcaaacttcctgctgatactaaagtacctgttttaatgattggacctggcacaggattggctccttttaggggtttcctgcaggaaagattggctctgaaagaagctggagcagaacttggacctgccatactattttttggatgcaggaatcgtcaaatggattacatttatgaggatgagctgaacaactttgttgaaactggtgcactctctgagcttattgtcgctttctcacgcgagggacccaaaaaggaatatgtgcaacataagatgatggagaaagcgtcggatatctggaacatgatttctcaggaaggatatatatatgtatgtggtgacgccaaaggcatggcgagggatgtccacagaacactacacactattgtgcaagagcagggatctctagacagctccaagactgaaagcatggtgaagaatctgcaaatgaatggaaggtatttgcgtgatgtgtggtgattagtaccctcaagttaacccatcataaagttggggcaaatgaaagaaaattatgtaatttatactggccgaggccaaattgccggggataaaagaaagcatgcagcaaggcaaagtgagaagattactcaccttcgctgccaattcttaatagtgatcagttctgtgattctttttactcttcttgtgcgaaggattttttggttcatgtaatttatatatatatacacacaatatgttgtagttataatagcagtaattgggaggcatttttactggactttctctctgtaattttactctaatgagcagataagttaattgattctggacaaaaaaaaaaa

SEQ ID NO: 41 - protein, 639 aa, Camptotheca acuminata
SSGKSGKVTEPPKPLMVKTEPEPEVDDGKKKVSIFYGTQTGTAEGFAKALAEEAKVRYEKASFKVIDLDDYAADDEEYEEKLKKETLTFFFLATYGDGEPTDNAARFYKWFMEGKERGDWLKNLHYGVFGLGNRQYEHFNRIAKVVDDTIAEQGGKRLIPVGLGDDDQCIEDDFAAWRELLWPELDQLLQDEDGTTVATPYTAAVLEYRVVFHDSPDASLLDKSFSKSNGHAVHDAQHPCRANVAVRRELHTPASDRSCTHLEFDISGTGLVYETGDHVGVYCENLIEVVEEAEMLLGLSPDTFFSIHTDKEDGTPLSGSSLPPPFPPCTLRRALTQYADLLSSPKKSSLLALAAHCSDPSEADRLRHLASPSGKDEYAQWVVASQRSLLEVMAEFPSAKPPIGAFFAGVAPRLQPRYYSISSSPRMAPSRIHVTCALVFEKTPVGRIHKGVCSTWMKNAVPLDESRDCSWAPIFVRQSNFKLPADTKVPVLMIGPGTGLAPFRGFLQERLALKEAGAELGPAILFFGCRNRQMDYIYEDELNNFVETGALSELIVAFSREGPKKEYVQHKMMEKASDIWNMISQEGYIYVCGDAKGMARDVHRTLHTIVQEQGSLDSSKTESMVKNLQMNGRYLRDVW

SEQ ID NO: 42 - DNA, 1920 bp, Camptotheca acuminata
tcgtcaggaaagtcggggaaagtgacagaacctccgaagccgctgatggtgaagactgagccggagccggaagttgatgacggcaagaagaaggtttctatcttctatggcacgcagaccggtaccgccgaaggtttcgcaaaggcactcgccgaggaagcaaaagtgagatacgaaaaggcgtcatttaaagtgatagatttggatgattatgccgccgacgatgaagaatacgaagagaaattgaagaaagaaactttaacatttttcttcttagctacatacggagatggagaaccaactgacaatgccgccagattctacaaatggtttatggagggaaaagagagaggggactggcttaagaatctccattacggagtatttggtctcggcaacaggcagtatgagcatttcaacaggattgcaaaggtggtggatgataccattgccgagcagggtgggaagcgcctcattcctgtgggccttggagatgatgatcaatgcattgaagatgattttgctgcatggcgggagttattgtggcccgagttggatcagttgcttcaagatgaagatggcacaactgttgctactccttacactgccgctgtattggaatatcgtgttgtattccatgacagcccagatgcatcattactggacaagagcttcagtaagtcaaatggtcatgctgttcatgatgctcaacatccatgcagagctaacgtggctgtgagaagggagcttcacactcccgcatctgatcgttcttgcactcatctggaatttgatatttctggcactggacttgtatatgaaactggggaccatgttggtgtgtattgtgagaatttaattgaagttgtggaggaggcagaaatgttattaggtttatcaccagatacctttttctccattcacactgataaggaggatggcacaccacttagtggaagctccttgccacctcctttccccccctgtactttaagaagagcgctgactcaatatgcagatcttttgagttctcccaaaaagtcctctttgcttgctctagcagctcattgttctgatccaagtgaagctgatcgattaagacaccttgcatctccttctggaaaggatgaatatgcacagtgggtagttgcaagtcagagaagtctccttgaggtcatggcagaatttccatcagcaaagcccccgattggagctttctttgccggagttgccccacgtctgcaacccagatactattcaatttcatcctccccaaggatggcaccatctagaatccacgttacttgtgcattagtttttgagaaaacacctgtaggacggattcacaagggtgtgtgttcaacttggatgaagaatgctgtgccactagatgagagccgtgattgcagctgggcacctatttttgttaggcaatctaacttcaaacttcctgctgatactaaagtacctgttttaatgattggacctggcacaggattggctccttttaggggtttcctgcaggaaagattggctctgaaagaagctggagcagaacttggacctgccatactattttttggatgcaggaatcgtcaaatggattacatttatgaggatgagctgaacaactttgttgaaactggtgcactctctgagcttattgtcgctttctcacgcgagggacccaaaaaggaatatgtgcaacataagatgatggagaaagcgtcggatatctggaacatgatttctcaggaaggatatatatatgtatgtggtgacgccaaaggcatggcgagggatgtccacagaacactacacactattgtgcaagagcagggatctctagacagctccaagactgaaagcatggtgaagaatctgcaaatgaatggaaggtatttgcgtgatgtgtggtga

SEQ ID NO: 43 - protein, 552 aa, P. cablin
MELYAQSVGVGAASRPLANFHPCVWGDKFIVYNPQSCQAGEREEAEELKVELKRELKEASDNYMRQLKMVDAIQRLGIDYLFVEDVDEALKNLFEMFDAFCKNNHDMHATALSFRLLRQHGYRVSCEVFEKFKDGKDGFKVPNEDGAVAVLEFFEATHLRVHGEDVLDNAFDFTRNYLESVYATLNDPTAKQVHNALNEFSFRRGLPRVEARKYISIYEQYASHHKGLLKLAKLDFNLVQALHRRELSEDSRWWKTLQVPTKLSFVRDRLVESYFWASGSYFEPNYSVARMILAKGLAVLSLMDDVYDAYGTFEELQMFTDAIERWDASCLDKLPDYMKIVYKALLDVFEEVDEELIKLGAPYRAYYGKEAMKYAARAYMEEAQWREQKHKPTTKEYMKLATKTCGYITLIILSCLGVEEGIVTKEAFDWVFSRPPFIEATLIIARLVNDITGHEFEKKREHVRTAVECYMEEHKVGKQEVVSEFYNQMESAWKDINEGFLRPVEFPIPLLYLILNSVRTLEVIYKEGDSYTHVGPAMQNIIKQLYLHPVPY

SEQ ID NO: 44 - DNA, 1659 bp, P. cablin
atggagttgtatgcccaaagtgttggagtgggtgctgcttctcgtcctcttgcgaattttcatccatgtgtgtggggagacaaattcattgtctacaacccacaatcatgccaggctggagagagagaagaggctgaggagctgaaagtggagctgaaaagagagctgaaggaagcatcagacaactacatgcggcaactgaaaatggtggatgcaatacaacgattaggcattgactatctttttgtggaagatgttgatgaagctttgaagaatctgtttgaaatgtttgatgctttctgcaagaataatcatgacatgcacgccactgctctcagctttcgccttctcagacaacatggatacagagtttcatgtgaagtttttgaaaagtttaaggatggcaaagatggatttaaggttccaaatgaggatggagcggttgcagtccttgaattcttcgaagccacgcatctcagagtccatggagaagacgtccttgataatgcttttgacttcactaggaactacttggaatcagtctatgcaactttgaacgatccaaccgcgaaacaagtccacaacgcattgaatgagttctcttttcgaagaggattgccacgcgtggaagcaaggaagtacatatcaatctacgagcaatacgcatctcatcacaaaggcttgctcaaacttgctaagctggatttcaacttggtacaagctttgcacagaagggagctgagtgaagattctaggtggtggaagactttacaagtgcccacaaagctatcattcgttagagatcgattggtggagtcctacttctgggcttcgggatcttatttcgaaccgaattattcggtagctaggatgattttagcaaaagggctggctgtattatctcttatggatgatgtgtatgatgcatatggtacttttgaggaattacaaatgttcacagatgcaatcgaaaggtgggatgcttcatgtttagataaacttccagattacatgaaaatagtatacaaggcccttttggatgtgtttgaggaagttgacgaggagttgatcaagctaggcgcaccatatcgagcctactatggaaaagaagccatgaaatacgccgcgagagcttacatggaagaggcccaatggagggagcaaaagcacaaacccacaaccaaggagtatatgaagctggcaaccaagacatgtggctacataactctaataatattatcatgtcttggagtggaagagggcattgtgaccaaagaagccttcgattgggtgttctcccgacctcctttcatcgaggctacattaatcattgccaggctcgtcaatgatattacaggacacgagtttgagaaaaaacgagagcacgttcgcactgcagtagaatgctacatggaagagcacaaagtggggaagcaagaggtggtgtctgaattctacaaccaaatggagtcagcatggaaggacattaatgaggggttcctcagaccagttgaatttccaatccctctactttatcttattctcaattcagtccgaacacttgaggttatttacaaagagggcgattcgtatacacacgtgggtcctgcaatgcaaaacatcatcaagcagttgtaccttcaccctgttccatattaa

SEQ ID NO: 45 - protein, 347 aa, Picea abies
MASNGIVDVKTKFEEIYLELKAQILNDPAFDYTEDARQWVEKMLDYTVPGGKLNRGLSVIDSYRLLKAGKEISEDEVFLGCVLGWCIEWLQAYFLILDDIMDSSHTRRGQPCWFRLPKVGLIAVNDGILLRNHICRILKKHFRTKPYYVDLLDLFNEVEFQTASGQLLDLITTHEGATDLSKYKMPTYVRIVQYKTAYYSFYLPVACALVMAGENLDNHVDVKNILVEMGTYFQVQDDYLDCFGDPEVIGKIGTDIEDFKCSWLVVQALERANESQLQRLYANYGKKDPSCVAEVKAVYRDLGLQDVFLEYERTSHKELISSIEAQENESLQLVLKSFLGKIYKRQK

SEQ ID NO: 46 - DNA, 1044 bp, Picea abies
atggcttcaaacggcatcgtcgacgtgaaaaccaagtttgaggaaatctatcttgagcttaaggctcagattctgaacgatcctgccttcgattacaccgaagacgcccgtcaatgggtcgagaagatgctggactacacggtgcccggaggaaagctgaaccgcggtctgtctgtaatagacagctacaggctattgaaagcaggaaaggaaatatcagaagatgaagtctttcttggatgtgtgcttggctggtgtattgaatggcttcaagcatatttcctcatattagatgacatcatggacagctctcacactaggcgtggacaaccttgttggttcagattacctaaggttggcttaattgctgttaatgatggaatattgcttcgtaaccacatatgcagaattctgaaaaagcattttcgcactaagccttactatgtggatctccttgatttattcaatgaggttgagtttcaaacagctagtggacagttgctggaccttatcactactcatgaaggagcaactgacctttcaaagtacaaaatgccaacttatgttcgtatagttcaatacaagactgcctactattcattctatctgccggttgcctgtgcactggtaatggcaggggaaaatttagataatcacgtagatgtcaagaatattttagtcgaaatgggaacctattttcaagtacaggatgattatcttgattgctttggtgatccagaagtgattgggaagattggaactgatatcgaagacttcaagtgctcttggttggtggtgcaagcccttgaacgggcaaatgagagccaacttcaacgattatatgccaattatggaaagaaagatccttcttgtgttgcagaagtgaaggctgtatatagggatcttggacttcaggatgtttttctggaatacgagcgtactagtcacaaggagctcatttcttccatcgaggctcaggagaatgaatctttgcagcttgttctgaagtccttcctagggaagatatacaagcgacagaagtaa

SEQ ID NO: 47 - protein, 354 aa, Gallus gallus
MSADGAKRTAAEREREEFVGFFPQIVRDLTEDGIGHPEVGDAVARLKEVLQYNAPGGKCNRGLTVVAAYRELSGPGQKDAESLRCALAVGWCIELFQAFFLVADDIMDQSLTRRGQLCWYKKEGVGLDAINDSFLLESSVYRVLKKYCGQRPYYVHLLELFLQTAYQTELGQMLDLITAPVSKVDLSHFSEERYKAIVKYKTAFYSFYLPVAAAMYMVGIDSKEEHENAKAILLEMGEYFQIQDDYLDCFGDPALTGKVGTDIQDNKCSWLVVQCLQRVTPEQRQLLEDNYGRKEPEKVAKVKELYEAVGMRAAFQQYEESSYRRLQELIEKHSNRLPKEIFLGLAQKIYKRQK

SEQ ID NO: 48 - DNA, 1330 bp, Gallus gallus
agaatgccccgcgcggcgccgggcggagcgcacggaaaggtcgcggggcaaaaagcggcgctgagcggacggggccgaacgcgtcggggtcgccatgagcgcggatggggcgaagcggacggcggccgagagggagagggaggagttcgtggggttcttcccgcagatcgtccgcgatctgaccgaggacggcatcggacacccggaggtgggcgacgctgtggcgcggctgaaggaggtgctgcaatacaacgctcccggtgggaaatgcaaccgtgggctgacggtggtggctgcgtaccgggagctgtcggggccggggcagaaggatgctgagagcctgcggtgcgcgctggccgtgggttggtgcatcgagttgttccaggccttcttcctggtggctgatgatatcatggatcagtccctcacgcgccgggggcagctgtgttggtataagaaggagggggtcggtttggatgccatcaacgactccttcctcctcgagtcctctgtgtacagagtgctgaagaagtactgcgggcagcggccgtattacgtgcatctgttggagctcttcctgcagaccgcctaccagactgagctcgggcagatgctggacctcatcacagctcccgtctccaaagtggatttgagtcacttcagcgaggagaggtacaaagccatcgttaagtacaagactgccttctactccttctacctacccgtggctgctgccatgtatatggttgggatcgacagtaaggaagaacacgagaatgccaaagccatcctgctggagatgggggaatacttccagatccaggatgattacctggactgctttggggacccggcgctcacggggaaggtgggcaccgacatccaggacaataaatgcagctggctcgtggtgcagtgcctgcagcgcgtcacgccggagcagcggcagctcctggaggacaactacggccgtaaggagcccgagaaggtggcgaaggtgaaggagctgtatgaggccgtggggatgagggctgcgttccagcagtacgaggagagcagctaccggcgcctgcaggaactgatagagaagcactcgaaccgcctcccgaaggagatcttcctcggcctggcacagaagatctacaaacgccagaaatgaggggtgggggcggcagcggctctgtgcttcgcgctgtgttgggtggcttcgcagccccggacccggtgctccccccacccgttatccccggagatgcggggggggggcggtgcggggcgcgcatccatcggtgccgtcagactgtgtgtcaataaacgttaatttattgcc

SEQ ID NO: 49 - protein, 180 aa, Arabidopsis thaliana
MASSMLSSATMVASPAQATMVAPFNGLKSSAAFPATRKANNDITSITSNGGRVNCMQVWPPIGKKKFETLSYLPDLTDSELAKEVDYLIRNKWIPCVEFELEHGFVYREHGNSPGYYDGRYWTMWKLPLFGCTDSAQVLKEVEECKKEYPNAFIRIIGFDNTRQVQCISFIAYKPPSFTG

SEQ ID NO: 50 - DNA, 1268 bp, Arabidopsis thaliana
ccaaggtaaaaaaaaggtatgaaagctctatagtaagtaaaatataaattccccataaggaaagggccaagtccaccaggcaagtaaaatgagcaagcaccactccaccatcacacaatttcactcatagataacgataagattcatggaattatcttccacgtggcattattccagcggttcaagccgataagggtctcaacacctctccttaggcctttgtggccgttaccaagtaaaattaacctcacacatatccacactcaaaatccaacggtgtagatcctagtccacttgaatctcatgtatcctagaccctccgatcactccaaagcttgttctcattgttgttatcattatatatagatgaccaaagcactagaccaaacctcagtcacacaaagagtaaagaagaacaatggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaactgcatgcaggtgtggcctccgattggaaagaagaagtttgagactctctcttaccttcctgaccttaccgattccgaattggctaaggaagttgactaccttatccgcaacaagtggattccttgtgttgaattcgagttggagcacggatttgtgtaccgtgagcacggtaactcacccggatactatgatggacggtactggacaatgtggaagcttcccttgttcggttgcaccgactccgctcaagtgttgaaggaagtggaagagtgcaagaaggagtaccccaatgccttcattaggatcatcggattcgacaacacccgtcaagtccagtgcatcagtttcattgcctacaagccaccaagcttcaccggttaatttccctttgcttttgtgtaaacctcaaaactttatcccccatctttgattttatcccttgtttttctgcttttttcttctttcttgggttttaatttccggacttaacgtttgttttccggtttgcgagacatattctatcggattctcaactgtctgatgaaataaatatgtaatgttctataagtctttcaatttgatatgcatatcaacaaaaagaaaataggacaatgcggctacaaatatgaaatttacaagtttaagaaccatgagtcgctaaagaaatcattaagaaaattagtttcac

SEQ ID NO: 51 - protein, 415 aa, Amaranthus hybridus
MGSLGAILKHPDEFYPLLKLKMAVKEAEKQIPSESHWGFCYSMLHKVSRSFALVIQQLGTELRNAVCVFYLVLRALDTVEDDTSIATDVKLPILKAFYQHIYDREWHFSCGTKHYKVLMDEFHQVSTAFLELERGYQLAIEDITKRMGAGMAKFICQEVETVSDYDEYCHYVAGLVGLGLSKLFHNAGLEDLASDDLSNSMGLFLQKTNIIRDYLEDINEIPKCRMFWPREIWSKYVNKLEDLKYEENSVKAVQCLNDMVTNALLHVEDCLKYMSALRDHAIFRFCAIPQIMAIGTLALCYNNVEVFRGVVKMRRGLTARVIDKTDSMPDVYGAFYDFACMIKPKVDKNDPNAMKTLSRIDAIEKICRDSGTLNKRKLHITSTKSAYTPIMVMVLFIVLAIFFNRLSESNRMINN

SEQ ID NO: 52 - protein, 374 aa, Amaranthus hybridus
MGSLGAILKHPDEFYPLLKLKMAVKEAEKQIPSESHWGFCYSMLHKVSRSFALVIQQLGTELRNAVCVFYLVLRALDTVEDDTSIATDVKLPILKAFYQHIYDREWHFSCGTKHYKVLMDEFHQVSTAFLELERGYQLAIEDITKRMGAGMAKFICQEVETVSDYDEYCHYVAGLVGLGLSKLFHNAGLEDLASDDLSNSMGLFLQKTNIIRDYLEDINEIPKCRMFWPREIWSKYVNKLEDLKYEENSVKAVQCLNDMVTNALLHVEDCLKYMSALRDHAIFRFCAIPQIMAIGTLALCYNNVEVFRGVVKMRRGLTARVIDKTDSMPDVYGAFYDFACMIKPKVDKNDPNAMKTLSRIDAIEKICRDSGTLN

SEQ ID NO: 53 - protein, 461 aa, Botryococcus braunii
MGMLRWGVESLQNPDELIPVLRMIYADKFGKIKPKDEDRGFCYEILNLVSRSFAIVIQQLPAQLRDPVCIFYLVLRALDTVEDDMKIAATTKIPLLRDFYEKISDRSFRMTAGDQKDYIRLLDQYPKVTSVFLKLTPREQEIIADITKRMGNGMADFVHKGVPDTVGDYDLYCHYVAGVVGLGLSQLFVASGLQSPSLTRSEDLSNHMGLFLQKTNIIRDYFEDINELPAPRMFWPREIWGKYANNLAEFKDPANKAAAMCCLNEMVTDALRHAVYCLQYMSMIEDPQIFNFCAIPQTMAFGTLSLCYNNYTIFTGPKAAVKLRRGTTAKLMYTSNNMFAMYRHFLNFAEKLEVRCNTETSEDPSVTTTLEHLHKIKAACKAGLARTKDDTFDELRSRLLALTGGSFYLAWTYNFLDLRGPGDLPTFLSVTQHWWSILIFLISIAVFFIPSRPSPRPTLSA

SEQ ID NO: 54 - DNA, 3076 bp, Botryococcus braunii
aacagcaacaagtcctctgcgtcaggcaaaacgtccgtttgtatggcttggcgcttgaaagctggtggggataaacgtcaaaagaaagaagctctgttcgggttcacgggtgtcgtttagtactttcccctacgacattgtcagccttggctcatcgcaatccaaccaaatatggggatgcttcgctggggagtggagtctttgcagaatccagatgaattaatcccggtcttgaggatgatttatgctgataagtttggaaagatcaagccaaaggacgaagaccggggcttctgctatgaaattttaaaccttgtttcaagaagttttgcaatcgtcatccaacagctccctgcacagctgagggacccagtctgcatattttaccttgtactacgcgccctggacacagtcgaagatgatatgaaaattgcagcaaccaccaagattcccttgctgcgtgacttttatgagaaaatttctgacaggtcattccgcatgacggccggagatcaaaaagactacatcaggctgttggatcagtaccccaaagtgacaagcgttttcttgaaattgaccccccgtgaacaagagataattgcagacattacaaagcggatggggaatggaatggctgacttcgtgcataagggtgttcccgacacagtgggggactacgacctttactgccactatgttgctggggtggtgggtctcgggctttcccagttgttcgttgcgagtggactacagtcaccctctttgacccgcagtgaagacctttccaatcacatgggcctcttccttcagaagaccaacatcatccgcgactactttgaggacatcaatgagctgcctgccccccggatgttctggcccagagagatctggggcaagtatgcgaacaacctcgctgagttcaaagacccggccaacaaggcggctgcaatgtgctgcctcaacgagatggtcacagatgcattgaggcacgcggtgtactgcctgcagtacatgtccatgattgaggatccgcagatcttcaacttctgtgccatccctcagaccatggccttcggcaccctgtctttgtgttacaacaactacactatcttcacagggcccaaagcggctgtgaagctgcgtaggggcaccactgccaagctgatgtacacctctaacaatatgtttgcgatgtaccgtcatttcctcaacttcgcagagaagctggaagtcagatgcaacaccgagaccagcgaggatcccagcgtgaccaccactctggaacacctgcataagatcaaagctgcctgcaaggctgggctggcacgcacaaaagatgacacctttgacgaattgaggagcaggttgttagcgctgacgggaggcagcttctacctcgcctggacctacaatttcctagaccttcgaggcccgggagacctgcccaccttcttatctgtaacccaacattggtggtctattctgatcttcctcatttcgattgccgtcttctttattccgtcgaggccctcacctagacccacactcagcgcctaatcctttggctctcgtcaattccggagtcccccattgttgtcagcacttggggaatttcgtggtcttcttgaccacactcttgtctctggcagaggtcaaggacactgtcagggacaagtgagtattctgacccccccccccccccccctctgctcctttcaccacccctccctatcatctggggcaaagcttgggaatgggcccgtccccctgttgtcccgctcagatgcaaagtttgggttatgtaactgggttgaacggctcggggcggtttgaagctgtcccttgttggagatggaaaattgcagggcccgggggggttaactggacacgctcttccgtcccgcagtgtccttctggctttattctgccgtggatgctgtgaacccgccccctctctgggccggctcaatatacaagtattagtttcggtgtttgtgtcaatcctttctcacaacttccctgttcgttggactggagacgcacccttaggtcctttgattgggaatgcggcccctttgggtctttaggctctcgggtagtctagtttgcaattgttgcatgggcgcggctttgcacagacgcctggaccttcattgagacacgtttcggaaaactcgacagttttgaggtaacctgctcgtgggcctcggtgtgtctggaggtgtcaggggcctgtgctccctgctgggatgttcccgctttgctgtaaaaagtcggacgtttgttatcctttgcgggggttcatctttgagtgggccctgcttctctgcccgtgtgatgtaatggtttgtattggataggtatgttgccttatctcgtgtatggaattcgtatggtacttgcagtattcaggagacttgagtaacgacatcgaggacaggtaacaagcgctccgattatgtgctctgttacacccgacttccaaagatttatgcgaggtcctggggaacgcagatttgacattggagagccccaattggccgtggcaatctgtagaatgtcaaaagagaaaacaggaaatcaggttttaaagtccgtgcctatcagcatcctgtgaaagctgatgcggttacgggatgaatgtcaggaatactcgctccagtattaacgtgcgcagattccgactgaagcaaatcgatgaaatttggggaggtgtcgtttttagaccttgacaacggccatgggtcgtacctttttgcaaagtatatatttatttgcactaactcattaggcacgttggttttttttgtccccctcggaacgcctttttaagatagttaactagtttggtcagggtattcgtcagaagcacgaagcacagaaggtttcttttgagatggcggcgattgttttccacgagagcagagtcaatctcacgcgtactcgagcaaacatcgttggtcaggacatggtgttgtctcttggccggccctgtaactttgatgcccccaaaaaaaaaaaaaaaaaaaaaaaaaaaaaaaaaaaaaaaaaaaaaaaaaaaaaaaaaaaaaaaaaa

SEQ ID NO: 55 - protein, 421 aa, Botryococcus braunii
MGMLRWGVESLQNPDELIPVLRMIYADKFGKIKPKDEDRGFCYEILNLVSRSFAIVIQQLPAQLRDPVCIFYLVLRALDTVEDDMKIAATTKIPLLRDFYEKISDRSFRMTAGDQKDYIRLLDQYPKVTSVFLKLTPREQEIIADITKRMGNGMADFVHKGVPDTVGDYDLYCHYVAGVVGLGLSQLFVASGLQSPSLTRSEDLSNHMGLFLQKTNIIRDYFEDINELPAPRMFWPREIWGKYANNLAEFKDPANKAAAMCCLNEMVTDALRHAVYCLQYMSMIEDPQIFNFCAIPQTMAFGTLSLCYNNYTIFTGPKAAVKLRRGTTAKLMYTSNNMFAMYRHFLNFAEKLEVRCNTETSEDPSVTTTLEHLHKIKAACKAGLARTKDDTFDELRSRLLALTGGSFYLAWTYNFLDLRGP

SEQ ID NO: 56 - protein, 378 aa, Botryococcus braunii
MGMLRWGVESLQNPDELIPVLRMIYADKFGKIKPKDEDRGFCYEILNLVSRSFAIVIQQLPAQLRDPVCIFYLVLRALDTVEDDMKIAATTKIPLLRDFYEKISDRSFRMTAGDQKDYIRLLDQYPKVTSVFLKLTPREQEIIADITKRMGNGMADFVHKGVPDTVGDYDLYCHYVAGVVGLGLSQLFVASGLQSPSLTRSEDLSNHMGLFLQKTNIIRDYFEDINELPAPRMFWPREIWGKYANNLAEFKDPANKAAAMCCLNEMVTDALRHAVYCLQYMSMIEDPQIFNFCAIPQTMAFGTLSLCYNNYTIFTGPKAAVKLRRGTTAKLMYTSNNMFAMYRHFLNFAEKLEVRCNTETSEDPSVTTTLEHLHKIKA

SEQ ID NO: 57 - protein, 410 aa, Euphorbia lathyris
MGSLGAILKHPDDFYPLLKLKMAAKHAEKQIPAQPHWGFCYSMLHKVSRSFSLVIQQLGTELRDAVCIFYLVLRALDTVEDDTSIPTDVKVPILIAFHKHIYDPEWHFSCGTKEYKVLMDQIHHLSTAFLELGKSYQEAIEDITKKMGAGMAKFICKEVETVDDYDEYCHYVAGLVGLGLSKLFDASGFEDLAPDDLSNSMGLFLQKTNIIRDYLEDINEIPKSRMFWPRQIWSKYVNKLEDLKYEENSVKAVQCLNDMVTNALIHMDDCLKYMSALRDPAIFRFCAIPQIMAIGTLALCYNNVEVFRGVVKMRRGLTAKVIDRTRTMADVYRAFFDFSCMMKSKVDRNDPNAEKTLNRLEAVQKTCKESGLLNKRRSYINESKPYNSTMVILLMIVLAIILAYLSKRAN

SEQ ID NO: 58 - DNA, 1768 bp, Euphorbia lathyris
gaaccttgtggcgtgcagagagagacagagagagacagagattgttgaatctctatttaattcatagtagcctcattggactcaatccgtcgttttcgtttccatctcctttaaaaaccagtcgatcgtttctcctcaatttcgacttcaactctttctttcgcttattcatttggtttttcaagggatctgaggataatggggagtttgggagcaattctgaagcatccggatgatttttacccgcttttgaagctgaaaatggctgctaaacatgctgagaagcagatcccagcacaacctcactggggtttctgttactccatgcttcataaggtctctcgtagcttttctcttgtcattcaacagcttggcactgagctccgtgacgctgtttgtatattctatttggttcttcgagcccttgatactgttgaggatgatacaagcatccctacagatgtgaaagtgccgatcttgatagcttttcacaagcacatatacgatcctgaatggcatttttcttgtggtactaaggaatataaagttctcatggaccagattcatcatctttcaactgcttttcttgagcttgggaaaagttatcaggaggcaatcgaggatatcacgaaaaaaatgggtgcaggaatggctaaattcatatgcaaagaggtggaaacagttgatgactacgatgaatattgccattatgttgcaggacttgttggactaggtctttccaagctttttgatgcctctggatttgaagatttggcaccagatgacctttccaactcgatggggttatttctccagaaaacaaacattatccgggattatttggaggatataaatgagatacctaagtcacgcatgttttggcctcgccagatctggagtaaatatgttaataaacttgaggacttgaaatatgaagaaaactcagtcaaggcagtgcaatgcttgaatgatatggttactaatgctttgatacatatggatgattgcttgaaatacatgtcggcactacgagatcctgctatatttcgtttttgtgccatccctcagattatggcaattggaaccctagcattgtgctacaacaacgttgaagtatttagaggtgtagtgaagatgaggcgtggtcttactgcaaaggtcattgacagaacaaggaccatggcagatgtctatcgggccttctttgacttctcatgtatgatgaaatccaaggttgacaggaatgatccaaatgcagaaaagacattgaacaggctggaagcagtgcaaaaaacttgcaaggagtctgggctgctaaacaaaaggagatcttacataaatgagagcaagccatataattctactatggttattctactgatgattgtattggcaatcattttggcttatctgagcaaacgggccaactaactagtgtaacttctgttaagtaatcagttgaggatttgaatccggttatcgtgaaaccgggttattgcaggatgtctacttctgtgaacaatttctgcagatggatggctagctagcaatgaaggtgcttgctggacttgttccaggagagttgtgaatttgatgtttcagtatatagtgtagtgccataacaatgtttgtgtccaatgtgccactaatgtgatcatattagtgttttgttctcgtgggttgttattatactccttaattatggaattgaagcaatatcttgaaggatcttctgaatatcttgattcaagtcgctgttattcacatc

SEQ ID NO: 59 - protein, 374 aa, Euphorbia lathyris
MGSLGAILKHPDDFYPLLKLKMAAKHAEKQIPAQPHWGFCYSMLHKVSRSFSLVIQQLGTELRDAVCIFYLVLRALDTVEDDTSIPTDVKVPILIAFHKHIYDPEWHFSCGTKEYKVLMDQIHHLSTAFLELGKSYQEAIEDITKKMGAGMAKFICKEVETVDDYDEYCHYVAGLVGLGLSKLFDASGFEDLAPDDLSNSMGLFLQKTNIIRDYLEDINEIPKSRMFWPRQIWSKYVNKLEDLKYEENSVKAVQCLNDMVTNALIHMDDCLKYMSALRDPAIFRFCAIPQIMAIGTLALCYNNVEVFRGVVKMRRGLTAKVIDRTRTMADVYRAFFDFSCMMKSKVDRNDPNAEKTLNRLEAVQKTCKESGLLN

SEQ ID NO: 60 - skipped

SEQ ID NO: 61 - protein, 467 aa, Ganoderma lucidum
MGATSMLTLLLTHPFEFRVLIQYKLWHEPKRDITQVSEHPTSGWDRPTMRRCWEFLDQTSRSFSGVIKEVEGDLARVICLFYLVLRGLDTIEDDMTLPDEKKQPILRQFHKLAVKPGWTFDECGPKEKDRQLLVEWTVVSEELNRLDACYRDIIIDIAEKMQTGMADYAHKAATTNSIYIGTVDEYNLYCHYVAGLVGEGLTRFWAASGKEAEWLGDQLELTNAMGLMLQKTNIIRDFREDAEERRFFWPREIWGRDAYGKAVGRANGFREMHELYERGNEKQALWVQSGMVVDVLGHATDSLDYLRLLTKQSIFCFCAIPQTMAMATLSLCFMNYDMFHNHIKIRRAEAASLIMRSTNPRDVAYIFRDYARKMHARALPEDPSFLRLSVACGKIEQWCERHYPSFVRLQQVSGGGIVFDPSDARTKVVEAAQARDNELAREKRLAELRDKTGKLERKLRWSQAPSS

SEQ ID NO: 62 - DNA, 1404 bp, Ganoderma lucidum
atgggcgcgacgtctatgctcaccctcctcctcacacaccccttcgagttccgcgtcctcatccaatacaagctctggcacgaaccaaaacgcgacattacccaagtctccgagcacccgacttcaggatgggaccgccctactatgcgacggtgttgggagttccttgaccagaccagccggagtttctctggggtcatcaaggaagtggagggtgatttagcaagagtgatctgcttattctacctggtgctacgaggcctggacacgatcgaagatgacatgacgcttcctgacgagaaaaaacaacccatactccgacaattccacaaactcgccgtgaagcccggttggacattcgacgagtgtggacccaaagaaaaggacaggcaactcctcgtcgagtggacagttgtcagcgaagagctcaaccgtctcgacgcatgctaccgcgatattattatcgacattgcggaaaagatgcagaccgggatggccgactacgcgcataaagcagcgaccacgaattcgatttacatcggaaccgtcgacgagtacaacctctactgccactacgtcgccggcctcgtcggcgagggcctcacgcgcttctgggccgcgtccggcaaggaggcggaatggctgggggaccagctcgagctgacgaacgcgatgggcctcatgctgcagaagacgaacattatccgtgacttccgcgaggacgccgaggagcgccgcttcttctggccgcgcgagatctgggggcgcgacgcatacggcaaggccgtcggccgcgcgaacgggttccgcgagatgcacgagctgtacgagcggggcaacgagaagcaggcgctgtgggtgcagagcgggatggtcgttgacgtgctcgggcacgctacagactcgctcgactatctccgcctactcacgaagcagagcatcttctgcttctgtgcgatcccacaaacgatggccatggccaccctcagcttgtgcttcatgaactacgacatgttccacaaccatatcaagatccgcagggctgaggctgcctcgcttattatgcggtcaacgaacccccgcgacgtcgcatacattttccgcgactacgcgcgcaagatgcacgcccgcgcgctgcccgaggacccctccttcctccgcctctccgtcgcgtgcggcaagatcgagcagtggtgcgagcgccactacccctcctttgtccgcctccagcaggtctcgggtgggggcatcgtgttcgacccgagcgacgcgcgcaccaaggtcgtcgaggccgcgcaggcccgcgacaacgagctcgcgcgcgagaagcgcctggccgagctccgtgacaagactggaaagcttgagcgcaagctgcggtggagtcaagccccatcgagctga

SEQ ID NO: 63 - protein, 406 aa, Ganoderma lucidum
MGATSMLTLLLTHPFEFRVLIQYKLWHEPKRDITQVSEHPTSGWDRPTMRRCWEFLDQTSRSFSGVIKEVEGDLARVICLFYLVLRGLDTIEDDMTLPDEKKQPILRQFHKLAVKPGWTFDECGPKEKDRQLLVEWTVVSEELNRLDACYRDIIIDIAEKMQTGMADYAHKAATTNSIYIGTVDEYNLYCHYVAGLVGEGLTRFWAASGKEAEWLGDQLELTNAMGLMLQKTNIIRDFREDAEERRFFWPREIWGRDAYGKAVGRANGFREMHELYERGNEKQALWVQSGMVVDVLGHATDSLDYLRLLTKQSIFCFCAIPQTMAMATLSLCFMNYDMFHNHIKIRRAEAASLIMRSTNPRDVAYIFRDYARKMHARALPEDPSFLRLSVACGKIEQWCERHYPSF

SEQ ID NO: 64 - protein, 437 aa, Ganoderma lucidum
MGATSMLTLLLTHPFEFRVLIQYKLWHEPKRDITQVSEHPTSGWDRPTMRRCWEFLDQTSRSFSGVIKEVEGDLARVICLFYLVLRGLDTIEDDMTLPDEKKQPILRQFHKLAVKPGWTFDECGPKEKDRQLLVEWTVVSEELNRLDACYRDIIIDIAEKMQTGMADYAHKAATTNSIYIGTVDEYNLYCHYVAGLVGEGLTRFWAASGKEAEWLGDQLELTNAMGLMLQKTNIIRDFREDAEERRFFWPREIWGRDAYGKAVGRANGFREMHELYERGNEKQALWVQSGMVVDVLGHATDSLDYLRLLTKQSIFCFCAIPQTMAMATLSLCFMNYDMFHNHIKIRRAEAASLIMRSTNPRDVAYIFRDYARKMHARALPEDPSFLRLSVACGKIEQWCERHYPSFVRLQQVSGGGIVFDPSDARTKVVEAAQARDN

SEQ ID NO: 65 - protein, 416 aa, Mortierella alpina
MASAILASLLHPSEVLALVQYKLSPKTQHDYSNDKTRQRLYHHLNMTSRSFSAVIQDLDEELKDAICLFYLVLRGLDTIEDDMTIDLDTKLPYLRTFHEIIYQKGWTFTKNGPNEKDRQLLVEFDAIIEGFLQLKPAYQTIIADITKRMGNGMAHYATAGIHVETNADYDEYCHYVAGLVGLGLSEMFSACGFESPLVAERKDLSNSMGLFLQKTNIARDYLEDLRDNRRFWPKEIWGQYAETMEDLVKPENKEKALQCLSHMIVNAMEHIRDVLEYLSMIKNPSCFKFCAIPQVMAMATLNLLHSNYKVFTHENIKIRKGETVWLMKESDSMDKVAAIFRLYARQINNKSNSLDPHFVDIGVICGEIEQICVGRFPGSTIEMKRMQAGVLGGKTGTVLAAAAAVAGAVVINNALA

SEQ ID NO: 66 - DNA, 1251 bp, Mortierella alpina
atggcttctgctatcctcgcctcgctcctccacccttccgaggtgttggccttggtccagtacaaactctcgccaaagacccaacacgactacagcaacgataaaaccaggcagcgcctctaccaccacttgaacatgacctcgcgtagtttctcagcggtcatccaggatctggacgaggaactgaaggatgcgatttgcttgttctacctcgtccttcgtggactcgataccattgaggacgatatgacgattgatttggacaccaagttgccatatctgaggacgttccacgaaatcatctaccagaagggatggacctttacgaagaatggtcctaacgaaaaagaccgccagttgctggttgagtttgacgccatcatcgagggattcttgcaactaaagccagcgtatcaaaccatcattgccgacatcactaaacgcatgggcaatggaatggctcactacgccactgcaggaattcacgttgagactaatgctgattatgacgaatactgccattacgtcgcgggccttgttggtctgggattgagcgagatgttcagcgcctgtggatttgaatcgcctttggtagccgagagaaaagacctctcaaactcgatgggtctgtttctccaaaagaccaacatcgcacgcgattatctcgaggatctgcgcgacaatcgccgtttctggccaaaggagatctggggccagtatgcggaaacgatggaggacctagtcaagcccgagaacaaggagaaggctctgcagtgtctgagccacatgatcgtcaacgccatggagcacatccgagatgtcctcgagtaccttagtatgatcaagaacccgtcctgctttaagttctgtgcgattccccaggttatggccatggcgactttgaacctcctccactccaactacaaggtttttacgcacgagaatatcaaaatccgcaagggcgagacagtgtggctgatgaaggagtcagacagcatggacaaggtggcagccatcttccgactttatgcgcgccagatcaacaacaagtcaaactctctggacccccactttgttgacatcggtgtcatttgcggcgagattgagcagatctgtgttggaaggttcccaggatccacgattgagatgaagcgcatgcaagctggagtgctgggcggcaaaaccggaaccgtgcttgctgcagctgcggctgttgcaggagctgttgttatcaacaatgcgctcgcataa

SEQ ID NO: 67 - protein, 379 aa, Mortierella alpina
MASAILASLLHPSEVLALVQYKLSPKTQHDYSNDKTRQRLYHHLNMTSRSFSAVIQDLDEELKDAICLFYLVLRGLDTIEDDMTIDLDTKLPYLRTFHEIIYQKGWTFTKNGPNEKDRQLLVEFDAIIEGFLQLKPAYQTIIADITKRMGNGMAHYATAGIHVETNADYDEYCHYVAGLVGLGLSEMFSACGFESPLVAERKDLSNSMGLFLQKTNIARDYLEDLRDNRRFWPKEIWGQYAETMEDLVKPENKEKALQCLSHMIVNAMEHIRDVLEYLSMIKNPSCFKFCAIPQVMAMATLNLLHSNYKVFTHENIKIRKGETVWLMKESDSMDKVAAIFRLYARQINNKSNSLDPHFVDIGVICGEIEQICVGRFPGS

SEQ ID NO: 68 - protein, 399 aa, Mortierella alpina
MASAILASLLHPSEVLALVQYKLSPKTQHDYSNDKTRQRLYHHLNMTSRSFSAVIQDLDEELKDAICLFYLVLRGLDTIEDDMTIDLDTKLPYLRTFHEIIYQKGWTFTKNGPNEKDRQLLVEFDAIIEGFLQLKPAYQTIIADITKRMGNGMAHYATAGIHVETNADYDEYCHYVAGLVGLGLSEMFSACGFESPLVAERKDLSNSMGLFLQKTNIARDYLEDLRDNRRFWPKEIWGQYAETMEDLVKPENKEKALQCLSHMIVNAMEHIRDVLEYLSMIKNPSCFKFCAIPQVMAMATLNLLHSNYKVFTHENIKIRKGETVWLMKESDSMDKVAAIFRLYARQINNKSNSLDPHFVDIGVICGEIEQICVGRFPGSTIEMKRMQAGVLGGKTGTVL

SEQ ID NO: 69 - protein, 430 aa, Arabidopsis thaliana; identical to SEQ ID NO: 29

SEQ ID NO: 70 - DNA, 1540 bp, Arabidopsis thaliana; identical to SEQ ID NO: 30

SEQ ID NO: 71 - protein, 35 aa, Arabidopsis thaliana
RESPPSSSSPLSCLSTDSASSTTTTTTSVSCNYLV

SEQ ID NO: 72 - protein, 35 aa, Arabidopsis thaliana
  VARIANT (1)...(35): Xaa = Any Amino Acid
REXPPXXSSPLXCLSTDSAXXTTTXXXXVSCNYLV

SEQ ID NO: 73 - protein, 415 aa, Brassica napus
MKRPLTTSPSTSSSTSSSACILPTQPETPRPKRAKRAKKSSIPTDVKPQNPTSPASTRRSSIYRGVTRHRWTGRYEAHLWDKSSWNSIQNKKGKQVYLGAYDSEEAAAHTYDLAALKYWGPDTILNFPAETYTKELEEMQRCTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVSQANHQEAVLAEAKQEVEAKEEPTEEVKQCVEKEEPQEAKEEKTEKKQQQQEVEEAVVTCCIDSSESNELAWDFCMMDSGFAPFLTDSNLSSENPIEYPELFNEMGFEDNIDFMFEEGKQDCLSLENLDCCDGVVVVGRESPTSLSSSPLSCLSTDSASSTTTTTITSVSCNYSV

SEQ ID NO: 74 - DNA, 1248 bp, Brassica napus
atgaagagacccttaaccacttctccttctacctcctcttctacttcttcttcggcttgtatacttccgactcaaccagagactccaaggcccaaacgagccaaaagggctaagaaatcttctattcctactgatgttaaaccacagaatcccaccagtcctgcctccaccagacgcagctctatctacagaggagtcactagacatagatggacagggagatacgaggctcatctatgggacaaaagctcgtggaattcgattcagaacaagaaaggcaaacaagtttatctgggagcatatgacagcgaggaagcagcagcgcatacgtacgatctagctgctctcaagtactggggtcccgacaccatcttgaactttccggctgagacgtacacaaaggagttggaggagatgcagagatgtacaaaggaagagtatttggcttctctccgccgccagagcagtggtttctctagaggcgtctctaaatatcgcggcgtcgccaggcatcaccataacggaagatgggaagctaggattggaagggtgtttggaaacaagtacttgtacctcggcacttataatacgcaggaggaagctgcagctgcatatgacatggcggctatagagtacagaggcgcaaacgcagtgaccaacttcgacattagtaactacatcgaccggttaaagaaaaaaggtgtcttcccattccctgtgagccaagccaatcatcaagaagctgttcttgctgaagccaaacaagaagtggaagctaaagaagagcctacagaagaagtgaagcagtgtgtcgaaaaagaagaaccgcaagaagctaaagaagagaagactgagaaaaaacaacaacaacaagaagtggaggaggcggtggtcacttgctgcattgattcttcggagagcaatgagctggcttgggacttctgtatgatggattcagggtttgctccgtttttgacggattcaaatctctcgagtgagaatcccattgagtatcctgagcttttcaatgagatggggtttgaggataacattgacttcatgttcgaggaagggaagcaagactgcttgagcttggagaatctggattgttgcgatggtgttgttgtggtgggaagagagagcccaacttcattgtcgtcttcaccgttgtcttgcttgtctactgactctgcttcatcaacaacaacaacaacaataacctctgtttcttgtaactattctgtctga

SEQ ID NO: 75 - protein, 37 aa, Brassica napus
RESPTSLSSSPLSCLSTDSASSTTTTTITSVSCNYSV

SEQ ID NO: 76 - protein, 37 aa, Brassica napus
  VARIANT (1)...(37): Xaa = Any Amino Acid
REXPXXLXXXPLXCLSTDSAXXXXXXXIXXVSCNYSV

SEQ ID NO: 77 - protein, 413 aa, Brassica napus
MKRPLTTSPSSSSSTSSSACILPTQSETPRPKRAKRAKKSSLRSDVKPQNPTSPASTRRSSIYRGVTRHRWTGRYEAHLWDKSSWNSIQNKKGKQVYLGAYDSEEAAAHTYDLAALKYWGPNTILNFPVETYTKELEEMQRCTKEEYLASLRRQSSGFSRGVSKYRGVARHHHNGRWEARIGRVFGNKYLYLGTYNTQEEAAAAYDMAAIEYRGANAVTNFDIGNYIDRLKKKGVFPFPVSQANHQEAVLAETKQEVEAKEEPTEEVKQCVEKEEAKEEKTEKKQQQEVEEAVITCCIDSSESNELAWDFCMMDSGFAPFLTDSNLSSENPIEYPELFNEMGFEDNIDFMFEEGKQDCLSLENLDCCDGVVVVGRESPTSLSSSPLSCLSTDSASSTTTTATTVTSVSWNYSV

SEQ ID NO: 78 - DNA, 1242 bp, Brassica napus
atgaagagacccttaaccacttctccttcttcctcctcttctacttcttcttcggcctgtatacttccgactcaatcagagactccaaggcccaaacgagccaaaagggctaagaaatcttctctgcgttctgatgttaaaccacagaatcccaccagtcctgcctccaccagacgcagctctatctacagaggagtcactagacatagatggacagggagatacgaagctcatctatgggacaaaagctcgtggaattcgattcagaacaagaaaggcaaacaagtttatctgggagcatatgacagcgaggaagcagcagcacatacgtacgatctagctgctctcaagtactggggtcccaacaccatcttgaactttccggttgagacgtacacaaaggagctggaggagatgcagagatgtacaaaggaagagtatttggcttctctccgccgccagagcagtggtttctctagaggcgtctctaaatatcgcggcgtcgccaggcatcaccataatggaagatgggaagctcggattggaagggtgtttggaaacaagtacttgtacctcggcacctataatacgcaggaggaagctgcagctgcatatgacatggcggctatagagtacagaggtgcaaacgcagtgaccaacttcgacattggtaactacatcgaccggttaaagaaaaaaggtgtcttcccgttccccgtgagccaagctaatcatcaagaagctgttcttgctgaaaccaaacaagaagtggaagctaaagaagagcctacagaagaagtgaagcagtgtgtcgaaaaagaagaagctaaagaagagaagactgagaaaaaacaacaacaagaagtggaggaggcggtgatcacttgctgcattgattcttcagagagcaatgagctggcttgggacttctgtatgatggattcagggtttgctccgtttttgactgattcaaatctctcgagtgagaatcccattgagtatcctgagcttttcaatgagatgggttttgaggataacattgacttcatgttcgaggaagggaagcaagactgcttgagcttggagaatcttgattgttgcgatggtgttgttgtggtgggaagagagagcccaacttcattgtcgtcttctccgttgtcctgcttgtctactgactctgcttcatcaacaacaacaacagcaacaacagtaacctctgtttcttggaactattctgtctga

SEQ ID NO: 79 - protein, 36 aa, Brassica napus
RESPTSLSSSPLSCLSTDSASSTTTTATTVTSVSWN

SEQ ID NO: 80 - protein, 36 aa, Brassica napus
  VARIANT (1)...(36): Xaa = Any Amino Acid
REXPXXLXSSPLXCLXTDSAXXXXXXAXXVXXVSWN

SEQ ID NO: 81 - protein, 395 aa, Zea mays
MERSQRQSPPPPSPSSSSSSVSADTVLVPPGKRRRAATAKAGAEPNKRIRKDPAAAAAGKRSSVYRGVTRHRWTGRFEAHLWDKHCLAALHNKKKGRQVYLGAYDSEEAAARAYDLAALKYWGPETLLNFPVEDYSSEMPEMEAVSREEYLASLRRRSSGFSRGVSKYRGVARHHHNGRWEARIGRVFGNKYLYLGTFDTQEEAAKAYDLAAIEYRGVNAVTNFDISCYLDHPLFLAQLQQEPQVVPALNQEPQPDQSETGTTEQEPESSEAKTPDGSAEPDENAVPDDTAEPLSTVDDSIEEGLWSPCMDYELDTMSRPNFGSSINLSEWFADADFDCNIGCLFDGCSAADEGSKDGVGLADFSLFEAGDVQLKDVLSDMEEGIQPPAMISVCN

SEQ ID NO: 82 - DNA, 1576 bp, Zea mays
ctcccccgcctcgccgccagtcagattcaccaccggctcccctgcacaaccgcgtccgcgctgcaccaccaccgttcatcgaggaggaggggggacggagaccacggacatggagagatctcaacggcagtctcctccgccaccgtcgccgtcctcctcctcgtcctccgtctccgcggacaccgtcctcgtccctcccggaaagaggcggagggcggcgacggccaaggccggcgccgagcctaataagaggatccgcaaggaccccgccgccgccgccgcggggaagaggagctccgtctacaggggagtcaccaggcacaggtggacgggcaggttcgaggcgcatctctgggacaagcactgcctcgccgcgctccacaacaagaagaaaggcaggcaagtctacctgggggcgtatgacagcgaggaggcagctgctcgtgcctatgacctcgcagctctcaagtactggggtcctgagactctgctcaacttccctgtggaggattactccagcgagatgccggagatggaggccgtttcccgggaggagtacctggcctccctccgccgcaggagcagcggcttctccaggggcgtctccaagtacagaggcgtcgccaggcatcaccacaacgggaggtgggaggcacggattgggcgagtctttgggaacaagtacctctacttgggaacatttgacactcaagaagaggcagccaaggcctatgaccttgcggccattgaataccgtggcgtcaatgctgtaaccaacttcgacatcagctgctacctggaccacccgctgttcctggcacagctccaacaggagccacaggtggtgccggcactcaaccaagaacctcaacctgatcagagcgaaaccggaactacagagcaagagccggagtcaagcgaagccaagacaccggatggcagtgcagaacccgatgagaacgcggtgcctgacgacaccgcggagcccctcagcacagtcgacgacagcatcgaagagggcttgtggagcccttgcatggattacgagctagacaccatgtcgagaccaaactttggcagctcaatcaatctgagcgagtggttcgctgacgcagacttcgactgcaacatcgggtgcctgttcgatgggtgttctgcggctgacgaaggaagcaaggatggtgtaggtctggcagatttcagtctgtttgaggcaggtgatgtccagctgaaggatgttctttcggatatggaagaggggatacaacctccagcgatgatcagtgtgtgcaactaattctggaacccgaggaggttttcgctttccaggtgtcctgtcttgggtaatccttgatctgtctaatgccacagtgccactgcaccagagcagctgagaactttcttgtagaaagcccatggcagtttggcgttagacaagtgtgtcgatgttctttaattctttgaatttgcccctaggctgcttggctaacgttaagggtttgtcattgtctcacttagcctagattcaactaatcacatcctgaatctgaaaaaaaaaaacaaaaaaaaaaaaaaa

SEQ ID NO: 83 - protein, 88 aa, Zea mays
HPLFLAQLQQEPQVVPALNQEPQPDQSETGTTEQEPESSEAKTPDGSAEPDENAVPDDTAEPLSTVDDSIEEGLWSPCMDYELDTMSR

SEQ ID NO: 84 - protein, 88 aa, Zea mays
  VARIANT (1)...(88): Xaa = Any Amino Acid
HPLFLAQLQQEPQVVPALNQEPQPDQXEXGXXEQEPEXXEAKXPDGXAEPDENAVPDDXAEPLXXVDDXIEEGLWXPCMDYELDXMXR

SEQ ID NO: 85 - protein, 393 aa, Zea mays
MTMERSQPQHQQSPPSPSSSSSCVSADTVLVPPGKRRRRAATAKANKRARKDPSDPPPAAGKRSSVYRGVTRHRWTGRFEAHLWDKHCLAALHNKKKGRQVYLGAYDGEEAAARAYDLAALKYWGPEALLNFPVEDYSSEMPEMEAASREEYLASLRRRSSGFSRGVSKYRGVARHHHNGRWEARIGRVLGNKYLYLGTFDTQEEAAKAYDLAAIEYRGANAVTNFDISCYLDHPLFLAQLQQEQPQVVPALDQEPQADQREPETTAQEPVSSQAKTPADDNAEPDDIAEPLITVDNSVEESLWSPCMDYELDTMSRSNFGSSINLSEWFTDADFDSDLGCLFDGRSAVDGGSKGGVGVADFSLFEAGDGQLKDVLSDMEEGIQPPTIISVCN

SEQ ID NO: 86 - DNA, 1561 bp, Zea mays
cgttcatgcatgaccatggagagatctcaaccgcagcaccagcagtctcctccgtcgccgtcgtcctcctcgtcctgcgtctccgcggacaccgtcctcgtccctccgggaaagaggcggcggagggcggcgacagccaaggccaataagagggcccgcaaggacccctctgatcctcctcccgccgccgggaagaggagctccgtatacagaggagtcaccaggcacaggtggacgggcaggttcgaggcgcatctctgggacaagcactgcctcgccgcgctccacaacaagaagaaaggcaggcaagtctatctgggggcgtacgacggcgaggaggcagcggctcgtgcctatgaccttgcagctctcaagtactggggtcctgaggctctgctcaacttccctgtggaggattactccagcgagatgccggagatggaggcagcgtcccgggaggagtacctggcctccctccgccgcaggagcagcggcttctccaggggggtctccaagtacagaggcgtcgccaggcatcaccacaacgggagatgggaggcacggatcgggcgagttttagggaacaagtacctctacttgggaacattcgacactcaagaagaggcagccaaggcctatgatcttgcggccatcgaataccgaggtgccaatgctgtaaccaacttcgacatcagctgctacctggaccacccactgttcctggcgcagctccagcaggagcagccacaggtggtgccagcgctcgaccaagaacctcaggctgatcagagagaacctgaaaccacagcccaagagcctgtgtcaagccaagccaagacaccggcggatgacaatgcagagcctgatgacatcgcggagcccctcatcacggtcgacaacagcgtcgaggagagcttatggagtccttgcatggattatgagctagacaccatgtcgagatctaactttggcagctcgatcaacctgagcgagtggttcactgacgcagacttcgacagcgacttgggatgcctgttcgacgggcgctctgcagttgatggaggaagcaagggtggcgtaggtgtggcggatttcagtttgtttgaagcaggtgatggtcagctgaaggatgttctttcggatatggaagaggggatacaacctccaacgataatcagtgtgtgcaattgattctgagacctatgcgtggcgtgcgacaagtgtcctgtctttgggtatacttggtttgtccaatgccacggtgccactgctgcgagtcagctgaacttcttgtagaaagcacatggcagcttggcattagacaagtgtgttggtgttccttaattctttggatatgctttaggcattgactaaccttaagggttcgtcactgtctcgcttagcttagattagactaatcacatccttgaatctgaagtagttgtgcagtatcacagtttcacatggcaattctgccaatgcagcatagatttgttcgtttgaacagctgtaactgtaaccctatagctccagattaaggaacagtttgtttttcatccat

SEQ ID NO: 87 - protein, 57 aa, Zea mays
REPETTAQEPVSSQAKTPADDNAEPDDIAEPLITVDNSVEESLWSPCMDYELDTMSR

SEQ ID NO: 88 - protein, 57 aa, Zea mays
  VARIANT (1)...(57): Xaa = Any Amino Acid
REPEXXAQEPVXXQAKXPADDNAEPDDIAEPLIXVDNXVEEXLWXPCMDYELDXMXR

SEQ ID NO: 89 - protein, 337 aa, Elaeis guineensis
MTLMKNSPPSTPLPPISPSSSASPSSYAPLSSPNMIPLNKCKKSKPKHKKAKNSDESSRRRSSIYRGVTRHRGTGRYEAHLWDKHWQHPVQNKKGRQVYLGAFTDELDAARAHDLAALKLWGPETILNFPVEMYREEYKEMQTMSKEEVLASVRRRSNGFARGTSKYRGVARHHKNGRWEARLSQDVGCKYIYLGTYATQEEAAQAYDLAALVHKGPNIVTNFASSVYKHRLQPFMQLLVKPETEPAQEDLGVLQMEATETIDQTMPNYDLPEISWTFDIDHDLGAYPLLDVPIEDDQHDILNDLNFEGNIEHLFEEFETFGGNESGSDGFSASKGA

SEQ ID NO: 90 - DNA, 1782 bp, Elaeis guineensis
agagagagagagattccaacacagggcagctgagattgagcacaaggcgccgtggaaaccacgagttccattggcaacatgggaaacctggtggccaagtgtagagctctctcacacaaacccatgcggccaacttgcagaccctcgagtcatttggactcttccaagctcaccagccgtagggttttttgacaagagggacctccagtaaacgttaaacaaactcgcagctcccacctttggatccattccatcgcttcaacggtgggttagaagcctccgcgccaaatgcacgagtgctcaacagcacgctcccctaatttttctctctccacctcctcacttctctatatataatcctctctttggtgaaccaccatcaaccaaaccaacggtatagtatacgtaggaaataatccctttctagaacatgactctcatgaagaaatctcctccctctactcctctcccaccaatatcgccttcctcttccgcttcaccatccagctatgcacccctttcttctcctaatatgatccctcttaacaagtgcaagaagtcgaagccaaaacataagaaagctaagaactcagatgaaagcagtaggagaagaagctctatctacagaggagtcacgaggcaccgagggactgggagatatgaagctcacctgtgggacaagcactggcagcatccggtccagaacaagaaaggcaggcaagtttacttgggagcctttactgatgagttggacgcagcacgagctcatgacttggctgcccttaagctctggggtccagagacaattttaaacttccctgtggaaatgtatagagaagagtacaaggagatgcaaaccatgtcaaaggaagaggtgctggcttcggttaggcgcaggagcaacggctttgccaggggtacctctaagtaccgtggggtggccaggcatcacaaaaacggccggtgggaggccaggcttagccaggacgttggctgcaagtacatctacttgggaacatacgcaactcaagaggaggctgcccaagcttatgatttagctgctctagtacacaaagggccaaatatagtgaccaactttgctagcagtgtctataagcatcgcctacagccattcatgcagctattagtgaagcctgagacggagccagcacaagaagacctgggggttatgcaaatggaagcaaccgagacaatcgatcagaccatgccaaattacgacctgccggagatctcatggaccttcgacatagaccatgacttaggtgcatatcctctccttgatgtcccaattgaggatgatcaacatgacatcttgaatgatctcaatttcgaggggaacattgagcacctctttgaagagtttgagaccttcggaggcaatgagagtggaagtgatggtttcagtgcaagcaaaggtgcctagcagaggaaagtggtttgaagatggaggacatggcatctaaagcgaactgagcctcctggcctcttcaaagtagtgtctgctttttagaaatcttggtgggtcgatttgagttaggagcccgatacttctatcaggggatatgtttagctacaattctagtttttttttcttttttttttttcagccggaagtctggtacttctgttgaatattatgatgtgcttcttgcttagttgttcctgttcttctcccttttagagttcagcatatttatgttttgatgtaatggggaatgttggcagacagcttgatatatggttatttcattctccattaaa

SEQ ID NO: 91 - protein, 42 aa, Elaeis guineensis
KPETEPAQEDLGVLQMEATETIDQTMPNYDLPEISWTFDIDH

SEQ ID NO: 92 - protein, 42 aa, Elaeis guineensis
  VARIANT (1)...(42): Xaa = Any Amino Acid
KPEXEPAQEDLGVLQMEAXEXIDQXMPNYDLPEIXWXFDIDH

SEQ ID NO: 93 - protein, 409 aa, Glycine max
MKRSPASSCSSSTSSVGFEAPIEKRRPKHPRRNNLKSQKCKQNQTTTGGRRSSIYRGVTRHRWTGRFEAHLWDKSSWNNIQSKKGRQGAYDTEESAARTYDLAALKYWGKDATLNFPIETYTKELEEMDKVSREEYLASLRRQSSGFSRGLSKYRGVARHHHNGRWEARIGRVCGNKYLYLGTYKTQEEAAVAYDMAAIEYRGVNAVTNFDISNYMDKIKKKNDQTQQQQTEAQTETVPNSSDSEEVEVEQQTTTITTPPPSENLHMPPQQHQVQYTPHVSPREEESSSLITIMDHVLEQDLPWSFMYTGLSQFQDPNLAFCKGDDDLVGMFDSAGFEEDIDFLFSTQPGDETESDVNNMSAVLDSVECGDTNGAGGSMMHVDNKQKIVSFASSPSSTTTVSCDYALDL

SEQ ID NO: 94 - DNA, 2206 bp, Glycine max
agtgttgctcaaattcaagccacttaattagccatggttgattgatcaagttaaattccaacccaaggttaaatcattactcccttctcatccttcccaaccccaacccccagaaatattacagattcaattgcttaattaaatactattttcccctccttctataataccctccaaaatctttttccttcttcattctccctttctctatgttttggcaaaccactttaggtaaccagattactactactattgcttcatatacaaagatgctatcgtaaaaaagagagaaacttgggaagtgggaacacattcaaaatccttgtttttctttttggtctaatttttcatctcaaaacacacacccattgagtatttttcatttttttgttcttttgggacaaaaaaggtgggtgttgttggcattattgaagatagaggcccccaaaatgaagaggtctccagcatcttcttgttcatcatctacttcctctgttgggtttgaagctcccattgaaaaaagaaggcctaagcatccaaggaggaataatttgaagtcacaaaaatgcaagcagaaccaaaccaccactggtggcagaagaagctctatctatagaggagttacaaggcataggtggacagggaggtttgaagctcacctatgggataagagctcttggaacaacattcagagcaagaagggtcgacaaggggcatatgatactgaagaatctgcagcccgtacctatgaccttgcagcccttaaatactggggaaaagatgcaaccctgaatttcccgatagaaacttataccaaggagctcgaggaaatggacaaggtttcaagagaagaatatttggcttctttgcggcgccaaagcagtggcttttctagaggcctgtctaagtaccgtggggttgctaggcatcatcataatggtcgctgggaagcacgaattggaagagtatgcggaaacaagtacctctacttggggacatataaaactcaagaggaggcagcagtggcatatgacatggcagcaatagagtaccgtggagtcaatgcagtgaccaattttgacataagcaactacatggacaaaataaagaagaaaaatgaccaaacccaacaacaacaaacagaagcacaaacggaaacagttcctaactcctctgactctgaagaagtagaagtagaacaacagacaacaacaataaccacaccacccccatctgaaaatctgcacatgccaccacagcagcaccaagttcaatacaccccccatgtctctccaagggaagaagaatcatcatcactgatcacaattatggaccatgtgcttgagcaggatctgccatggagcttcatgtacactggcttgtctcagtttcaagatccaaacttggctttctgcaaaggtgatgatgacttggtgggcatgtttgatagtgcagggtttgaggaagacattgattttctgttcagcactcaacctggtgatgagactgagagtgatgtcaacaatatgagcgcagttttggatagtgttgagtgtggagacacaaatggggctggtggaagcatgatgcatgtggataacaagcagaagatagtatcatttgcttcttcaccatcatctacaactacagtttcttgtgactatgctctagatctatgatctcttcagaagggtgatggatgacctacatggaatggaaccttgtgtagattattattgggtttgttatgcatgttgttggggtttgttgtgataggttggtggatgggtgtgacttgtgaaaatgttcattggttttaggattttcctttcatccatactccgttgtcgaaagaagaaaatgttcattttagacttggattttagtataaaaaaaaaggagaaaaaaccaaaaatgtgatttgggtgcaaacaatgttttgtttttctttttacttttggggtaaggagatgaagagaggggaaatttaaaccattcctattcttgggggataatgcagtataaattaagatcagactgtttttagcatatggagtgcaaactgcaaaggccaagtttcctttgtttaaacaatttaggctttcttttcctttgcctattttttttttatttttttttttgtattggggcatagcagttagtgttgtgttgagatctgaaatctgatctctggtttggtttgttc

SEQ ID NO: 95 - protein, 59 aa, Glycine max
DETESDVNNMSAVLDSVECGDTNGAGGSMMHVDNKQKIVSFASSPSSTTTVSCDYALDL

SEQ ID NO: 96 - protein, 59 aa, Glycine max
  VARIANT (1)...(59): Xaa = Any Amino Acid
DEXEXDVNNMXAVLDXVECGDXNGAGGXMMHVDNKQKIVXFAXXPXXXXXVXCDYALDL

SEQ ID NO: 97 - protein, 347 aa, Picea abies; identical to SEQ ID NO: 45

SEQ ID NO: 98 - DNA, 1044 bp, Picea abies; identical to SEQ ID NO: 46

SEQ ID NO: 99 - protein, 354 aa, Gallus gallus; identical to SEQ ID NO: 47

SEQ ID NO: 100 - DNA, 1330 bp, Gallus gallus; identical to SEQ ID NO: 48

SEQ ID NO: 101 - protein, 54 aa, Arabidopsis thaliana
MASSMLSSATMVASPAQATMVAPFNGLKSSAAFPATRKANNDITSITSNGGRVN

SEQ ID NO: 102 - DNA, 162 bp, Arabidopsis thaliana
atggcttcctctatgctctcttccgctactatggttgcctctccggctcaggccactatggtcgctcctttcaacggacttaagtcctccgctgccttcccagccacccgcaaggctaacaacgacattacttccatcacaagcaacggcggaagagttaac

SEQ ID NO: 103 - DNA, 15695 bp, Artificial Sequence
  A synthetic oligonucleotide sequence
cctgtggttggcatgcacatacaaatggacgaacggataaaccttttcacgcccttttaaatatccgattattctaataaacgctcttttctcttaggtttacccgccaatatatcctgtcaaacactgatagtttgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgttaattaagaattcgagctccaccgcggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagtcaaatgcagcagacgaagttgctactcaacttttgaattttgacttgctgaagttggctggtgatgttgagtcaaaccctggacctatgggcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtccggactcagatctcgagctcaagcttcgaattctgcagtcgacggtaccgcgggcccgggatcatcaacaagtttgtacaaaaaagcaggctccaccatggccggccccatcatgacctctgcgccctccgcgaccacgcccacgggcaagacaatgccgttcaagcagcctttcaagactgtggccacgctgtccgccaagactggcaacattaccaagcccatcgaccctgccatctccaagaccattgacttcgtctacaatggttactcgacggtcaagaccaaggttgacaaggcccctaaggtaaacccctacctgctcattgccggcggcctcgtcctctcgtgcatcatctccatgtgcctgctcgtcccggccgtgatcttcttccccgtcaccatcttcctgggtgtcgctacgtcgtttgcgctcattgcattggcccccgtggcttttgtgttcgggtggatcctgatctcctctgctccgatccaggataaggtggtggtgcccgccttggacaaggtgctggccaataagaaggtggcgaagttcctcctcaaggagtaatcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgccagcttggcgtaatcatggtcatagctgttgcgattaagaattcgagctcggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttcgactctagaggatccccttaaatcgatatttatgatttcgcctctggcatccgaggaggatgaggaaattgttaaatctgttgttaatggaacgattccttcgtattcgttggaatcgaagcttggggattgtaaaagagcggctgagattcgacgggaggctttgcagagaatgatggggaggtcgttggagggtttacctgttgaaggattcgattatgagtcgattttaggtcagtgctgtgaaatgcctgttggttatgtgcagattccggttggaattgctgggccgttgctgctagacgggcaagagtactctgttccgatggcgaccaccgagggttgtttggttgctagcactaatagagggtgtaaagcgatccatttgtcaggtggtgctagtagtgtcttgttgaaggatggcatgactagagctcccgttgttcgattcgcctcggccatgagggccgcggatttgaagtttttcttagagaatcctgagaatttcgatagcttgtccatcgctttcaataggtccagtagatttgcaaagctccaaagcatacaatgttctattgctggaaagaatctatatatgagattcacctgcagcactggtgatgcaatggggatgaacatggtttccaaaggggttcaaaacgttcttgacttccttcaaagtgatttccctgacatggatgttattggcatctcaggaaatttttgttcggacaagaagccagctgctgtgaactggattcaagggcgaggcaaatcggttgtttgcgaggcaattatcaaggaagaggtggtgaagaaggtattgaaatcaagtgttgcttcactagtagagctgaacatgctcaagaatcttactggttcagctattgctggagctcttggtggattcaatgcacatgctggcaacatagtctctgcaattttcattgccactggccaggatccagcccagaatgttgagagttctcattgcatcaccatgatggaagctgtcaatgatggaaaagatctccacatctctgtaaccatgccttcaatcgaggtaggaacagttggaggagggacacaactagcatcccaatcagcatgtctgaacctactcggtgtaaaaggagcaagtaaagaatcaccaggagcaaactcaaggctcctagccacaatagtagctggttcagtcctagctggtgaactctccctaatgtcagccatagcagcaggacaactagtccggagccacatgaagtacaacagatccagcaaagatgtaaccaaatttgcatcatcttcaaatgcagcagacgaagttgctactcaacttttgaattttgacttgctgaagttggctggtgatgttgagtcaaaccctggacctatggcggatctgaaatcaaccttcctcgacgtttactctgttctcaagtctgatctgcttcaagatccttcctttgaattcacccacgaatctcgtcaatggcttgaacggatgcttgactacaatgtacgcggagggaagctaaatcgtggtctctctgtggttgatagctacaagctgttgaagcaaggtcaagacttgacggagaaagagactttcctctcatgtgctcttggttggtgcattgaatggcttcaagcttatttccttgtgcttgatgacatcatggacaactctgtcacacgccgtggccagccttgttggtttagaaagccaaaggttggtatgattgccattaacgatgggattctacttcgcaatcatatccacaggattctcaaaaagcacttcagggaaatgccttactatgttgacctcgttgatttgtttaacgaggtagagtttcaaacagcttgcggccagatgattgatttgatcaccacctttgatggagaaaaagatttgtctaagtactccttgcaaatccatcggcgtattgttgagtacaaaacagcttattactcattttatcttcctgttgcttgcgcattgctcatggcgggagaaaatttggaaaaccatactgatgtgaagactgttcttgttgacatgggaatttactttcaagtacaggatgattatctggactgttttgctgatcctgagacacttggcaagatagggacagacatagaagatttcaaatgctcctggttggtagttaaggcattggaacgctgcagtgaagaacaaactaagatactatacgagaactatggtaaagccgaaccatcaaacgttgctaaggtgaaagctctctacaaagagcttgatctcgagggagcgttcatggaatatgagaaggaaagctatgagaagctgacaaagttgatcgaagctcaccagagtaaagcaattcaagcagtgctaaaatctttcttggctaagatctacaagaggcagaagaaatcctcatctaacgctgctgatgaggtggcaacacagttgctgaacttcgatcttttgaaacttgcaggagacgtggaatctaatccaggcccaatggccagtgctattcttgcttcattactccacccatcagaagtgttggcacttgtgcagtacaagctttcacccaaaacccagcatgattactctaacgacaaaactaggcaaagactttatcatcatcttaatatgacttcccgatccttctctgccgtcatacaggaccttgatgaagagttaaaggatgctatatgcttattctatctggtgctgagaggcttagatactatagaagacgacatgaccatcgaccttgacactaaattgccttaccttcgtacgttccacgaaatcatataccagaaaggctggactttcactaagaacggcccaaatgaaaaagataggcaattactggtagaatttgacgccatcatagagggcttccttcaattgaagccagcctatcagactatcattgccgatataaccaaacgtatggggaacggaatggcacactacgctacggcagggatacatgttgagaccaacgcagactacgacgagtactgccactatgtcgctggtttggtggggctgggtctctctgaaatgttttccgcatgtgggttcgaaagtcctcttgtggcagaaagaaaagaccttagcaacagcatgggacttttccttcagaagacgaacattgcacgtgattatcttgaagacctcagagacaatcgtcgattttggcccaaggaaatatgggggcagtatgctgagactatggaggacttggtaaagcccgaaaataaagaaaaggccctccaatgcctctcccatatgatcgtcaatgcaatggagcatatcagagacgttttggagtatctctctatgataaagaatccgagctgcttcaaattttgtgctattccacaagtcatggctatggccacattaaacctgcttcattccaactacaaagtgttcacgcatgagaatatcaagatccgtaaaggtgagacagtgtggcttatgaaagaaagtgacagtatggacaaggtagctgctatctttaggttgtacgcccgacaaattaacaacaagtccaactctcttgatccccattttgtggatataggggtgatttgcggtgagatcgagcaaatttgcgtaggaaggttccctggctccacaatagaaatgaagcgaatgcaggctggagtcttaggggggaaaactggaacggtcctgtaatcagcaattgggggagctcgaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatggcgcgtggccggcctacagtatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgcggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactgtaggccggccctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagagtagacaggctgtctcgcggccgaggggcgcagcccctgggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacactagtcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaactatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcaggtaacctcgcgcatacagccgggcagtgacgtcatcgtctgcgcggaaatggacgggcccccggcgccagatctggggaac

SEQ ID NO: 104 - protein, 855 aa, Artificial Sequence
  A synthetic polypeptide sequence
MKKRLTTSTCSSSPSSSVSSSTTTSSPIQSEAPRPKRAKRAKKSSPSGDKSHNPTSPASTRRSSIYRGVTRHRWTGRFEAHLWDKSSWNSIQNKKGKQVYLGAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESNAADEVATQLLNFDLLKLAGDVESNPGPMGKGEELFTGVVPILVELDGDVNGHKFSVSGEGEGDATYGKLTLKFICTTGKLPVPWPTLVTTFGYGLQCFARYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYKSGLRSRAQASNSAVDGTAGPGSSTSLYKKAGSTMAGPIMTSAPSATTPTGKTMPFKQPFKTVATLSAKTGNITKPIDPAISKTIDFVYNGYSTVKTKVDKAPKVNPYLLIAGGLVLSCIISMCLLVPAVIFFPVTIFLGVATSFALIALAPVAFVFGWILISSAPIQDKVVVPALDKVLANKKVAKFLLKE

SEQ ID NO: 105 - protein, 1227 aa, Artificial Sequence
  A synthetic polypeptide sequence
MISPLASEEDEEIVKSVVNGTIPSYSLESKLGDCKRAAEIRREALQRMMGRSLEGLPVEGFDYESILGQCCEMPVGYVQIPVGIAGPLLLDGQEYSVPMATTEGCLVASTNRGCKAIHLSGGASSVLLKDGMTRAPVVRFASAMRAADLKFFLENPENFDSLSIAFNRSSRFAKLQSIQCSIAGKNLYMRFTCSTGDAMGMNMVSKGVQNVLDFLQSDFPDMDVIGISGNFCSDKKPAAVNWIQGRGKSVVCEAIIKEEVVKKVLKSSVASLVELNMLKNLTGSAIAGALGGFNAHAGNIVSAIFIATGQDPAQNVESSHCITMMEAVNDGKDLHISVTMPSIEVGTVGGGTQLASQSACLNLLGVKGASKESPGANSRLLATIVAGSVLAGELSLMSAIAAGQLVRSHMKYNRSSKDVTKFASSSNAADEVATQLLNFDLLKLAGDVESNPGPMADLKSTFLDVYSVLKSDLLQDPSFEFTHESRQWLERMLDYNVRGGKLNRGLSVVDSYKLLKQGQDLTEKETFLSCALGWCIEWLQAYFLVLDDIMDNSVTRRGQPCWFRKPKVGMIAINDGILLRNHIHRILKKHFREMPYYVDLVDLFNEVEFQTACGQMIDLITTFDGEKDLSKYSLQIHRRIVEYKTAYYSFYLPVACALLMAGENLENHTDVKTVLVDMGIYFQVQDDYLDCFADPETLGKIGTDIEDFKCSWLVVKALERCSEEQTKILYENYGKAEPSNVAKVKALYKELDLEGAFMEYEKESYEKLTKLIEAHQSKAIQAVLKSFLAKIYKRQKKSSSNAADEVATQLLNFDLLKLAGDVESNPGPMASAILASLLHPSEVLALVQYKLSPKTQHDYSNDKTRQRLYHHLNMTSRSFSAVIQDLDEELKDAICLFYLVLRGLDTIEDDMTIDLDTKLPYLRTFHEIIYQKGWTFTKNGPNEKDRQLLVEFDAIIEGFLQLKPAYQTIIADITKRMGNGMAHYATAGIHVETNADYDEYCHYVAGLVGLGLSEMFSACGFESPLVAERKDLSNSMGLFLQKTNIARDYLEDLRDNRRFWPKEIWGQYAETMEDLVKPENKEKALQCLSHMIVNAMEHIRDVLEYLSMIKNPSCFKFCAIPQVMAMATLNLLHSNYKVFTHENIKIRKGETVWLMKESDSMDKVAAIFRLYARQINNKSNSLDPHFVDIGVICGEIEQICVGRFPGSTIEMKRMQAGVLGGKTGTVL

SEQ ID NO: 106 - DNA, 14792 bp, Artificial Sequence
  A synthetic oligonucleotide sequence
cctgtggttggcatgcacatacaaatggacgaacggataaaccttttcacgcccttttaaatatccgattattctaataaacgctcttttctcttaggtttacccgccaatatatcctgtcaaacactgatagtttgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgttaattaagaattcgagctccaccgcggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagtcaaatgcagcagacgaagttgctactcaacttttgaattttgacttgctgaagttggctggtgatgttgagtcaaaccctggacctatggccagtgctattcttgcttcattactccacccatcagaagtgttggcacttgtgcagtacaagctttcacccaaaacccagcatgattactctaacgacaaaactaggcaaagactttatcatcatcttaatatgacttcccgatccttctctgccgtcatacaggaccttgatgaagagttaaaggatgctatatgcttattctatctggtgctgagaggcttagatactatagaagacgacatgaccatcgaccttgacactaaattgccttaccttcgtacgttccacgaaatcatataccagaaaggctggactttcactaagaacggcccaaatgaaaaagataggcaattactggtagaatttgacgccatcatagagggcttccttcaattgaagccagcctatcagactatcattgccgatataaccaaacgtatggggaacggaatggcacactacgctacggcagggatacatgttgagaccaacgcagactacgacgagtactgccactatgtcgctggtttggtggggctgggtctctctgaaatgttttccgcatgtgggttcgaaagtcctcttgtggcagaaagaaaagaccttagcaacagcatgggacttttccttcagaagacgaacattgcacgtgattatcttgaagacctcagagacaatcgtcgattttggcccaaggaaatatgggggcagtatgctgagactatggaggacttggtaaagcccgaaaataaagaaaaggccctccaatgcctctcccatatgatcgtcaatgcaatggagcatatcagagacgttttggagtatctctctatgataaagaatccgagctgcttcaaattttgtgctattccacaagtcatggctatggccacattaaacctgcttcattccaactacaaagtgttcacgcatgagaatatcaagatccgtaaaggtgagacagtgtggcttatgaaagaaagtgacagtatggacaaggtagctgctatctttaggttgtacgcccgacaaattaacaacaagtccaactctcttgatccccattttgtggatataggggtgatttgcggtgagatcgagcaaatttgcgtaggaaggttccctggctccacaatagaaatgaagcgaatgcaggctggagtcttaggggggaaaactggaacggtcctgatggccggccccatcatgacctctgcgccctccgcgaccacgcccacgggcaagacaatgccgttcaagcagcctttcaagactgtggccacgctgtccgccaagactggcaacattaccaagcccatcgaccctgccatctccaagaccattgacttcgtctacaatggttactcgacggtcaagaccaaggttgacaaggcccctaaggtaaacccctacctgctcattgccggcggcctcgtcctctcgtgcatcatctccatgtgcctgctcgtcccggccgtgatcttcttccccgtcaccatcttcctgggtgtcgctacgtcgtttgcgctcattgcattggcccccgtggcttttgtgttcgggtggatcctgatctcctctgctccgatccaggataaggtggtggtgcccgccttggacaaggtgctggccaataagaaggtggcgaagttcctcctcaaggagtaatcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgccagcttggcgtaatcatggtcatagctgttgcgattaagaattcgagctcggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttcgactctagaggatccccttaaatcgatatttatgatttcgcctctggcatccgaggaggatgaggaaattgttaaatctgttgttaatggaacgattccttcgtattcgttggaatcgaagcttggggattgtaaaagagcggctgagattcgacgggaggctttgcagagaatgatggggaggtcgttggagggtttacctgttgaaggattcgattatgagtcgattttaggtcagtgctgtgaaatgcctgttggttatgtgcagattccggttggaattgctgggccgttgctgctagacgggcaagagtactctgttccgatggcgaccaccgagggttgtttggttgctagcactaatagagggtgtaaagcgatccatttgtcaggtggtgctagtagtgtcttgttgaaggatggcatgactagagctcccgttgttcgattcgcctcggccatgagggccgcggatttgaagtttttcttagagaatcctgagaatttcgatagcttgtccatcgctttcaataggtccagtagatttgcaaagctccaaagcatacaatgttctattgctggaaagaatctatatatgagattcacctgcagcactggtgatgcaatggggatgaacatggtttccaaaggggttcaaaacgttcttgacttccttcaaagtgatttccctgacatggatgttattggcatctcaggaaatttttgttcggacaagaagccagctgctgtgaactggattcaagggcgaggcaaatcggttgtttgcgaggcaattatcaaggaagaggtggtgaagaaggtattgaaatcaagtgttgcttcactagtagagctgaacatgctcaagaatcttactggttcagctattgctggagctcttggtggattcaatgcacatgctggcaacatagtctctgcaattttcattgccactggccaggatccagcccagaatgttgagagttctcattgcatcaccatgatggaagctgtcaatgatggaaaagatctccacatctctgtaaccatgccttcaatcgaggtaggaacagttggaggagggacacaactagcatcccaatcagcatgtctgaacctactcggtgtaaaaggagcaagtaaagaatcaccaggagcaaactcaaggctcctagccacaatagtagctggttcagtcctagctggtgaactctccctaatgtcagccatagcagcaggacaactagtccggagccacatgaagtacaacagatccagcaaagatgtaaccaaatttgcatcatcttcaaatgcagcagacgaagttgctactcaacttttgaattttgacttgctgaagttggctggtgatgttgagtcaaaccctggacctatggcggatctgaaatcaaccttcctcgacgtttactctgttctcaagtctgatctgcttcaagatccttcctttgaattcacccacgaatctcgtcaatggcttgaacggatgcttgactacaatgtacgcggagggaagctaaatcgtggtctctctgtggttgatagctacaagctgttgaagcaaggtcaagacttgacggagaaagagactttcctctcatgtgctcttggttggtgcattgaatggcttcaagcttatttccttgtgcttgatgacatcatggacaactctgtcacacgccgtggccagccttgttggtttagaaagccaaaggttggtatgattgccattaacgatgggattctacttcgcaatcatatccacaggattctcaaaaagcacttcagggaaatgccttactatgttgacctcgttgatttgtttaacgaggtagagtttcaaacagcttgcggccagatgattgatttgatcaccacctttgatggagaaaaagatttgtctaagtactccttgcaaatccatcggcgtattgttgagtacaaaacagcttattactcattttatcttcctgttgcttgcgcattgctcatggcgggagaaaatttggaaaaccatactgatgtgaagactgttcttgttgacatgggaatttactttcaagtacaggatgattatctggactgttttgctgatcctgagacacttggcaagatagggacagacatagaagatttcaaatgctcctggttggtagttaaggcattggaacgctgcagtgaagaacaaactaagatactatacgagaactatggtaaagccgaaccatcaaacgttgctaaggtgaaagctctctacaaagagcttgatctcgagggagcgttcatggaatatgagaaggaaagctatgagaagctgacaaagttgatcgaagctcaccagagtaaagcaattcaagcagtgctaaaatctttcttggctaagatctacaagaggcagaagtaaaaatcctcagcaattgggggagctcgaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatggcgcgtggccggcctacagtatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgcggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactgtaggccggccctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagagtagacaggctgtctcgcggccgaggggcgcagcccctgggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacactagtcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaactatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcaggtaacctcgcgcatacagccgggcagtgacgtcatcgtctgcgcggaaatggacgggcccccggcgccagatctggggaac

SEQ ID NO: 107 - protein, 983 aa, Artificial Sequence
  A synthetic polypeptdie sequence
MKKRLTTSTCSSSPSSSVSSSTTTSSPIQSEAPRPKRAKRAKKSSPSGDKSHNPTSPASTRRSSIYRGVTRHRWTGRFEAHLWDKSSWNSIQNKKGKQVYLGAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESNAADEVATQLLNFDLLKLAGDVESNPGPMASAILASLLHPSEVLALVQYKLSPKTQHDYSNDKTRQRLYHHLNMTSRSFSAVIQDLDEELKDAICLFYLVLRGLDTIEDDMTIDLDTKLPYLRTFHEIIYQKGWTFTKNGPNEKDRQLLVEFDAIIEGFLQLKPAYQTIIADITKRMGNGMAHYATAGIHVETNADYDEYCHYVAGLVGLGLSEMFSACGFESPLVAERKDLSNSMGLFLQKTNIARDYLEDLRDNRRFWPKEIWGQYAETMEDLVKPENKEKALQCLSHMIVNAMEHIRDVLEYLSMIKNPSCFKFCAIPQVMAMATLNLLHSNYKVFTHENIKIRKGETVWLMKESDSMDKVAAIFRLYARQINNKSNSLDPHFVDIGVICGEIEQICVGRFPGSTIEMKRMQAGVLGGKTGTVLMAGPIMTSAPSATTPTGKTMPFKQPFKTVATLSAKTGNITKPIDPAISKTIDFVYNGYSTVKTKVDKAPKVNPYLLIAGGLVLSCIISMCLLVPAVIFFPVTIFLGVATSFALIALAPVAFVFGWILISSAPIQDKVVVPALDKVLANKKVAKFLLKE

SEQ ID NO: 108 - protein, 796 aa, Artificial Sequence
  A synthetic polypeptdie sequence
MISPLASEEDEEIVKSVVNGTIPSYSLESKLGDCKRAAEIRREALQRMMGRSLEGLPVEGFDYESILGQCCEMPVGYVQIPVGIAGPLLLDGQEYSVPMATTEGCLVASTNRGCKAIHLSGGASSVLLKDGMTRAPVVRFASAMRAADLKFFLENPENFDSLSIAFNRSSRFAKLQSIQCSIAGKNLYMRFTCSTGDAMGMNMVSKGVQNVLDFLQSDFPDMDVIGISGNFCSDKKPAAVNWIQGRGKSVVCEAIIKEEVVKKVLKSSVASLVELNMLKNLTGSAIAGALGGFNAHAGNIVSAIFIATGQDPAQNVESSHCITMMEAVNDGKDLHISVTMPSIEVGTVGGGTQLASQSACLNLLGVKGASKESPGANSRLLATIVAGSVLAGELSLMSAIAAGQLVRSHMKYNRSSKDVTKFASSSNAADEVATQLLNFDLLKLAGDVESNPGPMADLKSTFLDVYSVLKSDLLQDPSFEFTHESRQWLERMLDYNVRGGKLNRGLSVVDSYKLLKQGQDLTEKETFLSCALGWCIEWLQAYFLVLDDIMDNSVTRRGQPCWFRKPKVGMIAINDGILLRNHIHRILKKHFREMPYYVDLVDLFNEVEFQTACGQMIDLITTFDGEKDLSKYSLQIHRRIVEYKTAYYSFYLPVACALLMAGENLENHTDVKTVLVDMGIYFQVQDDYLDCFADPETLGKIGTDIEDFKCSWLVVKALERCSEEQTKILYENYGKAEPSNVAKVKALYKELDLEGAFMEYEKESYEKLTKLIEAHQSKAIQAVLKSFLAKIYKRQK

SEQ ID NO: 109 - DNA, 14705 bp, Artificial Sequence
  A synthetic oligonucleotide sequence
cctgtggttggcatgcacatacaaatggacgaacggataaaccttttcacgcccttttaaatatccgattattctaataaacgctcttttctcttaggtttacccgccaatatatcctgtcaaacactgatagtttgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgttaattaagaattcgagctccaccgcggaaacctcctcggattccattgcccagctatctgtcactttattgagaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggtattaaaatcttaataggttttgataaaagcgaacgtggggaaacccgaaccaaaccttcttctaaactctctctcatctctcttaaagcaaacttctctcttgtctttcttgcgtgagcgatcttcaacgttgtcagatcgtgcttcggcaccagtacaacgttttctttcactgaagcgaaatcaaagatctctttgtggacacgtagtgcggcgccattaaataacgtgtacttgtcctattcttgtcggtgtggtcttgggaaaagaaagcttgctggaggctgctgttcagccccatacattacttgttacgattctgctgactttcggcgggtgcaatatctctacttctgcttgacgaggtattgttgcctgtacttctttcttcttcttcttgctgattggttctataagaaatctagtattttctttgaaacagagttttcccgtggttttcgaacttggagaaagattgttaagcttctgtatattctgcccaaattcgcgatgaagaagcgcttaaccacttccacttgttcttcttctccatcttcctctgtttcttcttctactactacttcctctcctattcagtcggaggctccaaggcctaaacgagccaaaagggctaagaaatcttctccttctggtgataaatctcataacccgacaagccctgcttctacccgacgcagctctatctacagaggagtcactagacatagatggactgggagattcgaggctcatctttgggacaaaagctcttggaattcgattcagaacaagaaaggcaaacaagtttatctgggagcatatgacagtgaagaagcagcagcacatacgtacgatctggctgctctcaagtactggggacccgacaccatcttgaattttccggcagagacgtacacaaaggaattggaagaaatgcagagagtgacaaaggaagaatatttggcttctctccgccgccagagcagtggtttctccagaggcgtctctaaatatcgcggcgtcgctaggcatcaccacaacggaagatgggaggctcggatcggaagagtgtttgggaacaagtacttgtacctcggcacctataatacgcaggaggaagctgctgcagcatatgacatggctgcgattgagtatcgaggcgcaaacgcggttactaatttcgacattagtaattacattgaccggttaaagaagaaaggtgttttcccgttccctgtgaaccaagctaaccatcaagagggtattcttgttgaagccaaacaagaagttgaaacgagagaagcgaaggaagagcctagagaagaagtgaaacaacagtacgtggaagaaccaccgcaagaagaagaagagaaggaagaagagaaagcagagcaacaagaagcagagattgtaggatattcagaagaagcagcagtggtcaattgctgcatagactcttcaaccataatggaaatggatcgttgtggggacaacaatgagctggcttggaacttctgtatgatggatacagggttttctccgtttttgactgatcagaatctcgcgaatgagaatcccatagagtatccggagctattcaatgagttagcatttgaggacaacatcgacttcatgttcgatgatgggaagcacgagtgcttgaacttggaaaatctggattgttgcgtggtgggaagagagtcaaatgcagcagacgaagttgctactcaacttttgaattttgacttgctgaagttggctggtgatgttgagtcaaaccctggacctatgatttcgcctctggcatccgaggaggatgaggaaattgttaaatctgttgttaatggaacgattccttcgtattcgttggaatcgaagcttggggattgtaaaagagcggctgagattcgacgggaggctttgcagagaatgatggggaggtcgttggagggtttacctgttgaaggattcgattatgagtcgattttaggtcagtgctgtgaaatgcctgttggttatgtgcagattccggttggaattgctgggccgttgctgctagacgggcaagagtactctgttccgatggcgaccaccgagggttgtttggttgctagcactaatagagggtgtaaagcgatccatttgtcaggtggtgctagtagtgtcttgttgaaggatggcatgactagagctcccgttgttcgattcgcctcggccatgagggccgcggatttgaagtttttcttagagaatcctgagaatttcgatagcttgtccatcgctttcaataggtccagtagatttgcaaagctccaaagcatacaatgttctattgctggaaagaatctatatatgagattcacctgcagcactggtgatgcaatggggatgaacatggtttccaaaggggttcaaaacgttcttgacttccttcaaagtgatttccctgacatggatgttattggcatctcaggaaatttttgttcggacaagaagccagctgctgtgaactggattcaagggcgaggcaaatcggttgtttgcgaggcaattatcaaggaagaggtggtgaagaaggtattgaaatcaagtgttgcttcactagtagagctgaacatgctcaagaatcttactggttcagctattgctggagctcttggtggattcaatgcacatgctggcaacatagtctctgcaattttcattgccactggccaggatccagcccagaatgttgagagttctcattgcatcaccatgatggaagctgtcaatgatggaaaagatctccacatctctgtaaccatgccttcaatcgaggtaggaacagttggaggagggacacaactagcatcccaatcagcatgtctgaacctactcggtgtaaaaggagcaagtaaagaatcaccaggagcaaactcaaggctcctagccacaatagtagctggttcagtcctagctggtgaactctccctaatgtcagccatagcagcaggacaactagtccggagccacatgaagtacaacagatccagcaaagatgtaaccaaatttgcatcatcttaatcgaggcctttaactctggtttcattaaattttctttagtttgaatttactgttattcggtgtgcatttctatgtttggtgagcggttttctgtgctcagagtgtgtttattttatgtaatttaatttctttgtgagctcctgtttagcaggtcgtcccttcagcaaggacacaaaaagattttaattttattaaaaaaaaaaaaaaaaaagaccgggaattcgatatcaagcttatcgacctgcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatctctagagtctcaagcttggcgcgccagcttggcgtaatcatggtcatagctgttgcgattaagaattcgagctcggtacccccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagcccaagcttcgactctagaggatccccttaaatcgatatttatggccagtgctattcttgcttcattactccacccatcagaagtgttggcacttgtgcagtacaagctttcacccaaaacccagcatgattactctaacgacaaaactaggcaaagactttatcatcatcttaatatgacttcccgatccttctctgccgtcatacaggaccttgatgaagagttaaaggatgctatatgcttattctatctggtgctgagaggcttagatactatagaagacgacatgaccatcgaccttgacactaaattgccttaccttcgtacgttccacgaaatcatataccagaaaggctggactttcactaagaacggcccaaatgaaaaagataggcaattactggtagaatttgacgccatcatagagggcttccttcaattgaagccagcctatcagactatcattgccgatataaccaaacgtatggggaacggaatggcacactacgctacggcagggatacatgttgagaccaacgcagactacgacgagtactgccactatgtcgctggtttggtggggctgggtctctctgaaatgttttccgcatgtgggttcgaaagtcctcttgtggcagaaagaaaagaccttagcaacagcatgggacttttccttcagaagacgaacattgcacgtgattatcttgaagacctcagagacaatcgtcgattttggcccaaggaaatatgggggcagtatgctgagactatggaggacttggtaaagcccgaaaataaagaaaaggccctccaatgcctctcccatatgatcgtcaatgcaatggagcatatcagagacgttttggagtatctctctatgataaagaatccgagctgcttcaaattttgtgctattccacaagtcatggctatggccacattaaacctgcttcattccaactacaaagtgttcacgcatgagaatatcaagatccgtaaaggtgagacagtgtggcttatgaaagaaagtgacagtatggacaaggtagctgctatctttaggttgtacgcccgacaaattaacaacaagtccaactctcttgatccccattttgtggatataggggtgatttgcggtgagatcgagcaaatttgcgtaggaaggttccctggctccacaatagaaatgaagcgaatgcaggctggagtcttaggggggaaaactggaacggtcctgatggccggccccatcatgacctctgcgccctccgcgaccacgcccacgggcaagacaatgccgttcaagcagcctttcaagactgtggccacgctgtccgccaagactggcaacattaccaagcccatcgaccctgccatctccaagaccattgacttcgtctacaatggttactcgacggtcaagaccaaggttgacaaggcccctaaggtaaacccctacctgctcattgccggcggcctcgtcctctcgtgcatcatctccatgtgcctgctcgtcccggccgtgatcttcttccccgtcaccatcttcctgggtgtcgctacgtcgtttgcgctcattgcattggcccccgtggcttttgtgttcgggtggatcctgatctcctctgctccgatccaggataaggtggtggtgcccgccttggacaaggtgctggccaataagaaggtggcgaagttcctcctcaaggagatggcggatctgaaatcaaccttcctcgacgtttactctgttctcaagtctgatctgcttcaagatccttcctttgaattcacccacgaatctcgtcaatggcttgaacggatgcttgactacaatgtacgcggagggaagctaaatcgtggtctctctgtggttgatagctacaagctgttgaagcaaggtcaagacttgacggagaaagagactttcctctcatgtgctcttggttggtgcattgaatggcttcaagcttatttccttgtgcttgatgacatcatggacaactctgtcacacgccgtggccagccttgttggtttagaaagccaaaggttggtatgattgccattaacgatgggattctacttcgcaatcatatccacaggattctcaaaaagcacttcagggaaatgccttactatgttgacctcgttgatttgtttaacgaggtagagtttcaaacagcttgcggccagatgattgatttgatcaccacctttgatggagaaaaagatttgtctaagtactccttgcaaatccatcggcgtattgttgagtacaaaacagcttattactcattttatcttcctgttgcttgcgcattgctcatggcgggagaaaatttggaaaaccatactgatgtgaagactgttcttgttgacatgggaatttactttcaagtacaggatgattatctggactgttttgctgatcctgagacacttggcaagatagggacagacatagaagatttcaaatgctcctggttggtagttaaggcattggaacgctgcagtgaagaacaaactaagatactatacgagaactatggtaaagccgaaccatcaaacgttgctaaggtgaaagctctctacaaagagcttgatctcgagggagcgttcatggaatatgagaaggaaagctatgagaagctgacaaagttgatcgaagctcaccagagtaaagcaattcaagcagtgctaaaatctttcttggctaagatctacaagaggcagaagtaaaaatcctcagcaattgggggagctcgaattcgctgaaatcaccagtctctctctacaaatctatctctctctattttctccataaataatgtgtgagtagtttcccgataagggaaattagggttcttatagggtttcgctcatgtgttgagcatataagaaacccttagtatgtatttgtatttgtaaaatacttctatcaataaaatttctaattcctaaaaccaaaatccagtactaaaatccagatctcctaaagtccctatagatctttgtcgtgaatataaaccagacacgagacgactaaacctggagcccagacgccgttcgaagctagaagtaccgcttaggcaggaggccgttagggaaaagatgctaaggcagggttggttacgttgactcccccgtaggtttggtttaaatatgatgaagtggacggaaggaaggaggaagacaaggaaggataaggttgcaggccctgtgcaaggtaagaagatggaaatttgatagaggtacgctactatacttatactatacgctaagggaatgcttgtatttataccctataccccctaataaccccttatcaatttaagaaataatccgcataagcccccgcttaaaaattggtatcagagccatgaataggtctatgaccaaaactcaagaggataaaacctcaccaaaatacgaaagagttcttaactctaaagataaaagatggcgcgtggccggcctacagtatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaccggatctggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccacgggatctctgcggaacaggcggtcgaaggtgccgatatcattacgacagcaacggccgacaagcacaacgccacgatcctgagcgacaatatgatcgcggcgtccacatcaacggcgtcggcggcgactgcccaggcaagaccgagatgcaccgcgatatcttgctgcgttcggatattttcgtggagttcccgccacagacccggatgatccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggactgtaggccggccctcactggtgaaaagaaaaaccaccccagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacagagtagacaggctgtctcgcggccgaggggcgcagcccctgggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaaggcgctcggtcttgccttgctcgtcggtgatgtacactagtcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcgagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccacaattatgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctatctctgctctcactgccgtaaaacatggcaactgcagttcacttacaccgcttctcaacccggtacgcaccagaaaatcattgatatggccatgaatggcgttggatgccgggcaacagcccgcattatgggcgttggcctcaacacgattttacgtcacttaaaaaactcaggccgcagtcggtaactatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcaggtaacctcgcgcatacagccgggcagtgacgtcatcgtctgcgcggaaatggacgggcccccggcgccagatctggggaac

SEQ ID NO: 110 - protein, 851 aa, Artificial Sequence
  A synthetic polypeptide sequence
MKKRLTTSTCSSSPSSSVSSSTTTSSPIQSEAPRPKRAKRAKKSSPSGDKSHNPTSPASTRRSSIYRGVTRHRWTGRFEAHLWDKSSWNSIQNKKGKQVYLGAYDSEEAAAHTYDLAALKYWGPDTILNFPAETYTKELEEMQRVTKEEYLASLRRQSSGFSRGVSKYRGVARHHHNGRWEARIGRVFGNKYLYLGTYNTQEEAAAAYDMAAIEYRGANAVTNFDISNYIDRLKKKGVFPFPVNQANHQEGILVEAKQEVETREAKEEPREEVKQQYVEEPPQEEEEKEEEKAEQQEAEIVGYSEEAAVVNCCIDSSTIMEMDRCGDNNELAWNFCMMDTGFSPFLTDQNLANENPIEYPELFNELAFEDNIDFMFDDGKHECLNLENLDCCVVGRESNAADEVATQLLNFDLLKLAGDVESNPGPMISPLASEEDEEIVKSVVNGTIPSYSLESKLGDCKRAAEIRREALQRMMGRSLEGLPVEGFDYESILGQCCEMPVGYVQIPVGIAGPLLLDGQEYSVPMATTEGCLVASTNRGCKAIHLSGGASSVLLKDGMTRAPVVRFASAMRAADLKFFLENPENFDSLSIAFNRSSRFAKLQSIQCSIAGKNLYMRFTCSTGDAMGMNMVSKGVQNVLDFLQSDFPDMDVIGISGNFCSDKKPAAVNWIQGRGKSVVCEAIIKEEVVKKVLKSSVASLVELNMLKNLTGSAIAGALGGFNAHAGNIVSAIFIATGQDPAQNVESSHCITMMEAVNDGKDLHISVTMPSIEVGTVGGGTQLASQSACLNLLGVKGASKESPGANSRLLATIVAGSVLAGELSLMSAIAAGQLVRSHMKYNRSSKDVTKFASS

SEQ ID NO: 111 - protein, 899 aa, Artificial Sequence
  A synthetic polypeptide sequence
MASAILASLLHPSEVLALVQYKLSPKTQHDYSNDKTRQRLYHHLNMTSRSFSAVIQDLDEELKDAICLFYLVLRGLDTIEDDMTIDLDTKLPYLRTFHEIIYQKGWTFTKNGPNEKDRQLLVEFDAIIEGFLQLKPAYQTIIADITKRMGNGMAHYATAGIHVETNADYDEYCHYVAGLVGLGLSEMFSACGFESPLVAERKDLSNSMGLFLQKTNIARDYLEDLRDNRRFWPKEIWGQYAETMEDLVKPENKEKALQCLSHMIVNAMEHIRDVLEYLSMIKNPSCFKFCAIPQVMAMATLNLLHSNYKVFTHENIKIRKGETVWLMKESDSMDKVAAIFRLYARQINNKSNSLDPHFVDIGVICGEIEQICVGRFPGSTIEMKRMQAGVLGGKTGTVLMAGPIMTSAPSATTPTGKTMPFKQPFKTVATLSAKTGNITKPIDPAISKTIDFVYNGYSTVKTKVDKAPKVNPYLLIAGGLVLSCIISMCLLVPAVIFFPVTIFLGVATSFALIALAPVAFVFGWILISSAPIQDKVVVPALDKVLANKKVAKFLLKEMADLKSTFLDVYSVLKSDLLQDPSFEFTHESRQWLERMLDYNVRGGKLNRGLSVVDSYKLLKQGQDLTEKETFLSCALGWCIEWLQAYFLVLDDIMDNSVTRRGQPCWFRKPKVGMIAINDGILLRNHIHRILKKHFREMPYYVDLVDLFNEVEFQTACGQMIDLITTFDGEKDLSKYSLQIHRRIVEYKTAYYSFYLPVACALLMAGENLENHTDVKTVLVDMGIYFQVQDDYLDCFADPETLGKIGTDIEDFKCSWLVVKALERCSEEQTKILYENYGKAEPSNVAKVKALYKELDLEGAFMEYEKESYEKLTKLIEAHQSKAIQAVLKSFLAKIYKRQK